(19) United States
(12) Patent Application Publication
(10) Pub. No.: US 2021/0122819 A1
(43) Pub. Date: Apr. 29, 2021

(54) ANTI-LILRB ANTIBODIES AND USES THEREOF

(71) Applicant: Adanate, Inc., La Jolla, CA (US)

(72) Inventors: Neil GIBSON, San Diego, CA (US); Graham THOMAS, San Diego, CA (US); Justin CHAPMAN, San Diego, CA (US); Safak YALCIN, La Jolla, CA (US)

(21) Appl. No.: 16/963,207

(22) PCT Filed: Jan. 18, 2019
(86) PCT No.: PCT/US2019/014361

§ 371 Date: Jul. 17, 2020

Related U.S. Application Data
(60) Provisional application No. 62/619,056, filed on Jan. 18, 2018.
(60) Provisional application No. 62/619,050, filed on Jan. 18, 2018.

(51) Int. Cl. C07K 16/28; C12N 5/0786; C12N 5/0783

(52) U.S. Cl. CPC C07K 16/2803; C12N 5/0645; C12N 5/0638; C12N 2501/24; C07K 2317/34; C12N 2501/50; C07K 2317/33

(57) ABSTRACT

Disclosed herein are specific and pan antibodies that interact with one or more members of the LILRB receptor family. In some instances, also described herein are pharmaceutical compositions that comprise one or more anti-LILRB antibodies and methods of modulating inflammatory macrophage activation, lymphocyte activation, and phagocytosis.

### CROSS-REFERENCE

This application claims the benefit of U.S. Provisional Application No. 62/619,050, filed Jan. 18, 2018, and U.S. Provisional Application No. 62/619,056, filed Jan. 18, 2018, each of which is incorporated herein by reference in its entirety.

### BACKGROUND OF THE DISCLOSURE

The immune system comprises different interdependent cell types that protect a host body from pathogenic infections and tumor growth. Upon activation, the immune response is further classified into two response types: innate response which encompasses recruitment of immune cells such as neutrophils, monocytes, and/or macrophages to a target site (e.g., a site of infection), activation of complement cascade, and identification and removal of foreign substances; and adaptive response which is characterized by antigen-specific reactions through T lymphocytes and B lymphocytes. In some instances, diseases such as cancer and disease causing pathogens have developed different mechanisms to evade the immune system.

### SUMMARY OF THE DISCLOSURE

Disclosed herein, in certain embodiments, are antibodies or binding fragments thereof that specifically bind to LILRB1, LILRB2, LILRB3, LILRB4, LILRB5, or a combination thereof. In certain embodiments, also disclosed herein are pan antibodies or binding fragments thereof that specifically bind to two or more LILRBs (e.g., LILRB1 and/or LILRB2 and further bind to LILRB3, LILRB4, and/or LILRB5).

Disclosed herein, in certain embodiments, is an antibody or binding fragment thereof that specifically binds to an epitope on the extracellular domain of LILRB1, an epitope on the extracellular domain of LILRB2, or a combination thereof, for the treatment of a proliferative disease, an infectious disease, or a neurological disease or disorder. In some embodiments, the antibody or binding fragment thereof specifically binds to an epitope on the extracellular domain of LILRB1 and weakly binds to an epitope on the extracellular domain of LILRB2. In some embodiments, the epitope comprises a peptide sequence within domain D1, D2, D3, or D4 of LILRB1. In some embodiments, the epitope comprises at least one peptide sequence within the region between the C-terminus of D4 domain and the N-terminus of the transmembrane domain of LILRB1. In some embodiments, the epitope comprises at least one peptide sequence within the D1 domain and at least one peptide sequence within the D2 domain of LILRB1. In some embodiments, the epitope comprises at least one peptide sequence within the D2 domain and at least one peptide sequence within the D3 domain of LILRB1. In some embodiments, the epitope comprises at least one peptide sequence within the D3 domain and at least one peptide sequence within the D4 domain of LILRB1. In some embodiments, the epitope comprises at least one peptide sequence within the D4 domain and at least one peptide sequence within the region between the C-terminus of D4 domain and the N-terminus of the transmembrane domain of LILRB1. In some embodiments, the antibody or binding fragment thereof specifically binds to an epitope of LILRB2 and weakly binds to an epitope on the extracellular domain of LILRB1. In some embodiments, the epitope comprises a peptide sequence within domain D1, D2, D3, or D4 of LILRB2. In some embodiments, the epitope comprises at least one peptide sequence within the region between the C-terminus of D4 domain and the N-terminus of the transmembrane domain of LILRB2. In some embodiments, the epitope comprises at least one peptide sequence within the D1 domain and at least one peptide sequence within the D2 domain of LILRB2. In some embodiments, the epitope comprises at least one peptide sequence within the D2 domain and at least one peptide sequence within the D3 domain of LILRB2. In some embodiments, the epitope comprises at least one peptide sequence within the D3 domain and at least one peptide sequence within the D4 domain of LILRB2. In some embodiments, the epitope comprises at least one peptide sequence within the D4 domain and at least one peptide sequence within the region between the C-terminus of D4 domain and the N-terminus of the transmembrane domain of LILRB2. In some embodiments, the antibody or binding fragment thereof comprises a humanized antibody or binding fragment thereof, chimeric antibody or binding fragment thereof, monoclonal antibody or binding fragment thereof, bispecific antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, the proliferative disease is cancer. In some embodiments, the cancer is a solid tumor. In some embodiments, the cancer is a hematologic malignancy. In some embodiments, the infectious disease is a viral infection. In some embodiments, the infectious disease is Dengue fever. In some embodiments, the infectious disease is AIDS. In some embodiments, the infectious disease is caused by a protozoan. In some embodiments, the infectious disease is malaria. In some embodiments, the neurological disease or disorder is a neurodegenerative disease or disorder. In some embodiments, the neurological disease or disorder is Alzheimer's disease. In some embodiments, the antibody or binding fragment thereof inhibits binding of a ligand of LILRB1 to LILRB1 and/or a ligand of LILRB2 to LILRB2 by at least 10%, 20%, 30%, 40%, 50%, 60%, 70%, 80%, 90%, 95%, or more. In some embodiments, the antibody or binding fragment thereof inhibits binding of a ligand of LILRB1 to LILRB1 and/or a ligand of LILRB2 to LILRB2 by about 2-fold, 3-fold, 4-fold, 5-fold, 6-fold, 7-fold, 8-fold, 9-fold, 10-fold, or more. In some embodiments, the ligand of LILRB1 is a natural ligand. In some embodiments, the ligand of LILRB2 is a natural ligand. In some embodiments, the natural ligand comprises HLA-A, HLA-B, HLA-C, HLA-E, HLA-G, CD1c, CD1d, MAG, ANGPTL1, ANGPTL2, ANGPTL3, ANGPTL4, ANGPTL5, ANGPTL6, ANGPTL7, ANGPTL8, RTN4, or OMgp. In some embodiments, the natural ligand comprises HLA-A. In some embodiments, the natural ligand comprises oligo Aβ oligomers. In some embodiments, the natural ligand comprises a pathogen. In some embodiments, the pathogen comprises Dengue, Escherichia coli, or Staphylococcus aureus. In some embodiments, the antibody or binding fragment thereof, when contacted to a plurality of peripheral blood mononuclear cells (PBMCs) comprising T cells, enhances cytotoxic T cell activation relative to a plurality of equivalent PBMCs and equivalent T cells in the absence of the antibody or binding fragment thereof. In some embodiments, the antibody or binding fragment thereof, when contacted to a plurality of peripheral blood mononuclear cells (PBMCs) comprising a macrophage, increases M1 activation of the macrophage relative to a plurality of equivalent PBMCs and an equivalent macrophage in the absence of the antibody or binding fragment thereof. In some embodiments, the antibody or binding fragment thereof, when contacted to a plurality of cells comprising APCs and a target cell, increases phagocytosis of the target cell relative to a plurality of equivalent cells in the absence of the antibody or binding fragment thereof. In some embodiments, the antibody or binding fragment thereof, when contacted to a plurality of cells, increases inflammatory cytokine production relative to a plurality of equivalent cells in the absence of the antibody or binding fragment thereof. In some embodiments, the inflammatory cytokine comprises TNFα, IFNγ, or a combination thereof. In some embodiments, the antibody or binding fragment thereof decreases tumor-infiltrating regulatory T cells when administered to a subject in need thereof, relative to a second subject in the absence of the antibody or binding fragment thereof.

Disclosed herein, in certain embodiments, is an antibody or binding fragment thereof that specifically binds to LILRB1 and modulates inflammatory macrophage activation and/or lymphocyte activation. In some embodiments, the antibody or binding fragment thereof decreases tumor-infiltrating regulatory T cells when administered to a subject in need thereof, relative to a second subject in the absence of the antibody or binding fragment thereof.

Disclosed herein, in certain embodiments, is an antibody or binding fragment thereof that specifically binds to an epitope on the extracellular domain of LILRB1 and increases phagocytosis of a target cell. In some embodiments, the antibody or binding fragment thereof decreases tumor-infiltrating regulatory T cells when administered to a subject in need thereof, relative to a second subject in the absence of the antibody or binding fragment thereof.

Disclosed herein, in certain embodiments, is an antibody or binding fragment thereof that specifically binds to an epitope on the extracellular domain of LILRB2 and modulates inflammatory macrophage activation and/or lymphocyte activation. In some embodiments, the antibody or binding fragment thereof decreases tumor-infiltrating regulatory T cells when administered to a subject in need thereof, relative to a second subject in the absence of the antibody or binding fragment thereof.

Disclosed herein, in certain embodiments, is an antibody or binding fragment thereof that specifically binds to an epitope on the extracellular domain of LILRB2 and increases phagocytosis of a target cell. In some embodiments, the antibody or binding fragment thereof decreases tumor-infiltrating regulatory T cells when administered to a subject in need thereof, relative to a second subject in the absence of the antibody or binding fragment thereof.

Disclosed herein, in certain embodiments, is a pan antibody or binding fragment thereof that specifically binds to an epitope on the extracellular domain of LILRB1 and at least an epitope on the extracellular domain of LILRB2, LILRB3, LILRB4, LILRB5, or a combination thereof, for the treatment of a proliferative disease, an infectious disease, or a neurological disease or disorder. In some embodiments, the pan antibody or binding fragment thereof specifically binds to an epitope on the extracellular domain of LILRB1 and at least an epitope on the extracellular domain of LILRB2, LILRB3, LILRB4, or a combination thereof, for the treatment of a proliferative disease, an infectious disease, or a neurological disease or disorder. In some embodiments, the pan antibody or binding fragment thereof specifically binds to an epitope on the extracellular domain of LILRB1 and at least an epitope on the extracellular domain of LILRB2, LILRB3, or a combination thereof, for the treatment of a proliferative disease, an infectious disease, or a neurological disease or disorder. In some embodiments, the pan antibody or binding fragment thereof specifically binds to an epitope on the extracellular domain of LILRB1 and at least an epitope on the extracellular domain of LILRB2, LILRB4, or a combination thereof, for the treatment of a proliferative disease, an infectious disease, or a neurological disease or disorder. In some embodiments, the pan antibody or binding fragment thereof specifically binds to an epitope on the extracellular domain of LILRB1 and at least an epitope on the extracellular domain of LILRB3, LILRB4, or a combination thereof, for the treatment of a proliferative disease, an infectious disease, or a neurological disease or disorder. In some embodiments, the epitope comprises: (i) a peptide sequence within domain D1, D2, D3, or D4 of LILRB1; (ii) at least one peptide sequence within the region between the C-terminus of D4 domain and the N-terminus of the transmembrane domain of LILRB1; (iii) at least one peptide sequence within the D1 domain and at least one peptide sequence within the D2 domain of LILRB1; (iv) at least one peptide sequence within the D2 domain and at least one peptide sequence within the D3 domain of LILRB1; (v) at least one peptide sequence within the D3 domain and at least one peptide sequence within the D4 domain of LILRB1; or (vi) at least one peptide sequence within the D4 domain and at least one peptide sequence within the region between the C-terminus of D4 domain and the N-terminus of the transmembrane domain of LILRB1. In some embodiments, the epitope comprises: (i) a peptide sequence within domain D1, D2, D3, or D4 of LILRB2; (ii) at least one peptide sequence within the region between the C-terminus of D4 domain and the N-terminus of the transmembrane domain of LILRB2; (iii) at least one peptide sequence within the D1 domain and at least one peptide sequence within the D2 domain of LILRB2; (iv) at least one peptide sequence within the D2 domain and at least one peptide sequence within the D3 domain of LILRB2; (v) at least one peptide sequence within the D3 domain and at least one peptide sequence within the D4 domain of LILRB2; or (vi) at least one peptide sequence within the D4 domain and at least one peptide sequence within the region between the C-terminus of D4 domain and the N-terminus of the transmembrane domain of LILRB2. In some embodiments, the epitope comprises: (i) a peptide sequence within domain D1, D2, D3, or D4 of LILRB3; (ii) at least one peptide sequence within the region between the C-terminus of D4 domain and the N-terminus of the transmembrane domain of LILRB3; (iii) at least one peptide sequence within the D1 domain and at least one peptide sequence within the D2 domain of LILRB3; (iv) at least one peptide sequence within the D2 domain and at least one peptide sequence within the D3 domain of LILRB3; (v) at least one peptide sequence within the D3 domain and at least one peptide sequence within the D4 domain of LILRB3; (vi) at least one peptide sequence within the D4 domain and at least one peptide sequence within the region between the C-terminus of D4 domain and the N-terminus of the transmembrane domain of LILRB3. In some embodiments, the epitope comprises: (i) a peptide sequence within domain D1 or D2 of LILRB4; (ii) at least one peptide sequence within the region between the C-terminus of D2 domain and the N-terminus of the transmembrane domain of LILRB4; (iii) at least one peptide sequence within the D1 domain and at least one peptide sequence within the D2 domain of LILRB4; or (iv) at least one peptide sequence within the D2 domain and at least one peptide sequence within the region between the C-terminus of D2 domain and the N-terminus of the transmembrane domain of LILRB4. In some embodiments, the epitope comprises: (i) a peptide sequence within domain D1, D2, D3, or D4 of LILRB5; (ii) at least one peptide sequence within the region between the C-terminus of D4 domain and the N-terminus of the transmembrane domain of LILRB5; (iii) at least one peptide sequence within the D1 domain and at least one peptide sequence within the D2 domain of LILRB5; (iv) at least one peptide sequence within the D2 domain and at least one peptide sequence within the D3 domain of LILRB5; (v) at least one peptide sequence within the D3 domain and at least one peptide sequence within the D4 domain of LILRB5; or (vi) at least one peptide sequence within the D4 domain and at least one peptide sequence within the region between the C-terminus of D4 domain and the N-terminus of the transmembrane domain of LILRB5. In some embodiments, the proliferative disease is cancer. In some embodiments, the cancer is a solid tumor. In some embodiments, the cancer is a hematologic malignancy. In some embodiments, the infectious disease is a viral infection. In some embodiments, the infectious disease is Dengue fever. In some embodiments, the infectious disease is AIDS. In some embodiments, the infectious disease is caused by a protozoan. In some embodiments, the infectious disease is malaria. In some embodiments, the neurological disease or disorder is a neurodegenerative disease or disorder. In some embodiments, the neurological disease or disorder is Alzheimer's disease. In some embodiments, the antibody or binding fragment thereof inhibits binding of a ligand of LILRB1 to LILRB1 and/or a ligand of LILRB2 to LILRB2 by at least 10%, 20%, 30%, 40%, 50%, 60%, 70%, 80%, 90%, 95%, or more. In some embodiments, the antibody or binding fragment thereof inhibits binding of a ligand of LILRB1 to LILRB1 and/or a ligand of LILRB2 to LILRB2 by about 2-fold, 3-fold, 4-fold, 5-fold, 6-fold, 7-fold, 8-fold, 9-fold, 10-fold, or more. In some embodiments, the ligand of LILRB1 is a natural ligand. In some embodiments, the ligand of LILRB2 is a natural ligand. In some embodiments, the natural ligand comprises HLA-A, HLA-B, HLA-C, HLA-E, HLA-F, HLA-G, HLA-H, HLA-I, CD1d, MAG, ANGPTL1, ANGPTL2, ANGPTL3, ANGPTL4, ANGPTL5, ANGPTL6, ANGPTL7, ANGPTL8, RTN4, S100A8, S100A9, Nogo, or OMgp. In some embodiments, the natural ligand comprises HLA-A.

Disclosed herein, in certain embodiments, is a pan antibody or binding fragment thereof that specifically binds to an epitope on the extracellular domain of LILRB2 and at least an epitope on the extracellular domain of LILRB3, LILRB4, LILRB5, or a combination thereof, for the treatment of a proliferative disease, an infectious disease, or a neurological disease or disorder. In some embodiments, the pan antibody or binding fragment thereof specifically binds to an epitope on the extracellular domain of LILRB2 and at least an epitope on the extracellular domain of LILRB3, LILRB4, or a combination thereof, for the treatment of a proliferative disease, an infectious disease, or a neurological disease or disorder. In some embodiments, the pan antibody or binding fragment thereof specifically binds to an epitope on the extracellular domain of LILRB2, at least an epitope on the extracellular domain of LILRB3, and at least an epitope on the extracellular domain of LILRB4, for the treatment of a proliferative disease, an infectious disease, or a neurological disease or disorder. In some embodiments, the epitope comprises: (i) a peptide sequence within domain D1, D2, D3, or D4 of LILRB2; (ii) at least one peptide sequence within the region between the C-terminus of D4 domain and the N-terminus of the transmembrane domain of LILRB2; (iii) at least one peptide sequence within the D1 domain and at least one peptide sequence within the D2 domain of LILRB2; (iv) at least one peptide sequence within the D2 domain and at least one peptide sequence within the D3 domain of LILRB2; (v) at least one peptide sequence within the D3 domain and at least one peptide sequence within the D4 domain of LILRB2; or (vi) at least one peptide sequence within the D4 domain and at least one peptide sequence within the region between the C-terminus of D4 domain and the N-terminus of the transmembrane domain of LILRB2. In some embodiments, the epitope comprises: (i) a peptide sequence within domain D1, D2, D3, or D4 of LILRB3; (ii) at least one peptide sequence within the region between the C-terminus of D4 domain and the N-terminus of the transmembrane domain of LILRB3; (iii) at least one peptide sequence within the D1 domain and at least one peptide sequence within the D2 domain of LILRB3; (iv) at least one peptide sequence within the D2 domain and at least one peptide sequence within the D3 domain of LILRB3; (v) at least one peptide sequence within the D3 domain and at least one peptide sequence within the D4 domain of LILRB3; (vi) at least one peptide sequence within the D4 domain and at least one peptide sequence within the region between the C-terminus of D4 domain and the N-terminus of the transmembrane domain of LILRB3. In some embodiments, the epitope comprises: (i) a peptide sequence within domain D1 or D2 of LILRB4; (ii) at least one peptide sequence within the region between the C-terminus of D2 domain and the N-terminus of the transmembrane domain of LILRB4; (iii) at least one peptide sequence within the D1 domain and at least one peptide sequence within the D2 domain of LILRB4; or (iv) at least one peptide sequence within the D2 domain and at least one peptide sequence within the region between the C-terminus of D2 domain and the N-terminus of the transmembrane domain of LILRB4. In some embodiments, the epitope comprises: (i) a peptide sequence within domain D1, D2, D3, or D4 of LILRB5; (ii) at least one peptide sequence within the region between the C-terminus of D4 domain and the N-terminus of the transmembrane domain of LILRB5; (iii) at least one peptide sequence within the D1 domain and at least one peptide sequence within the D2 domain of LILRB5; (iv) at least one peptide sequence within the D2 domain and at least one peptide sequence within the D3 domain of LILRB5; (v) at least one peptide sequence within the D3 domain and at least one peptide sequence within the D4 domain of LILRB5; or (vi) at least one peptide sequence within the D4 domain and at least one peptide sequence within the region between the C-terminus of D4 domain and the N-terminus of the transmembrane domain of LILRB5. In some embodiments, the proliferative disease is cancer. In some embodiments, the cancer is a solid tumor. In some embodiments, the cancer is a hematologic malignancy. In some embodiments, the infectious disease is a viral infection. In some embodiments, the infectious disease is Dengue fever. In some embodiments, the infectious disease is AIDS. In some embodiments, the infectious disease is caused by a protozoan. In some embodiments, the infectious disease is malaria. In some embodiments, the neurological disease or disorder is a neurodegenerative disease or disorder. In some embodiments, the neurological disease or disorder is Alzheimer's disease. In some embodiments, the antibody or binding fragment thereof inhibits binding of a ligand of LILRB1 to LILRB1 and/or a ligand of LILRB2 to LILRB2 by at least 10%, 20%, 30%, 40%, 50%, 60%, 70%, 80%, 90%, 95%, or more. In some embodiments, the antibody or binding fragment thereof inhibits binding of a ligand of LILRB1 to LILRB1 and/or a ligand of LILRB2 to LILRB2 by about 2-fold, 3-fold, 4-fold, 5-fold, 6-fold, 7-fold, 8-fold, 9-fold, 10-fold, or more. In some embodiments, the ligand of LILRB1 is a natural ligand. In some embodiments, the ligand of LILRB2 is a natural ligand. In some embodiments, the natural ligand comprises HLA-A, HLA-B, HLA-C, HLA-E, HLA-F, HLA-G, HLA-H, HLA-I, CD1d, MAG, ANGPTL1, ANGPTL2, ANGPTL3, ANGPTL4, ANGPTL5, ANGPTL6, ANGPTL7, ANGPTL8, RTN4, S100A8, S100A9, Nogo, or OMgp. In some embodiments, the natural ligand comprises HLA-A.

Disclosed herein, in certain embodiments, is a pharmaceutical composition, comprising: an anti-LILRB1 antibody or binding fragment thereof, an anti-LILRB2 antibody or binding fragment thereof, or a pan anti-LILRB antibody or binding fragment thereof; and a pharmaceutically acceptable excipient. In some embodiments, the pharmaceutical composition is formulated for systemic administration. In some embodiments, the pharmaceutical composition is formulated for parenteral administration.

Disclosed herein, in certain embodiments, is a vector comprising a nucleic acid molecule that encodes an anti-LILRB1 antibody or binding fragment thereof, an anti-LILRB2 antibody or binding fragment thereof, or a pan anti-LILRB antibody or binding fragment thereof.

Disclosed herein, in certain embodiments, is a host cell comprising a nucleic acid molecule that encodes an anti-LILRB1 antibody or binding fragment thereof, an anti-LILRB2 antibody or binding fragment thereof, or a pan anti-LILRB antibody or binding fragment thereof.

Disclosed herein, in certain embodiments, is a method of modulating a macrophage to undergo M1 activation, comprising: (a) contacting a plurality of antigen presenting cells (APCs) comprising a macrophage with an anti-LILRB1 antibody or binding fragment thereof, an anti-LILRB2 antibody or binding fragment thereof, or a pan anti-LILRB antibody or binding fragment thereof; (b) binding the antibody or binding fragment thereof or the pan antibody or binding fragment thereof to one or more LILRB receptors expressed on at least one APC within the plurality of APCs, thereby inducing the APC to produce a plurality of TNFα and interferons; and (c) contacting the plurality of TNFα and interferons with the plurality of APCs comprising the macrophage to induce M1 activation of the macrophage. In some embodiments, the interferon is IFNγ. In some embodiments, the interferon is IFNβ. In some embodiments, the antibody or binding fragment thereof or the pan antibody or binding fragment thereof decreases M2 activation of the macrophage. In some embodiments, the antibody or binding fragment thereof or the pan antibody or binding fragment thereof decreases formation of a tumor associate macrophage. In some embodiments, the APCs further comprise dendritic cells, B cells, or a combination thereof.

Disclosed herein, in certain embodiments, is a method of inducing phagocytosis of a target cell, comprising: (a) incubating a plurality of antigen presenting cells (APCs) comprising a macrophage with an anti-LILRB1 antibody or binding fragment thereof, an anti-LILRB2 antibody or binding fragment thereof, or a pan anti-LILRB antibody or binding fragment thereof, thereby inducing the macrophage to undergo M1 polarization; and (b) contacting the M1 macrophage to a target cell for a time sufficient to induce phagocytosis of the target cell. In some embodiments, the APCs further comprise dendritic cells, B cells, or a combination thereof. In some embodiments, the target cell is a cancer cell. In some embodiments, the target cell is a cell infected by a pathogen.

Disclosed herein, in certain embodiments, is a method of activating a cytotoxic T cell, comprising (a) incubating a plurality of peripheral blood mononuclear cells (PBMCs) comprising naïve T cells with an anti-LILRB1 antibody or binding fragment thereof, an anti-LILRB2 antibody or binding fragment thereof, or a pan anti-LILRB antibody or binding fragment thereof, thereby stimulating the secretion of a plurality of inflammatory cytokines; and (b) contacting the plurality of inflammatory cytokines with the naïve T cells to activate a cytotoxic T cell. In some embodiments, the plurality of inflammatory cytokines comprises TNFα, IFNγ, or IFNβ. In some embodiments, the naïve T cells comprise naïve CD8⁺ T cells. In some embodiments, the PBMCs comprise antigen presenting cells (APCs), NK cells, and/or CD4 T cells. In some embodiments, the CD4 T cells comprise activated CD4⁺ helper T cells. In some embodiments, the APCs comprise B cells and/or dendritic cells.

Disclosed herein, in certain embodiments, is an antibody or binding fragment thereof that specifically binds to an epitope on the extracellular domain of LILRB3, an epitope on the extracellular domain of LILRB4, an epitope on the extracellular domain of LILRB5, or a combination thereof, for the treatment of a proliferative disease, an infectious disease, or an autoimmune disease. In some embodiments, the antibody or binding fragment thereof specifically binds to an epitope on the extracellular domain of LILRB3, an epitope on the extracellular domain of LILRB4, or a combination thereof. In some embodiments, the antibody or binding fragment thereof specifically binds to an epitope on the extracellular domain of LILRB3 and weakly binds to an epitope on the extracellular domain of LILRB4 and the extracellular domain of LILRB5. In some embodiments, the epitope comprises a peptide sequence within domain D1, D2, D3, or D4 of LILRB3. In some embodiments, the epitope comprises at least one peptide sequence within the region between the C-terminus of D4 domain and the N-terminus of the transmembrane domain of LILRB3. In some embodiments, the epitope comprises at least one peptide sequence within the D1 domain and at least one peptide sequence within the D2 domain of LILRB3. In some embodiments, the epitope comprises at least one peptide sequence within the D2 domain and at least one peptide sequence within the D3 domain of LILRB3. In some embodiments, the epitope comprises at least one peptide sequence within the D3 domain and at least one peptide sequence within the D4 domain of LILRB3. In some embodiments, the epitope comprises at least one peptide sequence within the D4 domain and at least one peptide sequence within the region between the C-terminus of D4 domain and the N-terminus of the transmembrane domain of LILRB3. In some embodiments, the antibody or binding fragment thereof specifically binds to an epitope of LILRB4 and weakly binds to an epitope on the extracellular domain of LILRB3 and the extracellular domain of LILRB5. In some embodiments, the epitope comprises a peptide sequence within domain D1 or D2 of LILRB4. In some embodiments, the epitope comprises at least one peptide sequence within the region between the C-terminus of D2 domain and the N-terminus of the transmembrane domain of LILRB4. In some embodiments, the epitope comprises at least one peptide sequence within the D1 domain and at least one peptide sequence within the D2 domain of LILRB4. In some embodiments, the epitope comprises at least one peptide sequence within the D2 domain and at least one peptide sequence within the region between the C-terminus of D2 domain and the N-terminus of the transmembrane domain of LILRB4. In some embodiments, the antibody or binding fragment thereof specifically binds to an epitope of LILRB5 and weakly binds to an epitope on the extracellular domain of LILRB3 and the extracellular domain of LILRB4. In some embodiments, the epitope comprises a peptide sequence within domain D1, D2, D3, or D4 of LILRB5. In some embodiments, the epitope comprises at least one peptide sequence within the region between the C-terminus of D4 domain and the N-terminus of the transmembrane domain of LILRB5. In some embodiments, the epitope comprises at least one peptide sequence within the D1 domain and at least one peptide sequence within the D2 domain of LILRB5. In some embodiments, the epitope comprises at least one peptide sequence within the D2 domain and at least one peptide sequence within the D3 domain of LILRB5. In some embodiments, the epitope comprises at least one peptide sequence within the D3 domain and at least one peptide sequence within the D4 domain of LILRB5. In some embodiments, the epitope comprises at least one peptide sequence within the D4 domain and at least one peptide sequence within the region between the C-terminus of D4 domain and the N-terminus of the transmembrane domain of LILRB5. In some embodiments, the antibody or binding fragment thereof comprises a humanized antibody or binding fragment thereof, chimeric antibody or binding fragment thereof, monoclonal antibody or binding fragment thereof, bispecific antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, the proliferative disease is cancer. In some embodiments, the cancer is a solid tumor. In some embodiments, the cancer is a hematologic malignancy. In some embodiments, the infectious disease is a viral infection. In some embodiments, the infectious disease is a bacterial infection. In some embodiments, the infectious disease is caused by a protozoan. In some embodiments, the autoimmune disease is graft-versus-host disease (GVHD). In some embodiments, the antibody or binding fragment thereof inhibits binding of a ligand of LILRB3 to LILRB3, a ligand of LILRB4 to LILRB4, and/or a ligand of LILRB5 to LILRB5 by at least 10%, 20%, 30%, 40%, 50%, 60%, 70%, 80%, 90%, 95%, or more. In some embodiments, the antibody or binding fragment thereof inhibits binding of a ligand of LILRB3 to LILRB3, a ligand of LILRB4 to LILRB4, and/or a ligand of LILRB5 to LILRB5 by about 2-fold, 3-fold, 4-fold, 5-fold, 6-fold, 7-fold, 8-fold, 9-fold, 10-fold, or more. In some embodiments, the ligand of LILRB3 is a natural ligand. In some embodiments, the ligand of LILRB4 is a natural ligand. In some embodiments, the ligand of LILRB5 is a natural ligand. In some embodiments, the natural ligand comprises HLA-B7, B27, ANGPTL1, ANGPTL2, ANGPTL3, ANGPTL4, ANGPTL5, ANGPTL6, ANGPTL7, ANGPTL8, or CD166.

Disclosed herein, in certain embodiments, is a pan antibody or binding fragment thereof that specifically binds to an epitope on the extracellular domain of LILRB3 and at least an epitope on the extracellular domain of LILRB1, LILRB2, LILRB4, LILRB5, or a combination thereof, for the treatment of a proliferative disease, an infectious disease, or an autoimmune disease. In some embodiments, the pan antibody or binding fragment thereof specifically binds to an epitope on the extracellular domain of LILRB3 and at least an epitope on the extracellular domain of LILRB1, LILRB2, LILRB4, or a combination thereof, for the treatment of a proliferative disease, an infectious disease, or an autoimmune disease. In some embodiments, the pan antibody or binding fragment thereof specifically binds to an epitope on the extracellular domain of LILRB3 and at least an epitope on the extracellular domain of LILRB1, LILRB2, or a combination thereof, for the treatment of a proliferative disease, an infectious disease, or an autoimmune disease. In some embodiments, the pan antibody or binding fragment thereof specifically binds to an epitope on the extracellular domain of LILRB3 and at least an epitope on the extracellular domain of LILRB1, LILRB4, or a combination thereof, for the treatment of a proliferative disease, an infectious disease, or an autoimmune disease. In some embodiments, the pan antibody or binding fragment thereof specifically binds to an epitope on the extracellular domain of LILRB3, at least an epitope on the extracellular domains of LILRB1, at least an epitope on the extracellular domains of LILRB2, and at least an epitope on the extracellular domains of LILRB4, for the treatment of a proliferative disease, an infectious disease, or an autoimmune disease. In some embodiments, the epitope comprises: (i) a peptide sequence within domain D1, D2, D3, or D4 of LILRB3; (ii) at least one peptide sequence within the region between the C-terminus of D4 domain and the N-terminus of the transmembrane domain of LILRB3; (iii) at least one peptide sequence within the D1 domain and at least one peptide sequence within the D2 domain of LILRB3; (iv) at least one peptide sequence within the D2 domain and at least one peptide sequence within the D3 domain of LILRB3; (v) at least one peptide sequence within the D3 domain and at least one peptide sequence within the D4 domain of LILRB3; (vi) at least one peptide sequence within the D4 domain and at least one peptide sequence within the region between the C-terminus of D4 domain and the N-terminus of the transmembrane domain of LILRB3. In some embodiments, the epitope comprises: (i) a peptide sequence within domain D1, D2, D3, or D4 of LILRB1; (ii) at least one peptide sequence within the region between the C-terminus of D4 domain and the N-terminus of the transmembrane domain of LILRB1; (iii) at least one peptide sequence within the D1 domain and at least one peptide sequence within the D2 domain of LILRB1; (iv) at least one peptide sequence within the D2 domain and at least one peptide sequence within the D3 domain of LILRB1; (v) at least one peptide sequence within the D3 domain and at least one peptide sequence within the D4 domain of LILRB1; or (vi) at least one peptide sequence within the D4 domain and at least one peptide sequence within the region between the C-terminus of D4 domain and the N-terminus of the transmembrane domain of LILRB1. In some embodiments, the epitope comprises: (i) a peptide sequence within domain D1, D2, D3, or D4 of LILRB2; (ii) at least one peptide sequence within the region between the C-terminus of D4 domain and the N-terminus of the transmembrane domain of LILRB2; (iii) at least one peptide sequence within the D1 domain and at least one peptide sequence within the D2 domain of LILRB2; (iv) at least one peptide sequence within the D2 domain and at least one peptide sequence within the D3 domain of LILRB2; (v) at least one peptide sequence within the D3 domain and at least one peptide sequence within the D4 domain of LILRB2; or (vi) at least one peptide sequence within the D4 domain and at least one peptide sequence within the region between the C-terminus of D4 domain and the N-terminus of the transmembrane domain of LILRB2. In some embodiments, the epitope comprises: (i) a peptide sequence within domain D1 or D2 of LILRB4; (ii) at least one peptide sequence within the region between the C-terminus of D2 domain and the N-terminus of the transmembrane domain of LILRB4; (iii) at least one peptide sequence within the D1 domain and at least one peptide sequence within the D2 domain of LILRB4; or (iv) at least one peptide sequence within the D2 domain and at least one peptide sequence within the region between the C-terminus of D2 domain and the N-terminus of the transmembrane domain of LILRB4. In some embodiments, the epitope comprises: (i) a peptide sequence within domain D1, D2, D3, or D4 of LILRB5; (ii) at least one peptide sequence within the region between the C-terminus of D4 domain and the N-terminus of the transmembrane domain of LILRB5; (iii) at least one peptide sequence within the D1 domain and at least one peptide sequence within the D2 domain of LILRB5; (iv) at least one peptide sequence within the D2 domain and at least one peptide sequence within the D3 domain of LILRB5; (v) at least one peptide sequence within the D3 domain and at least one peptide sequence within the D4 domain of LILRB5; or (vi) at least one peptide sequence within the D4 domain and at least one peptide sequence within the region between the C-terminus of D4 domain and the N-terminus of the transmembrane domain of LILRB5. In some embodiments, the proliferative disease is cancer. In some embodiments, the cancer is a solid tumor. In some embodiments, the cancer is a hematologic malignancy. In some embodiments, the infectious disease is a viral infection. In some embodiments, the infectious disease is a bacterial infection. In some embodiments, the infectious disease is caused by a protozoan. In some embodiments, the autoimmune disease is graft-versus-host disease (GVHD). In some embodiments, the antibody or binding fragment thereof inhibits binding of a ligand of LILRB3 to LILRB3, a ligand of LILRB4 to LILRB4, and/or a ligand of LILRB5 to LILRB5 by at least 10%, 20%, 30%, 40%, 50%, 60%, 70%, 80%, 90%, 95%, or more. In some embodiments, the antibody or binding fragment thereof inhibits binding of a ligand of LILRB3 to LILRB3, a ligand of LILRB4 to LILRB4, and/or a ligand of LILRB5 to LILRB5 by about 2-fold, 3-fold, 4-fold, 5-fold, 6-fold, 7-fold, 8-fold, 9-fold, 10-fold, or more. In some embodiments, the ligand of LILRB3 is a natural ligand. In some embodiments, the ligand of LILRB4 is a natural ligand. In some embodiments, the ligand of LILRB5 is a natural ligand. In some embodiments, the natural ligand comprises HLA-B7, B27, ANGPTL1, ANGPTL2, ANGPTL3, ANGPTL4, ANGPTL5, ANGPTL6, ANGPTL7, ANGPTL8, or CD166.

Disclosed herein, in certain embodiments, is a pan antibody or binding fragment thereof that specifically binds to an epitope on the extracellular domain of LILRB4 and at least an epitope on the extracellular domain of LILRB1, LILRB3, LILRB5, or a combination thereof, for the treatment of a proliferative disease, an infectious disease, or an autoimmune disease. In some embodiments, the pan antibody or binding fragment thereof specifically binds to an epitope on the extracellular domain of LILRB4 and at least an epitope on the extracellular domain of LILRB1, LILRB3, or a combination thereof, for the treatment of a proliferative disease, an infectious disease, or an autoimmune disease. In some embodiments, the pan antibody or binding fragment thereof specifically binds to an epitope on the extracellular domain of LILRB4, at least an epitope on the extracellular domain of LILRB1, and at least an epitope on the extracellular domain of LILRB3, for the treatment of a proliferative disease, an infectious disease, or an autoimmune disease. In some embodiments, the epitope comprises: (i) a peptide sequence within domain D1 or D2 of LILRB4; (ii) at least one peptide sequence within the region between the C-terminus of D2 domain and the N-terminus of the transmembrane domain of LILRB4; (iii) at least one peptide sequence within the D1 domain and at least one peptide sequence within the D2 domain of LILRB4; or (iv) at least one peptide sequence within the D2 domain and at least one peptide sequence within the region between the C-terminus of D2 domain and the N-terminus of the transmembrane domain of LILRB4. In some embodiments, the epitope comprises: (i) a peptide sequence within domain D1, D2, D3, or D4 of LILRB1; (ii) at least one peptide sequence within the region between the C-terminus of D4 domain and the N-terminus of the transmembrane domain of LILRB1; (iii) at least one peptide sequence within the D1 domain and at least one peptide sequence within the D2 domain of LILRB1; (iv) at least one peptide sequence within the D2 domain and at least one peptide sequence within the D3 domain of LILRB1; (v) at least one peptide sequence within the D3 domain and at least one peptide sequence within the D4 domain of LILRB1; or (vi) at least one peptide sequence within the D4 domain and at least one peptide sequence within the region between the C-terminus of D4 domain and the N-terminus of the transmembrane domain of LILRB1. In some embodiments, the epitope comprises: (i) a peptide sequence within domain D1, D2, D3, or D4 of LILRB3; (ii) at least one peptide sequence within the region between the C-terminus of D4 domain and the N-terminus of the transmembrane domain of LILRB3; (iii) at least one peptide sequence within the D1 domain and at least one peptide sequence within the D2 domain of LILRB3; (iv) at least one peptide sequence within the D2 domain and at least one peptide sequence within the D3 domain of LILRB3; (v) at least one peptide sequence within the D3 domain and at least one peptide sequence within the D4 domain of LILRB3; (vi) at least one peptide sequence within the D4 domain and at least one peptide sequence within the region between the C-terminus of D4 domain and the N-terminus of the transmembrane domain of LILRB3. In some embodiments, the epitope comprises: (i) a peptide sequence within domain D1, D2, D3, or D4 of LILRB5; (ii) at least one peptide sequence within the region between the C-terminus of D4 domain and the N-terminus of the transmembrane domain of LILRB5; (iii) at least one peptide sequence within the D1 domain and at least one peptide sequence within the D2 domain of LILRB5; (iv) at least one peptide sequence within the D2 domain and at least one peptide sequence within the D3 domain of LILRB5; (v) at least one peptide sequence within the D3 domain and at least one peptide sequence within the D4 domain of LILRB5; or (vi) at least one peptide sequence within the D4 domain and at least one peptide sequence within the region between the C-terminus of D4 domain and the N-terminus of the transmembrane domain of LILRB5. In some embodiments, the proliferative disease is cancer. In some embodiments, the cancer is a solid tumor. In some embodiments, the cancer is a hematologic malignancy. In some embodiments, the infectious disease is a viral infection. In some embodiments, the infectious disease is a bacterial infection. In some embodiments, the infectious disease is caused by a protozoan. In some embodiments, the autoimmune disease is graft-versus-host disease (GVHD). In some embodiments, the antibody or binding fragment thereof inhibits binding of a ligand of LILRB3 to LILRB3, a ligand of LILRB4 to LILRB4, and/or a ligand of LILRB5 to LILRB5 by at least 10%, 20%, 30%, 40%, 50%, 60%, 70%, 80%, 90%, 95%, or more. In some embodiments, the antibody or binding fragment thereof inhibits binding of a ligand of LILRB3 to LILRB3, a ligand of LILRB4 to LILRB4, and/or a ligand of LILRB5 to LILRB5 by about 2-fold, 3-fold, 4-fold, 5-fold, 6-fold, 7-fold, 8-fold, 9-fold, 10-fold, or more. In some embodiments, the ligand of LILRB3 is a natural ligand. In some embodiments, the ligand of LILRB4 is a natural ligand. In some embodiments, the ligand of LILRB5 is a natural ligand. In some embodiments, the natural ligand comprises HLA-B7, B27, ANGPTL1, ANGPTL2, ANGPTL3, ANGPTL4, ANGPTL5, ANGPTL6, ANGPTL7, ANGPTL8, or CD166.

Disclosed herein, in certain embodiments, is a pharmaceutical composition, comprising: an anti-LILRB3 antibody or binding fragment thereof, an anti-LILRB4 antibody or binding fragment thereof, an anti-LILRB5 antibody or binding fragment thereof, or a pan anti-LILRB antibody or binding fragment thereof; and a pharmaceutically acceptable excipient. In some embodiments, the pharmaceutical composition is formulated for systemic administration. In some embodiments, the pharmaceutical composition is formulated for parenteral administration.

Disclosed herein, in certain embodiments, is an anti-LILRB antibody that specifically binds to an epitope on the extracellular domain of LILRB1, an epitope on the extracellular domain of LILRB2, an epitope on the extracellular domain of LILRB3, an epitope on the extracellular domain of LILRB4, or an epitope on the extracellular domain of LILRB5, for the treatment of a proliferative disease, an infectious disease, or a neurological disease or disorder. In some embodiments, the epitope comprises a peptide sequence within domain D1, D2, D3, or D4, or a combination thereof of a LILRB protein. In some embodiments, the epitope comprises a peptide sequence within domain D1, D2, D3, or D4, or a combination thereof of LILRB2. In some embodiments, the epitope comprises a peptide sequence within domain D1 or D2, or a combination thereof of LILRB2, wherein D1 comprises an amino acid region that corresponds to residues 22-110 of SEQ ID NO: 9 and D2 comprises an amino acid region that corresponds to residues 111-229 of SEQ ID NO: 9. In some embodiments, the epitope comprises a peptide sequence within domain D3 or D4, or a combination thereof of LILRB2, wherein D3 comprises an amino acid region that corresponds to residues 230-318 of SEQ ID NO: 9, and D4 comprises an amino acid region that corresponds to residues 319-419 of SEQ ID NO: 9. In some embodiments, if the anti-LILRB antibody specifically binds to an epitope within D3 or within D4, or to an epitope within D3 and an epitope within D4, the anti-LILRB antibody further weakly binds to an epitope within D1 or D2. In some embodiments, the anti-LILRB antibody specifically binds to a conformational epitope. In some embodiments, the conformational epitope is: within D1, D2, D3, or D4; within D1 or D2; within D2 or D3; or within D3 or D4. In some embodiments, the conformational epitope comprises: at least one peptide sequence from D1 and at least one peptide sequence from D2; or at least one peptide sequence from D3 and at least one peptide sequence from D4. In some embodiments, the anti-LILRB antibody is a pan antibody that specifically binds to LILRB1, LILRB2, and LILRB3. In some embodiments, the pan antibody specifically binds: to one or more LILRB1 isoforms selected from isoforms 1-6; or to a LILRB1 encoded by a sequence comprising at least 90%, 95%, 96%, 97%, 98%, 99%, or 100% sequence identity to SEQ ID NOs: 33-35. In some embodiments, the pan antibody specifically binds: to one or more LILRB2 isoforms selected from isoforms 1-5; or to a LILRB2 encoded by a sequence comprising at least 90%, 95%, 96%, 97%, 98%, 99%, or 100% sequence identity to SEQ ID NOs: 36-39. In some embodiments, the pan antibody specifically binds: to one or more LILRB3 isoforms selected from isoforms 1-3; or to a LILRB3 encoded by a sequence comprising at least 90%, 95%, 96%, 97%, 98%, 99%, or 100% sequence identity to SEQ ID NO: 40 or 41. In some embodiments, the pan antibody further specifically binds to: LILRB5; LILRA1, LILRA3, LILRA5, LILRA6, or a combination thereof; LILRA1, LILRA3, LILRA5, and LILRA6; or LILRA1, LILRA3, and LILRA6. In some embodiments, the anti-LILRB antibody is an anti-LILRB2 antibody that specifically binds to LILRB2 and weakly binds to an epitope on the extracellular domain of LILRB1, LILRB3, LILRB4, and LILRB5. In some embodiments, the anti-LILRB2 antibody weakly binds or does not bind to an LILRA. In some embodiments, the anti-LILRB antibody is a pan antibody that specifically binds to: LILRB1, LILRB2, LILRB4, and LILRB5; LILRB1, LILRB2, LILRB3, and LILRB4; LILRB1, LILRB2, and LILRB5; or LILRB1 and LILRB3. In some embodiments, the anti-LILRB antibody blocks HLA-G binding to a cell expressing a LILRB receptor, blocks HLA-A binding to the cell expressing a LILRB receptor, or a combination thereof. In some embodiments, the anti-LILRB antibody enhances HLA-G binding to a cell expressing a LILRB receptor. In some embodiments, the anti-LILRB antibody does not modulate HLA-G binding or HLA-A binding to a cell expressing a LILRB receptor. In some embodiments, the anti-LILRB antibody comprises a full-length antibody or a binding fragment thereof, optionally comprising a humanized antibody or binding fragment thereof, chimeric antibody or binding fragment thereof, monoclonal antibody or binding fragment thereof, bispecific antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, the proliferative disease is cancer. In some embodiments, the cancer is a solid tumor or a hematologic malignancy. In some embodiments, the infectious disease is a viral infection. In some embodiments, the infectious disease is Dengue fever or AIDS. In some embodiments, the infectious disease is caused by a protozoan. In some embodiments, the infectious disease is malaria. In some embodiments, the neurological disease or disorder is a neurodegenerative disease or disorder. In some embodiments, the neurological disease or disorder is Alzheimer's disease. In some embodiments, the anti-LILRB antibody inhibits binding of a ligand of LILRB to LILRB by at least 10%, 20%, 30%, 40%, 50%, 60%, 70%, 80%, 90%, 95%, or more. In some embodiments, the anti-LILRB antibody inhibits binding of a ligand of LILRB to LILRB by about 2-fold, 3-fold, 4-fold, 5-fold, 6-fold, 7-fold, 8-fold, 9-fold, 10-fold, or more. In some embodiments, the ligand of LILRB is a natural ligand. In some embodiments, the natural ligand comprises: HLA-A, HLA-B, HLA-C, HLA-E, HLA-G, CD1c, CD1d, MAG, ANGPTL1, ANGPTL2, ANGPTL3, ANGPTL4, ANGPTL5, ANGPTL6, ANGPTL7, ANGPTL8, RTN4, or OMgp; or HLA-A; oligo A oligomers; or a pathogen, optionally selected from Dengue, Escherichia coli, or Staphylococcus aureus. In some embodiments, the anti-LILRB antibody is 5G11.G8, 5G11.H6, 9C9.D3, 9C9.E6, 16D11.D10, 6G6.H7, 6G6.H2, 6H9.A3, 2B3.A10, 4D11.B10, or 11D9.E7. In some embodiments, the anti-LILRB antibody, when contacted to a plurality of peripheral blood mononuclear cells (PBMCs) comprising T cells, enhances cytotoxic T cell activation relative to a plurality of equivalent PBMCs and equivalent T cells in the absence of the anti-LILRB antibody. In some embodiments, the anti-LILRB antibody, when contacted to a plurality of peripheral blood mononuclear cells (PBMCs) comprising a macrophage, increases M1 activation of the macrophage relative to a plurality of equivalent PBMCs and an equivalent macrophage in the absence of the anti-LILRB antibody. In some embodiments, the anti-LILRB antibody, when contacted to a plurality of cells, increases inflammatory cytokine production relative to a plurality of equivalent cells in the absence of the anti-LILRB antibody. In some embodiments, the inflammatory cytokine comprises TNFα, IFNγ, or a combination thereof. In some embodiments, the anti-LILRB antibody, when contacted to a plurality of cells comprising PBMCs and tumor cells, decreases tumor cell proliferation relative to a plurality of equivalent cells comprising PBMCs and tumor cells in the absence of the anti-LILRB antibody. In some embodiments, the anti-LILRB antibody, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the anti-LILRB antibody. In some embodiments, the anti-LILRB antibody decreases regulatory T cells when administered to a subject in need thereof, relative to a second subject in the absence of the antibody or binding fragment thereof.

Disclosed herein, in certain embodiments, is a pan anti-LILRB antibody that specifically binds to at least one epitope on the extracellular domain of LILRB1, at least one epitope on the extracellular domain of LILRB2, or at least one epitope on the extracellular domain of LILRB3, for the treatment of a proliferative disease, an infectious disease, or a neurological disease or disorder. In some embodiments, the pan anti-LILRB antibody further specifically binds to an epitope on the extracellular domain of LILRB4 or an epitope on the extracellular domain of LILRB5. In some embodiments, the pan anti-LILRB antibody further specifically binds to: LILRA1, LILRA3, LILRA5, LILRA6, or a combination thereof, LILRA1, LILRA3, LILRA5, and LILRA6; or LILRA1, LILRA3, and LILRA6. In some embodiments, the at least one epitope on the extracellular domain of LILRB2 comprises a peptide sequence within D3, a peptide sequence within D4, or a combination thereof. In some embodiments, the at least one epitope on the extracellular domain of LILRB2 comprises a peptide sequence within D1, a peptide sequence within D2, or a combination thereof. In some embodiments, the at least one epitope on the extracellular domain of LILRB2 comprises a conformational epitope. In some embodiments, the conformational epitope: is within D3 and comprises at least one peptide sequence; is within D4 and comprises at least one peptide sequence; comprises at least one peptide sequence from D1 and at least one peptide sequence from D2; or comprises at least one peptide sequence from D3 and at least one peptide sequence from D4. In some embodiments, the pan anti-LILRB antibody blocks HLA-G binding to a cell expressing a LILRB receptor. In some embodiments, the pan anti-LILRB antibody comprises a full-length antibody or a binding fragment thereof, optionally comprising a humanized antibody or binding fragment thereof, chimeric antibody or binding fragment thereof, monoclonal antibody or binding fragment thereof, bispecific antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, the pan anti-LILRB antibody inhibits binding of a ligand of LILRB1 to LILRB1 and/or a ligand of LILRB2 to LILRB2 by at least 10%, 20%, 30%, 40%, 50%, 60%, 70%, 80%, 90%, 95%, or more. In some embodiments, the pan anti-LILRB antibody inhibits binding of a ligand of LILRB1 to LILRB1 and/or a ligand of LILRB2 to LILRB2 by about 2-fold, 3-fold, 4-fold, 5-fold, 6-fold, 7-fold, 8-fold, 9-fold, 10-fold, or more. In some embodiments, the ligand of LILRB1 and the ligand of LILRB2 are each independently a natural ligand. In some embodiments, the natural ligand comprises: HLA-A, HLA-B, HLA-C, HLA-E, HLA-G, CD1c, CD1d, MAG, ANGPTL1, ANGPTL2, ANGPTL3, ANGPTL4, ANGPTL5, ANGPTL6, ANGPTL7, ANGPTL8, RTN4, or OMgp; HLA-A; oligo A oligomers; or a pathogen, optionally selected from Dengue, Escherichia coli, or Staphylococcus aureus. In some embodiments, the pan anti-LILRB antibody is 5G11.G8, 5G11.H6, 9C9.D3, 9C9.E6, 16D11.D10, or 11D9.E7. In some embodiments, the pan anti-LILRB antibody, when contacted to a plurality of peripheral blood mononuclear cells (PBMCs) comprising a macrophage, increases M1 activation of the macrophage relative to a plurality of equivalent PBMCs and an equivalent macrophage in the absence of the pan anti-LILRB antibody. In some embodiments, the pan anti-LILRB antibody, when contacted to a plurality of cells, increases inflammatory cytokine production relative to a plurality of equivalent cells in the absence of the pan anti-LILRB antibody. In some embodiments, the inflammatory cytokine comprises TNFα, IFNγ, or a combination thereof. In some embodiments, the pan anti-LILRB antibody, when contacted to a plurality of cells comprising PBMCs and tumor cells, decreases tumor cell proliferation relative to a plurality of equivalent cells comprising PBMCs and tumor cells in the absence of the pan anti-LILRB antibody. In some embodiments, the pan anti-LILRB antibody, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the pan anti-LILRB antibody.

Disclosed herein, in certain embodiments, is a pharmaceutical composition, comprising: an anti-LILRB antibody described above or a pan anti-LILRB antibody described above; and a pharmaceutically acceptable excipient. In some embodiments, the pharmaceutical composition is formulated for systemic administration. In some embodiments, the pharmaceutical composition is formulated for parenteral administration.

Disclosed herein, in certain embodiments, is a method of modulating a macrophage to undergo M1 activation, comprising: (a) contacting a plurality of antigen presenting cells (APCs) comprising a macrophage with an anti-LILRB antibody described above or a pan anti-LILRB antibody described above; (b) binding the antibody or binding fragment thereof or the pan antibody or binding fragment thereof to one or more LILRB receptors expressed on at least one APC within the plurality of APCs, thereby inducing the APC to produce a plurality of TNFα and interferons; and (c) contacting the plurality of TNFα and interferons with the plurality of APCs comprising the macrophage to induce M1 activation of the macrophage. In some embodiments, the interferon is IFNγ or IFNβ. In some embodiments, the anti-LILRB antibody or the pan anti-LILRB antibody decreases M2 activation of the macrophage. In some embodiments, the anti-LILRB antibody or the pan anti-LILRB antibody decreases formation of a tumor associate macrophage. In some embodiments, the APCs further comprise dendritic cells, B cells, or a combination thereof.

Disclosed herein, in certain embodiments, is a method of inducing phagocytosis of a target cell, comprising: (a) incubating a plurality of antigen presenting cells (APCs) comprising a macrophage with an anti-LILRB antibody described above or a pan anti-LILRB antibody described above, thereby inducing the macrophage to undergo M1 polarization; and (b) contacting the M1 macrophage to a target cell for a time sufficient to induce phagocytosis of the target cell. In some embodiments, the APCs further comprise dendritic cells, B cells, or a combination thereof. In some embodiments, the target cell is a cancer cell. In some embodiments, the target cell is a cell infected by a pathogen.

Disclosed herein, in certain embodiments, is a method of activating a cytotoxic T cell, comprising: (a) incubating a plurality of peripheral blood mononuclear cells (PBMCs) comprising naïve T cells with an anti-LILRB antibody described above or a pan anti-LILRB antibody described above, thereby stimulating the secretion of a plurality of inflammatory cytokines; and (b) contacting the plurality of inflammatory cytokines with the naïve T cells to activate a cytotoxic T cell. In some embodiments, the plurality of inflammatory cytokines comprises TNFα, IFNγ, or IFNβ. In some embodiments, the naïve T cells comprise naïve CD8⁺ T cells. In some embodiments, the PBMCs comprise antigen presenting cells (APCs), NK cells, and/or CD4 T cells. In some embodiments, the CD4 T cells comprise activated CD4⁺ helper T cells. In some embodiments, the APCs comprise B cells and/or dendritic cells.

Disclosed herein, in certain embodiments, is a vector comprising a nucleic acid molecule that encodes an anti-LILRB3 antibody or binding fragment thereof, an anti-LILRB4 antibody or binding fragment thereof, an anti-LILRB5 antibody or binding fragment thereof, or a pan anti-LILRB antibody or binding fragment thereof.

Disclosed herein, in certain embodiments, is a host cell comprising a nucleic acid molecule that encodes an anti-LILRB3 antibody or binding fragment thereof, an anti-LILRB4 antibody or binding fragment thereof, an anti-LILRB5 antibody or binding fragment thereof, or a pan anti-LILRB antibody or binding fragment thereof.

Disclosed herein, in certain embodiments, is a kit comprising an anti-LILRB1 antibody or binding fragment thereof, an anti-LILRB2 antibody or binding fragment thereof, a pan anti-LILRB antibody or binding fragment thereof, or a pharmaceutical composition comprising an anti-LILRB antibody described above. In some embodiments, also described herein is a kit comprising an anti-LILRB3 antibody or binding fragment thereof, an anti-LILRB4 antibody or binding fragment thereof, an anti-LILRB5 antibody or binding fragment thereof, a pan anti-LILRB antibody or binding fragment thereof, or a pharmaceutical composition comprising an anti-LILRB antibody or binding fragment described here.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below:

FIG. 1 illustrates exemplary cartoons of LILRBs 1-5 domain structure and respective exemplary natural ligands.

FIG. 2 illustrates M1 activating properties of exemplary anti-LILRB antibodies described herein.

FIG. 3 illustrates multiple binding and functional properties of exemplary anti-LILRB antibodies described herein.

FIG. 4 shows proliferation of T cells in a mixed lymphocyte reaction (MLR) setting by exemplary anti-LILRB2 antibodies 13H1.G2 and 6G6.H7.

FIG. 5 shows IFNγ production under a 2-way mixed lymphocyte reaction (MLR) setting by exemplary anti-LILRB2 antibodies 13H1.G2 and 6G6.H7 and pan anti-LILRB1/2/3 antibody 9C9.E6.

FIG. 6A illustrates HLA-G binding profile of exemplary anti-LILRB antibodies. FIG. 6A top panel illustrates antibody binding profiles with respect to primary monocytes. FIG. 6A bottom panel illustrates binding of HLA-G tetramer to primary monocytes. The analysis was carried out by FACS.

FIG. 6B shows HLA-G-*01:01-PE tetramer binding to primary CD14⁺ monocytes as determined by flow cytometry.

FIG. 7A shows HLA-A*02:01-PE tetramer unmasking assay, binding to primary CD14⁺ monocytes as determined by flow cytometry.

FIG. 7B shows HLA-A*02:01-PE tetramer blocking assay, binding to primary CD14⁺ monocytes as determined by flow cytometry.

FIG. 8A-FIG. 8N show ELISA binding of HLA-G tetramer to LILRB1-Fc and LILRB2-Fc proteins in the presence of HLA-G blocking antibodies. FIGS. 8A and 8B: antibody 5G11.H6; FIGS. 8C and 8D: antibody 5G11.G8; FIGS. 8E and 8F: antibody 9C9.D3; FIGS. 8G and 8H: antibody 9C9.E6; FIGS. 8I and 8J: antibody 16D11.D10; FIGS. 8K and 8L: antibody 6G6.H7; FIGS. 8M and 8N: antibody 6G6.H2.

FIG. 9A-FIG. 9E ELISA binding of anti-LILRB antibodies to full-length extracellular LILRB1 proteins Lilrb1_01 (SEQ ID NO: 33), Lilrb1_02 (SEQ ID NO: 34), and Lilrb1_03 (SEQ ID NO: 35). FIG. 9A: antibody 5G11.H6; FIG. 9B: antibody 5G11.G8; FIG. 9C: antibody 9C9.D3; FIG. 9D: antibody 9C9.E6; and FIG. 9E: antibody 16D11.D10.

FIG. 10A-FIG. 10G show ELISA binding of anti-LILRB antibodies to full-length extracellular LILRB2 proteins Lilrb2_01 (SEQ ID NO: 36), Lilrb2_02 (SEQ ID NO: 37), Lilrb2_03 (SEQ ID NO: 38), and Lilrb2_04 (SEQ ID NO: 39). FIG. 10A: antibody 5G11.H6; FIG. OB: antibody 5G11.G8; FIG. 10C: antibody 9C9.D3; FIG. 10D: antibody 9C9.E6; FIG. 10E: antibody 16D11.D10; FIG. 10F: antibody 6G6.H2; and FIG. OG: antibody 6G6.H7.

FIG. 11A-FIG. 11E show ELISA binding of anti-LILRB antibodies to full-length extracellular LILRB3 proteins Lilrb3_01 (SEQ ID NO: 40) and Lilrb3_05 (SEQ ID NO: 41). FIG. 11A: antibody 5G11.H6; FIG. 11B: antibody 5G11.G8; FIG. 11C: antibody 9C9.D3; FIG. 11D: antibody 9C9.E6; and FIG. 11E: antibody 16D11.D10.

FIG. 12 shows binding profile of exemplary anti-LILRB antibodies with respect to LILRBs 1-5 and LILRAs 1-6.

FIG. 13A-FIG. 13B show macrophage LPS activation. FIG. 13A: HLA-G blocking, HLA-G enhancing, and HLA-A neural antibodies; FIG. 13B: commercial antibodies #287219 (R&D Systems), 42D1 (Biolegend), and ZM4.1.

FIG. 14 shows macrophage IFNγ activation.

FIG. 15 shows MLR activity of exemplary anti-LILRB antibodies. This set of antibodies was shown to block HLA-G binding in FIG. 6A.

FIG. 16 shows MLR activity of exemplary anti-LILRB antibodies. This set of antibodies was shown to enhance HLA-G binding in FIG. 6A.

FIG. 17 illustrates the MLR activity of exemplary anti-LILRB antibodies.

FIG. 18 shows the ability of exemplary anti-LILRB antibodies to restore HLA-G induced suppression.

FIG. 19 shows a two-way MLR assay with HLA-G. The two-way MLR was established using PBMC cells from two unrelated donors in the presence of HLA-G and 1 μg/mL of HLA blocking anti-LILRB antibodies or IgG isotype controls was added to the PBMC cells.

FIG. 20A-FIG. 20B show suppressive function of HLA-G induced CD33⁺CD11b⁺ MDSCs on allogenic T cells in the present of HLA-G blocking antibodies or IgG isotype controls (FIG. 20A: CD8+ T cell; FIG. 20B: CD4+ T cell). T cell proliferation index was determined by normalizing data with average of CD3/CD28 stimulated T cells.

FIG. 21A-FIG. 21G illustrate ELISA binding of exemplary anti-LILRB antibodies to full-length extracellular LILRB2-Fc (d1-d4), LILRB2_d1d2-Fc, or LILRB2_d3d4-Fc proteins. FIG. 21A: antibody 5G11.G8; FIG. 21B: antibody 5G11.H6; FIG. 21C: antibody 9C9.D3; FIG. 21D: antibody 9C9.E6; FIG. 21E: antibody 16D11.D10; FIG. 21F: antibody 6G6.H2; and FIG. 21G: antibody 6G6.H7. These antibodies were shown to block HLA-G binding in FIG. 6A.

FIG. 22A-FIG. 22D illustrate ELISA binding of exemplary anti-LILRB antibodies to full-length extracellular LILRB2-Fc (d1-d4), LILRB2_d1d2-Fc, or LILRB2_d3d4-Fc proteins. FIG. 22A: antibody 8E8.D2; FIG. 22B: antibody 14B7.A4; FIG. 22C: antibody 8F7.C3; and FIG. 22D: antibody 6H9.A3. These antibodies were shown to enhance HLA-G binding in FIG. 6A.

FIG. 23A-FIG. 23G illustrate ELISA binding of exemplary anti-LILRB antibodies to full-length extracellular LILRB2-Fc (d1-d4), LILRB2_d1d2-Fc, or LILRB2_d3d4-Fc proteins. FIG. 23A: antibody 5H9.A10; FIG. 23B: antibody 2B3.A10; FIG. 23C: antibody 4D11.B10; FIG. 23D: antibody 5B6.A1; FIG. 23E: antibody 11D9.E7; FIG. 23F: antibody IgG1; and FIG. 23G: antibody IgG2b. These antibodies were shown to be neutral with respect to HLA-G binding in FIG. 6A.

FIG. 24 shows ELISA binding of HLA-G tetramer to full-length extracellular Lilrb2-Fc, Lilrb2_d1d2-Fc or Lilrb2_d3d4-Fc protein showing that HLA-G tetramer binding to Lilrb2_d1d2-Fc is equivalent to Lilrb2-Fc.

FIG. 25A-FIG. 25E show linear peptide epitope mapping of exemplary anti-LILRB antibodies. The linear peptides cover the full-length of the wild-type LILRB2 protein. FIG. 25A: antibody 5G11.H6; FIG. 25B: antibody 9C9.E6; FIG. 25C: antibody 16D11.D10; FIG. 25D: antibody 9C9.D3; and FIG. 26E: antibody 5G11.G8.

FIG. 26 shows LILRB binding and HLA-G and HLA-A binding properties of exemplary anti-LILRB antibodies.

### DETAILED DESCRIPTION OF THE DISCLOSURE

Immuno-oncology is a treatment method that utilizes the body's immune system to target and attack a tumor. Checkpoint inhibitors such as monoclonal antibodies Ipilimumab (anti-CTLA4 inhibitor), Pembrolizumab (anti-PD-1 inhibitor), Nivolumab (anti-PD-1 inhibitor), Atezolizumab (anti-PD-L1 inhibitor), Avelumab (anti-PD-L1 antibody), and Durvalumab (anti-PD-L1 inhibitor) enable regulation of immune surveillance, immunoediting, and immunoescape mechanisms, thereby retargeting a body's defense system toward tumor cells.

In some instances, only a sub-population of patients is observed to respond to checkpoint inhibitor treatments. In addition, a subset of patients who initially responded to checkpoint inhibitors later relapses and develops therapy resistance (or acquired resistance). Furthermore, some patients have primary resistance to checkpoint inhibitors, i.e., they do not respond to a checkpoint inhibitor treatment.

The leukocyte immunoglobulin-like receptor (LILR) family comprises immunomodulatory receptors that express on myeloid and lymphocyte cell populations. The LILR family comprises two subfamilies, the inhibitory leukocyte Ig-like receptor subfamily B (LILRB) receptors and the activating LILR subfamily A (LILRA) receptors. The LILRB receptors modulate immune response via the cytoplasmic immunoreceptor tyrosine-based inhibitory motifs (ITIMs). In some cases, the LILRBs are proposed to be members of the immune checkpoint family of proteins, although several LILRB members are expressed on a broader array of cell types than the classical immune checkpoint proteins PD-1 and CTLA4.

The LILRA receptors are involved in modulating innate and adaptive immune responses. The LILRA receptors generally encode a signal peptide, two or four immunoglubulin (Ig)-like domains, a transmembrane domain and a cytoplasmic tail that associates with the Fc receptor γ chain (FcRγ) chain containing immunoreceptor tyrosine-based activation motifs (ITAMs). Based on the interaction with human leukocyte antigen (HLA) class I molecules, human LILRAs are further categorized into LILRA group 1 (LILRA1-3) and LILRA group 2 (LILRA 4-6). In some instances, studies have shown that LILRAs play a role in infection and autoimmune diseases.

Disclosed herein, in certain embodiments, are antibodies or binding fragments that interact with one or more LILRBs. In some instances, the antibodies or binding fragments are specific antibodies and interact with an epitope on the extracellular domain of specific LILRB with minimal or no cross-reactivity to a second LILRB. In other instances, the antibodies or binding fragments are pan antibodies and interact with two or more epitopes on the extracellular domain of the respective LILRBs.

Leukocyte Immunoglobulin-Like Receptor Subfamily B (LILRB)

The leukocyte Ig-like receptor subfamily B (LILRB) is a group of type I transmembrane glycoproteins under the leukocyte Ig-like receptor (LILR) family. LILRB comprises five members, LILRB1, LILRB2, LILRB3, LILRB4, and LILRB5; also known as CD85J, CD85D, CD85A, CD85K, and CD85C, respectively; or leukocyte Ig-like receptors LIR1, LIR2, LIR3, LIR5, and LIR8, respectively. LILRBs 1-4 are also named Ig-like transcripts ILT2, ILT4, ILT5, and ILT3, respectively.

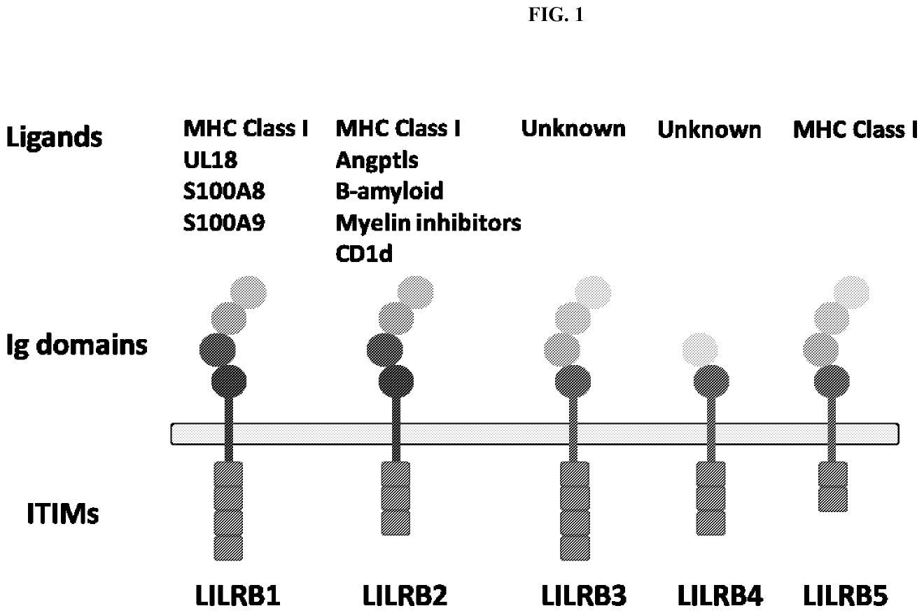

LILRBs comprise an extracellular N-terminal signaling peptide, two to four extracellular Ig-like domains that interact with ligands, intracellular immunoreceptor tyrosine-based inhibitory motifs (ITIMs), and a transmembrane domain bridging the Ig-like domains and the ITIMs. The signaling peptide is further cleaved to generate the mature form which interacts with the respective ligands. FIG. 1 shows exemplary cartoons of LILRBs 1-5 domain structure and their respective exemplary natural ligands.

LILRB1 (also known as CD85J, ILT2, LIR1, and MIR7) comprises 4 intracellular immunoreceptor tyrosine-based inhibitory motifs (ITIMs) and 4 extracellular Ig-like domains, named respectively as D1, D2, D3, and D4 domain. In some instances, LILRB1 is widely expressed on selective Natural Killer (NK) cells, monocytes, macrophages, eosinophils, basophils, dendritic cells (DCs), subsets of T cells, B cells, decidual macrophages, progenitor mast cells, and osteoclasts. In some cases, LILRB1 is uniformly expressed on monocytes and B cells. Ligands that interact with LILRB1 include, but are not limited to, HLA class I molecules (e.g., HLA-A, HLA-B, HLA-C, HLA-E, HLA-F, and HLA-G); UL18, an HLA class I homologue encoded by human cytomegalovirus; α3 domain and β2-microglubulin of class I proteins; calcium-binding proteins S100A8 and S100A9; and pathogenic ligands such as Dengue virus, Escherichia coli, and Staphylococcus aureus.

Cancer cells such as acute myeloid leukemia (AML) cells, neoplastic B cells (e.g., B cell leukemia, B cell lymphoma, and multiple myeloma cells), T cell leukemia and lymphoma cells, and gastric cancer cells have been observed to express LILRB1. Indeed, studies have shown that LILRB1 protects primary cutaneous CD8+ and CD56+ T cell lymphomas from cell death and that expression on human gastric cancer cells contribute to enhanced tumor growth (see Urosevic, et al., “Primary cutaneous CD8+ and CD56+ T-cell lymphomas express HLA-G and killer-cell inhibitory ligand, ILT2,” Blood 103:1796-1798 (2004); Zhang, et al., “Expression of immunoglobulin-like transcript (ILT)2 and ILT3 in human gastric cancer and its clinical significance,” Mol Med Rep 5:910-916 (2012)).

LILRB2 (also known as CD85D, ILT4, LIR2, and MIR10) comprises 3 intracellular ITIMs and 4 extracellular Ig-like domains, named respectively as D1, D2, D3, and D4 domain. LILRB2 is expressed on hematopoietic stem cells, monocytes, macrophages, DCs, basophils, decidual macrophages, mass cell progenitors, endothelial cells, and osteoclasts. In some instances, LILRB2 is not expressed on lymphoid cells. Exemplary ligands recognized by LILRB2 include, but are not limited to, HLA class I molecules (e.g., HLA-A, HLA-B, HLA-C, HLA-E, HLA-F, and HLA-G); cluster of differentiation family of glycoproteins CD1d and CD1c; angiopoietin-like protein ANGPTLs such as ANGPTL1, ANGPTL2, ANGPTL3, ANGPTL4, ANGPTL5, ANGPTL6, ANGPTL7, and ANGPTL8; myelin inhibitors such as Nogo66, myelin associated glycoprotein (MAG), oligodendrocyte myelin glycoprotein (OMgp), and reticulon 4 (RTN4; also known as ASY or NOGO); and β-amyloid.

Cancer cells such as acute myeloid leukemia (AML) cells, chronic lymphoblastic leukemia (CLL) cells, primary ductal and lobular breast cancer cells, and non-small cell lung cancer cells have been observed to express LILRB2. See, e.g., Kang, et al., “The ITIM-containing receptor LAIR1 is essential for acute myeloid leukaemia development,” Nat Cell Biol 17:665-677 (2015); Liu, et al., “ANGPTL2/LILRB2 signaling promotes the propagation of lung cancer cells,” Oncotarget 6:21004-21015 (2015); Wang, et al., “Co-expression of immunoglobulin-like transcript 4 and angiopoietin-like proteins in human non-small cell lung cancer,” Mol Med Rep 11:2789-2796 (2015); Zhang, et al., “IL, T4 drives B7-H3 expression via PI3K/AKT/mTOR signaling and ILT4/B7-H3 co-expression correlates with poor prognosis in non-small cell lung cancer,” FEBS Lett 589:2248-2256 (2015); Colovial, et al., “Expression of inhibitory receptor ILT3 on neoplastic B cells is associated with lymphoid tissue involvement in chronic lymphocytic leukemia,” Cytometry B Clin Cytom 72:354-362 (2007); Sun et al., “Expression of Ig-like transcript 4 inhibitory receptor in human non-small cell lung cancer,” Chest 134:783-788 (2008). Furthermore, in lung cancer, one study has shown that LILRB2 supports cancer cell development and survival (Liu, et al., “ANGPTL2/LILRB2 signaling promotes the propagation of lung cancer cells,” Oncotarget 6:21004-21015 (2015)).

LILRB3 (also known as CD85A, ILT5, LIR3, and HL9) comprises 4 intracellular ITIMs and 4 extracellular Ig-like domains, named respectively as D1, D2, D3, and D4 domain. LILRB3 is expressed on monocytes, monocyte-derived osteoclasts, neutrophils, eosinophils, basophils, osteoclasts, and progenitor mast cells. A pathogenic ligand, S. aureus, has been identified to interact with LILRB3. Cells such as myeloid leukemia, B lymphoid leukemia, and myeloma cells have been shown to express LILRB3 (Pfistershammer, et al., “Allogeneic disparities in immunoglobulin-like transcript 5 induce potent antibody responses in hematopoietic stem cell transplant recipients,” Blood 114:2323-2332 (2009)).

LILRB4 (also known as CD85K, ILT3, LIRS, and HM18) comprises 3 intracellular ITIMs and 2 extracellular Ig-like domains, named respectively as D1 and D2 domains. The D2 domain of LILRB4 shares a sequence homology to D4 domain of LILRBs 1-3 and LILRB5. LILRB4 is expressed on dendritic cells, monocytes, macrophages, progenitor mast cells, endothelial cells, and osteoclasts. A non-limiting example of a LILRB4 ligand is CD166, which mediates interactions between LILRB4 and activated T cells (Xu, et al., “ILT3.Fc-CD166 interaction induces inactivation of p70 S6 kinase and inhibits tumor cell growth,” Journal of Immunology (Baltimore, Md.: 1950), Dec. 20, 2017). Cancer cells such as AML cells, CLL cells, gastric cancer cells, colorectal carcinoma, pancreatic carcinomas, and melanoma have been to express LILRB4 (Dobrowolska, et al., “Expression of immune inhibitory receptor ILT3 in acute myeloid leukemia with monocytic differentiation,” Cytometry B Clin Cytom 84:21-29 (2013); Colovai, et al., “Expression of inhibitory receptor ILT3 on neoplastic B cells is associated with lymphoid tissue involvement in chronic lymphocytic leukemia,” Cytometry B Clin Cytom 72:354-362 (2007); Zhang, et al., “Expression of immunoglobulin-like transcript (ILT)2 and ILT3 in human gastric cancer and its clinical significance,” Mol Med Rep 5:910-916 (2012); Suciu-Foca, et al., “Soluble Ig-like transcript 3 inhibits tumor allograft rejection in humanized SCID mice and T cell responses in cancer patients,” J Immunol 178:7432-7441 (2007); Cortesini, et al., “Pancreas cancer and the role of soluble immunoglobulin-like transcript 3 (ILT3) JOP 8:697-703 (2007)).

LILRB5 (also known as CD85C and LIR8) comprises 2 intracellular ITIMs and 4 extracellular Ig-like domains, named respectively as D1, D2, D3, and D4 domain. LILRB5 is expressed on subpopulations of monocytes, NK cells, and mast cell granules. Exemplary ligands recognized by LILRB5 include, but are not limited to, heavy chains of HLA-B7 and HLA-B27; and ANGPTLs such as ANGPTL1, ANGPTL2, ANGPTL3, ANGPTL4, ANGPTL5, ANGPTL6, ANGPTL7, and ANGPTL8.

In some instances, the LILRBs are further grouped into Class I LILRBs and Class II LILRBs. The Class I LILRBs include LILRB1 and LILRB2. The Class II LILRBs include LILRB3, LILRB4, and LILRB5.

### Anti-LILRB Antibodies

In certain embodiments, disclosed herein are antibodies or binding fragments thereof that bind to a LILRB described above. In some embodiments, the antibodies or binding fragments thereof bind to LILRB1. In some embodiments, the antibodies or binding fragments thereof bind to LILRB2. In other embodiments, the antibodies or binding fragments thereof bind to LILRB3. In additional embodiments, the antibodies or binding fragments thereof bind to LILRB4. In further embodiments, the antibodies or binding fragments thereof bind to LILRB5.

In some instances, the antibodies or binding fragments thereof are pan antibodies. In such instances, a pan antibody binds to LILRB1 and/or LILRB2, and optionally one or more additional LILRBs such as LILRB3, LILRB4, and/or LILRB5; or binds to LILRB3, LILRB4, and/or LILRB5, and optionally one or more additional LILRBs such as LILRB1 and/or LILRB2.

In some instances, the pan anti-LILRB antibody further binds to one or more of LILRAs, e.g., LILRA1, LILRA2, LILRA3, LILRA4, LILRA5, LILRA6, or a combination thereof.

In some cases, the antibody or binding fragment thereof comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof.

### Anti-LILRB1 Antibodies

In some embodiments, described herein are anti-LILRB1 antibodies or binding fragments thereof that specifically bind to an epitope on the extracellular domain of LILRB1, for the treatment of a proliferative disease, an infectious disease, or a neurological disease or disorder. In some instances, the epitope comprises a peptide sequence within domain D1, D2, D3, or D4 of LILRB1. In some cases, the epitope comprises a peptide sequence within domain D1. In some cases, the epitope comprises a peptide sequence within domain D2. In some cases, the epitope comprises a peptide sequence within domain D3. In some cases, the epitope comprises a peptide sequence within domain D4.

In other instances, the epitope comprises at least one peptide sequence within the region between the C-terminus of D4 domain and the N-terminus of the transmembrane domain of LILRB1.

In additional instances, the epitope comprises a domain junction. For example in some cases, the epitope comprises at least one peptide sequence within the D1 domain and at least one peptide sequence within the D2 domain of LILRB1. In other cases, the epitope comprises at least one peptide sequence within the D2 domain and at least one peptide sequence within the D3 domain of LILRB1. In additional cases, the epitope comprises at least one peptide sequence within the D3 domain and at least one peptide sequence within the D4 domain of LILRB1. In further cases, the epitope comprises at least one peptide sequence within the D4 domain and at least one peptide sequence within the region between the C-terminus of D4 domain and the N-terminus of the transmembrane domain of LILRB1. In some instances, the epitope comprises (i) two or more peptide sequences within D1 domain and two or more peptide sequences within D2 domain; (ii) two or more peptide sequences within D2 domain and two or more peptide sequences within D3 domain; (iii) two or more peptide sequences within D3 domain and two or more peptide sequences within D4 domain; or (iv) two or more peptide sequences within D4 domain and two or more peptide sequences within the region between the C-terminus of D4 domain and the N-terminus of the transmembrane domain of LILRB1.

In some embodiments, LILRB1 comprises 6 isoforms (see e.g., SEQ ID NOs: 1-6 in Table 1). In some cases, the anti-LILRB1 antibodies or binding fragments thereof specifically bind to an epitope on the extracellular domain of LILRB1 isoform 1 (SEQ ID NO: 1). In some cases, the anti-LILRB1 antibodies or binding fragments thereof specifically bind to an epitope on the extracellular domain of LILRB1 isoform 2 (SEQ ID NO: 2). In some cases, the anti-LILRB1 antibodies or binding fragments thereof specifically bind to an epitope on the extracellular domain of LILRB1 isoform 3 (SEQ ID NO: 3). In some cases, the anti-LILRB1 antibodies or binding fragments thereof specifically bind to an epitope on the extracellular domain of LILRB1 isoform 4 (SEQ ID NO: 4). In some cases, the anti-LILRB1 antibodies or binding fragments thereof specifically bind to an epitope on the extracellular domain of LILRB1 isoform 5 (SEQ ID NO: 5). In some cases, the anti-LILRB1 antibodies or binding fragments thereof specifically bind to an epitope on the extracellular domain of LILRB1 isoform 6 (SEQ ID NO: 6).

In some cases, the anti-LILRB1 antibodies or binding fragments thereof specifically bind to an epitope on the extracellular domain of LILRB1 comprising a sequence as set forth in SEQ ID NO: 8.

In some instances, D1 domain of LILRB1 comprises an amino acid region that is equivalent to amino acid residues 24-115 of SEQ ID NO: 1. In some cases, an anti-LILRB1 antibody or binding fragment thereof specifically binds to an epitope within D1 in which the amino acid sequence of D1 is equivalent to amino acid residues 24-115 of SEQ ID NO: 1.

In some instances, D2 domain of LILRB1 comprises an amino acid region that is equivalent to amino acid residues 116-221 of SEQ ID NO: 1. In some cases, an anti-LILRB1 antibody or binding fragment thereof specifically binds to an epitope within D2 in which the amino acid sequence of D2 is equivalent to amino acid residues 116-221 of SEQ ID NO: 1.

In some instances, D3 domain of LILRB1 comprises an amino acid region that is equivalent to amino acid residues 222-312 of SEQ ID NO: 1. In some cases, an anti-LILRB1 antibody or binding fragment thereof specifically binds to an epitope within D3 in which the amino acid sequence of D3 is equivalent to amino acid residues 222-312 of SEQ ID NO: 1.

In some instances, D4 domain of LILRB1 comprises an amino acid region that is equivalent to amino acid residues 313-409 of SEQ ID NO: 1. In some cases, an anti-LILRB1 antibody or binding fragment thereof specifically binds to an epitope within D4 in which the amino acid sequence of D4 is equivalent to amino acid residues 313-409 of SEQ ID NO: 1.

In some instances, the region between the C-terminus of D4 domain and the N-terminus of the transmembrane domain of LILRB1 comprises an amino acid region that is equivalent to amino acid residues 410-461 of SEQ ID NO: 1. In some cases, an anti-LILRB1 antibody or binding fragment thereof specifically binds to an epitope within the region between the C-terminus of D4 domain and the N-terminus of the transmembrane domain of LILRB1 in which the amino acid region is equivalent to amino acid residues 410-461 of SEQ ID NO: 1.

In some instances, the epitope is a conformational epitope. In some cases, the conformational epitope is formed of amino acid residues that are discontinuous in the protein sequence but which are brought together upon folding of the LILRB protein into its three-dimensional structure. The conformational epitope differs from a linear epitope, which is formed by a continuous sequence of amino acids in the LILRB protein. In some instances, the conformational epitope comprises at least one peptide sequence within D1, D2, D3, or D4.

In some cases, the conformational epitope comprises at least one peptide sequence within D1 domain and at least one peptide sequence within D2 domain of LILRB1. In such cases, D1 domain of LILRB1 comprises an amino acid region that is equivalent to amino acid residues 24-115 of SEQ ID NO: 1 and D2 domain of LILRB1 comprises an amino acid region that is equivalent to amino acid residues 116-221 of SEQ ID NO: 1.

In some cases, the conformational epitope comprises at least one peptide sequence within D2 domain and at least one peptide sequence within D3 domain of LILRB1. In such cases, D2 domain of LILRB1 comprises an amino acid region that is equivalent to amino acid residues 116-221 of SEQ ID NO: 1 and D3 domain of LILRB1 comprises an amino acid region that is equivalent to amino acid residues 222-312 of SEQ ID NO: 1.

In some cases, the conformational epitope comprises at least one peptide sequence within D3 domain and at least one peptide sequence within D4 domain of LILRB1. In such cases, D3 domain of LILRB1 comprises an amino acid region that is equivalent to amino acid residues 222-312 of SEQ ID NO: 1 and D4 domain of LILRB1 comprises an amino acid region that is equivalent to amino acid residues 313-409 of SEQ ID NO: 1.

In some cases, the conformational epitope comprises at least one peptide sequence within the D4 domain and at least one peptide sequence within the region between the C-terminus of D4 domain and the N-terminus of the transmembrane domain of LILRB1. In such cases, D4 domain of LILRB1 comprises an amino acid region that is equivalent to amino acid residues 313-409 of SEQ ID NO: 1 and the region between the C-terminus of D4 domain and the N-terminus of the transmembrane domain of LILRB1 comprises an amino acid region that is equivalent to amino acid residues 410-461 of SEQ ID NO: 1.

In some embodiments, the anti-LILRB1 antibodies or binding fragments thereof weakly bind to an epitope on the extracellular domain of LILRB2, LILRB3, LILRB4, LILRB5, or one or more LILRAs. As used herein, the term “weakly” refers to a reduced binding affinity toward a non-LILRB1 protein (e.g., LILRB2, LILRB3, LILRB4, LILRB5, or one or more LILRAs) relative to binding affinity to LILRB. In some instances, the reduced binding affinity is about a 10-fold lower in binding affinity toward a non-LILRB1 protein (e.g., LILRB2, LILRB3, LILRB4, LILRB5, or one or more LILRAs) relative to the binding affinity to LILRB1. In some cases, the reduced binding affinity is about a 15-fold lower in binding affinity, about a 20-fold lower in binding affinity, about a 30-fold lower in binding affinity, about a 40-fold lower in binding affinity, about a 50-fold lower in binding affinity, about a 100-fold lower in binding affinity, or more.

In some instances, the anti-LILRB1 antibodies or binding fragments thereof inhibit binding of a ligand of LILRB1 to LILRB1 by at least 10%, 20%, 30%, 40%, 50%, 60%, 70%, 80%, 90%, 95%, or more. In some cases, the anti-LILRB1 antibodies or binding fragments thereof inhibit binding of a ligand of LILRB1 to LILRB1 by at least 10% or more. In some cases, the anti-LILRB1 antibodies or binding fragments thereof inhibit binding of a ligand of LILRB1 to LILRB1 by at least 20% or more. In some cases, the anti-LILRB1 antibodies or binding fragments thereof inhibit binding of a ligand of LILRB1 to LILRB1 by at least 30% or more. In some cases, the anti-LILRB1 antibodies or binding fragments thereof inhibit binding of a ligand of LILRB1 to LILRB1 by at least 40% or more. In some cases, the anti-LILRB1 antibodies or binding fragments thereof inhibit binding of a ligand of LILRB1 to LILRB1 by at least 50% or more. In some cases, the anti-LILRB1 antibodies or binding fragments thereof inhibit binding of a ligand of LILRB1 to LILRB1 by at least 60% or more. In some cases, the anti-LILRB1 antibodies or binding fragments thereof inhibit binding of a ligand of LILRB1 to LILRB1 by at least 70% or more. In some cases, the anti-LILRB1 antibodies or binding fragments thereof inhibit binding of a ligand of LILRB1 to LILRB1 by at least 80% or more. In some cases, the anti-LILRB1 antibodies or binding fragments thereof inhibit binding of a ligand of LILRB1 to LILRB1 by at least 90% or more. In some cases, the anti-LILRB1 antibodies or binding fragments thereof inhibit binding of a ligand of LILRB1 to LILRB1 by at least 95% or more. In some cases, the ligand of LILRB1 is a natural ligand. In some cases, the ligand comprises HLA-A, HLA-B, HLA-C, HLA-E, HLA-F, HLA-G, UL18, α3 domain and β2-microglubulin of class I protein, S100A8, or S100A9. In some cases, the ligand comprises a pathogen such as Dengue virus, Escherichia coli, or Staphylococcus aureus.

In some instances, the anti-LILRB1 antibodies or binding fragments thereof inhibit binding of a ligand of LILRB1 to LILRB1 by about 2-fold, 3-fold, 4-fold, 5-fold, 6-fold, 7-fold, 8-fold, 9-fold, 10-fold, or more. In some instances, the anti-LILRB1 antibodies or binding fragments thereof inhibit binding of a ligand of LILRB1 to LILRB1 by about 2-fold or more. In some instances, the anti-LILRB1 antibodies or binding fragments thereof inhibit binding of a ligand of LILRB1 to LILRB1 by about 3-fold or more. In some instances, the anti-LILRB1 antibodies or binding fragments thereof inhibit binding of a ligand of LILRB1 to LILRB1 by about 4-fold or more. In some instances, the anti-LILRB1 antibodies or binding fragments thereof inhibit binding of a ligand of LILRB1 to LILRB1 by about 5-fold or more. In some instances, the anti-LILRB1 antibodies or binding fragments thereof inhibit binding of a ligand of LILRB1 to LILRB1 by about 6-fold or more. In some instances, the anti-LILRB1 antibodies or binding fragments thereof inhibit binding of a ligand of LILRB1 to LILRB1 by about 7-fold or more. In some instances, the anti-LILRB1 antibodies or binding fragments thereof inhibit binding of a ligand of LILRB1 to LILRB1 by about 8-fold or more. In some instances, the anti-LILRB1 antibodies or binding fragments thereof inhibit binding of a ligand of LILRB1 to LILRB1 by about 9-fold or more. In some instances, the anti-LILRB1 antibodies or binding fragments thereof inhibit binding of a ligand of LILRB1 to LILRB1 by about 10-fold or more. In some cases, the ligand of LILRB1 is a natural ligand. In some cases, the ligand comprises HLA-A, HLA-B, HLA-C, HLA-E, HLA-F, HLA-G, UL18, α3 domain and β2-microglubulin of class I protein, S100A8, or S100A9. In some cases, the ligand comprises a pathogen such as Dengue virus, Escherichia coli, or Staphylococcus aureus.

In some embodiments, an anti-LILRB1 antibody or binding fragment thereof described above comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. In some cases, the anti-LILRB1 antibody or binding fragment thereof comprises a humanized antibody or binding fragment thereof. In some cases, the anti-LILRB1 antibody or binding fragment thereof comprises a murine antibody or binding fragment thereof. In some cases, the anti-LILRB1 antibody or binding fragment thereof comprises a chimeric antibody or binding fragment thereof. In some cases, the anti-LILRB1 antibody or binding fragment thereof comprises a monoclonal antibody or binding fragment thereof. In some cases, the anti-LILRB1 antibody or binding fragment thereof comprises a bispecific antibody or binding fragment thereof. In some cases, the anti-LILRB1 antibody or binding fragment thereof comprises a monovalent Fab′, a divalent Fab2, or F(ab)′3 fragments. In some cases, the anti-LILRB1 antibody or binding fragment thereof comprises a single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, or camelid antibody or binding fragment thereof. In some cases, the anti-LILRB1 antibody or binding fragment thereof comprises a chemically modified derivative thereof.

In some embodiments, the anti-LILRB1 antibody or binding fragment thereof, when contacted to a plurality of peripheral blood mononuclear cells (PBMCs) comprising T cells, enhances cytotoxic T cell activation relative to a plurality of equivalent PBMCs and equivalent T cells in the absence of the antibody or binding fragment thereof.

In some embodiments, the anti-LILRB1 antibody or binding fragment thereof, when contacted to a plurality of peripheral blood mononuclear cells (PBMCs) comprising a macrophage, increases M1 activation of the macrophage relative to a plurality of equivalent PBMCs and an equivalent macrophage in the absence of the antibody or binding fragment thereof.

In some embodiments, the anti-LILRB1 antibody or binding fragment thereof, when contacted to a plurality of cells comprising APCs and a target cell, increases phagocytosis of the target cell relative to a plurality of equivalent cells in the absence of the antibody or binding fragment thereof.

In some embodiments, the anti-LILRB1 antibody or binding fragment thereof, when contacted to a plurality of cells increases inflammatory cytokine production relative to a plurality of equivalent cells in the absence of the antibody or binding fragment thereof. In some cases, the inflammatory cytokine comprises TNFα, IFNγ, or a combination thereof.

In some embodiments, the anti-LILRB1 antibody or binding fragment thereof, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the anti-LILRB1 antibody or binding fragment thereof.

In some embodiments, also described herein include a vector comprising a nucleic acid molecule that encodes an anti-LILRB1 antibody or binding fragment thereof.

In some embodiments, further described herein include a host cell comprising a nucleic acid molecule that encodes an anti-LILRB1 antibody or binding fragment thereof.

Anti-LILRB2 Antibodies

In some embodiments, described herein are anti-LILRB2 antibodies or binding fragments thereof that specifically bind to an epitope on the extracellular domain of LILRB2, for the treatment of a proliferative disease, an infectious disease, or a neurological disease or disorder. In some instances, the epitope comprises a peptide sequence within domain D1, D2, D3, or D4 of LILRB2. In some cases, the epitope comprises a peptide sequence within domain D1. In some cases, the epitope comprises a peptide sequence within domain D2. In some cases, the epitope comprises a peptide sequence within domain D3. In some cases, the epitope comprises a peptide sequence within domain D4.

In other instances, the epitope comprises at least one peptide sequence within the region between the C-terminus of D4 domain and the N-terminus of the transmembrane domain of LILRB2.

In additional instances, the epitope comprises a domain junction. For example in some cases, the epitope comprises at least one peptide sequence within the D1 domain and at least one peptide sequence within the D2 domain of LILRB2. In other cases, the epitope comprises at least one peptide sequence within the D2 domain and at least one peptide sequence within the D3 domain of LILRB2. In additional cases, the epitope comprises at least one peptide sequence within the D3 domain and at least one peptide sequence within the D4 domain of LILRB2. In further cases, the epitope comprises at least one peptide sequence within the D4 domain and at least one peptide sequence within the region between the C-terminus of D4 domain and the N-terminus of the transmembrane domain of LILRB2. In some instances, the epitope comprises (i) two or more peptide sequences within D1 domain and two or more peptide sequences within D2 domain; (ii) two or more peptide sequences within D2 domain and two or more peptide sequences within D3 domain; (iii) two or more peptide sequences within D3 domain and two or more peptide sequences within D4 domain; or (iv) two or more peptide sequences within D4 domain and two or more peptide sequences within the region between the C-terminus of D4 domain and the N-terminus of the transmembrane domain of LILRB2.

In some embodiments, LILRB2 comprises 5 isoforms (see e.g., SEQ ID NOs: 9-13 in Table 1). In some cases, the anti-LILRB2 antibodies or binding fragments thereof specifically bind to an epitope on the extracellular domain of LILRB2 isoform 1 (SEQ ID NO: 9). In some cases, the anti-LILRB2 antibodies or binding fragments thereof specifically bind to an epitope on the extracellular domain of LILRB2 isoform 2 (SEQ ID NO: 10). In some cases, the anti-LILRB2 antibodies or binding fragments thereof specifically bind to an epitope on the extracellular domain of LILRB2 isoform 3 (SEQ ID NO: 11). In some cases, the anti-LILRB2 antibodies or binding fragments thereof specifically bind to an epitope on the extracellular domain of LILRB2 isoform 4 (SEQ ID NO: 12). In some cases, the anti-LILRB2 antibodies or binding fragments thereof specifically bind to an epitope on the extracellular domain of LILRB2 isoform 5 (SEQ ID NO: 13).

In some cases, the anti-LILRB2 antibodies or binding fragments thereof specifically bind to an epitope on the extracellular domain of LILRB2 comprising a sequence as set forth in SEQ ID NO: 15.

In some instances, D1 domain of LILRB2 comprises an amino acid region that is equivalent to amino acid residues 22-110 of SEQ ID NO: 9. In some cases, an anti-LILRB2 antibody or binding fragment thereof specifically binds to an epitope within D1 in which the amino acid sequence of D1 is equivalent to amino acid residues 22-110 of SEQ ID NO: 9.

In some instances, D2 domain of LILRB2 comprises an amino acid region that is equivalent to amino acid residues 111-229 of SEQ ID NO: 9. In some cases, an anti-LILRB2 antibody or binding fragment thereof specifically binds to an epitope within D2 in which the amino acid sequence of D2 is equivalent to amino acid residues 111-229 of SEQ ID NO: 9.

In some instances, D3 domain of LILRB2 comprises an amino acid region that is equivalent to amino acid residues 230-318 of SEQ ID NO: 9. In some cases, an anti-LILRB2 antibody or binding fragment thereof specifically binds to an epitope within D3 in which the amino acid sequence of D3 is equivalent to amino acid residues 230-318 of SEQ ID NO: 9.

In some instances, D4 domain of LILRB2 comprises an amino acid region that is equivalent to amino acid residues 319-419 of SEQ ID NO: 9. In some cases, an anti-LILRB2 antibody or binding fragment thereof specifically binds to an epitope within D4 in which the amino acid sequence of D4 is equivalent to amino acid residues 319-419 of SEQ ID NO: 9.

In some instances, the region between the C-terminus of D4 domain and the N-terminus of the transmembrane domain of LILRB2 comprises an amino acid region that is equivalent to amino acid residues 420-462 of SEQ ID NO: 9 or amino acid residues 420-461 of SEQ ID NO: 10. In some cases, an anti-LILRB2 antibody or binding fragment thereof specifically binds to an epitope within the region between the C-terminus of D4 domain and the N-terminus of the transmembrane domain of LILRB2 in which the amino acid sequence is equivalent to amino acid residues 420-462 of SEQ ID NO: 9 or amino acid residues 420-461 of SEQ ID NO: 10.

In some instances, the epitope is a conformational epitope. In some instances, the conformational epitope comprises at least one peptide sequence within D1, D2, D3, or D4. In some instances, the conformational epitope comprises at least one peptide sequence within D3 or D4. In some cases, the conformational epitope within D3 comprises at least one peptide sequence from region 223-231, region 236-248, region 258-262, region 269-290, or region 298-314, or a combination thereof, wherein the residue numberings correspond to positions 223-231, 236-248, 258-262, 269-290, and 298-314 of SEQ ID NO: 9. In some cases, the conformational epitope within D4 comprises at least one peptide sequence from region 336-340, region 362-368, region 379-393, region 400-403, or region 412-419, or a combination thereof, wherein the residue numberings correspond to positions 336-340, 362-368, 379-393, 400-403, and 412-419 of SEQ ID NO: 9.

In some cases, the conformational epitope comprises at least one peptide sequence within D1 domain and at least one peptide sequence within D2 domain of LILRB2. In such cases, D1 domain of LILRB2 comprises an amino acid region that is equivalent to amino acid residues 22-110 of SEQ ID NO: 9 and D2 domain of LILRB2 comprises an amino acid region that is equivalent to amino acid residues 111-229 of SEQ ID NO: 9.

In some cases, the conformational epitope comprises at least one peptide sequence within D2 domain and at least one peptide sequence within D3 domain of LILRB2. In such cases, D2 domain of LILRB2 comprises an amino acid region that is equivalent to amino acid residues 111-229 of SEQ ID NO: 9 and D3 domain of LILRB2 comprises an amino acid region that is equivalent to amino acid residues 230-318 of SEQ ID NO: 9.

In some cases, the conformational epitope comprises at least one peptide sequence within D3 domain and at least one peptide sequence within D4 domain of LILRB2. In such cases, D3 domain of LILRB2 comprises an amino acid region that is equivalent to amino acid residues 230-318 of SEQ ID NO: 9 and D4 domain of LILRB2 comprises an amino acid region that is equivalent to amino acid residues 319-419 of SEQ ID NO: 9. In additional cases, the conformational epitope comprises at least one peptide sequence from region 223-231, region 236-248, region 258-262, region 269-290, or region 298-314, or a combination thereof of D3 and at least one peptide sequence from region 336-340, region 362-368, region 379-393, region 400-403, or region 412-419, or a combination thereof of D4, wherein the residue numberings correspond to positions 223-231, 236-248, 258-262, 269-290, 298-314, 336-340, 362-368, 379-393, 400-403, and 412-419 of SEQ ID NO: 9.

In some cases, the conformational epitope comprises at least one peptide sequence within D4 domain and at least one peptide sequence within the region between the C-terminus of D4 domain and the N-terminus of the transmembrane domain of LILRB2. In such cases, D4 domain of LILRB2 comprises an amino acid region that is equivalent to amino acid residues 319-419 of SEQ ID NO: 9 and the region between the C-terminus of D4 domain and the N-terminus of the transmembrane domain of LILRB2 comprises an amino acid region that is equivalent to amino acid residues 420-462 of SEQ ID NO: 9 or amino acid residues 420-461 of SEQ ID NO: 10.

In some embodiments, the anti-LILRB2 antibodies or binding fragments thereof weakly bind to an epitope on the extracellular domain of LILRB1, LILRB3, LILRB4, LILRB5, or one or more LILRAs. As used herein, the term “weakly” refers to a reduced binding affinity toward a non-LILRB2 protein (e.g., LILRB1, LILRB3, LILRB4, LILRB5, or one or more LILRAs) relative to binding affinity to LILRB2. In some instances, the reduced binding affinity is about a 10-fold lower in binding affinity toward a non-LILRB2 protein (e.g., LILRB1, LILRB3, LILRB4, LILRB5, or one or more LILRAs) relative to the binding affinity to LILRB2. In some cases, the reduced binding affinity is about a 15-fold lower in binding affinity, about a 20-fold lower in binding affinity, about a 30-fold lower in binding affinity, about a 40-fold lower in binding affinity, about a 50-fold lower in binding affinity, about a 100-fold lower in binding affinity, or more.

In some instances, the anti-LILRB2 antibodies or binding fragments thereof inhibit binding of a ligand of LILRB2 to LILRB2 by at least 10%, 20%, 30%, 40%, 50%, 60%, 70%, 80%, 90%, 95%, or more. In some cases, the anti-LILRB2 antibodies or binding fragments thereof inhibit binding of a ligand of LILRB2 to LILRB2 by at least 10% or more. In some cases, the anti-LILRB2 antibodies or binding fragments thereof inhibit binding of a ligand of LILRB2 to LILRB2 by at least 20% or more. In some cases, the anti-LILRB2 antibodies or binding fragments thereof inhibit binding of a ligand of LILRB2 to LILRB2 by at least 30% or more. In some cases, the anti-LILRB2 antibodies or binding fragments thereof inhibit binding of a ligand of LILRB2 to LILRB2 by at least 40% or more. In some cases, the anti-LILRB2 antibodies or binding fragments thereof inhibit binding of a ligand of LILRB2 to LILRB2 by at least 50% or more. In some cases, the anti-LILRB2 antibodies or binding fragments thereof inhibit binding of a ligand of LILRB2 to LILRB2 by at least 60% or more. In some cases, the anti-LILRB2 antibodies or binding fragments thereof inhibit binding of a ligand of LILRB2 to LILRB2 by at least 70% or more. In some cases, the anti-LILRB2 antibodies or binding fragments thereof inhibit binding of a ligand of LILRB2 to LILRB2 by at least 80% or more. In some cases, the anti-LILRB2 antibodies or binding fragments thereof inhibit binding of a ligand of LILRB2 to LILRB2 by at least 90% or more. In some cases, the anti-LILRB2 antibodies or binding fragments thereof inhibit binding of a ligand of LILRB2 to LILRB2 by at least 95% or more. In some cases, the ligand of LILRB2 is a natural ligand. In some cases, the ligand comprises HLA-A, HLA-B, HLA-C, HLA-E, HLA-F, HLA-G, CD1d, CD1c, ANGPTL1, ANGPTL2, ANGPTL3, ANGPTL4, ANGPTL5, ANGPTL6, ANGPTL7, ANGPTL8, Nogo66, MAG, OMgp, RTN4, or β-amyloid.

In some instances, the anti-LILRB2 antibodies or binding fragments thereof inhibit binding of a ligand of LILRB2 to LILRB2 by about 2-fold, 3-fold, 4-fold, 5-fold, 6-fold, 7-fold, 8-fold, 9-fold, 10-fold, or more. In some instances, the anti-LILRB2 antibodies or binding fragments thereof inhibit binding of a ligand of LILRB2 to LILRB2 by about 2-fold or more. In some instances, the anti-LILRB2 antibodies or binding fragments thereof inhibit binding of a ligand of LILRB2 to LILRB2 by about 3-fold or more. In some instances, the anti-LILRB2 antibodies or binding fragments thereof inhibit binding of a ligand of LILRB2 to LILRB2 by about 4-fold or more. In some instances, the anti-LILRB2 antibodies or binding fragments thereof inhibit binding of a ligand of LILRB2 to LILRB2 by about 5-fold or more. In some instances, the anti-LILRB2 antibodies or binding fragments thereof inhibit binding of a ligand of LILRB2 to LILRB2 by about 6-fold or more. In some instances, the anti-LILRB2 antibodies or binding fragments thereof inhibit binding of a ligand of LILRB2 to LILRB2 by about 7-fold or more. In some instances, the anti-LILRB2 antibodies or binding fragments thereof inhibit binding of a ligand of LILRB2 to LILRB2 by about 8-fold or more. In some instances, the anti-LILRB2 antibodies or binding fragments thereof inhibit binding of a ligand of LILRB2 to LILRB2 by about 9-fold or more. In some instances, the anti-LILRB2 antibodies or binding fragments thereof inhibit binding of a ligand of LILRB2 to LILRB2 by about 10-fold or more. In some cases, the ligand of LILRB2 is a natural ligand. In some cases, the ligand comprises HLA-A, HLA-B, HLA-C, HLA-E, HLA-F, HLA-G, CD1d, CD1c, ANGPTL1, ANGPTL2, ANGPTL3, ANGPTL4, ANGPTL5, ANGPTL6, ANGPTL7, ANGPTL8, Nogo66, MAG, OMgp, RTN4, or β-amyloid.

In some embodiments, an anti-LILRB2 antibody or binding fragment thereof described above comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. In some cases, the anti-LILRB2 antibody or binding fragment thereof comprises a humanized antibody or binding fragment thereof. In some cases, the anti-LILRB2 antibody or binding fragment thereof comprises a murine antibody or binding fragment thereof. In some cases, the anti-LILRB2 antibody or binding fragment thereof comprises a chimeric antibody or binding fragment thereof. In some cases, the anti-LILRB2 antibody or binding fragment thereof comprises a monoclonal antibody or binding fragment thereof. In some cases, the anti-LILRB2 antibody or binding fragment thereof comprises a bispecific antibody or binding fragment thereof. In some cases, the anti-LILRB2 antibody or binding fragment thereof comprises a monovalent Fab′, a divalent Fab2, or F(ab)′3 fragments. In some cases, the anti-LILRB2 antibody or binding fragment thereof comprises a single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, or camelid antibody or binding fragment thereof. In some cases, the anti-LILRB2 antibody or binding fragment thereof comprises a chemically modified derivative thereof.

In some embodiments, the anti-LILRB2 antibody or binding fragment thereof, when contacted to a plurality of peripheral blood mononuclear cells (PBMCs) comprising T cells, enhances cytotoxic T cell activation relative to a plurality of equivalent PBMCs and equivalent T cells in the absence of the antibody or binding fragment thereof.

In some embodiments, the anti-LILRB2 antibody or binding fragment thereof, when contacted to a plurality of peripheral blood mononuclear cells (PBMCs) comprising a macrophage, increases M1 activation of the macrophage relative to a plurality of equivalent PBMCs and an equivalent macrophage in the absence of the antibody or binding fragment thereof.

In some embodiments, the anti-LILRB2 antibody or binding fragment thereof, when contacted to a plurality of cells comprising APCs and a target cell, increases phagocytosis of the target cell relative to a plurality of equivalent cells in the absence of the antibody or binding fragment thereof.

In some embodiments, the anti-LILRB2 antibody or binding fragment thereof, when contacted to a plurality of cells increases inflammatory cytokine production relative to a plurality of equivalent cells in the absence of the antibody or binding fragment thereof. In some cases, the inflammatory cytokine comprises TNFα, IFNγ, or a combination thereof.

In some embodiments, the anti-LILRB2 antibody or binding fragment thereof, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the anti-LILRB2 antibody or binding fragment thereof.

In some embodiments, also described herein include a vector comprising a nucleic acid molecule that encodes an anti-LILRB2 antibody or binding fragment thereof.

In some embodiments, further described herein include a host cell comprising a nucleic acid molecule that encodes an anti-LILRB2 antibody or binding fragment thereof.

### Anti-LILRB3 Antibodies

In some embodiments, described herein are anti-LILRB3 antibodies or binding fragments thereof that specifically bind to an epitope on the extracellular domain of LILRB3, for the treatment of a proliferative disease, an infectious disease, or an autoimmune disease. In some instances, the epitope comprises a peptide sequence within domain D1, D2, D3, or D4 of LILRB3. In some cases, the epitope comprises a peptide sequence within domain D1. In some cases, the epitope comprises a peptide sequence within domain D2. In some cases, the epitope comprises a peptide sequence within domain D3. In some cases, the epitope comprises a peptide sequence within domain D4.

In other instances, the epitope comprises at least one peptide sequence within the region between the C-terminus of D4 domain and the N-terminus of the transmembrane domain of LILRB3.

In additional instances, the epitope comprises a domain junction. For example in some cases, the epitope comprises at least one peptide sequence within the D1 domain and at least one peptide sequence within the D2 domain of LILRB3. In other cases, the epitope comprises at least one peptide sequence within the D2 domain and at least one peptide sequence within the D3 domain of LILRB3. In additional cases, the epitope comprises at least one peptide sequence within the D3 domain and at least one peptide sequence within the D4 domain of LILRB3. In further cases, the epitope comprises at least one peptide sequence within the D4 domain and at least one peptide sequence within the region between the C-terminus of D4 domain and the N-terminus of the transmembrane domain of LILRB3. In some instances, the epitope comprises (i) two or more peptide sequences within D1 domain and two or more peptide sequences within D2 domain; (ii) two or more peptide sequences within D2 domain and two or more peptide sequences within D3 domain; (iii) two or more peptide sequences within D3 domain and two or more peptide sequences within D4 domain; or (iv) two or more peptide sequences within D4 domain and two or more peptide sequences within the region between the C-terminus of D4 domain and the N-terminus of the transmembrane domain of LILRB3.

In some embodiments, LILRB3 comprises 3 isoforms (see e.g., SEQ ID NOs: 21-23 in Table 1). In some cases, the anti-LILRB3 antibodies or binding fragments thereof specifically bind to an epitope on the extracellular domain of LILRB3 isoform 1 (SEQ ID NO: 21). In some cases, the anti-LILRB3 antibodies or binding fragments thereof specifically bind to an epitope on the extracellular domain of LILRB3 isoform 2 (SEQ ID NO: 22). In some cases, the anti-LILRB3 antibodies or binding fragments thereof specifically bind to an epitope on the extracellular domain of LILRB3 isoform 3 (SEQ ID NO: 23).

In some cases, the anti-LILRB3 antibodies or binding fragments thereof specifically bind to an epitope on the extracellular domain of LILRB3 comprising a sequence as set forth in SEQ ID NO: 17.

In some instances, D1 domain of LILRB3 comprises an amino acid region that is equivalent to amino acid residues 25-110 of SEQ ID NO: 21. In some cases, an anti-LILRB3 antibody or binding fragment thereof specifically binds to an epitope within D1 in which the amino acid sequence of D1 is equivalent to amino acid residues 25-110 of SEQ ID NO: 21. In some cases, D1 domain comprises a sequence having about 90% or more sequence identity to amino acid residues 25-110 of SEQ ID NO: 21. In some cases, D1 domain comprises a sequence having about 90%, 95%, 96%, 97%, 98%, 99%, or more sequence identity to amino acid residues 25-110 of SEQ ID NO: 21.

In some instances, D2 domain of LILRB3 comprises an amino acid region that is equivalent to amino acid residues 111-223 of SEQ ID NO: 21. In some cases, an anti-LILRB3 antibody or binding fragment thereof specifically binds to an epitope within D2 in which the amino acid sequence of D2 is equivalent to amino acid residues 111-223 of SEQ ID NO: 21. In some cases, D2 domain comprises a sequence having about 90% or more sequence identity to amino acid residues 111-223 of SEQ ID NO: 21. In some cases, D2 domain comprises a sequence having about 90%, 95%, 96%, 97%, 98%, 99%, or more sequence identity to amino acid residues 111-223 of SEQ ID NO: 21.

In some instances, D3 domain of LILRB3 comprises an amino acid region that is equivalent to amino acid residues 224-323 of SEQ ID NO: 21. In some cases, an anti-LILRB3 antibody or binding fragment thereof specifically binds to an epitope within D3 in which the amino acid sequence of D3 is equivalent to amino acid residues 224-323 of SEQ ID NO: 21. In some cases, D3 domain comprises a sequence having about 90% or more sequence identity to amino acid residues 224-323 of SEQ ID NO: 21. In some cases, D3 domain comprises a sequence having about 90%, 95%, 96%, 97%, 98%, 99%, or more sequence identity to amino acid residues 224-323 of SEQ ID NO: 21.

In some instances, D4 domain of LILRB3 comprises an amino acid region that is equivalent to amino acid residues 324-419 of SEQ ID NO: 21. In some cases, an anti-LILRB3 antibody or binding fragment thereof specifically binds to an epitope within D4 in which the amino acid sequence of D4 is equivalent to amino acid residues 324-419 of SEQ ID NO: 21. In some cases, D4 domain comprises a sequence having about 90% or more sequence identity to amino acid residues 324-419 of SEQ ID NO: 21. In some cases, D4 domain comprises a sequence having about 90%, 95%, 96%, 97%, 98%, 99%, or more sequence identity to amino acid residues 324-419 of SEQ ID NO: 21.

In some instances, the region between the C-terminus of D4 domain and the N-terminus of the transmembrane domain of LILRB3 comprises an amino acid region that is equivalent to amino acid residues 420-443 of SEQ ID NO: 21. In some cases, an anti-LILRB3 antibody or binding fragment thereof specifically binds to an epitope within the region between the C-terminus of D4 domain and the N-terminus of the transmembrane domain of LILRB3 in which the amino acid region is equivalent to amino acid residues 420-443 of SEQ ID NO: 21. In some cases, the region between the C-terminus of D4 domain and the N-terminus of the transmembrane domain of LILRB3 comprises a sequence having about 90% or more sequence identity to amino acid residues 420-443 of SEQ ID NO: 21. In some cases, the region between the C-terminus of D4 domain and the N-terminus of the transmembrane domain of LILRB3 comprises a sequence having about 90%, 95%, 96%, 97%, 98%, 99%, or more sequence identity to amino acid residues 420-443 of SEQ ID NO: 21.

In some instances, the epitope is a conformational epitope. In some instances, the conformational epitope comprises at least one peptide sequence within D1, D2, D3, or D4. In some cases, the conformational epitope comprises at least one peptide sequence within D3. In some cases, the conformational epitope comprises at least one peptide sequence within D4.

In some cases, the conformational epitope comprises at least one peptide sequence within D1 domain and at least one peptide sequence within D2 domain of LILRB3. In such cases, D1 domain of LILRB3 comprises an amino acid region that is equivalent to amino acid residues 25-110 of SEQ ID NO: 21 and D2 domain of LILRB1 comprises an amino acid region that is equivalent to amino acid residues 111-223 of SEQ ID NO: 21. In some cases, D1 domain comprises a sequence having about 90% or more sequence identity to amino acid residues 25-110 of SEQ ID NO: 21. In some cases, D2 domain comprises a sequence having about 90% or more sequence identity to amino acid residues 111-223 of SEQ ID NO: 21.

In some cases, the conformational epitope comprises at least one peptide sequence within D2 domain and at least one peptide sequence within D3 domain of LILRB1. In such cases, D2 domain of LILRB3 comprises an amino acid region that is equivalent to amino acid residues 111-223 of SEQ ID NO: 21 and D3 domain of LILRB3 comprises an amino acid region that is equivalent to amino acid residues 224-323 of SEQ ID NO: 21. In some cases, D2 domain comprises a sequence having about 90% or more sequence identity to amino acid residues 111-223 of SEQ ID NO: 21. In some cases, D3 domain comprises a sequence having about 90% or more sequence identity to amino acid residues 224-323 of SEQ ID NO: 21.

In some cases, the conformational epitope comprises at least one peptide sequence within D3 domain and at least one peptide sequence within D4 domain of LILRB3. In such cases, D3 domain of LILRB3 comprises an amino acid region that is equivalent to amino acid residues 224-323 of SEQ ID NO: 21 and D4 domain of LILRB3 comprises an amino acid region that is equivalent to amino acid residues 324-419 of SEQ ID NO: 21. In some cases, D3 domain comprises a sequence having about 90% or more sequence identity to amino acid residues 224-323 of SEQ ID NO: 21. In some cases, D4 domain comprises a sequence having about 90% or more sequence identity to amino acid residues 324-419 of SEQ ID NO: 21.

In some cases, the conformational epitope comprises at least one peptide sequence within D4 domain and at least one peptide sequence within the region between the C-terminus of D4 domain and the N-terminus of the transmembrane domain of LILRB3. In such cases, D4 domain of LILRB3 comprises an amino acid region that is equivalent to amino acid residues 324-419 of SEQ ID NO: 21 and the region between the C-terminus of D4 domain and the N-terminus of the transmembrane domain of LILRB3 comprises an amino acid region that is equivalent to amino acid residues 420-443 of SEQ ID NO: 21. In some cases, D4 domain comprises a sequence having about 90% or more sequence identity to amino acid residues 324-419 of SEQ ID NO: 21 and the region between the C-terminus of D4 domain and the N-terminus of the transmembrane domain of LILRB3 comprises a sequence having about 90% or more sequence identity to amino acid residues 420-443 of SEQ ID NO: 21.

In some embodiments, the anti-LILRB3 antibodies or binding fragments thereof weakly bind to an epitope on the extracellular domain of LILRB1, LILRB2, LILRB4, LILRB5, or one or more LILRAs. As used herein, the term “weakly” refers to a reduced binding affinity toward a non-LILRB2 protein (e.g., LILRB1, LILRB2, LILRB4, LILRB5, or one or more LILRAs) relative to binding affinity to LILRB3. In some instances, the reduced binding affinity is about a 10-fold lower in binding affinity toward a non-LILRB2 protein (e.g., LILRB1, LILRB2, LILRB4, LILRB5, or one or more LILRAs)relative to the binding affinity to LILRB3. In some cases, the reduced binding affinity is about a 15-fold lower in binding affinity, about a 20-fold lower in binding affinity, about a 30-fold lower in binding affinity, about a 40-fold lower in binding affinity, about a 50-fold lower in binding affinity, about a 100-fold lower in binding affinity, or more.

In some instances, the anti-LILRB3 antibodies or binding fragments thereof inhibit binding of a ligand of LILRB3 to LILRB3 by at least 10%, 20%, 30%, 40%, 50%, 60%, 70%, 80%, 90%, 95%, or more. In some cases, the anti-LILRB3 antibodies or binding fragments thereof inhibit binding of a ligand of LILRB3 to LILRB3 by at least 10% or more. In some cases, the anti-LILRB3 antibodies or binding fragments thereof inhibit binding of a ligand of LILRB3 to LILRB3 by at least 20% or more. In some cases, the anti-LILRB3 antibodies or binding fragments thereof inhibit binding of a ligand of LILRB3 to LILRB3 by at least 30% or more. In some cases, the anti-LILRB3 antibodies or binding fragments thereof inhibit binding of a ligand of LILRB3 to LILRB3 by at least 40% or more. In some cases, the anti-LILRB3 antibodies or binding fragments thereof inhibit binding of a ligand of LILRB3 to LILRB3 by at least 50% or more. In some cases, the anti-LILRB3 antibodies or binding fragments thereof inhibit binding of a ligand of LILRB3 to LILRB3 by at least 60% or more. In some cases, the anti-LILRB3 antibodies or binding fragments thereof inhibit binding of a ligand of LILRB3 to LILRB3 by at least 70% or more. In some cases, the anti-LILRB3 antibodies or binding fragments thereof inhibit binding of a ligand of LILRB3 to LILRB3 by at least 80% or more. In some cases, the anti-LILRB3 antibodies or binding fragments thereof inhibit binding of a ligand of LILRB3 to LILRB3 by at least 90% or more. In some cases, the anti-LILRB3 antibodies or binding fragments thereof inhibit binding of a ligand of LILRB3 to LILRB3 by at least 95% or more. In some cases, the ligand comprises a pathogen such as Staphylococcus aureus.

In some instances, the anti-LILRB3 antibodies or binding fragments thereof inhibit binding of a ligand of LILRB3 to LILRB3 by about 2-fold, 3-fold, 4-fold, 5-fold, 6-fold, 7-fold, 8-fold, 9-fold, 10-fold, or more. In some instances, the anti-LILRB3 antibodies or binding fragments thereof inhibit binding of a ligand of LILRB3 to LILRB3 by about 2-fold or more. In some instances, the anti-LILRB3 antibodies or binding fragments thereof inhibit binding of a ligand of LILRB3 to LILRB3 by about 3-fold or more. In some instances, the anti-LILRB3 antibodies or binding fragments thereof inhibit binding of a ligand of LILRB3 to LILRB3 by about 4-fold or more. In some instances, the anti-LILRB3 antibodies or binding fragments thereof inhibit binding of a ligand of LILRB3 to LILRB3 by about 5-fold or more. In some instances, the anti-LILRB3 antibodies or binding fragments thereof inhibit binding of a ligand of LILRB3 to LILRB3 by about 6-fold or more. In some instances, the anti-LILRB3 antibodies or binding fragments thereof inhibit binding of a ligand of LILRB3 to LILRB3 by about 7-fold or more. In some instances, the anti-LILRB3 antibodies or binding fragments thereof inhibit binding of a ligand of LILRB3 to LILRB3 by about 8-fold or more. In some instances, the anti-LILRB3 antibodies or binding fragments thereof inhibit binding of a ligand of LILRB3 to LILRB3 by about 9-fold or more. In some instances, the anti-LILRB3 antibodies or binding fragments thereof inhibit binding of a ligand of LILRB3 to LILRB3 by about 10-fold or more. In some cases, the ligand comprises a pathogen such as Staphylococcus aureus.

In some embodiments, an anti-LILRB3 antibody or binding fragment thereof described above comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. In some cases, the anti-LILRB3 antibody or binding fragment thereof comprises a humanized antibody or binding fragment thereof. In some cases, the anti-LILRB3 antibody or binding fragment thereof comprises a murine antibody or binding fragment thereof. In some cases, the anti-LILRB3 antibody or binding fragment thereof comprises a chimeric antibody or binding fragment thereof. In some cases, the anti-LILRB3 antibody or binding fragment thereof comprises a monoclonal antibody or binding fragment thereof. In some cases, the anti-LILRB3 antibody or binding fragment thereof comprises a bispecific antibody or binding fragment thereof. In some cases, the anti-LILRB3 antibody or binding fragment thereof comprises a monovalent Fab′, a divalent Fab2, or F(ab)′3 fragments. In some cases, the anti-LILRB3 antibody or binding fragment thereof comprises a single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, or camelid antibody or binding fragment thereof. In some cases, the anti-LILRB3 antibody or binding fragment thereof comprises a chemically modified derivative thereof.

In some embodiments, also described herein include a vector comprising a nucleic acid molecule that encodes an anti-LILRB3 antibody or binding fragment thereof.

In some embodiments, further described herein include a host cell comprising a nucleic acid molecule that encodes an anti-LILRB3 antibody or binding fragment thereof.

Anti-LILRB4 Antibodies. In some embodiments, described herein are anti-LILRB4 antibodies or binding fragments thereof that specifically bind to an epitope on the extracellular domain of LILRB4, for the treatment of a proliferative disease, an infectious disease, or an autoimmune disease. In some instances, the epitope comprises a peptide sequence within domain D1 or D2 of LILRB4. In some cases, the epitope comprises a peptide sequence within domain D1. In some cases, the epitope comprises a peptide sequence within domain D2.

In other instances, the epitope comprises at least one peptide sequence within the region between the C-terminus of D2 domain and the N-terminus of the transmembrane domain of LILRB4.

In additional instances, the epitope comprises a domain junction. For example in some cases, the epitope comprises at least one peptide sequence within the D1 domain and at least one peptide sequence within the D2 domain of LILRB4. In additional cases, the epitope comprises two or more peptide sequences within D1 domain and two or more peptide sequences within D2 domain. In further cases, the epitope comprises two or more peptide sequences within D2 domain and two or more peptide sequences within the region between the C-terminus of D2 domain and the N-terminus of the transmembrane domain of LILRB4.

In some embodiments, LILRB4 comprises 5 isoforms (see e.g., SEQ ID NOs: 24-28 in Table 1). In some cases, the anti-LILRB4 antibodies or binding fragments thereof specifically bind to an epitope on the extracellular domain of LILRB4 isoform 1 (SEQ ID NO: 24). In some cases, the anti-LILRB4 antibodies or binding fragments thereof specifically bind to an epitope on the extracellular domain of LILRB4 isoform 2 (SEQ ID NO: 25). In some cases, the anti-LILRB4 antibodies or binding fragments thereof specifically bind to an epitope on the extracellular domain of LILRB4 isoform 3 (SEQ ID NO: 26). In some cases, the anti-LILRB4 antibodies or binding fragments thereof specifically bind to an epitope on the extracellular domain of LILRB4 isoform 4 (SEQ ID NO: 27). In some cases, the anti-LILRB4 antibodies or binding fragments thereof specifically bind to an epitope on the extracellular domain of LILRB4 isoform 5 (SEQ ID NO: 28).

In some cases, the anti-LILRB4 antibodies or binding fragments thereof specifically bind to an epitope on the extracellular domain of LILRB4 comprising a sequence as set forth in SEQ ID NO: 19.

In some instances, D1 domain of LILRB4 comprises an amino acid region that is equivalent to amino acid residues 22-121 of SEQ ID NO: 24. In some cases, an anti-LILRB4 antibody or binding fragment thereof specifically binds to an epitope within D1 in which the amino acid sequence of D1 is equivalent to amino acid residues 22-121 of SEQ ID NO: 24.

In some instances, D2 domain of LILRB4 comprises an amino acid region that is equivalent to amino acid residues 122-218 of SEQ ID NO: 24. In some cases, an anti-LILRB4 antibody or binding fragment thereof specifically binds to an epitope within D2 in which the amino acid sequence of D2 is equivalent to amino acid residues 122-218 of SEQ ID NO: 24.

In some instances, the region between the C-terminus of D2 domain and the N-terminus of the transmembrane domain of LILRB4 comprises an amino acid region that is equivalent to amino acid residues 219-259 of SEQ ID NO: 24. In some cases, an anti-LILRB4 antibody or binding fragment thereof specifically binds to an epitope within the region between the C-terminus of D2 domain and the N-terminus of the transmembrane domain of LILRB4 in which the amino acid region is equivalent to amino acid residues 219-259 of SEQ ID NO: 24.

In some instances, the epitope is a conformational epitope. In some instances, the conformational epitope comprises at least one peptide sequence within D1 or D2. In some cases, the conformational epitope comprises at least one peptide sequence within D1 domain and at least one peptide sequence within D2 domain of LILRB4. In such cases, D1 domain of LILRB4 comprises an amino acid region that is equivalent to amino acid residues 22-121 of SEQ ID NO: 24 and D2 domain of LILRB4 comprises an amino acid region that is equivalent to amino acid residues 122-218 of SEQ ID NO: 24.

In some cases, the conformational epitope comprises at least one peptide sequence within D2 domain and at least one peptide sequence within the region between the C-terminus of D2 domain and the N-terminus of the transmembrane domain of LILRB4. In such cases, D2 domain of LILRB4 comprises an amino acid region that is equivalent to amino acid residues 122-218 of SEQ ID NO: 24 and the region between the C-terminus of D2 domain and the N-terminus of the transmembrane domain of LILRB4 comprises an amino acid region that is equivalent to amino acid residues 219-259 of SEQ ID NO: 24.

In some embodiments, the anti-LILRB4 antibodies or binding fragments thereof weakly bind to an epitope on the extracellular domain of LILRB1, LILRB2, LILRB3, LILRB5, or one or more LILRAs. As used herein, the term “weakly” refers to a reduced binding affinity toward a non-LILRB4 protein (e.g., LILRB1, LILRB2, LILRB3, LILRB5, or one or more LILRAs) relative to binding affinity to LILRB4. In some instances, the reduced binding affinity is about a 10-fold lower in binding affinity toward a non-LILRB4 protein (e.g., LILRB1, LILRB2, LILRB3, LILRB5, or one or more LILRAs) relative to the binding affinity to LILRB4. In some cases, the reduced binding affinity is about a 15-fold lower in binding affinity, about a 20-fold lower in binding affinity, about a 30-fold lower in binding affinity, about a 40-fold lower in binding affinity, about a 50-fold lower in binding affinity, about a 100-fold lower in binding affinity, or more.

In some instances, the anti-LILRB4 antibodies or binding fragments thereof inhibit binding of a ligand of LILRB4 to LILRB4 by at least 10%, 20%, 30%, 40%, 50%, 60%, 70%, 80%, 90%, 95%, or more. In some cases, the anti-LILRB4 antibodies or binding fragments thereof inhibit binding of a ligand of LILRB4 to LILRB4 by at least 10% or more. In some cases, the anti-LILRB4 antibodies or binding fragments thereof inhibit binding of a ligand of LILRB4 to LILRB4 by at least 20% or more. In some cases, the anti-LILRB4 antibodies or binding fragments thereof inhibit binding of a ligand of LILRB4 to LILRB4 by at least 30% or more. In some cases, the anti-LILRB4 antibodies or binding fragments thereof inhibit binding of a ligand of LILRB4 to LILRB4 by at least 40% or more. In some cases, the anti-LILRB4 antibodies or binding fragments thereof inhibit binding of a ligand of LILRB4 to LILRB4 by at least 50% or more. In some cases, the anti-LILRB4 antibodies or binding fragments thereof inhibit binding of a ligand of LILRB4 to LILRB4 by at least 60% or more. In some cases, the anti-LILRB4 antibodies or binding fragments thereof inhibit binding of a ligand of LILRB4 to LILRB4 by at least 70% or more. In some cases, the anti-LILRB4 antibodies or binding fragments thereof inhibit binding of a ligand of LILRB4 to LILRB4 by at least 80% or more. In some cases, the anti-LILRB4 antibodies or binding fragments thereof inhibit binding of a ligand of LILRB4 to LILRB4 by at least 90% or more. In some cases, the anti-LILRB4 antibodies or binding fragments thereof inhibit binding of a ligand of LILRB4 to LILRB4 by at least 95% or more. In some cases, the ligand comprises CD166.

In some instances, the anti-LILRB4 antibodies or binding fragments thereof inhibit binding of a ligand of LILRB4 to LILRB4 by about 2-fold, 3-fold, 4-fold, 5-fold, 6-fold, 7-fold, 8-fold, 9-fold, 10-fold, or more. In some instances, the anti-LILRB4 antibodies or binding fragments thereof inhibit binding of a ligand of LILRB4 to LILRB4 by about 2-fold or more. In some instances, the anti-LILRB4 antibodies or binding fragments thereof inhibit binding of a ligand of LILRB4 to LILRB4 by about 3-fold or more. In some instances, the anti-LILRB4 antibodies or binding fragments thereof inhibit binding of a ligand of LILRB4 to LILRB4 by about 4-fold or more. In some instances, the anti-LILRB4 antibodies or binding fragments thereof inhibit binding of a ligand of LILRB4 to LILRB4 by about 5-fold or more. In some instances, the anti-LILRB4 antibodies or binding fragments thereof inhibit binding of a ligand of LILRB4 to LILRB4 by about 6-fold or more. In some instances, the anti-LILRB4 antibodies or binding fragments thereof inhibit binding of a ligand of LILRB4 to LILRB4 by about 7-fold or more. In some instances, the anti-LILRB4 antibodies or binding fragments thereof inhibit binding of a ligand of LILRB4 to LILRB4 by about 8-fold or more. In some instances, the anti-LILRB4 antibodies or binding fragments thereof inhibit binding of a ligand of LILRB4 to LILRB4 by about 9-fold or more. In some instances, the anti-LILRB4 antibodies or binding fragments thereof inhibit binding of a ligand of LILRB4 to LILRB4 by about 10-fold or more. In some cases, the ligand comprises CD166.

In some embodiments, an anti-LILRB4 antibody or binding fragment thereof described above comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. In some cases, the anti-LILRB4 antibody or binding fragment thereof comprises a humanized antibody or binding fragment thereof. In some cases, the anti-LILRB4 antibody or binding fragment thereof comprises a murine antibody or binding fragment thereof. In some cases, the anti-LILRB4 antibody or binding fragment thereof comprises a chimeric antibody or binding fragment thereof. In some cases, the anti-LILRB4 antibody or binding fragment thereof comprises a monoclonal antibody or binding fragment thereof. In some cases, the anti-LILRB4 antibody or binding fragment thereof comprises a bispecific antibody or binding fragment thereof. In some cases, the anti-LILRB4 antibody or binding fragment thereof comprises a monovalent Fab′, a divalent Fab2, or F(ab)′3 fragments. In some cases, the anti-LILRB4 antibody or binding fragment thereof comprises a single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, or camelid antibody or binding fragment thereof. In some cases, the anti-LILRB4 antibody or binding fragment thereof comprises a chemically modified derivative thereof.

In some embodiments, also described herein include a vector comprising a nucleic acid molecule that encodes an anti-LILRB4 antibody or binding fragment thereof.

In some embodiments, further described herein include a host cell comprising a nucleic acid molecule that encodes an anti-LILRB4 antibody or binding fragment thereof.

Anti-LILRB5 Antibodies. In some embodiments, described herein are anti-LILRB5 antibodies or binding fragments thereof that specifically bind to an epitope on the extracellular domain of LILRB5, for the treatment of a proliferative disease, an infectious disease, or an autoimmune disease. In some instances, the epitope comprises a peptide sequence within domain D1, D2, D3, or D4 of LILRB5. In some cases, the epitope comprises a peptide sequence within domain D1. In some cases, the epitope comprises a peptide sequence within domain D2. In some cases, the epitope comprises a peptide sequence within domain D3. In some cases, the epitope comprises a peptide sequence within domain D4.

In other instances, the epitope comprises at least one peptide sequence within the region between the C-terminus of D4 domain and the N-terminus of the transmembrane domain of LILRB5.

In additional instances, the epitope comprises a domain junction. For example in some cases, the epitope comprises at least one peptide sequence within the D1 domain and at least one peptide sequence within the D2 domain of LILRB5. In other cases, the epitope comprises at least one peptide sequence within the D2 domain and at least one peptide sequence within the D3 domain of LILRB5. In additional cases, the epitope comprises at least one peptide sequence within the D3 domain and at least one peptide sequence within the D4 domain of LILRB5. In further cases, the epitope comprises at least one peptide sequence within the D4 domain and at least one peptide sequence within the region between the C-terminus of D4 domain and the N-terminus of the transmembrane domain of LILRB5. In some cases, the epitope comprises (i) two or more peptide sequences within D1 domain and two or more peptide sequences within D2 domain; (ii) two or more peptide sequences within D2 domain and two or more peptide sequences within D3 domain; (iii) two or more peptide sequences within D3 domain and two or more peptide sequences within D4 domain; or (iv) two or more peptide sequences within D4 domain and two or more peptide sequences within the region between the C-terminus of D4 domain and the N-terminus of the transmembrane domain of LILRB5.

In some embodiments, LILRB5 comprises 4 isoforms (see e.g., SEQ ID NOs: 29-32 in Table 1). In some cases, the anti-LILRB5 antibodies or binding fragments thereof specifically bind to an epitope on the extracellular domain of LILRB5 isoform 1 (SEQ ID NO: 29). In some cases, the anti-LILRB5 antibodies or binding fragments thereof specifically bind to an epitope on the extracellular domain of LILRB5 isoform 2 (SEQ ID NO: 30). In some cases, the anti-LILRB5 antibodies or binding fragments thereof specifically bind to an epitope on the extracellular domain of LILRB5 isoform 3 (SEQ ID NO: 31). In some cases, the anti-LILRB5 antibodies or binding fragments thereof specifically bind to an epitope on the extracellular domain of LILRB5 isoform 4 (SEQ ID NO: 32).

In some cases, the anti-LILRB5 antibodies or binding fragments thereof specifically bind to an epitope on the extracellular domain of LILRB5 comprising a sequence as set forth in SEQ ID NO: 20.

In some instances, D1 domain of LILRB5 comprises an amino acid region that is equivalent to amino acid residues 27-110 of SEQ ID NO: 20. In some cases, an anti-LILRB5 antibody or binding fragment thereof specifically binds to an epitope within D1 in which the amino acid sequence of D1 is equivalent to amino acid residues 27-110 of SEQ ID NO: 20.

In some instances, D2 domain of LILRB5 comprises an amino acid region that is equivalent to amino acid residues 111-223 of SEQ ID NO: 20. In some cases, an anti-LILRB5 antibody or binding fragment thereof specifically binds to an epitope within D2 in which the amino acid sequence of D2 is equivalent to amino acid residues 111-223 of SEQ ID NO: 20.

In some instances, D3 domain of LILRB5 comprises an amino acid region that is equivalent to amino acid residues 224-320 of SEQ ID NO: 20. In some cases, an anti-LILRB5 antibody or binding fragment thereof specifically binds to an epitope within D3 in which the amino acid sequence of D3 is equivalent to amino acid residues 224-320 of SEQ ID NO: 20.

In some instances, D4 domain of LILRB5 comprises an amino acid region that is equivalent to amino acid residues 321-418 of SEQ ID NO: 20. In some cases, an anti-LILRB5 antibody or binding fragment thereof specifically binds to an epitope within D4 in which the amino acid sequence of D4 is equivalent to amino acid residues 321-418 of SEQ ID NO: 20.

In some instances, the region between the C-terminus of D4 domain and the N-terminus of the transmembrane domain of LILRB5 comprises an amino acid region that is equivalent to amino acid residues 419-458 of SEQ ID NO: 20. In some cases, an anti-LILRB5 antibody or binding fragment thereof specifically binds to an epitope within the region between the C-terminus of D4 domain and the N-terminus of the transmembrane domain of LILRB5 in which the amino acid sequence is equivalent to amino acid residues 419-458 of SEQ ID NO: 20.

In some instances, the epitope is a conformational epitope. In some instances, the conformational epitope comprises at least one peptide sequence within D1, D2, D3, or D4. In some cases, the conformational epitope comprises at least one peptide sequence within D1 domain and at least one peptide sequence within D2 domain of LILRB5. In such cases, D1 domain of LILRB5 comprises an amino acid region that is equivalent to amino acid residues 27-110 of SEQ ID NO: 20 and D2 domain of LILRB2 comprises an amino acid region that is equivalent to amino acid residues 111-223 of SEQ ID NO: 20.

In some cases, the conformational epitope comprises at least one peptide sequence within D2 domain and at least one peptide sequence within D3 domain of LILRB5. In such cases, D2 domain of LILRB5 comprises an amino acid region that is equivalent to amino acid residues 111-223 of SEQ ID NO: 20 and D3 domain of LILRB5 comprises an amino acid region that is equivalent to amino acid residues 224-320 of SEQ ID NO: 20.

In some cases, the conformational epitope comprises at least one peptide sequence within D3 domain and at least one peptide sequence within D4 domain of LILRB5. In such cases, D3 domain of LILRB5 comprises an amino acid region that is equivalent to amino acid residues 224-320 of SEQ ID NO: 20 and D4 domain of LILRB5 comprises an amino acid region that is equivalent to amino acid residues 321-418 of SEQ ID NO: 20.

In some cases, the conformational epitope comprises at least one peptide sequence within D4 domain and at least one peptide sequence within the region between the C-terminus of D4 domain and the N-terminus of the transmembrane domain of LILRB5. In such cases, D4 domain of LILRB5 comprises an amino acid region that is equivalent to amino acid residues 321-418 of SEQ ID NO: 20 and the region between the C-terminus of D4 domain and the N-terminus of the transmembrane domain of LILRB5 comprises an amino acid region that is equivalent to amino acid residues 419-458 of SEQ ID NO: 20.

In some embodiments, the anti-LILRB5 antibodies or binding fragments thereof weakly bind to an epitope on the extracellular domain of LILRB1, LILRB2, LILRB3, LILRB4, or one or more LILRAs. As used herein, the term “weakly” refers to a reduced binding affinity toward a non-LILRB1 protein (e.g., LILRB1, LILRB2, LILRB3, LILRB4, or one or more LILRAs) relative to the binding affinity to LILRB5. In some instances, the reduced binding affinity is about a 10-fold lower in binding affinity toward a non-LILRB1 protein (e.g., LILRB1, LILRB2, LILRB3, LILRB4, or one or more LILRAs) relative to the binding affinity to LILRB5. In some cases, the reduced binding affinity is about a 15-fold lower in binding affinity, about a 20-fold lower in binding affinity, about a 30-fold lower in binding affinity, about a 40-fold lower in binding affinity, about a 50-fold lower in binding affinity, about a 100-fold lower in binding affinity, or more.

In some instances, the anti-LILRB5 antibodies or binding fragments thereof inhibit binding of a ligand of LILRB5 to LILRB5 by at least 10%, 20%, 30%, 40%, 50%, 60%, 70%, 80%, 90%, 95%, or more. In some cases, the anti-LILRB5 antibodies or binding fragments thereof inhibit binding of a ligand of LILRB5 to LILRB5 by at least 10% or more. In some cases, the anti-LILRB5 antibodies or binding fragments thereof inhibit binding of a ligand of LILRB5 to LILRB5 by at least 20% or more. In some cases, the anti-LILRB5 antibodies or binding fragments thereof inhibit binding of a ligand of LILRB5 to LILRB5 by at least 30% or more. In some cases, the anti-LILRB5 antibodies or binding fragments thereof inhibit binding of a ligand of LILRB5 to LILRB5 by at least 40% or more. In some cases, the anti-LILRB5 antibodies or binding fragments thereof inhibit binding of a ligand of LILRB5 to LILRB5 by at least 50% or more. In some cases, the anti-LILRB5 antibodies or binding fragments thereof inhibit binding of a ligand of LILRB5 to LILRB5 by at least 60% or more. In some cases, the anti-LILRB5 antibodies or binding fragments thereof inhibit binding of a ligand of LILRB5 to LILRB5 by at least 70% or more. In some cases, the anti-LILRB5 antibodies or binding fragments thereof inhibit binding of a ligand of LILRB5 to LILRB5 by at least 80% or more. In some cases, the anti-LILRB5 antibodies or binding fragments thereof inhibit binding of a ligand of LILRB5 to LILRB5 by at least 90% or more. In some cases, the anti-LILRB5 antibodies or binding fragments thereof inhibit binding of a ligand of LILRB5 to LILRB5 by at least 95% or more. In some cases, the ligand of LILRB5 is a natural ligand. In some cases, the ligand comprises HLA-B7, HLA-B27, ANGPTL1, ANGPTL2, ANGPTL3, ANGPTL4, ANGPTL5, ANGPTL6, ANGPTL7, or ANGPTL8.

In some instances, the anti-LILRB5 antibodies or binding fragments thereof inhibit binding of a ligand of LILRB5 to LILRB5 by about 2-fold, 3-fold, 4-fold, 5-fold, 6-fold, 7-fold, 8-fold, 9-fold, 10-fold, or more. In some instances, the anti-LILRB5 antibodies or binding fragments thereof inhibit binding of a ligand of LILRB5 to LILRB5 by about 2-fold or more. In some instances, the anti-LILRB5 antibodies or binding fragments thereof inhibit binding of a ligand of LILRB5 to LILRB5 by about 3-fold or more. In some instances, the anti-LILRB5 antibodies or binding fragments thereof inhibit binding of a ligand of LILRB5 to LILRB5 by about 4-fold or more. In some instances, the anti-LILRB5 antibodies or binding fragments thereof inhibit binding of a ligand of LILRB5 to LILRB5 by about 5-fold or more. In some instances, the anti-LILRB5 antibodies or binding fragments thereof inhibit binding of a ligand of LILRB5 to LILRB5 by about 6-fold or more. In some instances, the anti-LILRB5 antibodies or binding fragments thereof inhibit binding of a ligand of LILRB5 to LILRB5 by about 7-fold or more. In some instances, the anti-LILRB5 antibodies or binding fragments thereof inhibit binding of a ligand of LILRB5 to LILRB5 by about 8-fold or more. In some instances, the anti-LILRB5 antibodies or binding fragments thereof inhibit binding of a ligand of LILRB5 to LILRB5 by about 9-fold or more. In some instances, the anti-LILRB5 antibodies or binding fragments thereof inhibit binding of a ligand of LILRB5 to LILRB5 by about 10-fold or more. In some cases, the ligand of LILRB5 is a natural ligand. In some cases, the ligand comprises HLA-B7, HLA-B27, ANGPTL1, ANGPTL2, ANGPTL3, ANGPTL4, ANGPTL5, ANGPTL6, ANGPTL7, or ANGPTL8.

In some embodiments, an anti-LILRB5 antibody or binding fragment thereof described above comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. In some cases, the anti-LILRB5 antibody or binding fragment thereof comprises a humanized antibody or binding fragment thereof. In some cases, the anti-LILRB5 antibody or binding fragment thereof comprises a murine antibody or binding fragment thereof. In some cases, the anti-LILRB5 antibody or binding fragment thereof comprises a chimeric antibody or binding fragment thereof. In some cases, the anti-LILRB5 antibody or binding fragment thereof comprises a monoclonal antibody or binding fragment thereof. In some cases, the anti-LILRB5 antibody or binding fragment thereof comprises a bispecific antibody or binding fragment thereof. In some cases, the anti-LILRB5 antibody or binding fragment thereof comprises a monovalent Fab′, a divalent Fab2, or F(ab)′3 fragments. In some cases, the anti-LILRB5 antibody or binding fragment thereof comprises a single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, or camelid antibody or binding fragment thereof. In some cases, the anti-LILRB5 antibody or binding fragment thereof comprises a chemically modified derivative thereof.

In some embodiments, also described herein include a vector comprising a nucleic acid molecule that encodes an anti-LILRB5 antibody or binding fragment thereof.

In some embodiments, further described herein include a host cell comprising a nucleic acid molecule that encodes an anti-LILRB5 antibody or binding fragment thereof.

### Pan Anti-LILRB Antibodies

In some embodiments, described herein are pan anti-LILRB antibodies or binding fragments thereof. In some instances, a pan anti-LILRB antibody or binding fragments thereof specifically binds to an epitope on the extracellular domain of LILRB1 and at least an epitope on the extracellular domain of LILRB2, LILRB3, LILRB4, LILRB5, or a combination thereof, for the treatment of a proliferative disease, an infectious disease, or a neurological disease or disorder. In some instances, the pan antibody or binding fragment thereof specifically binds to an epitope on the extracellular domain of LILRB1 and at least an epitope on the extracellular domain of LILRB2, LILRB3, LILRB4, or a combination thereof. In some instances, the pan antibody or binding fragment thereof specifically binds to an epitope on the extracellular domain of LILRB1 and at least an epitope on the extracellular domain of LILRB2, LILRB3, or a combination thereof. In some instances, the pan antibody or binding fragment thereof specifically binds to an epitope on the extracellular domain of LILRB1 and at least an epitope on the extracellular domain of LILRB2, LILRB4, or a combination thereof. In some instances, the pan antibody or binding fragment thereof specifically binds to an epitope on the extracellular domain of LILRB1 and at least an epitope on the extracellular domain of LILRB3, LILRB4, or a combination thereof.

In some embodiments, also described herein is a pan anti-LILRB2 antibody or binding fragments thereof that specifically bind to an epitope on the extracellular domain of LILRB2 and at least an epitope on the extracellular domain of LILRB3, LILRB4, LILRB5, or a combination thereof, for the treatment of a proliferative disease, an infectious disease, or a neurological disease or disorder. In some cases, the pan antibody or binding fragment thereof specifically binds to an epitope on the extracellular domain of LILRB2 and at least an epitope on the extracellular domain of LILRB3, LILRB4, or a combination thereof. In some cases, the pan antibody or binding fragment thereof specifically binds to an epitope on the extracellular domain of LILRB2, at least an epitope on the extracellular domain of LILRB3, and at least an epitope on the extracellular domain of LILRB4.

In some instances, further described herein is a pan anti-LILRB antibody or binding fragments thereof specifically binds to an epitope on the extracellular domain of LILRB3 and at least an epitope on the extracellular domain of LILRB1, LILRB2, LILRB4, LILRB5, or a combination thereof, for the treatment of a proliferative disease, an infectious disease, or an autoimmune disease. In some instances, the pan antibody or binding fragment thereof specifically binds to an epitope on the extracellular domain of LILRB3 and at least an epitope on the extracellular domain of LILRB1, LILRB2, LILRB4, or a combination thereof. In some instances, the pan antibody or binding fragment thereof specifically binds to an epitope on the extracellular domain of LILRB3 and at least an epitope on the extracellular domain of LILRB1, LILRB2, or a combination thereof. In some instances, the pan antibody or binding fragment thereof specifically binds to an epitope on the extracellular domain of LILRB3 and at least an epitope on the extracellular domain of LILRB1, LILRB4, or a combination thereof. In some instances, the pan antibody or binding fragment thereof specifically binds to an epitope on the extracellular domain of LILRB3, at least an epitope on the extracellular domains of LILRB1, at least an epitope on the extracellular domains of LILRB2, and at least an epitope on the extracellular domains of LILRB4.

In some embodiments, additionally described herein is a pan anti-LILRB4 antibody or binding fragments thereof that specifically bind to an epitope on the extracellular domain of LILRB4 and at least an epitope on the extracellular domain of LILRB1, LILRB3, LILRB5, or a combination thereof, for the treatment of a proliferative disease, an infectious disease, or an autoimmune disease. In some instances, the pan antibody or binding fragment thereof specifically binds to an epitope on the extracellular domain of LILRB4 and at least an epitope on the extracellular domain of LILRB1, LILRB3, or a combination thereof. In some cases, the pan antibody or binding fragment thereof specifically binds to an epitope on the extracellular domain of LILRB4, at least an epitope on the extracellular domain of LILRB1, and at least an epitope on the extracellular domain of LILRB3.

In some embodiments, the epitope of LILRB1 comprises (i) a peptide sequence within domain D1, D2, D3, or D4 of LILRB1; (ii) at least one peptide sequence within the region between the C-terminus of D4 domain and the N-terminus of the transmembrane domain of LILRB1; (iii) at least one peptide sequence within the D1 domain and at least one peptide sequence within the D2 domain of LILRB1; (iv) at least one peptide sequence within the D2 domain and at least one peptide sequence within the D3 domain of LILRB1; (v) at least one peptide sequence within the D3 domain and at least one peptide sequence within the D4 domain of LILRB1; or (vi) at least one peptide sequence within the D4 domain and at least one peptide sequence within the region between the C-terminus of D4 domain and the N-terminus of the transmembrane domain of LILRB1. In some instances, the epitope is a conformational epitope. In some cases, the epitope comprises a peptide sequence from D3 of LILRB1, or a peptide sequence from D4 of LILRB1, or two or more peptide sequences within D3 and D4 of LILRB1. In some cases, D1 domain comprises an amino acid region that is equivalent to amino acid residues 24-115 of SEQ ID NO: 1. In some cases, D2 domain comprises an amino acid region that is equivalent to amino acid residues 116-221 of SEQ ID NO: 1. In some cases, D3 domain comprises an amino acid region that is equivalent to amino acid residues 222-312 of SEQ ID NO: 1. In some cases, D4 domain comprises an amino acid region that is equivalent to amino acid residues 313-409 of SEQ ID NO: 1. In some cases, the region between the C-terminus of D4 domain and the N-terminus of the transmembrane domain of LILRB1 comprises an amino acid region that is equivalent to amino acid residues 410-461 of SEQ ID NO: 1

In some instances, the epitope of LILRB2 comprises (i) a peptide sequence within domain D1, D2, D3, or D4 of LILRB2; (ii) at least one peptide sequence within the region between the C-terminus of D4 domain and the N-terminus of the transmembrane domain of LILRB2; (iii) at least one peptide sequence within the D1 domain and at least one peptide sequence within the D2 domain of LILRB2; (iv) at least one peptide sequence within the D2 domain and at least one peptide sequence within the D3 domain of LILRB2; (v) at least one peptide sequence within the D3 domain and at least one peptide sequence within the D4 domain of LILRB2; or (vi) at least one peptide sequence within the D4 domain and at least one peptide sequence within the region between the C-terminus of D4 domain and the N-terminus of the transmembrane domain of LILRB2. In some instances, the epitope is a conformational epitope. In some cases, the epitope comprises a peptide sequence from D3 of LILRB2, or a peptide sequence from D4 of LILRB2, or two or more peptide sequences within D3 and D4 of LILRB2. In some cases, D1 domain comprises an amino acid region that is equivalent to amino acid residues 22-110 of SEQ ID NO: 9. In some cases, D2 domain comprises an amino acid region that is equivalent to amino acid residues 111-229 of SEQ ID NO: 9. In some cases, D3 domain comprises an amino acid region that is equivalent to amino acid residues 230-318 of SEQ ID NO: 9. In some cases, D4 domain of LILRB2 comprises an amino acid region that is equivalent to amino acid residues 319-419 of SEQ ID NO: 9. In some cases, the region between the C-terminus of D4 domain and the N-terminus of the transmembrane domain of LILRB2 comprises an amino acid region that is equivalent to amino acid residues 420-462 of SEQ ID NO: 9 or amino acid residues 420-461 of SEQ ID NO: 10.

In some instances, the epitope of LILRB3 comprises (i) a peptide sequence within domain D1, D2, D3, or D4 of LILRB3; (ii) at least one peptide sequence within the region between the C-terminus of D4 domain and the N-terminus of the transmembrane domain of LILRB3; (iii) at least one peptide sequence within the D1 domain and at least one peptide sequence within the D2 domain of LILRB3; (iv) at least one peptide sequence within the D2 domain and at least one peptide sequence within the D3 domain of LILRB3; (v) at least one peptide sequence within the D3 domain and at least one peptide sequence within the D4 domain of LILRB3; (vi) at least one peptide sequence within the D4 domain and at least one peptide sequence within the region between the C-terminus of D4 domain and the N-terminus of the transmembrane domain of LILRB3. In some instances, the epitope is a conformational epitope. In some instances, D1 domain comprises an amino acid region that is equivalent to amino acid residues 2-87 of SEQ ID NO: 17. In some cases, D1 domain comprises a sequence having about 90% or more (e.g., 90%, 95%, 96%, 97%, 98%, 99%, or more) sequence identity to amino acid residues 2-87 of SEQ ID NO: 17. In some instances, D2 domain comprises an amino acid region that is equivalent to amino acid residues 88-200 of SEQ ID NO: 17. In some cases, D2 domain comprises a sequence having about 90% or more (e.g., 90%, 95%, 96%, 97%, 98%, 99%, or more) sequence identity to amino acid residues 88-200 of SEQ ID NO: 17. In some instances, D3 domain comprises an amino acid region that is equivalent to amino acid residues 201-300 of SEQ ID NO: 17. In some cases, D3 domain comprises a sequence having about 90% or more (e.g., 90%, 95%, 96%, 97%, 98%, 99%, or more) sequence identity to amino acid residues 201-300 of SEQ ID NO: 17. In some instances, D4 domain comprises an amino acid region that is equivalent to amino acid residues 301-396 of SEQ ID NO: 17. In some cases, D4 domain comprises a sequence having about 90% or more (e.g., 90%, 95%, 96%, 97%, 98%, 99%, or more) sequence identity to amino acid residues 301-396 of SEQ ID NO: 17. In some cases, the region between the C-terminus of D4 domain and the N-terminus of the transmembrane domain of LILRB3 comprises an amino acid region that is equivalent to amino acid residues 397-420 of SEQ ID NO: 17. In some cases, the region between the C-terminus of D4 domain and the N-terminus of the transmembrane domain of LILRB3 comprises a sequence having about 90% or more (e.g., 90%, 95%, 96%, 97%, 98%, 99%, or more) sequence identity to amino acid residues 397-420 of SEQ ID NO: 17.

In some instances, the epitope of LILRB4 comprises (i) a peptide sequence within domain D1 or D2 of LILRB4; (ii) at least one peptide sequence within the region between the C-terminus of D2 domain and the N-terminus of the transmembrane domain of LILRB4; (iii) at least one peptide sequence within the D1 domain and at least one peptide sequence within the D2 domain of LILRB4; or (iv) at least one peptide sequence within the D2 domain and at least one peptide sequence within the region between the C-terminus of D2 domain and the N-terminus of the transmembrane domain of LILRB4. In some instances, the epitope is a conformational epitope. In some cases, D1 domain comprises an amino acid region that is equivalent to amino acid residues 1-100 of SEQ ID NO: 19. In some cases, D2 domain comprises an amino acid region that is equivalent to amino acid residues 101-197 of SEQ ID NO: 19. In some cases, the region between the C-terminus of D2 domain and the N-terminus of the transmembrane domain of LILRB4 comprises an amino acid region that is equivalent to amino acid residues 198-238 of SEQ ID NO: 19.

In some instances, the epitope of LILRB5 comprises (i) a peptide sequence within domain D1, D2, D3, or D4 of LILRB5; (ii) at least one peptide sequence within the region between the C-terminus of D4 domain and the N-terminus of the transmembrane domain of LILRB5; (iii) at least one peptide sequence within the D1 domain and at least one peptide sequence within the D2 domain of LILRB5; (iv) at least one peptide sequence within the D2 domain and at least one peptide sequence within the D3 domain of LILRB5; (v) at least one peptide sequence within the D3 domain and at least one peptide sequence within the D4 domain of LILRB5; or (vi) at least one peptide sequence within the D4 domain and at least one peptide sequence within the region between the C-terminus of D4 domain and the N-terminus of the transmembrane domain of LILRB5. In some instances, the epitope is a conformational epitope. In some cases, D1 domain comprises an amino acid region that is equivalent to amino acid residues 27-110 of SEQ ID NO: 20. In some cases, D2 domain comprises an amino acid region that is equivalent to amino acid residues 111-223 of SEQ ID NO: 20. In some cases, D3 domain comprises an amino acid region that is equivalent to amino acid residues 224-320 of SEQ ID NO: 20. In some cases, D4 domain comprises an amino acid region that is equivalent to amino acid residues 321-418 of SEQ ID NO: 20. In some cases, the region between the C-terminus of D2 domain and the N-terminus of the transmembrane domain of LILRB5 comprises an amino acid region that is equivalent to amino acid residues 419-458 of SEQ ID NO: 20.

In some embodiments, disclosed herein is a pan anti-LILRB antibody that specifically binds to at least one epitope on the extracellular domain of LILRB1, at least one epitope on the extracellular domain of LILRB2, and at least one epitope on the extracellular domain of LILRB3, for the treatment of a proliferative disease, an infectious disease, or a neurological disease or disorder. In some instances, the pan anti-LILRB antibody further binds specifically to an epitope on the extracellular domain of LILRB4 or an epitope on the extracellular domain of LILRB5. In some cases, the pan anti-LILRB antibody also binds specifically to LILRA1, LILRA3, LILRA5, LILRA6, or a combination thereof. In some cases, the pan anti-LILRB antibody also binds specifically to LILRA1, LILRA3, LILRA5, and LILRA6. In some cases, the pan anti-LILRB antibody also binds specifically to LILRA1, LILRA3, and LILRA6.

In some instances, the at least one epitope on the extracellular domain of LILRB2 comprises a peptide sequence within D3, a peptide sequence within D4, or a combination thereof.

In some instances, the at least one epitope on the extracellular domain of LILRB2 comprises a conformational epitope. In some cases, the conformational epitope is within D3 and comprises at least one peptide sequence; is within D4 and comprises at least one peptide sequence; or comprises at least one peptide sequence from D3 and at least one peptide sequence from D4. In some cases, the conformational epitope within D3 comprises at least one peptide sequence from region 223-231, region 236-248, region 258-262, region 269-290, or region 298-314, or a combination thereof, wherein the residue numberings correspond to positions 223-231, 236-248, 258-262, 269-290, and 298-314 of SEQ ID NO: 9. In some cases, the conformational epitope within D4 comprises at least one peptide sequence from region 336-340, region 362-368, region 379-393, region 400-403, or region 412-419, or a combination thereof, wherein the residue numberings correspond to positions 336-340, 362-368, 379-393, 400-403, and 412-419 of SEQ ID NO: 9.

In some instances, the pan antibody specifically binds to one or more LILRB3 isoforms selected from isoforms 1-3; or to a LILRB3 encoded by a sequence comprising at least 90%, 95%, 96%, 97%, 98%, 99%, or 100% sequence identity to SEQ ID NO: 40 or 41.

In some instances, the pan anti-LILRB antibody blocks HLA-G binding to a cell expressing a LILRB receptor.

In some instances, the pan anti-LILRB antibody blocks HLA-A binding to a cell expressing a LILRB receptor.

In some instances, the pan anti-LILRB antibody is 5G11.G8, 5G11.H6, 9C9.D3, 9C9.E6, 16D11.D10, or 11D9.E7. In some cases, the pan anti-LILRB antibody is 5G11.G8, 5G11.H6, 9C9.D3, 9C9.E6, or 16D11.D10. In some cases, the pan anti-LILRB antibody is 5G11.G8. In some cases, the pan anti-LILRB antibody is 5G11.H6. In some cases, the pan anti-LILRB antibody is 9C9.D3. In some cases, the pan anti-LILRB antibody is 9C9.E6. In some cases, the pan anti-LILRB antibody is 16D11.D10. In some cases, the pan anti-LILRB antibody is 16D11.D10.

In some embodiments, also described herein is an anti-LILRB antibody that specifically binds to an epitope within LILRB2 domain D3, an epitope within LILRB2 domain D4, or a combination thereof for the treatment of a proliferative disease, an infectious disease, or a neurological disease or disorder, wherein D3 comprises an amino acid region that corresponds to residues 230-318 of SEQ ID NO: 9, and D4 comprises an amino acid region that corresponds to residues 319-419 of SEQ ID NO: 9. In some instances, the anti-LILRB antibody specifically binds to an epitope within D3 or within D4. In some instances, the anti-LILRB antibody specifically binds to an epitope within D3 and an epitope within D4.

In some cases, the anti-LILRB antibody weakly binds to an epitope within LILRB2 domain D1 or D2. As used herein, the term “weakly” refers to a reduced binding affinity toward D1 and/or D2 relative to the binding affinity to D3 and/or D4. In some instances, the reduced binding affinity is about a 10-fold lower in binding affinity toward D1 and/or D2 relative to the binding affinity to D3 and/or D4. In some cases, the reduced binding affinity is about a 15-fold lower in binding affinity, about a 20-fold lower in binding affinity, about a 30-fold lower in binding affinity, about a 40-fold lower in binding affinity, about a 50-fold lower in binding affinity, about a 100-fold lower in binding affinity, or more.

In some instances, the anti-LILRB antibody specifically binds to a conformational epitope. In some cases, the conformational epitope is within D3 and comprises at least one peptide sequence. In some cases, the conformational epitope is within D4 and comprises at least one peptide sequence. In some cases, the conformational epitope comprises at least one peptide sequence from D3 and at least one peptide sequence from D4. In some cases, the conformational epitope within D3 comprises at least one peptide sequence from region 223-231, region 236-248, region 258-262, region 269-290, or region 298-314, or a combination thereof, wherein the residue numberings correspond to positions 223-231, 236-248, 258-262, 269-290, and 298-314 of SEQ ID NO: 9. In some cases, the conformational epitope within D4 comprises at least one peptide sequence from region 336-340, region 362-368, region 379-393, region 400-403, or region 412-419, or a combination thereof, wherein the residue numberings correspond to positions 336-340, 362-368, 379-393, 400-403, and 412-419 of SEQ ID NO: 9.

In some embodiments, the anti-LILRB antibody is a pan antibody that specifically binds to LILRB1, LILRB2, and LILRB3.

In some instances, the pan antibody specifically binds to one or more LILRB1 isoforms selected from isoforms 1-6; or to a LILRB1 encoded by a sequence comprising at least 90%, 95%, 96%, 97%, 98%, 99%, or 100% sequence identity to SEQ ID NOs: 33-35.

In some instances, the pan antibody specifically binds to one or more LILRB2 isoforms selected from isoforms 1-5; or to a LILRB2 encoded by a sequence comprising at least 90%, 95%, 96%, 97%, 98%, 99%, or 100% sequence identity to SEQ ID NOs: 36-39.

In some instances, the pan antibody specifically binds to one or more LILRB3 isoforms selected from isoforms 1-3; or to a LILRB3 encoded by a sequence comprising at least 90%, 95%, 96%, 97%, 98%, 99%, or 100% sequence identity to SEQ ID NO: 40 or 41.

In some instances, the pan antibody further binds specifically to LILRB5.

In additional instances, the pan antibody specifically binds to LILRA1, LILRA3, LILRA5, LILRA6, or a combination thereof. In such instances, the pan antibody specifically binds to LILRA1, LILRA3, LILRA5, and LILRA6. In such instances, the pan antibody specifically binds to LILRA1, LILRA3, and LILRA6.

In some embodiments, the anti-LILRB antibody is an anti-LILRB2 antibody that specifically binds to LILRB2 and weakly binds to an epitope on the extracellular domain of LILRB1, LILRB3, LILRB4, and LILRB5.

In some embodiments, the anti-LILRB2 antibody weakly binds or does not bind to an LILRA.

In some embodiments, the anti-LILRB antibody is a pan antibody that specifically binds to LILRB1, LILRB2, LILRB4, and LILRB5; LILRB1, LILRB2, LILRB3, and LILRB4; LILRB1, LILRB2, and LILRB5; or LILRB1 and LILRB3.

In some cases, the anti-LILRB antibody blocks HLA-G binding to a cell expressing a LILRB receptor, blocks HLA-A binding to the cell expressing a LILRB receptor, or a combination thereof.

In some cases, the anti-LILRB antibody enhances HLA-G binding to a cell expressing a LILRB receptor.

In some cases, the anti-LILRB antibody does not modulate HLA-G binding or HLA-A binding to a cell expressing a LILRB receptor.

In some embodiments, the anti-LILRB antibody is 5G11.G8, 5G11.H6, 9C9.D3, 9C9.E6, 16D11.D10, 6G6.H7, 6G6.H2, 6H9.A3, 2B3.A10, 4D11.B10, or 11D9.E7. In some cases, the anti-LILRB antibody is 5G11.G8, 5G11.H6, 9C9.D3, 9C9.E6, 16D11.D10, 6G6.H7, or 6G6.H2. In some cases, the anti-LILRB antibody is 5G11.G8, 5G11.H6, 9C9.D3, 9C9.E6, or 16D11.D10. In some cases, the anti-LILRB antibody is 6H9.A3, 2B3.A10, 4D11.B10, or 11D9.E7. In some cases, the anti-LILRB antibody is 5G11.G8. In some cases, the anti-LILRB antibody is 5G11.H6. In some cases, the anti-LILRB antibody is 9C9.D3. In some cases, the anti-LILRB antibody is 9C9.E6. In some cases, the anti-LILRB antibody is 16D11.D10. In some cases, the anti-LILRB antibody is 6G6.H7. In some cases, the anti-LILRB antibody is 6G6.H2. In some cases, the anti-LILRB antibody is 6H9.A3. In some cases, the anti-LILRB antibody is 2B3.A10. In some cases, the anti-LILRB antibody is 4D11.B10. In some cases, the anti-LILRB antibody is 11D9.E7.

In some embodiments, disclosed herein is a pan anti-LILRB antibody that specifically binds to at least one epitope on the extracellular domain of LILRB1, at least one epitope on the extracellular domain of LILRB2, LILRB3, LILRB4, or LILRB5, or a combination thereof. In some instances, the pan anti-LILRB antibody blocks HLA-G binding to a cell expressing a LILRB receptor. In some instances, the pan anti-LILRB antibody further blocks binding of HLA-A to a cell expressing a LILRB receptor. In additional instances, the pan anti-LILRB antibody induces inflammatory cytokine production (e.g., TNFα, IFNγ, or a combination thereof) when contacted to a plurality of cells. In some cases, the pan anti-LILRB antibody further binds specifically to LILRA1, LILRA3, LILRA5, LILRA6, or a combination thereof. In some cases, the pan anti-LILRB antibody binds specifically to LILRA1, LILRA3, LILRA5, and LILRA6. In some cases, the pan anti-LILRB antibody binds specifically to LILRA1, LILRA3, and LILRA6. In some instances, the at least one epitope on the extracellular domain of LILRB2 comprises a peptide sequence within D3, a peptide sequence within D4, or a combination thereof. In some instances, the at least one epitope on the extracellular domain of LILRB2 comprises a conformational epitope. In some cases, the conformational epitope is within D3 and comprises at least one peptide sequence; is within D4 and comprises at least one peptide sequence; or comprises at least one peptide sequence from D3 and at least one peptide sequence from D4. In some cases, the conformational epitope within D3 comprises at least one peptide sequence from region 223-231, region 236-248, region 258-262, region 269-290, or region 298-314, or a combination thereof, wherein the residue numberings correspond to positions 223-231, 236-248, 258-262, 269-290, and 298-314 of SEQ ID NO: 9. In some cases, the conformational epitope within D4 comprises at least one peptide sequence from region 336-340, region 362-368, region 379-393, region 400-403, or region 412-419, or a combination thereof, wherein the residue numberings correspond to positions 336-340, 362-368, 379-393, 400-403, and 412-419 of SEQ ID NO: 9. In some instances, the pan antibody specifically binds to one or more LILRB1 isoforms selected from isoforms 1-6; or to a LILRB1 encoded by a sequence comprising at least 90%, 95%, 96%, 97%, 98%, 99%, or 100% sequence identity to SEQ ID NOs: 33-35. In some instances, the pan antibody specifically binds to one or more LILRB2 isoforms selected from isoforms 1-5; or to a LILRB2 encoded by a sequence comprising at least 90%, 95%, 96%, 97%, 98%, 99%, or 100% sequence identity to SEQ ID NOs: 36-39. In some instances, the pan antibody specifically binds to one or more LILRB3 isoforms selected from isoforms 1-3; or to a LILRB3 encoded by a sequence comprising at least 90%, 95%, 96%, 97%, 98%, 99%, or 100% sequence identity to SEQ ID NO: 40 or 41. In some instances, the pan anti-LILRB antibody is 5G11.H6, 9C9.D3, 9C9.E6, 5G11.G8, or 16D11.D10.

In some embodiments, disclosed herein is a pan anti-LILRB antibody that specifically binds to at least one epitope on the extracellular domain of LILRB1, at least one epitope on the extracellular domain of LILRB2, or at least one epitope on the extracellular domain of LILRB4. In some instances, the pan anti-LILRB antibody enhances HLA-G binding to a cell expressing a LILRB receptor. In some instances, the pan anti-LILRB antibody further blocks binding of HLA-A to a cell expressing a LILRB receptor. In additional instances, the pan anti-LILRB antibody induces inflammatory cytokine production (e.g., TNFα, IFNγ, or a combination thereof) when contacted to a plurality of cells. In some cases, the pan anti-LILRB antibody further binds specifically to LILRA1, LILRA3, LILRA5, LILRA6, or a combination thereof. In some cases, the pan anti-LILRB antibody binds specifically to LILRA1, LILRA3, LILRA5, and LILRA6. In some cases, the pan anti-LILRB antibody binds specifically to LILRA1, LILRA3, and LILRA6. In some instances, the epitope is within domain D1, D2, D3, or D4 of the respective LILRB. In some instances, the epitope is a conformational epitope. In some instances, the pan anti-LILRB antibody is 8F7.D2, 8B11.E12, 5H5.A3, 8E8.D2, 8F7.C3, 13H1.G2, 14B7.C2, 6H9.A3, 13H1.G2, 14B7.A4, 8E8.C4, 9B11.D3, or 9B11.D5.

In some embodiments, disclosed herein is an anti-LILRB antibody, when contacted to a cell expressing a LILRB receptor, enhances the cell's binding to HLA-G. In some instances, the anti-LILRB antibody further induces inflammatory cytokine production (e.g., TNFα, IFNγ, or a combination thereof) by the cell. In some cases, the anti-LILRB antibody is an anti-LILRB1 antibody, an anti-LILRB2 antibody, an anti-LILRB3 antibody, an anti-LILRB4 antibody, or an anti-LILRB5 antibody. In some cases, the anti-LILRB antibody is an anti-LILRB1 antibody. In some cases, the anti-LILRB antibody is an anti-LILRB2 antibody. In some cases, the anti-LILRB antibody is an anti-LILRB3 antibody. In some cases, the anti-LILRB antibody is an anti-LILRB4 antibody. In some cases, the anti-LILRB antibody is a pan anti-LILRB antibody that specifically binds, e.g., LILRB1 in combination with any of LILRB2, LILRB3, LILRB4, or LILRB5. In some cases, the pan anti-LILRB antibody specifically binds to LILRB1 and LILRB2. In some cases, the pan anti-LILRB antibody specifically binds to LILRB1, LILRB2, and LILRB3. In some cases, the pan anti-LILRB antibody specifically binds to LILRB1 and LILRB2 and further to one or more of LILRB3, LILRB4, and LILRB5. In some cases, the pan anti-LILRB antibody specifically binds to LILRB1 and LILRB3 and further to one or more of LILRB4 and LILRB5. In some cases, the pan anti-LILRB antibody specifically binds to LILRB1, LILRB2, LILRB3, and LILRB4. In some instances, the anti-LILRB antibody induces a higher level of inflammatory cytokine production relative to a control. In some instances, the control is an IgG1 or IgG2 antibody. In some cases, the control is antibody 287219.

In some embodiments, disclosed herein is an anti-LILRB antibody, when contacted to a cell expressing a LILRB receptor, blocks the cell's binding to HLA-G. In some instances, the anti-LILRB antibody further blocks HLA-A binding to the cell. In additional instances, the anti-LILRB antibody induces inflammatory cytokine production (e.g., TNFα, IFNγ, or a combination thereof) by the cell. In some cases, the anti-LILRB antibody is an anti-LILRB1 antibody, an anti-LILRB2 antibody, an anti-LILRB3 antibody, an anti-LILRB4 antibody, or an anti-LILRB5 antibody. In some cases, the anti-LILRB antibody is an anti-LILRB1 antibody. In some cases, the anti-LILRB antibody is an anti-LILRB2 antibody. In some cases, the anti-LILRB antibody is an anti-LILRB3 antibody. In some cases, the anti-LILRB antibody is an anti-LILRB4 antibody. In some cases, the anti-LILRB antibody is a pan anti-LILRB antibody that specifically binds, e.g., LILRB1 in combination with any of LILRB2, LILRB3, LILRB4, or LILRB5. In some cases, the pan anti-LILRB antibody specifically binds to LILRB1 and LILRB2. In some cases, the pan anti-LILRB antibody specifically binds to LILRB1, LILRB2, and LILRB3. In some cases, the pan anti-LILRB antibody specifically binds to LILRB1 and LILRB2 and further to one or more of LILRB3, LILRB4, and LILRB5. In some cases, the pan anti-LILRB antibody specifically binds to LILRB1 and LILRB3 and further to one or more of LILRB4 and LILRB5. In some cases, the pan anti-LILRB antibody specifically binds to LILRB1, LILRB2, LILRB3, and LILRB4. In some instances, the anti-LILRB antibody induces a higher level of inflammatory cytokine production relative to a control. In some instances, the control is an IgG1 or IgG2 antibody. In some cases, the control is antibody 42D1 or antibody ZM4.1.

In some instances, the pan anti-LILRB antibodies or binding fragments thereof inhibit binding of a ligand of LILRBs (e.g., LILRB1, LILRB2, LILRB3, LILRB4, and/or LILRB5) by at least 10%, 20%, 30%, 40%, 50%, 60%, 70%, 80%, 90%, 95%, or more. In some cases, the pan anti-LILRB antibodies or binding fragments thereof inhibit binding of a ligand of LILRBs by at least 10% or more. In some cases, the pan anti-LILRB antibodies or binding fragments thereof inhibit binding of a ligand of LILRBs by at least 20% or more. In some cases, the pan anti-LILRB antibodies or binding fragments thereof inhibit binding of a ligand of LILRBs by at least 30% or more. In some cases, the pan anti-LILRB antibodies or binding fragments thereof inhibit binding of a ligand of LILRBs by at least 40% or more. In some cases, the pan anti-LILRB antibodies or binding fragments thereof inhibit binding of a ligand of LILRBs by at least 50% or more. In some cases, the pan anti-LILRB antibodies or binding fragments thereof inhibit binding of a ligand of LILRBs by at least 60% or more. In some cases, the pan anti-LILRB antibodies or binding fragments thereof inhibit binding of a ligand of LILRBs by at least 70% or more. In some cases, the pan anti-LILRB antibodies or binding fragments thereof inhibit binding of a ligand of LILRBs by at least 80% or more. In some cases, the pan anti-LILRB antibodies or binding fragments thereof inhibit binding of a ligand of LILRBs by at least 90% or more. In some cases, the pan anti-LILRB antibodies or binding fragments thereof inhibit binding of a ligand of LILRBs by at least 95% or more. In some cases, the ligand is a natural ligand. In some cases, the ligand comprises HLA-A, HLA-B, HLA-C, HLA-E, HLA-F, HLA-G, UL18, α3 domain and β2-microglubulin of class I protein, S100A8, S100A9, CD1d, CD1c, ANGPTL1, ANGPTL2, ANGPTL3, ANGPTL4, ANGPTL5, ANGPTL6, ANGPTL7, ANGPTL8, Nogo66, MAG, OMgp, RTN4, or β-amyloid. In some cases, the ligand comprises HLA-B7, HLA-B27, ANGPTL1, ANGPTL2, ANGPTL3, ANGPTL4, ANGPTL5, ANGPTL6, ANGPTL7, or ANGPTL8. In some cases, the ligand comprises a pathogen such as Dengue virus, Escherichia coli, or Staphylococcus aureus. In some cases, the ligand comprises a pathogen such as Staphylococcus aureus.

In some instances, the pan anti-LILRB antibodies or binding fragments thereof inhibit binding of a ligand of LILRBs (e.g., LILRB1, LILRB2, LILRB3, LILRB4, and/or LILRB5) by about 2-fold, 3-fold, 4-fold, 5-fold, 6-fold, 7-fold, 8-fold, 9-fold, 10-fold, or more. In some instances, the pan anti-LILRB antibodies or binding fragments thereof inhibit binding of a ligand of LILRBs by about 2-fold or more. In some instances, the pan anti-LILRB antibodies or binding fragments thereof inhibit binding of a ligand of LILRBs by about 3-fold or more. In some instances, the pan anti-LILRB antibodies or binding fragments thereof inhibit binding of a ligand of LILRBs by about 4-fold or more. In some instances, the pan anti-LILRB antibodies or binding fragments thereof inhibit binding of a ligand of LILRBs by about 5-fold or more. In some instances, the pan anti-LILRB antibodies or binding fragments thereof inhibit binding of a ligand of LILRBs by about 6-fold or more. In some instances, the pan anti-LILRB antibodies or binding fragments thereof inhibit binding of a ligand of LILRBs by about 7-fold or more. In some instances, the pan anti-LILRB antibodies or binding fragments thereof inhibit binding of a ligand of LILRBs by about 8-fold or more. In some instances, the pan anti-LILRB antibodies or binding fragments thereof inhibit binding of a ligand of LILRBs by about 9-fold or more. In some instances, the pan anti-LILRB antibodies or binding fragments thereof inhibit binding of a ligand of LILRBs by about 10-fold or more. In some cases, the ligand is a natural ligand. In some cases, the ligand comprises HLA-A, HLA-B, HLA-C, HLA-E, HLA-F, HLA-G, UL18, α3 domain and β2-microglubulin of class I protein, S100A8, S100A9, CD1d, CD1c, ANGPTL1, ANGPTL2, ANGPTL3, ANGPTL4, ANGPTL5, ANGPTL6, ANGPTL7, ANGPTL8, Nogo66, MAG, OMgp, RTN4, or β-amyloid. In some cases, the ligand comprises HLA-B7, HLA-B27, ANGPTL1, ANGPTL2, ANGPTL3, ANGPTL4, ANGPTL5, ANGPTL6, ANGPTL7, or ANGPTL8. In some cases, the ligand comprises a pathogen such as Dengue virus, Escherichia coli, or Staphylococcus aureus. In some cases, the ligand comprises a pathogen such as Staphylococcus aureus.

In some embodiments, a pan anti-LILRB antibody or binding fragment thereof described above comprises a humanized antibody or binding fragment thereof, murine antibody or binding fragment thereof, chimeric antibody or binding fragment thereof, monoclonal antibody or binding fragment thereof, bispecific antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. In some cases, the pan anti-LILRB antibody or binding fragment thereof comprises a humanized antibody or binding fragment thereof. In some cases, the pan anti-LILRB antibody or binding fragment thereof comprises a murine antibody or binding fragment thereof. In some cases, the pan anti-LILRB antibody or binding fragment thereof comprises a chimeric antibody or binding fragment thereof. In some cases, the pan anti-LILRB antibody or binding fragment thereof comprises a monoclonal antibody or binding fragment thereof. In some cases, the pan anti-LILRB antibody or binding fragment thereof comprises a bispecific antibody or binding fragment thereof. In some cases, the pan anti-LILRB antibody or binding fragment thereof comprises a monovalent Fab′, a divalent Fab2, or F(ab)′3 fragments. In some cases, the pan anti-LILRB antibody or binding fragment thereof comprises a single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, or camelid antibody or binding fragment thereof. In some cases, the pan anti-LILRB antibody or binding fragment thereof comprises a chemically modified derivative thereof.

In some embodiments, the pan anti-LILRB antibody or binding fragment thereof, when contacted to a plurality of peripheral blood mononuclear cells (PBMCs) comprising T cells, enhances cytotoxic T cell activation relative to a plurality of equivalent PBMCs and equivalent T cells in the absence of the antibody or binding fragment thereof.

In some embodiments, the pan anti-LILRB antibody or binding fragment thereof, when contacted to a plurality of peripheral blood mononuclear cells (PBMCs) comprising a macrophage, increases M1 activation of the macrophage relative to a plurality of equivalent PBMCs and an equivalent macrophage in the absence of the antibody or binding fragment thereof.

In some embodiments, the pan anti-LILRB antibody or binding fragment thereof, when contacted to a plurality of cells comprising APCs and a target cell, increases phagocytosis of the target cell relative to a plurality of equivalent cells in the absence of the antibody or binding fragment thereof.

In some embodiments, the pan anti-LILRB antibody or binding fragment thereof, when contacted to a plurality of cells increases inflammatory cytokine production relative to a plurality of equivalent cells in the absence of the antibody or binding fragment thereof. In some cases, the inflammatory cytokine comprises TNFα, IFNγ, or a combination thereof.

In some embodiments, the pan anti-LILRB antibody or binding fragment thereof, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the pan anti-LILRB antibody or binding fragment thereof.

In some embodiments, also described herein include a vector comprising a nucleic acid molecule that encodes a pan anti-LILRB antibody or binding fragment thereof.

In some embodiments, further described herein include a host cell comprising a nucleic acid molecule that encodes a pan anti-LILRB antibody or binding fragment thereof.

### Proliferative Diseases

In some embodiments, a proliferative disease described herein is a cancer. In some instances, the cancer is a solid tumor. In some instances, the cancer is a hematologic malignancy. In some instances, the cancer is a relapsed or refractory cancer, or a metastatic cancer. In some instances, the solid tumor is a relapsed or refractory solid tumor, or a metastatic solid tumor. In some cases, the hematologic malignancy is a relapsed or refractory hematologic malignancy, or a metastatic hematologic malignancy.

In some embodiments, the cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the cancer is a hematologic malignancy. In some instances, the hematologic malignancy is a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, described herein include an anti-LILRB1 antibody or binding fragment thereof for the treatment of a cancer.

In some cases, described herein include an anti-LILRB2 antibody or binding fragment thereof for the treatment of a cancer.

In some instances, described herein include an anti-LILRB3 antibody or binding fragment thereof for the treatment of a cancer.

In some instances, described herein include a pan anti-LILRB antibody or binding fragment thereof for the treatment of a cancer.

### Infectious Diseases

In some embodiments, a pathogen that causes an infectious disease described herein comprises a virus, bacterium, protozoan, helminth, prion, or fungus. In some instances, the pathogen is a virus, e.g., DNA viruses such as single-stranded or double-stranded DNA viruses; RNA viruses such as single-stranded RNA viruses (e.g., sense strand or antisense strand) and double-stranded RNA viruses; or retroviruses. Exemplary viruses include those from the family: Adenoviridae, Flaviviridae, Hepadnaviridae, Herpesviridae, Orthomyxoviridae, Papovaviridae, Paramyxoviridae, Picornaviridae, Polyomavirus, Retroviridae, Rhabdoviridae, or Togaviridae. Exemplary infectious diseases include, but are not limited to, Dengue fever (caused by the Dengue virus) and acquired immune deficiency syndrome (AIDS) (caused by the human immunodeficiency virus (HIV)).

In some instances, described herein include an anti-LILRB1 antibody or binding fragment thereof and/or a pan anti-LILRB antibody or binding fragment thereof for the treatment of Dengue fever.

In some cases, described herein include an anti-LILRB2 antibody or binding fragment thereof and/or a pan anti-LILRB antibody or binding fragment thereof for the treatment of AIDS.

In some embodiments, the pathogen is a bacterium, e.g., a Gram-positive or Gram-negative bacterium. Exemplary bacteria include those from the Genus: Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, or Yersinia.

In some instances, described herein include an anti-LILRB1 antibody or binding fragment thereof and/or a pan anti-LILRB antibody or binding fragment thereof for the treatment of an infection caused by Staphylococcus aureus.

In some instances, described herein include an anti-LILRB3 antibody or binding fragment thereof and/or a pan anti-LILRB antibody or binding fragment thereof for the treatment of an infection caused by Staphylococcus aureus.

In some instances, described herein include an anti-LILRB1 antibody or binding fragment thereof and/or a pan anti-LILRB antibody or binding fragment thereof for the treatment of sepsis.

In some instances, described herein include an anti-LILRB3 antibody or binding fragment thereof and/or a pan anti-LILRB antibody or binding fragment thereof for the treatment of sepsis.

In some embodiments, the pathogen is a protozoan. Exemplary parasitic protozoa include, but are not limited to, alveolates belonging to the genus Plasmodium (which causes malaria), amoebas of the genus Entamoeba (which causes amoebiasis), Giardia lamblia (which causes giardiasis or beaver fever), Toxoplasma gondii (which causes toxoplasmosis), Cryptosporidium (which causes cryptosporidiosis), Trichomonas vaginalis (which causes trichomoniasis), Trypanosoma cruzi (which causes Chagas disease or American trypanosomiasis), Leishmania (which causes leishmaniasis), Trypanosoma brucei (which causes African trypanosomiasis or sleeping sickness), and Naegleria fowleri (which causes naegleriasis).

In some instances, described herein include an anti-LILRB1 antibody or binding fragment thereof and/or a pan anti-LILRB antibody or binding fragment thereof for the treatment of malaria.

In some embodiments, the pathogen is a helminth. Exemplary helminths include, but are not limited to, flatworms (Platyhelminthes) such as cestodes (tapeworms) and trematodes (flukes), and roundworms (nematodes).

In some embodiments, the pathogen is a fungus. Exemplary pathogenic fungi include those from the genus: Aspergillus, Candida, Cryptococcus, Histoplasma, Pneumocystis, or Stachybotrys.

### Neurological Diseases or Disorders

In some embodiments, a neurological disease or disorder described herein comprises a disease or condition characterized by a physically damaged nerve, or peripheral nerve damage caused by a physical injury. Such disease or disorder includes, for example, neurodegenerative diseases such as Alzheimer's disease, amyotrophic lateral sclerosis (AML), Parkinson's disease, or Huntington's disease; a disease or condition associated with stroke; or a disease or condition associated with a brain injury (e.g., an injury to the central nervous system).

In some instances, described herein include an anti-LILRB1 antibody or binding fragment thereof, an anti-LILRB2 antibody or binding fragment thereof, and/or a pan anti-LILRB antibody or binding fragment thereof for the treatment of a neurological disease or disorder, e.g., as such a disease or condition associated with stroke.

In some instances, described herein include an anti-LILRB2 antibody or binding fragment thereof and/or a pan anti-LILRB antibody or binding fragment thereof for the treatment of a neurodegenerative disease such as Alzheimer's disease.

### Autoimmune Diseases

In some embodiments, an autoimmune disease described herein comprises a disease in which the autoimmune system attacks cells and/or tissues of the self to cause inflammation and damages of the cells and/or tissues. In some instances, the autoimmune disease is graft versus host disease (GVHD). In some cases, GVHD comprises acute GVHD, which is developed with the first 100 days post-transplantation, and chronic GVHD, which is developed more than 100 days post-transplantation.

In some instances, described herein include an anti-LILRB4 antibody or binding fragment thereof and/or a pan anti-LILRB antibody or binding fragment thereof for the treatment of GVHD.

### Production of Antibodies or Binding Fragments Thereof

In some embodiments, polypeptides described herein (e.g., antibodies and their binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible, or a tissue specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA, or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter sequences, enhancer sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, Proc. Nat. Acad. Sci. USA 48:202, 1992), and adenine phosphoribosyltransferase (Lowy et al., 1980, Cell 22:817) genes are employed in tk-, hgprt- or aprt- cells, respectively. Also, antimetabolite resistance is used as the basis of selection for the following genes: dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J Mol. Biol. 150:1).

In some instances, the expression levels of an antibody are increased by vector amplification (for a review, see Bebbington and Hentschel, The use of vectors based on gene amphification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

In some embodiments, an antibody or its binding fragment is further modified using conventional techniques known in the art, for example, by using amino acid deletion, insertion, substitution, addition, and/or by recombination and/or any other modification (e.g. posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. Methods for introducing such modifications in the nucleic acid sequence underlying the amino acid sequence of an antibody or its binding fragment is well known to the person skilled in the art.

### Methods of Use

In some embodiments, described herein are anti-LILRB1 antibodies or binding fragments thereof, anti-LILRB2 antibodies or binding fragments thereof, and pan anti-LILRB antibodies or binding fragments thereof that modulate inflammatory macrophage activation and/or lymphocyte activation. In some instances, the anti-LILRB1 antibodies or binding fragments thereof, anti-LILRB2 antibodies or binding fragments thereof, and pan anti-LILRB antibodies or binding fragments thereof further modulate phagocytosis of a target cell. In additional instances, the anti-LILRB1 antibodies or binding fragments thereof, anti-LILRB2 antibodies or binding fragments thereof, and pan anti-LILRB antibodies or binding fragments thereof decrease tumor-infiltrating regulatory T cells.

In some embodiments, described herein is a method of modulating inflammatory macrophage activation, comprising (a) contacting a plurality of peripheral blood mononuclear cells (PBMCs) comprising at least one antigen presenting cell (APC) and a macrophage with an anti-LILRB1 antibody or binding fragment thereof, an anti-LILRB2 antibody or binding fragment thereof, or a pan anti-LILRB antibody or binding fragment thereof, (b) binding the antibody or binding fragment thereof to one or more LILRB receptors expressed on the at least one APC, thereby inducing the APC to produce a plurality of TNFα, interferons, lipopolysaccharide (LPS), and/or GM-CSF; and (c) contacting the plurality of TNFα, interferons, LPS, and/or GM-CSF with the plurality of PBMCs comprising the macrophage to induce inflammatory macrophage activation.

In some instances, inflammatory macrophage activation refers to macrophage activation (or macrophage polarization) that promotes a pro-inflammatory response. For example, the activated macrophage is optionally characterized by the production of pro-inflammatory cytokines, an ability to mediate resistance to pathogens, high production of reactive nitrogen and oxygen intermediates relative to an un-activated macrophage, and/or promotion of Th1 responses. In some cases, inflammatory macrophage activation comprises the classically activated or M1 classification.

In some embodiments, inflammatory macrophage activation differs and does not encompass alternatively activated macrophages (or M2-polarized macrophages). As used herein, alternatively activated macrophages (or M2-polarized macrophages) comprise M2a, M2b, M2c and M2d subtypes, promotes anti-inflammatory responses, and are activated by Th2 cytokines (e.g., IL-4, IL-10, and/or IL-13). In some instances, the alternatively activated macrophages are further characterized by their involvement in parasite control, tissue remodeling, immune regulation, tumor promotion, and phagocytic activity. In some cases, the alternatively activated macrophages encompass tumor-associated macrophages (TAMs). In some cases, the classically activated macrophage does not comprise TAMs.

In some instances, described herein is a method of modulating a macrophage to undergo M1 activation, comprising (a) contacting a plurality of antigen presenting cells (APCs) comprising a macrophage with an anti-LILRB1 antibody or binding fragment thereof, an anti-LILRB2 antibody or binding fragment thereof, or a pan anti-LILRB antibody or binding fragment thereof, (b) binding the antibody or binding fragment thereof or the pan antibody or binding fragment thereof to one or more LILRB receptors expressed on at least one APC within the plurality of APCs, thereby inducing the APC to produce a plurality of TNFα and interferons; and (c) contacting the plurality of TNFα and interferons with the plurality of APCs comprising the macrophage to induce M1 activation of the macrophage.

In some instances, the interferons comprise IFNγ. In other instances, the interferons comprise IFNβ.

In some instances, the PBMCs further comprise antigen presenting cells (APCs), NK cells, and/or T cells. In some instances, the APCs further comprise dendritic cells, B cells, or a combination thereof.

In some cases, the antibody or binding fragment thereof or the pan antibody or binding fragment thereof decreases M2 activation of the macrophage.

In additional cases, the antibody or binding fragment thereof or the pan antibody or binding fragment thereof decreases formation of a tumor associate macrophage.

In additional embodiments, described herein is a method of inducing phagocytosis of a target cell, comprising (a) incubating a plurality of peripheral blood mononuclear cells (PBMCs) comprising a macrophage with an anti-LILRB1 antibody or binding fragment thereof, an anti-LILRB2 antibody or binding fragment thereof, or a pan anti-LILRB antibody or binding fragment thereof, thereby inducing the macrophage to undergo activation to an inflammatory phenotype; and (b) contacting the activated macrophage to a target cell for a time sufficient to induce phagocytosis of the target cell.

In some instances, the activated macrophage comprises a classically activated or M1-polarized phenotype. In such instances, the method comprises (a) incubating a plurality of antigen presenting cells (APCs) comprising a macrophage with an anti-LILRB1 antibody or binding fragment thereof, an anti-LILRB2 antibody or binding fragment thereof, or a pan anti-LILRB antibody or binding fragment thereof, thereby inducing the macrophage to undergo M1 polarization; and (b) contacting the M1 macrophage to a target cell for a time sufficient to induce phagocytosis of the target cell.

In some instances, the time sufficient to induce phagocytosis comprises at least 30 seconds, 1 minute, 2 minutes, 5 minutes, 10 minutes, 30 minutes, 1 hour, 2 hours, 12 hours, 24 hours, or more.

In some cases, the target cell is a cancer cell. In other instances, the target cell is a cell infected by a pathogen, e.g., by a virus, bacterium, protozoan, helminth, prion, or fungus.

In some cases, the PBMCs further comprise antigen presenting cells (APCs), NK cells, and/or T cells. In some instances, the APCs further comprise dendritic cells, B cells, or a combination thereof.

In further embodiments, described herein is a method of activating a lymphocyte, comprising (a) incubating a plurality of peripheral blood mononuclear cells (PBMCs) comprising a lymphocyte with an anti-LILRB1 antibody or binding fragment thereof, an anti-LILRB2 antibody or binding fragment thereof, or a pan anti-LILRB antibody or binding fragment thereof, thereby stimulating the secretion of a plurality of cytokines; and (b) contacting the plurality of cytokines with the lymphocyte to induce activation. In some instances, lymphocyte activation comprises activation of T cells such as cytotoxic (CD8+) T cells and/or CD4⁺ T cells, B cells, and/or natural killer (NK) cells. In some cases, the plurality of cytokines comprises TNFα, IFNγ, IFNβ, IL-2, IL-4, IL-5, IL-6, IL-12, IL-15, IL-18, and/or CCL5.

In some embodiments, the method comprises activation of a cytotoxic T cell. In such instances, the method comprises (a) incubating a plurality of peripheral blood mononuclear cells (PBMCs) comprising naive T cells with an anti-LILRB1 antibody or binding fragment thereof, an anti-LILRB2 antibody or binding fragment thereof, or a pan anti-LILRB antibody or binding fragment thereof, thereby stimulating the secretion of a plurality of inflammatory cytokines; and (b) contacting the plurality of inflammatory cytokines with the naïve T cells to activate a cytotoxic T cell. In some cases, the inflammatory cytokines comprises TNFα, IFNγ, or IFNβ. In some cases, the naïve T cells comprise naïve CD8⁺ T cells. In some cases, the PBMCs comprise antigen presenting cells (APCs), NK cells, and/or CD4 T cells. In some instances, the CD4 T cells comprise activated CD4⁺ helper T cells. In some cases, the APCs comprise B cells and/or dendritic cells.

Pharmaceutical Compositions

In some embodiments, an anti-LILRB antibody or binding fragment thereof (e.g., an anti-LILRB1 antibody or binding fragment thereof, an anti-LILRB2 antibody or binding fragment thereof, anti-LILRB3 antibodies or binding fragments thereof, anti-LILRB4 antibodies or binding fragments thereof, anti-LILRB5 antibodies or binding fragments thereof, and/or a pan anti-LILRB antibody or binding fragment hereof) is further formulated as a pharmaceutical composition. In some instances, the pharmaceutical composition is formulated for administration to a subject by multiple administration routes, including but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. Exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle. In some cases, a nanoparticle has at least one dimension of less than about 500 nm, 400 nm, 300 nm, 200 nm, or 100 nm.

In some embodiments, the pharmaceutical compositions include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical compositions include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PHi01, Avicel® PH102, Avicel® PH105, Elcema® P100, Emcocelo, Vivacelo, Ming Tia, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical compositions include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical compositions described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotexo), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Silo, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity-enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the improved disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-LILRB1 antibody or binding fragment thereof, an anti-LILRB2 antibody or binding fragment thereof, anti-LILRB3 antibodies or binding fragments thereof, anti-LILRB4 antibodies or binding fragments thereof, anti-LILRB5 antibodies or binding fragments thereof, or a pan anti-LILRB antibody or binding fragment thereof as disclosed herein, host cells for producing one or more antibodies or binding fragments described herein, and/or vectors comprising nucleic acid molecules that encode the antibodies or binding fragments described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “peptide sequence” comprises at least one amino acid residue of an LILRB. In some instances, the peptide sequence comprises 2 or more residues, 3 or more residues, 4 or more residues, 5 or more residues, 10 or more residues, 15 or more residues, 20 or more residues, 25 or more residues, or 30 or more residues. In some instances, the peptide sequence comprises from about 2 to about 30 residues, from about 5 to about 25 residues, from about 5 to about 20 residues, from about 5 to about 15 residues, from about 5 to about 10 residues, from about 10 to about 25 residues, from about 10 to about 20 residues, or from about 15 to about 25 residues. In some instances, the peptide sequence is a linear sequence, formed by a continuous sequence of amino acids in the LILRB protein. The amino acid residue comprises both natural amino acid and modified amino acid, e.g., by post-translational modification or chemical modification.

As used herein, the term “conformational epitope” refers to a set of amino acid residues that are discontinuous in the protein sequence but which are brought together upon folding of the LILRB protein into its three-dimensional structure. The conformational epitope differs from a linear epitope, which is formed by a continuous sequence of amino acids in the LILRB protein. In some instances, the conformational epitope comprises a set of amino acid residues in which the amino acid residues are from two or more different peptide sequences within a LILRB protein. For instances, an exemplary conformational epitope may comprise one or more amino acid residues from a peptide sequence within, e.g., D3 of LILRB2, and one or more amino acid residues from a peptide sequence within, e.g., D4 of LILRB2. Alternatively, an exemplary conformational epitope may comprise one or more amino acid residues from a first peptide sequence within, e.g., D3 of LILRB2, and one or more amino acid residues from a second peptide sequence within, e.g., D3 of LILRB2.

As used herein, the term “equivalent” in the context of the extracellular Ig-like domains refers to amino acids of a sequence of interest that shares a sequence homology to amino acids of a reference sequence. For example, the sequence of interest optionally shares a sequence homology of about 90%, 95%, 99%, or higher relative to the reference sequence. Sequence homology encompasses conservative substitutions such as those illustrated in the following chart, and optionally comprises modified amino acids such as by post-translational modification or chemical modification.

| Original Residue | Conserved Substitutions |
|---|---|
| Ala | Ser, Gly, Thr, Cys, Val |
| Arg | Lys, Gln, His, Asn, Glu |
| Asn | Gln, His, Asp, Lys, Ser, Thr, Arg, Glu |
| Asp | Glu, Asn, Gln, Ser |
| Cys | Ser, Ala |
| Gln | Asn, Arg, Glu, His, Lys Met, Asp, Ser |
| Glu | Asp, Gln, Lys, Arg, Asn, His, Ser |
| Gly | Pro, Ala, Ser |
| His | Asn, Gln, Arg, Tyr, Glu |
| Ile | Leu, Val, Met, Phe |
| Leu | Ile, Val, Met, Phe |

In some cases, sequence homology further encompasses variants such as polymorphic variants and interspecies homologs. In some cases, the sequence of interest shares a sequence identity of about 90%, 95%, 99%, or higher relative to the reference sequence.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, an antibody binding fragment described herein further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such embodiments, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some embodiments, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its binding fragment is capable of specifically interacting with and/or binding to at least one amino acid of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further embodiments, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or its binding fragment or a reduced off-target effect. For example, the antibody or its binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

### EXAMPLES

These examples are provided for illustrative purposes only and not to limit the scope of the claims provided herein.

### Example 1. Production of Anti-LILRB Antibodies

LILRB Construct Design

LILRB Fc constructs were generated for immunization. LILRB extracellular domain constructs were cloned with a short Gly-Ser linker into the human IgG pFUSEN vector (Invivogen) using BamHI and KpnI restriction sites (NEB). Ligated constructs were transformed into oneShot Top10 chemically competent E. coli (Thermo Fisher) and grown on Zeocin agar plates (Teknova). Protein expression was obtained by transfecting FreeStyle 293F cells (Thermo Fisher) with FectoPro transfection reagent. Expressed protein was purified using FPLC using protein A coated columns (MabSelect Sure, GE healthcare) and eluted using IgG elution buffer (Thermo Fisher). Protein concentration and buffer exchange in Sodium Phosphate buffer (pH 7.2) was performed using Amicon Ultra 30 kDa molecular weight columns.

Production of Anti-LILRB Antibodies

Mice (BALB/c or SJL) were immunized with LILRB1-Fc tagged, LILRB2-Fc tagged, LILRB3-Fc tagged, or LILRB4-Fc tagged construct using the method described in Kohler and Milstein, Nature 256:495-497(1975). In brief, three female Balb/C mice were immunized with 100 g of the LILRB1-Fc tagged, LILRB2-Fc tagged, LILRB3-Fc tagged, or LILRB4-Fc tagged construct in complete Freund's adjuvant, administered by intraperitoneal (IP) injection. Three subsequent injections were administered IP comprising 50 g of the LILRB1-Fc tagged, LILRB2-Fc tagged, LILRB3-Fc tagged, or LILRB4-Fc tagged construct in incomplete Freund's adjuvant on days 10, 20, and 30 after initial injection. Serum was collected on day 37 and the presence of antibodies reactive to the antigen was determined by solid phase ELISA.

Hybridoma Generation

The mouse demonstrating the highest serum antibody titer (defined as the dilution of serum at 50% maximum signal) received booster injection comprising 25 g of antigen. Three days later, the mouse was sacrificed and its spleen cells were isolated and fused with NS1 myeloma cells following a standard fusion protocol. These mixtures of clones, called parental clones, were screened at 10 days after the fusion by solid-phase ELISA against LILRB1-Fc tagged, LILRB2-Fc tagged, LILRB3-Fc tagged, or LILRB4-Fc tagged construct to identify parental clones that secreted antibodies capable of binding LILRB1, LILRB2, LILRB3, or LILBR4, respectively. Positive parental clones were selected for further analysis.

These positive parental clones originating from the fusion were expanded from the 96 well plates to 24 well plates and were rescreened 3 days later to ensure that the clones were still producing antibody. From the rescreen, selected wells with positive hybridomas, (i.e., hybridomas that secreted antibodies capable of binding the LILRB1-Fc tagged, LILRB2-Fc tagged, LILRB3-Fc tagged, or LILRB4-Fc tagged construct and that did not bind to metabolite-1-BSA, metabolite-2-BSA) were frozen for future use and sub-cloned to isolate positive cell lines.

The selected positive parent clones that secreted antibodies capable of binding LILRB1-Fc tagged, LILRB2-Fc tagged, LILRB3-Fc tagged, or LILRB4-Fc tagged construct were subcloned by limiting dilution to obtain monoclonal hybridoma cell lines. Approximately 10 days after subcloning, small volumes of media were removed from the wells and screened by solid-phase ELISA to identify antibody-producing clones. Selected positive clones were expanded and rescreened to ensure that the clones were still producing antibody and confirm specificity. Up to two positive clones per parental line were expanded for freezing 2 vials each. 1 ml supernatant was also collected for testing.

Solid Phase ELISA

The LILRB1-Fc tagged, LILRB2-Fc tagged, LILRB3-Fc tagged, or LILRB4-Fc tagged construct was immobilized to wells of a microbiter plate. Microtiter plates were coated with 2-10 μg ml LILRB1-Fc tagged, LILRB2-Fc tagged, LILRB3-Fc tagged, or LILRB4-Fc tagged construct in a coating buffer (0.2 M carbonate buffer (BuPH carbonate-bicarbonate buffer pack, Pierce item #28382) for 1-2 hours at room temperature or overnight at 4° C., then saturated with a blocking buffer (PBS containing 1% (w/v) BSA) for 1 hour at room temperature or overnight at 4° C., and washed 3× with washing buffer (PBS containing 0.5% (v/v) Tween 20). The antibody samples (i.e., the mouse serum or the hybridoma supernatants) to be screened were diluted in a diluent (0.1% solution of BSA in PBS). The diluted antibody samples were added to microtiter plate and the plate was incubated for 1-2 hours at room temperature. After having washed away any unbound substances with washing buffer, the level of bound anti-LILRB1 antibody, anti-LILRB2 antibody, anti-L ILRB3 antibody, or anti-LILRB4 antibody was determined using rabbit or goat anti-mouse peroxidase-conjugated secondary antibody (IgG specific) at recommended or experimentally derived dilution (usually 1/1000-1/10,000) in the diluent. After incubation for 30 minutes at room temperature and 3× wash with washing buffer, the chromogenic substrate, OPD (o-phenylenediamine), was added, then color was developed for 20 minutes, and stopped by adding 50 μL 2 N sulfuric acid. Absorbance at 490 nm was measured.

### Example 2. LPS Macrophage Activation Assay

Mature M-CSF derived macrophages were plated in 96 well cell-culture treated plates at a density of 2*10⁴ per well in cRPMI 1640 (+10% HI-FCS, 1× Glutamax, 1× Pen/Strep, 1× Beta-mercaptoethanol) and pre-treated with antibody at 20 g/ml for 15 minutes unless otherwise stated. LPS-EB ultrapure (Invivogen) was reconstituted according the manufacturer's recommendation and prepared to working concentration by dilution in cRPMI. Prior to dilution frozen LPS aliquots were briefly sonicated. Stimulation assays were performed under standard cell culture conditions for 16 hours at which point cell culture supernatants were retained for ELISA. TNFα ELISAs were performed using Human TNFα Ready-SET-Go ELISA kits (Thermo Fisher) in accordance with the manufacturer's guidelines.

### Example 3. HLA-A Blocking Assay

In this assay, anti-LILRB antibodies (e.g., anti-LILRB1 antibodies, anti-LILRB2 antibodies, and pan antibodies described herein) were screened in the presence of antibody 8E8.C4, a pan anti-LILRB1/2 antibody that increases HLA tetramer binding to the monocytes. This assay screens for antibodies that reduce HLA tetramer binding in the presence of antibody 8E8.C4.

Thawing Monocytes

A cryovial comprising about 1.25*10⁷ monocytes was placed in a 37° C. water bath until a portion of the ice remained in the cryovial. At such point, the cryovial was then placed on ice. About 1 mL of ice cold RPMI 1640 was added dropwise to the thawed monocytes in the cryovial. Next, the cell suspension was transferred to a 15 ml falcon tube and about 10 mL of ice cold cRPMI 1640 (10% HI-FCS, 1% P/S, 1% L-Glut, 0.1% b-ME) was subsequently added to the cell suspension. An additional 1 mL of ice cold cRPMI 1640 was further added to the cryovial and the solution was transferred to the 15 ml falcon tube. The tube was centrifuged for about 10 minutes at 300×g at 4° C. After the supernatant was removed, the cell pellet was re-suspended in 5 mL cold cRPMI 1640.

FACS Staining Protocol

The cells were counted, spin down at 300×g for 8 minutes, and then re-suspended to a concentration of about 1*10⁶ cells/mL in FACS buffer. About 100 μL of CD14+ monocytes were plated per well of a 96-well plate (1*10⁵ cells total). The cells were blocked in a final volume of 25 ul containing 40 ug/ml of antibody 8E8.C4 (a 1/50 dilution of 1 mg/mL of 8E8.C4) and 1/300 BD Fc block (Human BD Fc Block). 1 μL of an anti-LILRB test antibody at a concentration of 1 mg/mL was added to the blocking solution to give a final concentration of test antibody of 40 ug/ml. The 96-well plate was vortexed briefly to mix the solutions, and then incubated on ice for about 30 minutes. About 25 μL/well of a 1/50 dilution of HLA-A-PE tetramer (at a 1/100 final concentration) was added to each well. The plate was spin down for about 10 see at 500×g and vortexed briefly to mix the solution, and then incubated on ice for about 30 minutes. The cells were then washed with 1 mL FACS buffer and re-suspended in 75 μL of the FACS buffer. A Novocyte flow cytometer system was used for data acquisition.

FACS Assay Controls

The following control sets were used for the FACS analysis:
mouse IgG1 and IgG2A isotype controls with the HLA-A-PE tetramer;
anti-LILRB2 antibodies 42D1 and 287219 with the HLA-A-PE tetramer;
anti-LILRB1/2 antibody 8E8.C4 at 1 μg/mL concentration with the HLA-A-PE tetramer; and
cell only control.

The data was interpreted as a percentage (%) change in tetramer mean fluorescence intensity (MFI) compared to antibody 8E8.C4.

### Example 4. HLA-A Unmasking Assay

In this assay, anti-LILRB antibodies (e.g., anti-LILRB1 antibodies, anti-LILRB2 antibodies, and pan antibodies described herein) were screened without a pre-treatment step with antibody 8E8.C4. This assay screens for antibodies that block HLA tetramer binding, have no effect on binding, or induce an increased HLA tetramer binding to LILRB receptors.

Thawing Monocytes

A cryovial comprising about 1.25*10⁷ monocytes was placed in a 37° C. water bath until a portion of the ice remained in the cryovial. At such point, the cryovial was then placed on ice. About 1 mL of ice cold RPMI 1640 was added dropwise to the thawed monocytes in the cryovial. Next, the cell suspension was transferred to a 15 ml falcon tube and about 10 mL of ice cold cRPMI 1640 (10% HI-FCS, 1% P/S, 1% L-Glut, 0.1% b-ME) was subsequently added to the cell suspension. An additional 1 mL of ice cold cRPMI 1640 was further added to the cryovial and the solution was transferred to the 15 ml falcon tube. The tube was centrifuged for about 10 minutes at 300×g at 4° C. After the supernatant was removed, the cell pellet was re-suspended in 5 mL cold cRPMI 1640.

FACS Staining Protocol

The cells were counted, spin down at 300×g for 8 minutes, and then re-suspended to a concentration of about 1*10⁶ cells/mL in FACS buffer. About 100 μL of CD14+ monocytes were plated per well of a 96-well plate (1*10⁴ cells total). The cells were blocked in a final volume of 25 ul containing 1/300 BD Fc block (Human BD Fc Block). About 1 μL of an anti-LILRB test antibody was added at a final concentration of 1 mg/mL. The 96-well plate was vortexed briefly to mix the solutions, and then incubated on ice for about 30 minutes. About 25 μL/well of a 1/50 dilution of HLA-A-PE tetramer (at a 1/100 final concentration) was added to each well. The plate was spin down for about 10 see at 500×g and vortexed briefly to mix the solution, and then incubated on ice for about 30 minutes. The cells were then washed with 1 mL FACS buffer and re-suspended in 75 μL of the FACS buffer. A Novocyte flow cytometer system was used for data acquisition.

FACS Assay Controls

The following control sets were used for the FACS analysis:
mouse IgG1 and IgG2A isotype controls with the HLA-A-PE tetramer;
anti-LILRB2 antibodies 42D1 and 287219 with the HLA-A-PE tetramer;
anti-LILRB1/2 antibody 8E8.C4 at 1 μg/mL concentration with the HLA-A-PE tetramer; and
cell only control.

The data was interpreted as a fold increase in HLA-A-PE tetramer binding compared to HLA-A-PE tetramer only control.

### Example 5. Isolation of Human PBMCs and Selection of CD14+ Monocytes

Buffers:

MACS buffer
D-PBS
0.5% BSA
2 mM EDTA

Lymphoprep buffer
D-PBS
2% BSA

Cell Freezing Buffer
90% HI-FCS
10% DMSO

Whole blood was processed to generate PBMCs. Lymphoprep and SepMate tubes (Stemcell Technologies Cat #07801 & 85450) were used to isolate PBMCs. Specifically, about 15 mL Lymphoprep density gradient medium was aseptically transferred into a 50 mL SepMate tube through center hole in the insert, and the tubes were then allowed to warm to room temperature (about 20° C.). Blood sample was diluted with equal volume of PBS+2% (v/v) BSA, and was then gently transferred along the side of the SepMate tube so that it lays on top of the density gradient (SepMate tube keeps them separate). The sample was centrifuged at 1200×g for 15 minutes at 20° C. After centrifugation, the middle layer above insert contained PBMCs and was transferred into a separate falcon tube. The bottom layer below insert contained red blood cells (RBCs) and granulocytes and was subsequently discarded. The collected PBMCs and plasma was further centrifuged for about 8 minutes at 300×g and plasma supernatant was discarded (or transfer to new falcon tube if required). The PBMC pellet was washed twice with 50 mL PBS+2% (v/v) BSA and was subsequently re-suspended and centrifuged at 300×g for 8 minutes. PBMCs were further re-suspended in 3 mL ice cold MACS buffer (PBS 0.5% (v/v) BSA+2 mM EDTA) and counted at ˜1/100 dilution on a Countess cell counter.

MACS Positive Selection of CD14+ Monocytes

All solutions were pre-cooled and kept on ice in TC hood.

Cells were labeled with superparamagnetic beads. Specifically, cells were adjusted to a final concentration of 40 ul buffer/10⁷ total PBMCs (and spin at 300×g for 5 mins and re-suspend pellet if necessary after counting). About 10 ul of CD14 microbeads were added per 10⁷ total cells. The cell solution was then incubated for 15 min at 4° C. with continuous mixing.

PBMCs were isolated from human blood and were subsequently used for macrophage culture. Cell sample of 50 mL total volume was prepared with ice cold MACS buffer, and then centrifuged at 400×g for about 5 minutes at 4° C. The cell pellet was re-suspended in 500 μL MACS buffer per 108 total cells.

A magnetic separation kit was prepared by adding about 3 mL of MACS buffer to each LS column and let it run through. Next, about 3 mL of cell suspension was added to the LS column and let it run through. The column was washed 3 times with 3 mL of MACS buffer each. The cells were eluted with 2 volumes of 5 ml MACS buffer each by removing column from magnetic field and applying plunger force.

Monocyte Adherence and Macrophage Culture

Monocytes was transferred in serum free RPMI to T-25 flask, or single well of 6-well Nunc Delta (Costar Cat #140675) plate and cultured for about 2 h to serum shock monocytes. At 2 h, cRPMI supplemented was added with an additional 10% HI-FCS (20% FCS total) to bring the final FCS concentration to about 10%. About 50 ng/ml M-CSF (Peprotech) was then added to the culture. This would be d0. M-CSF was supplemented on d3. Cytokines was supplemented on d5+50% additional media (e.g. about 2.5 mL of cRPMI was added to 5 ml in T-25+ enough M-CSF for 7.5 ml total).

Macrophage Harvest

The macrophages were harvested at d7 post culture, and washed with 2× sterile room temperatured D-PBS. The harvested macrophages were then incubated on ice for about 45 minutes with ice-cold DBPS+4 mM EDTA. Using a cell scraper, macrophages were scrapped off and transferred to a 15 ml Falcon tube. Macrophages were then centrifuged at 300×g for about 8 minutes at 4° C. Supernatant was discarded. The cell pellet was re-suspended in 1 ml cRMPI (ice cold), counted and prepared for experiment.

### Example 6. HLA-G Binding Assay

HLA-G Tetramer Generation

PE conjugated HLA-G tetramer (HLA-G*01:01) folder in the presence of human beta-2-microglobulin and HLA-G binding nonamer peptide RIIPRHLQL (derived from human HIST1H2AG 78-86) was provided by the NIH tetramer core facility.

Thawing of Monocytes

A cryovial comprising about 1.25*10⁷ monocytes was placed in a 37° C. water bath until a portion of the ice remained in the cryovial. At such point, the cryovial was then placed on ice. About 1 mL of ice cold RPMI 1640 was added dropwise to the thawed monocytes in the cryovial. Next, the cell suspension was transferred to a 15 ml falcon tube and about 10 mL of ice cold cRPMI 1640 (10% HI-FCS, 1× Glutamax, 1× Pen/Strep, 1× beta-mercaptoethanol) was subsequently added to the cell suspension. An additional 1 mL of ice cold cRPMI 1640 was further added to the cryovial and the solution was transferred to the 15 ml falcon tube. The tube was centrifuged for about 10 minutes at 300×g at 4° C. After the supernatant was removed, the cell pellet was re-suspended in 5 mL cold cRPMI 1640.

FACS Staining Protocol

Thawed monocytes were washed twice in 1 mL FACS buffer (D-PBS+2% BSA, 2 mM EDTA) and resuspended to 2×10⁶/mL in FACS buffer. 1 L each anti-Lilrb test antibody at a stock concentration of 1 mg/mL was added to the bottom of a v-bottom plate, followed by 50 uL monocytes and antibody allowed to bind on ice for 30 minutes before addition of tetramer. PE conjugated tetramer was then added to a final concentration of 1 μg/mL and stained on ice, protected from light, for 30 minutes. Cells were washed twice by addition of 200 μL FACS buffer and centrifugation for about 10 minutes at 300×g at 4° C. Data was acquired on a Novocyte 3000 flow cytometer.

### Example 7. HLA-G Conditioned DC MLR Assay

Preparing PBMCs

PBMCs were obtained from in-house blood donors. Blood was drawn into K2EDTA tubes (BD Cat #366643) and PBMCs isolated from the buffy coat of Lymphoprep (Stemcell Cat #07801) separations using Sepmate-50 tubes (Stemcell Cat #85450) in accordance with manufacturer's guidelines. Briefly, blood was diluted in an equal volume D-PBS supplemented with 2% w/v BSA (Sigma #A7906) and centrifuged at 1200×g for 15 minutes at room temperature on 15 mL Lymphoprep in a Sepmate-50 tube. Buffy coat and serum layer were then isolated and PBMCs washed 2× in 20 mL D-PBS+0.5% (v/v) BSA+2 mM EDTA prior to use. Responder PBMCs for MLR assays were frozen in Cryostor CS-10 media (Sigma #C2874) at 1.5×10⁷/mL at −80° C. overnight and stored in the liquid phase of LN2 tank until later use. DCs were prepared from monocytes isolated from freshly prepared PBMCs.

Thawing of PBMCs

A cryovial comprising 1-5*10⁷ PBMCs was placed in a 37° C. water bath until a portion of the ice remained in the cryovial. At such point, the cryovial was then placed on ice. About 1 mL of ice cold RPMI 1640 was added dropwise to the thawed monocytes in the cryovial. Next, the cell suspension was transferred to a 15 ml falcon tube and about 10 mL of ice cold cRPMI 1640 (10% HI-FCS, 1× Glutamax, 1× Pen/Strep, 1× beta-mercaptoethanol) was subsequently added to the cell suspension. An additional 1 mL of ice cold cRPMI 1640 was further added to the cryovial and the solution was transferred to the 15 ml falcon tube. The tube was centrifuged for about 10 minutes at 300×g at 4° C. After the supernatant was removed, the cell pellet was re-suspended in 5 mL cold cRPMI 1640.

Generation ofHLA-G5 Overexpression Supernatant

HLA-G5 mRNA sequence was cloned into a modified pCMV6 vector containing an N-terminal FLAG/His sequence separated by a short Gly-Ser linker using BsiWI and BamHI restriction enzymes. Ligated constructs were transformed into oneShot Top10 chemically competent E. coli (Thermo Fisher) and grown on Carbenicillin agar plates (Teknova). Protein expression was obtained by transfecting expiCHO cells (Thermo Fisher) using the Maxcyte transfection system and overexpression supernatant used for functional assays.

Maturation of HLA-G Conditioned Monocyte-Derided DCs

DCs for MLR reactions were prepared from freshly isolated PBMCs by purifying monocytes from peripheral blood using CD14 positive selection beads (Miltenyi cat #130-050-201). PBMCs were adjusted to 40 L/10⁷ cells in MACS buffer (D-PBS+0.5% (v/v) BSA+2 mM EDTA) and 10 μL beads added and incubated at 4° C. for 15 mins. Cells were washed twice with MACS buffer and resuspended to a final concentration of 2×10⁸/mL and isolated using MS columns (Myltenyi #130-042-201). DCs were differentiated by culturing at a final density of 1×10⁶/mL in 3 mL cRPMI (10% HI-FCS, 1× Glutamax, 1× Pen/Strep, 1× beta-mercaptoethanol) supplemented with 50 ng/mL GM-CSF (Peprotech #300-03) in 6-well TC treated plates (Costar #140675). Cells were fed on days 2 and 4 by replacement of 50% volume cRPMI supplemented with fresh GM-CSF. On day 5 immature DCs were recovered by pipetting and transferred to 6-well plates. DCs were matured by the addition of DC maturation supplement (Stemcell #10989) to 1× concentration for 2 days. Day 7 mature DCs were conditioned for a further 48 h in 96-well u-bottom plates (Thermo #163320) by addition of HLA-G5 overexpression supernatant generated using expiCHO cells at a 1:25 dilution following 1-hour pre-treatment with test antibodies or isotype controls at a final concentration of 20 μg/mL.

Setup of MLR Assay

Responder PBMCs were thawed from frozen vials as per the protocol above and cultured in the presence of HLA-G conditioned DCs at 1:50 DC: PBMC ratio using 2×10⁵ PBMCs/well. MLRs were performed in u-bottom tissue culture treated plates (Thermo #163320) at a final culture volume of 200 uL cRPMI (10% HI-FCS, 1× Glutamax, 1× Pen/Strep, 1× beta-mercaptoethanol) in the presence of anti-Lilrb antibody (20 ug/mL). On day 7 supernatants were harvested by centrifuging cells at 300×g for 8 minutes. Secreted IFNγ was measured by ELISA in culture supernatants using Ready-Set-Go anti-human IFNγ ELISA kits (Thermo Fisher #88-8314-76) in accordance with manufacturer's instructions.

### Example 8. PBMC Mediated Tumor Killing Assay

Preparing PBMCs

PBMCs were obtained from in-house blood donors. Blood was drawn into K2EDTA tubes (BD Cat #366643) and PBMCs isolated from the buffy coat of Lymphoprep (Stemcell Cat #07801) separations using Sepmate-50 tubes (Stemcell Cat #85450) in accordance with manufacturer's guidelines. Briefly, blood was diluted in an equal volume D-PBS supplemented with 2% w/v BSA (Sigma #) and spun at 1200×g for 15 minutes at room temperature on 15 mL Lymphoprep in a Sepmate-50 tube. Buffy coat and serum layer were then isolated and PBMCs washed 2× in 20 mL D-PBS+0.5% (v/v) BSA+2 mM EDTA prior to use. PBMCs for cytotoxicity assays were frozen in Cryostor CS-10 media (Sigma #C2874) at 1.5×10⁷/mL at −80° C. overnight and stored in the liquid phase of LN2 tank until later use.

Thawing PBMCs

A cryovial comprising 1-5*10⁷ PBMCs was placed in a 37° C. water bath until a portion of the ice remained in the cryovial. At such point, the cryovial was then placed on ice. About 1 mL of room temperature RPMI 1640 was added dropwise to the thawed monocytes in the cryovial. Next, the cell suspension was transferred to a 15 ml falcon tube and about 10 mL of room temperature cRPMI 1640 (10% HI-FCS, 1× Glutamax, 1× Pen/Strep, 1× beta-mercaptoethanol) was subsequently added to the cell suspension. An additional 1 mL of room temperature cRPMI 1640 was further added to the cryovial and the solution was transferred to the 15 ml falcon tube. The tube was centrifuged for about 10 minutes at 300×g at room temperature. After the supernatant was removed, the cell pellet was re-suspended in 2×10⁶ cells/mL in cRPMI.

### Example 9. Binding of Antibodies to LILRB1, LILRB2 and LILRB3 Common Variants

Lilrb loci are highly polymorphic, based on published data and 1000 Genomes datasets we identified 3 haplotypes for Lilrb extracellular domain protein that comprise 76-92% of sequence diversity, denoted Lilrb1_01-Lilrb1_03. Similarly, four such haplotypes were identified for Lilrb2 that collectively comprise 77% of population sequence heterogeneity, denoted Lilrb2_01-Lilrb2_05. The LILRB3 gene is highly polymorphic, based upon published data (Bashirova et al, Immunogenetics. 2014, 66-1) 13 unique alleles for LILRB3 were identified, however, within ectodomain sequences, LILRB3 alleles LILRB3_01 and LILRB3_05 comprise 79% of common variants observed and were used to represent the diversity in haplotype binding for LILRB3.

mRNA sequences for LILRB1_01-LILRB1_03, LILRB2_01-LILRB2_04, LILRB3_01 and LILRB3_05 extracellular domains were cloned into a modified pCMV6 vector containing an N-terminal FLAG/His sequence separated by a short Gly-Ser linker using BsiWI and BamHI restriction enzymes. Ligated constructs were transformed into oneShot Top10 chemically competent E. coli (Thermo Fisher) and grown on Carbenicillin agar plates (Teknova). Protein expression was obtained by transfecting 293F cells (Thermo Fisher) with Fectopro (Polyplus) transfection reagent. Expressed protein was purified using FPLC using Ni2 coated columns (HisTrap Sure, GE healthcare) and eluted using 20 mM NaPO4 pH 7.2, 500 mM NaCl, 500 mM Imidazole. Protein concentration and buffer exchange in Sodium Phosphate buffer (pH 7.2) was performed using Amicon Ultra 30 kDa molecular weight columns. Purified proteins were coated onto 96 well ELISA assay plates (Corning #9018) in carbonate-bicarbonate buffer pH 9.2 (Thermo Scientific #28382) at 2 μg/mL (200 ng/well) overnight at 4° C. Plates were washed 3×400 μL TBS Tween-20 (Boston Bioproducts) and blocked in Superblock-T20 (Thermo Fisher) for 1 hour at room temperature. Antibodies were then added at the appropriate concentration in 100 μL Superblock-T20 and incubated at room temperature for 2 h. Following washing, secondary HRP conjugated goat-anti-mouse-F(ab)′ (Thermo Fisher #31436) antibody was added at 400 ng/mL and incubated for 30 minutes at room temperature. Plates were further washed 5×400 μL TBS Tween-20 and ELISA assay was developed using TMB substrate solution (Sigma) by eye and assay quenched using 2M H2SO4 solution. Optical densities were measured using a spectramax M3. ELISAs were quantified by subtracting absorbance at 450 nm-570 nm.

### Example 10. Binding of Antibodies to LILRA Proteins

mRNA sequences for LILRA1, LILRA2. LILRA3, LILRA4 and LILRA5 extracellular were cloned into a modified pCMV6 vector containing an N-terminal FLAG/His sequence separated by a short Gly-Ser linker using BsiWI and BamHI restriction enzymes. Ligated constructs were transformed into oneShot Top10 chemically competent E. coli (Thermo Fisher) and grown on Carbenicillin agar plates (Teknova). Protein expression was obtained by transfecting 293F cells (Thermo Fisher) with Fectopro (Polyplus) transfection reagent. Expressed protein was purified using FPLC using Ni2 coated columns (HisTrap Sure, GE healthcare) and eluted using 20 mM NaPO4 pH 7.2, 500 mM NaCl, 500 mM Imidazole. Protein concentration and buffer exchange in Sodium Phosphate buffer (pH 7.2) was performed using Amicon Ultra 30 kDa molecular weight columns. Purified proteins were coated onto 96 well ELISA assay plates (Corning #9018) in carbonate-bicarbonate buffer pH 9.2 (Thermo Scientific #28382) at 2 μg/mL (200 ng/well) overnight at 4° C. Plates were washed 3×400 μL TBS Tween-20 (Boston Bioproducts) and blocked in Superblock-T20 (Thermo Fisher) for 1 hour at room temperature. Antibodies were then added at the appropriate concentration in 100 μL Superblock-T20 and incubated at room temperature for 2 h. Following washing, secondary HRP conjugated goat-anti-mouse-F(ab)′ (Thermo Fisher #31436) antibody was added at 400 ng/mL and incubated for 30 minutes at room temperature. Plates were further washed 5×400 μL TBS Tween-20 and ELISA assay was developed using TMB substrate solution (Sigma) by eye and assay quenched using 2M H2SO4 solution. Optical densities were measured using a spectramax M3. ELISAs were quantified by subtracting absorbance at 450 nm-570 nm.

### Example 11. Binding of Antibodies to LILRB2 d1d2 and LILRB2 d3d4 Proteins

mRNA sequences for LILRB2-d1d2 and LILRB2-d3d4 regions defined by Uniprot annotations were cloned with a short Gly-Ser linker into the human IgG pFUSEN vector (Invivogen) using BamHI and KpnI restriction sites (NEB). Ligated constructs were transformed into oneShot Top10 chemically competent E. coli (Thermo Fisher) and grown on Zeocin agar plates (Teknova). Protein expression was obtained by transfecting FreeStyle 293F cells (Thermo Fisher) with FectoPro transfection reagent. Expressed protein was purified using FPLC using protein A coated columns (MabSelect Sure, GE healthcare) and eluted using IgG elution buffer (Thermo Fisher). Protein concentration and buffer exchange in Sodium Phosphate buffer (pH 7.2) was performed using Amicon Ultra 30 kDa molecular weight columns. Purified proteins were coated onto 96 well ELISA assay plates (Corning #9018) in carbonate-bicarbonate buffer pH 9.2 (Thermo Scientific #28382) at 2 μg/mL (200 ng/well) overnight at 4° C. Plates were washed 3×400 μL TBS Tween-20 (Boston Bioproducts) and blocked in Superblock-T20 (Thermo Fisher) for 1 hour at room temperature. Antibodies were then added at the appropriate concentration in 100 μL Superblock-T20 and incubated at room temperature for 2 h. Following washing, secondary HRP conjugated goat-anti-mouse-F(ab)′ (Thermo Fisher #31436) antibody was added at 400 ng/mL and incubated for 30 minutes at room temperature. Plates were further washed 5×400 μL TBS Tween-20 and ELISA assay was developed using TMB substrate solution (Thermo Fisher #34029) by eye and assay quenched using 2M H2SO4 solution. Optical densities were measured using a spectramax M3. ELISAs were quantified by subtracting absorbance at 450 nm-570 nm.

### Example 12. ELISA Binding of HLA-G Tetramer to Extracellular LILRB1-Fc, LILRB2-Fc, LILRB2_d1d2_Fc or LILRB2_d3d4-Fc and Anti-LILRB HLA-G Blocking ELISA Assay

HLA-G tetramer binding ELISAs to LILRB proteins were all performed in a similar manner. Appropriate LILRB-Fc tagged protein (described above) were coated onto 96 well ELISA assay plates (Corning #9018) in carbonate-bicarbonate buffer pH 9.2 (Thermo Scientific #28382) at 2 μg/mL (200 ng/well) overnight at 4° C. Plates were washed 3×400 μL TBS Tween-20 (Boston Bioproducts) and blocked in Superblock-T20 (Thermo Fisher) for 1 hour at room temperature.

For antibody blockade experiments test antibodies were pre-incubated with LILRB-coated plates at the indicated concentrations for 1 h at room temperature in 100 μL prior to the addition of HLA-G tetramer. HLA-G tetramer diluted in Superblock-T20 was then added assay plates alongside supplemental anti-Lilrb antibody to maintain antibody concentration if necessary. HLA-G tetramer was added at a concentration of 1 μg/mL for LILRB1 assays and 6 μg/mL for LILRB2 assays in a final volume of 200 μL and incubated at room temperature for 1 hour. Plates were washed 3×400 μL TBS Tween-20 and HLA-G tetramer detected via the addition of MEM-G/9-biotin (Thermo Fisher #MA1-19513) in 100 μL at a concentration of 2 μg/mL for 1 h. Plates were washed 3×400 μL TBS Tween-20 and incubated with streptavidin-HRP (Thermo Fisher) for 1 hour, washed 5×400 μL TBS Tween-20, and then developed using TMB substrate solution (Thermo Fisher #34029). Assay was quenched using 2M H2SO4 solution and absorbance at 450 nm and 570 nm measured using a spectramax M3.

### Example 13. Linear Peptide Epitope Mapping

Forty four N-terminal Biotin-Ahx-modified length of 15 residues linear peptides were designed to cover whole sequence of human LILRB2 protein. Each peptide shares 5 overlapping residues with adjacent one and were synthesized by Genscript.

Neutravidin were coated onto ELISA plates at a concentration of 2 g/ml and incubated at 4° C. overnight. Next day the coating solution was removed, and the wells blocked with Superblock T20 (Pierce). After 30 minutes, the blocking solution was removed, the wells washed three times with 350 μl of TBS-T and then 2 g/ml of biotinylated peptides were added in a final volume of 100 d in Superblock T20. After 1 hour incubation at room temperature, the wells were washed with TBS-T three times, and plate incubated with 3 g/ml Detection Antibody for 1 hour. Next, the plates were washed with TBS-T as before and incubated with 100 μl of Goat Anti-mouse IgG (H+L)-HRP conjugate (Invitrogen) at final dilution of 1/2000 in SuperBlock T20 for 45 minutes. The plates were washes as before and 100 μl of Supersensitive Liquid Substrate TMB for ELISA (Sigma) added and color allowed to develop. Reaction stopped by adding 100 μl 1N H₂SO₄ and O.D. values were determined at 450 nm.

### Example 14. A 2-Way MLR Protocol

PBMCs from two donors were mixed together 1:1 ratio in X-Vivo medium and plated into 96-well U-bottom plates at a density of 50,000 cells per donor in final volume of 200 μL media. The starting concentration of 10 μg/mL of Adanate antibodies or isotype controls added with 10-fold serial dilutions in the presence of HLA-G5 supernatant (1:40 final dilution). Cell supernatants are harvested on day 6 for ELISA analysis of MLR response using IFNγ ELISA kit (Biolegend).

### Example 15. In Vitro Generation and Isolation of CD33⁺ MDSCs

For MDSC generation, human PBMCs were cultured at a density of 1×10⁶/mL in RPMI 1640 with 10% HI-FCS (Sigma-Aldrich), 1× Glutamax, 1× β-mercaptoethanol containing HLA-G5 overexpression supernatant (described previously) at final dilution of 1:40 dilution in 10 cm² ultra low binding plates in the absence or present of 5 μg of anti-LILRB antibodies for 7 days.

For CD33⁺ MDSC cell isolation, cultured PBMCs were harvested at day 7 and washed with 2MACS buffer (PBS with 0.5% BSA and 2 mM EDTA) once and incubated with anti-human CD33 magnetic microbeads (Miltenyi Biotec) in MACS buffer for 15 minutes. Cells were washed with MACS buffer once and CD33⁺ cells were separated using MS column separation (Miltenyi Biotec), per manufacturer's instructions. The purity of the purified cells was checked by flow cytometry staining with anti-human CD33 antibody (Biolegend) in PBS 1% BSA (FACS buffer) and populations that were >90% pure were used for the experiments described herein.

### Example 16. CD33⁺ MDSC Cells-Derived Suppression Assay

The suppressive function of CD33⁺ cells was measured by their ability to inhibit the proliferation of autologous T cells. T cells isolated from PBMCs of autologous donors by anti-human CD3 magnetic microbeads and MS column separation (Miltenyi Biotec) following manufacturer's instruction. Next, T cells were labelled with 5 μM of CellTrace FarRed Dye (Invitrogen) and seeded at 1×10⁵ cells/well in 96-well plates and 1×10⁵ CD33⁺ cells were added into each well at 1:1 ratio with T cells. T cell stimulation was provided by anti-CD3/CD28 stimulation beads (Invitrogen) and IL-2 (100 U/ml PeproTech). Suppression assay wells were analyzed by flow cytometry for proliferation of T cells after 3 days. For each assay run, controls included T cells cultured alone with and without T cell stimulation, and T cells cultured with CD33⁺ cells from medium-only without antibody. Each CD33⁺ sample was run in triplicate, and data were acquired as percentage of proliferation for minimum 15,000 events of live, CD4 and CD8 lymphoid-gated cells. Samples were run on a Novocyte flow cytometer (ACEA Biosciences), and data analysis was performed using FlowJo software (FlowJo). T cell proliferation index was determined by normalizing data with average of stimulated T cell controls.

### Example 17. Anti-LILRB Antibody Binding and Functional Properties

FIG. 2 illustrates M1 activating properties of exemplary anti-LILRB antibodies described herein. As shown in this figure, anti-LILRB2/3/4 antibodies show a range of M1 activating properties. The assay was carried out overnight.

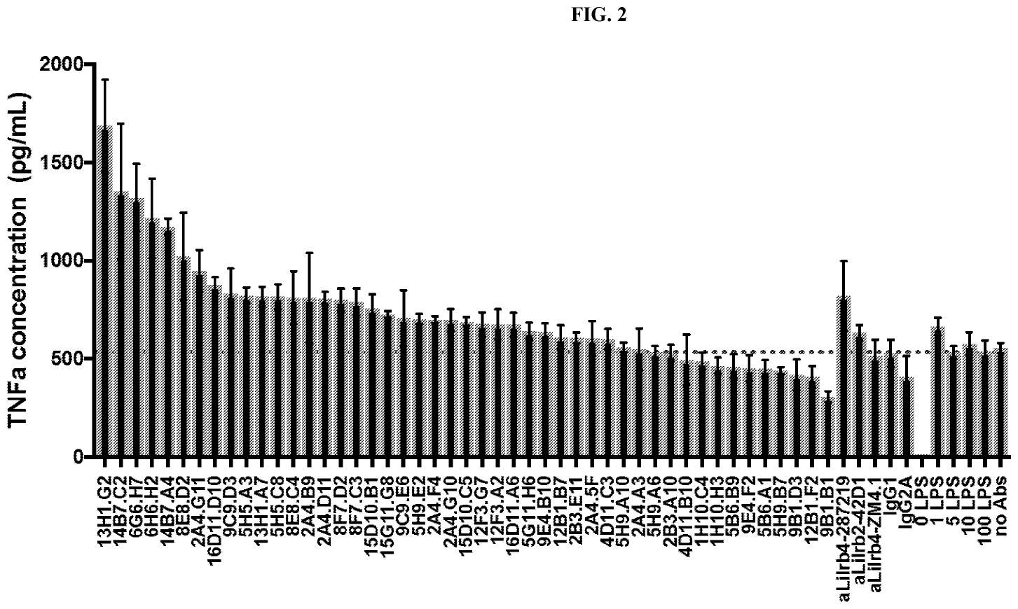

FIG. 3 illustrates multiple binding and functional properties of exemplary anti-LILRB antibodies described herein.

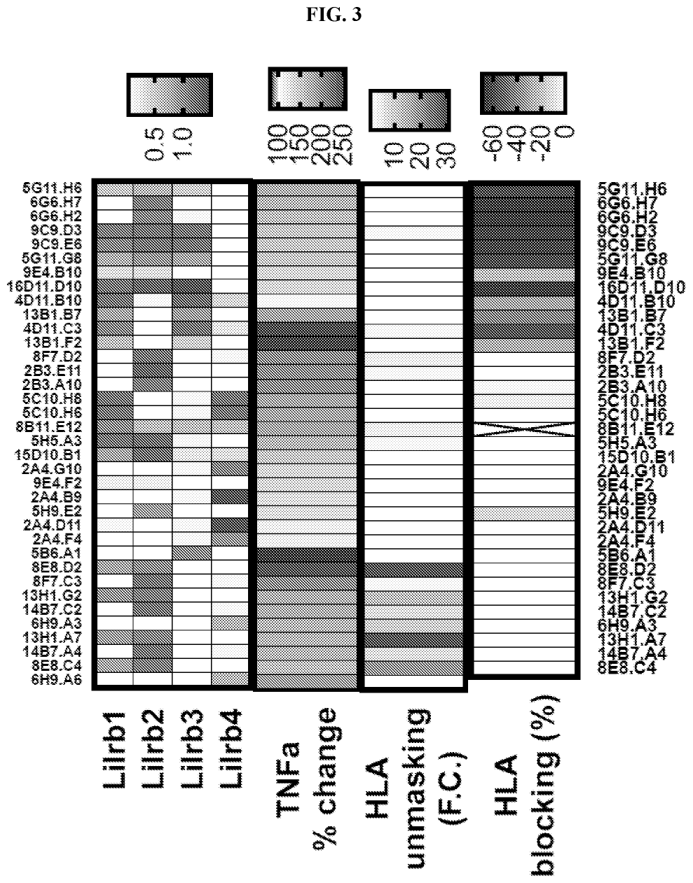

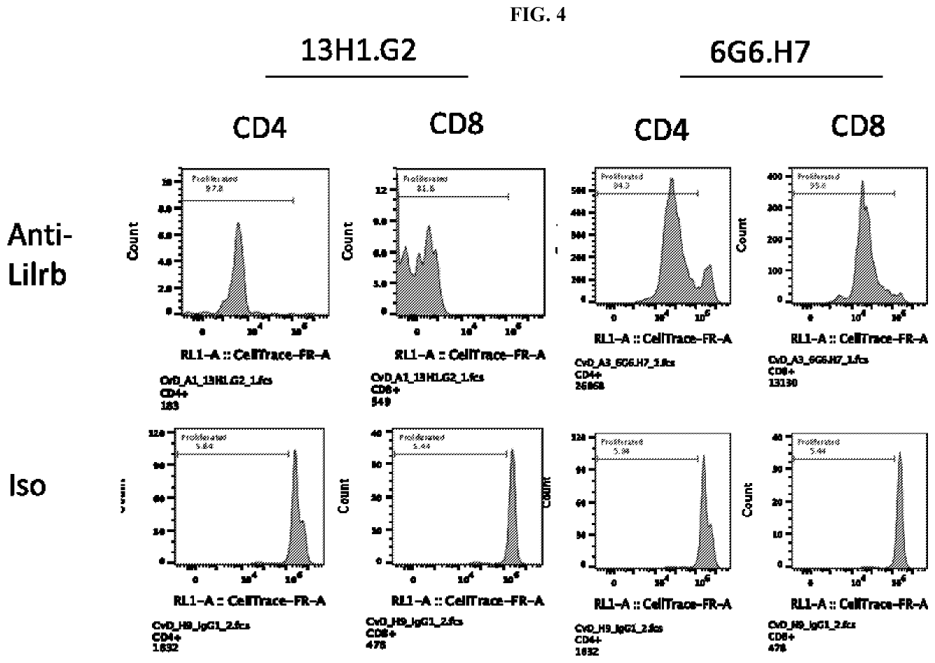

FIG. 4 shows proliferation of T cells in a mixed lymphocyte reaction (MLR) setting by exemplary anti-LILRB2 antibodies 13H1.G2 and 6G6.H7.

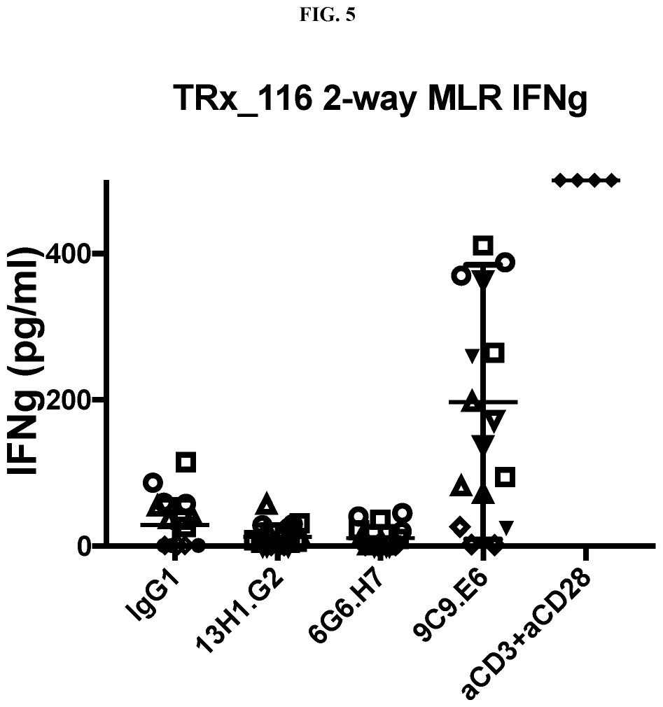

FIG. 5 shows IFNγ production under a 2-way mixed lymphocyte reaction (MLR) setting by exemplary anti-LILRB2 antibodies 13H1.G2 and 6G6.H7 and pan anti-LILRB1/2/3 antibody 9C9.E6.

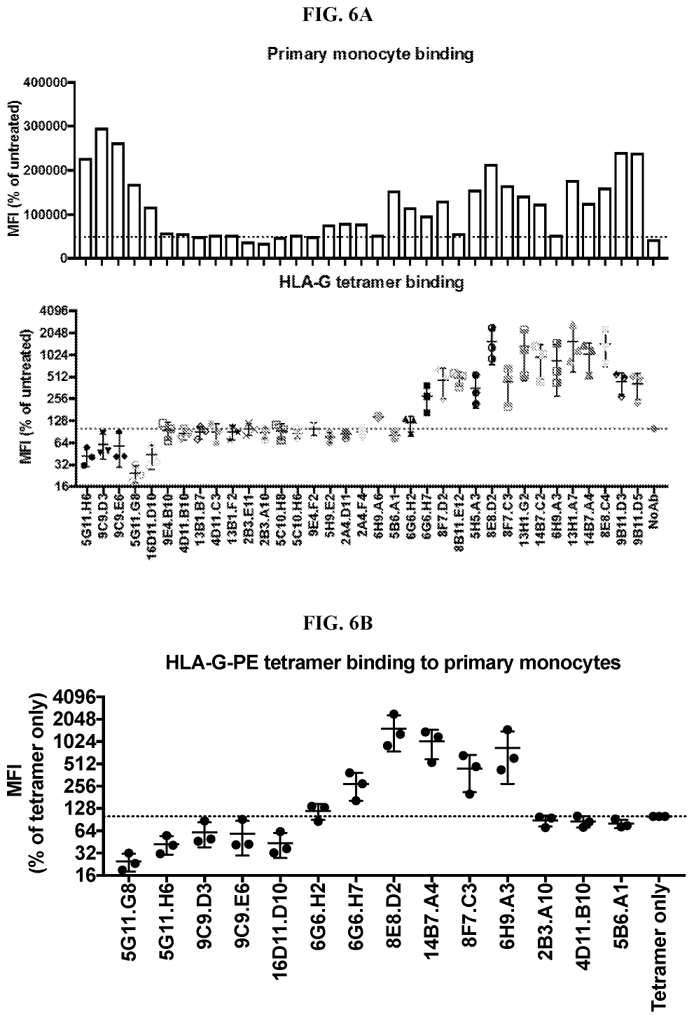

FIG. 6A illustrates HLA-G binding profile of exemplary anti-LILRB antibodies. FIG. 6A top panel illustrates antibody binding profiles with respect to primary monocytes. FIG. 6A bottom panel illustrates binding profiles of HLA-G tetramer to primary monocytes. The analysis was carried out by FACS.

FIG. 6B shows HLA-G-*01:01-PE tetramer binding to primary CD14⁺ monocytes as determined by flow cytometry. Antibody concentrations used were 20 μg/mL. MFI values of tetramer staining are expressed as a ratio relative to tetramer only control. Error bars represent mean+/−standard deviation of n=3 independent donors.

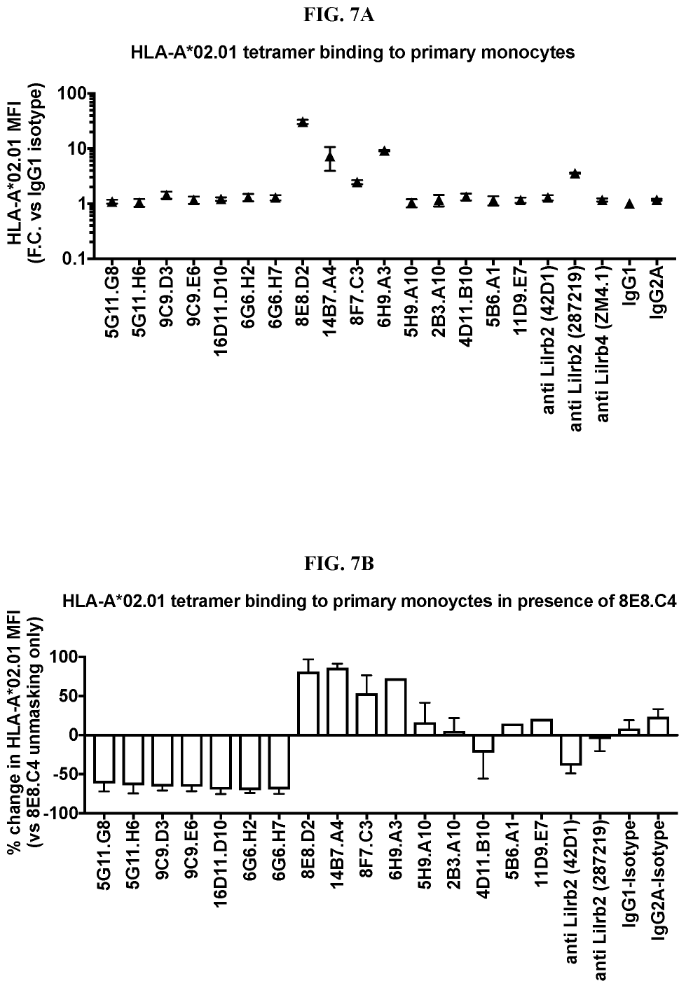

FIG. 7A shows HLA-A*02:01-PE tetramer unmasking assay, binding to primary CD14⁺ monocytes as determined by flow cytometry. Antibody concentrations used were 20 μg/mL. MFI values of tetramer staining are expressed as a ratio relative to IgG1 isotype control. Error bars represent mean+/−standard deviation of n=3 independent donors. Antibodies #287219 (R&D Systems), 42D1 (Biolegend), and ZM4.1 are commercial antibodies.

FIG. 7B shows HLA-A*02:01-PE tetramer blocking assay, binding to primary CD14⁺ monocytes as determined by flow cytometry. HLA-A tetramer blocking assays are performed in the presence of unmasking antibody clone 8C8.C4 (20 μg/mL) to maximize background tetramer staining intensity. HLA-A blocking is calculated as the percentage of signal obtained from 8E8.C4 only treatment. Values shown are mean+/−standard deviation of n=3 independent donors. Antibodies #287219 (R&D Systems) and 42D1 (Biolegend) are commercial antibodies.

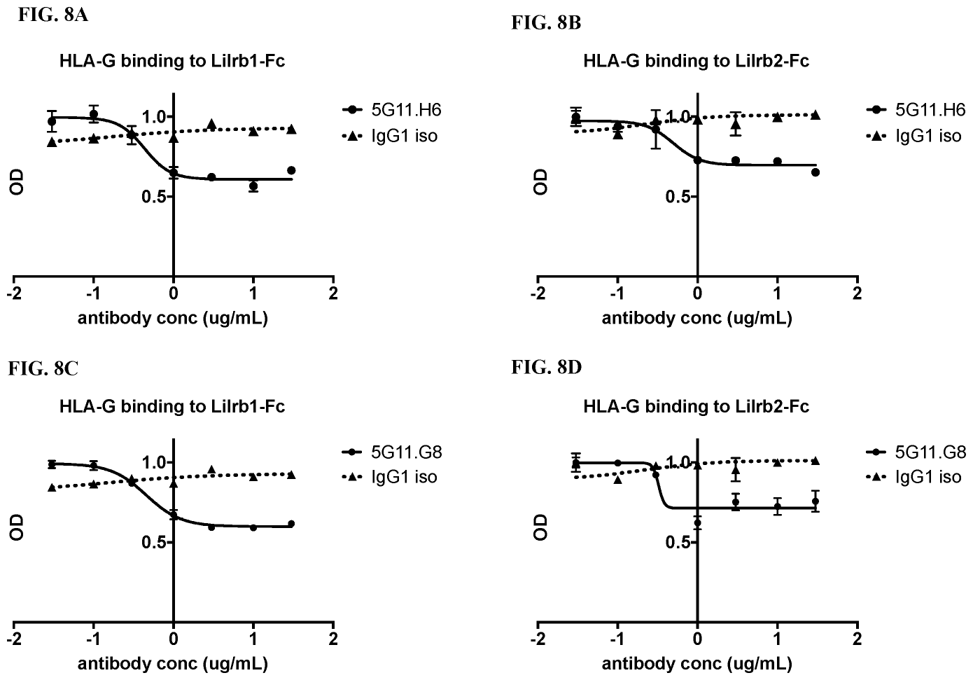

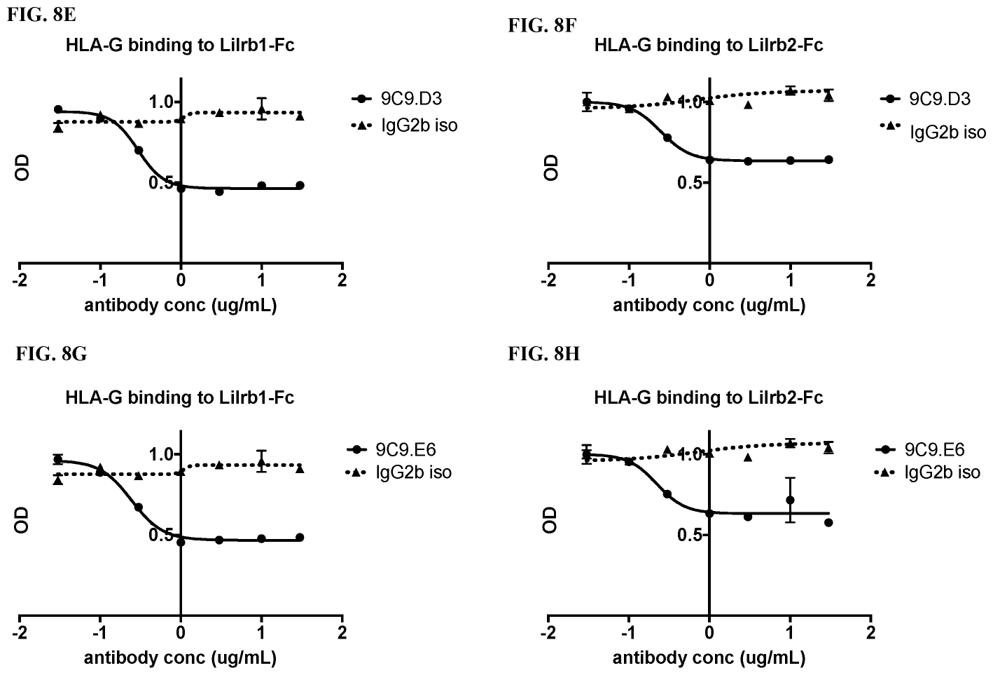

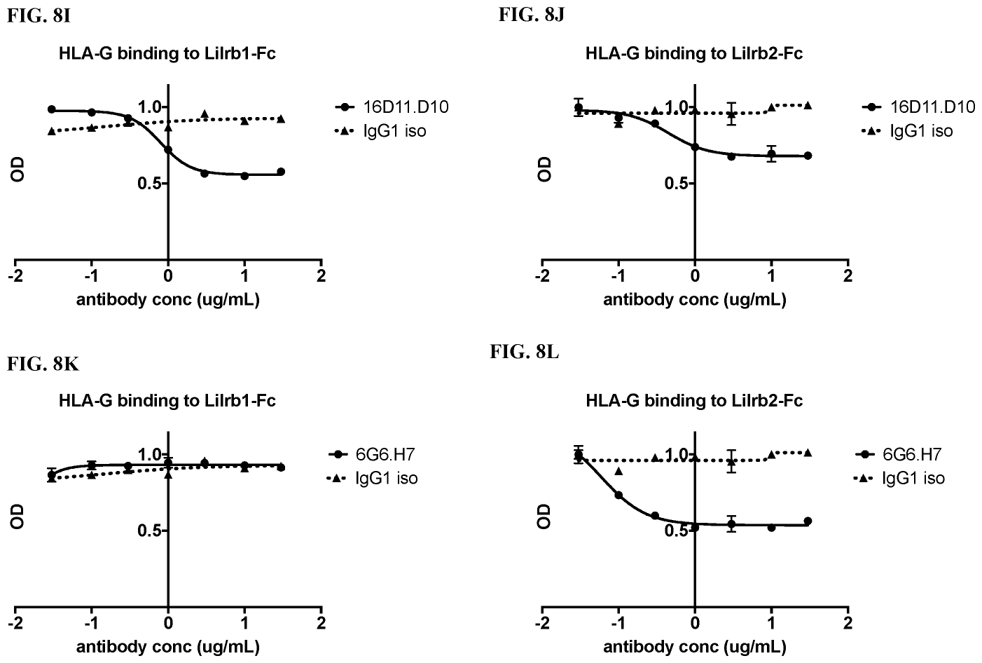

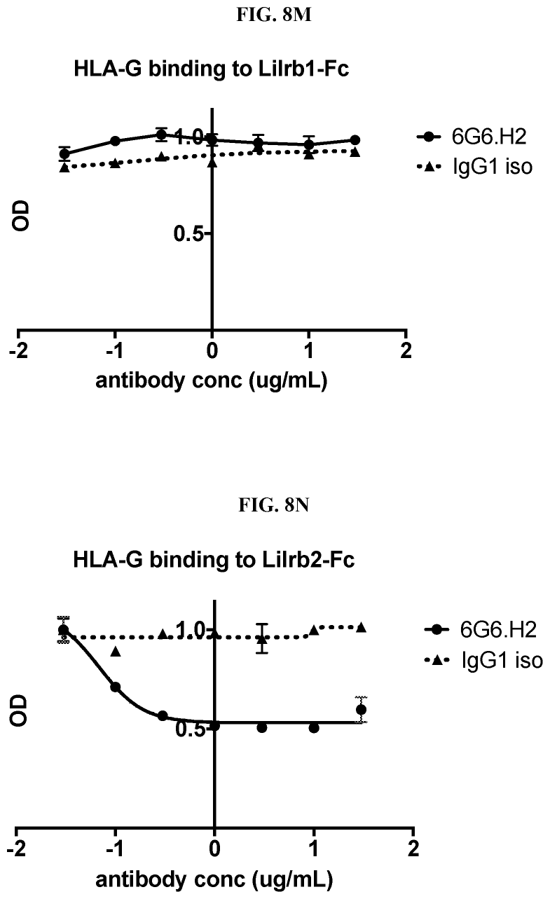

FIG. 8A-FIG. 8N show ELISA binding of HLA-G tetramer to LILRB1-Fc and LILRB2-Fc proteins in the presence of HLA-G blocking antibodies. FIGS. 8A and 8B: antibody 5G11.H6; FIGS. 8C and 8D: antibody 5G11.G8; FIGS. 8E and 8F: antibody 9C9.D3; FIGS. 8G and 8H: antibody 9C9.E6; FIGS. 8I and 8J: antibody 16D11.D10; FIGS. 8K and 8L: antibody 6G6.H7; FIGS. 8M and 8N: antibody 6G6.H2. ELISA plates coated with Lilrb-Fc protein (200 ng/well) were incubated with test antibody at the indicated concentration for 1 h prior to the addition of HLA-G tetramer at 1 μg/mL and 6 μg/mL for LILRB1 and LILRB2 respectively. Data shown are mean+/−standard deviation of triplicate measurements.

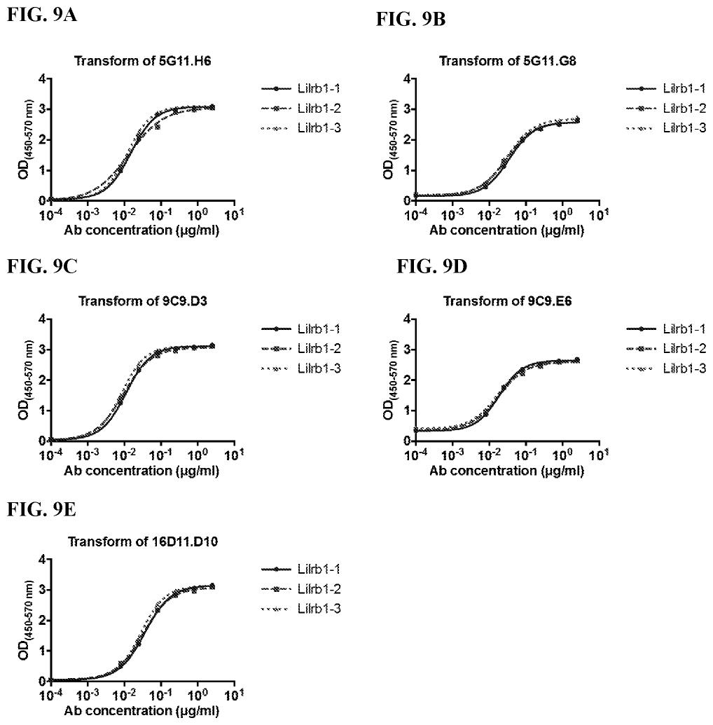

FIG. 9A-FIG. 9E ELISA binding of anti-LILRB antibodies to full-length extracellular LILRB1 proteins Lilrb1_01 (SEQ ID NO: 33), Lilrb1_02 (SEQ ID NO: 34), and Lilrb1_03 (SEQ ID NO: 35). FIG. 9A: antibody 5G11.H6; FIG. 9B: antibody 5G11.G8; FIG. 9C: antibody 9C9.D3; FIG. 9D: antibody 9C9.E6; and FIG. 9E: antibody 16D11.D10.

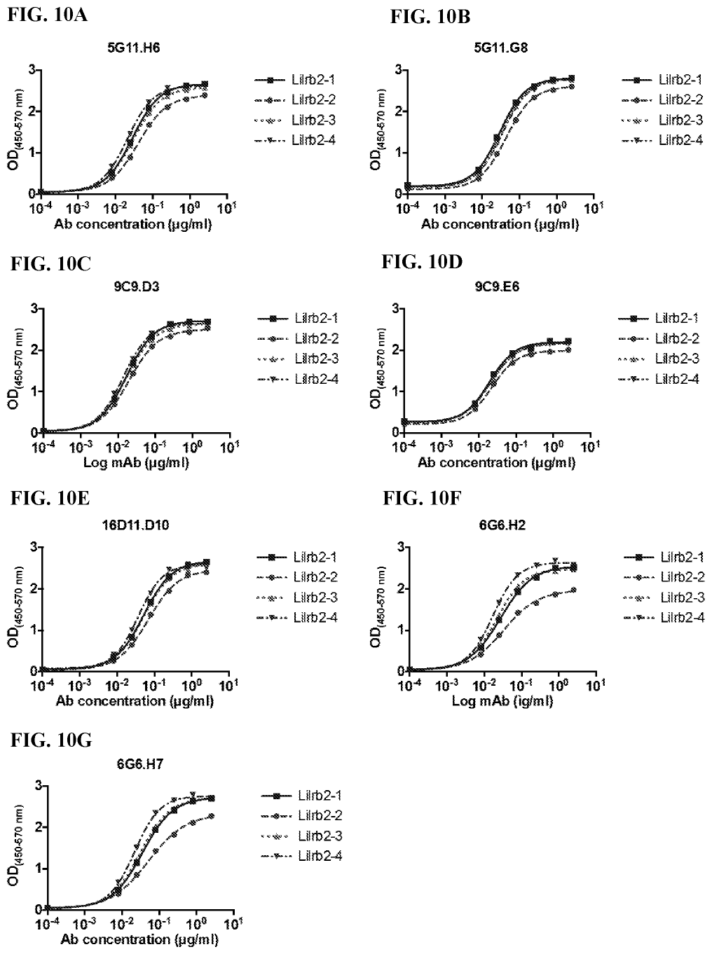

FIG. 10A-FIG. 10G show ELISA binding of anti-LILRB antibodies to full-length extracellular LILRB2 proteins Lilrb2_01 (SEQ ID NO: 36), Lilrb2_02 (SEQ ID NO: 37), Lilrb2_03 (SEQ ID NO: 38), and Lilrb2_04 (SEQ ID NO: 39). FIG. 10A: antibody 5G11.H6; FIG. OB: antibody 5G11.G8; FIG. 10C: antibody 9C9.D3; FIG. 10D: antibody 9C9.E6; FIG. 10E: antibody 16D11.D10;

FIG. 10F: antibody 6G6.H2; and FIG. OG: antibody 6G6.H7.

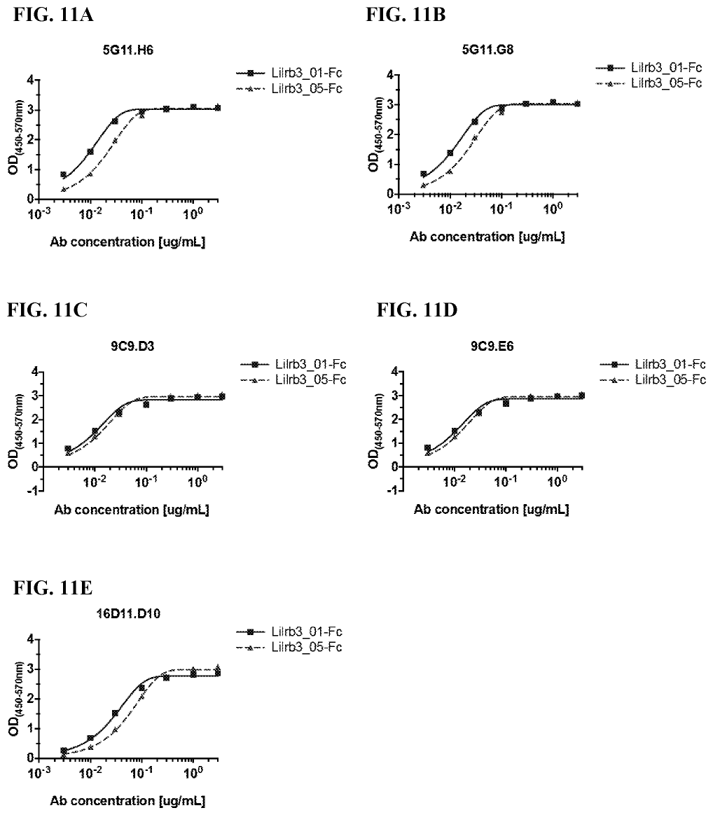

FIG. 11A-FIG. 11E show ELISA binding of anti-LILRB antibodies to full-length extracellular LILRB3 proteins Lilrb3_01 (SEQ ID NO: 40) and Lilrb3_05 (SEQ ID NO: 41). FIG. 11A: antibody 5G11.H6; FIG. 11B: antibody 5G11.G8; FIG. 11C: antibody 9C9.D3; FIG. 11D: antibody 9C9.E6; and FIG. 11E: antibody 16D11.D10.

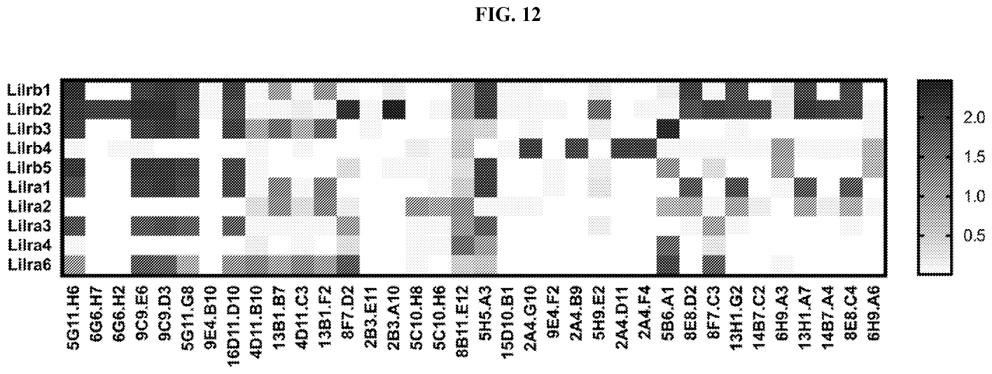

FIG. 12 shows binding profile of exemplary anti-LILRB antibodies with respect to LILRBs 1-5 and LILRAs 1-6.

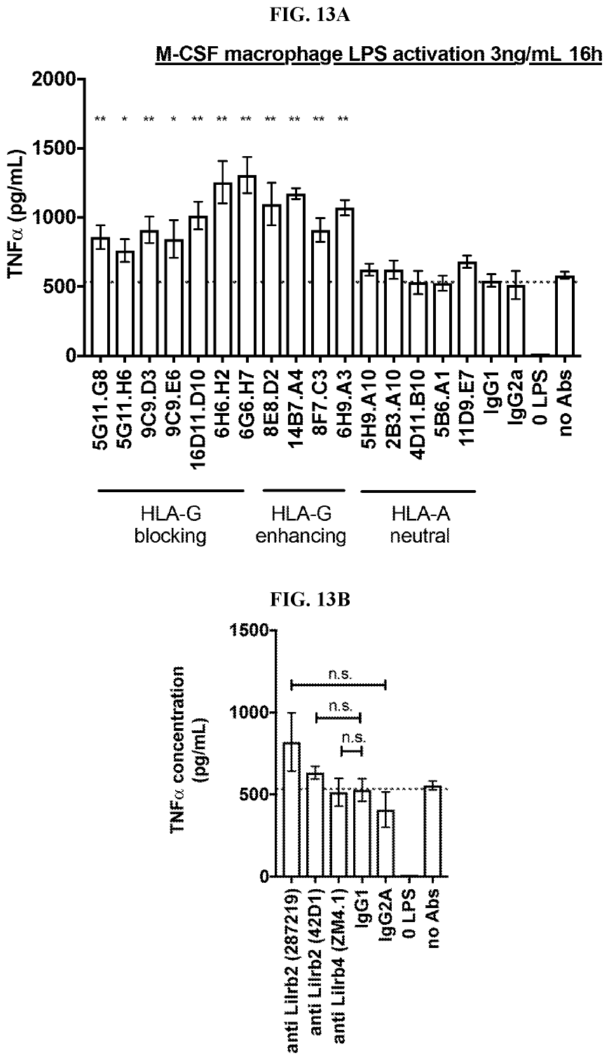

FIG. 13A-FIG. 13B show macrophage LPS activation. M-CSF macrophage secretion of TNFα following 16-hour LPS stimulation (3 ng/mL) was measured on Day 7. Antibody treatment concentration was 20 μg/mL. Data are presented as mean+/−standard deviation of 3 independent donors, each donor was performed in duplicate. Statistical comparison is between each antibody treatment and no antibody (LPS treated) control by performing t-tests, correction for multiple comparisons utilized the Benjamini & Kreiger method with a false discovery rate of 5%; *=p<0.05, **=p<0.01. Antibodies #287219 (R&D Systems), 42D1 (Biolegend), and ZM4.1 illustrated in FIG. 13B are commercial antibodies.

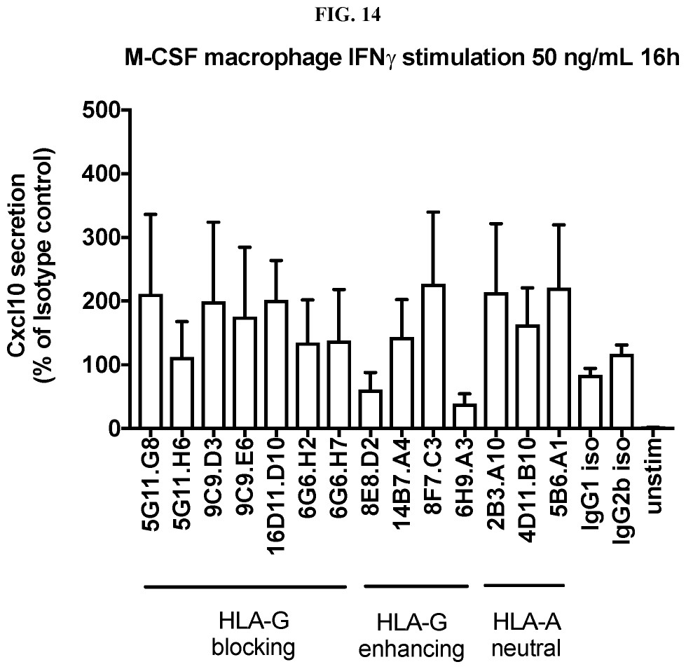

FIG. 14 shows macrophage IFNγ activation. M-CSF macrophage secretion of Cxc110 following 16-hour IFNγ stimulation (50 ng/mL) was measured on Day 7. Antibody treatment concentration was 20 μg/mL. Data are presented as mean+/−standard deviation of 4 independent donors.

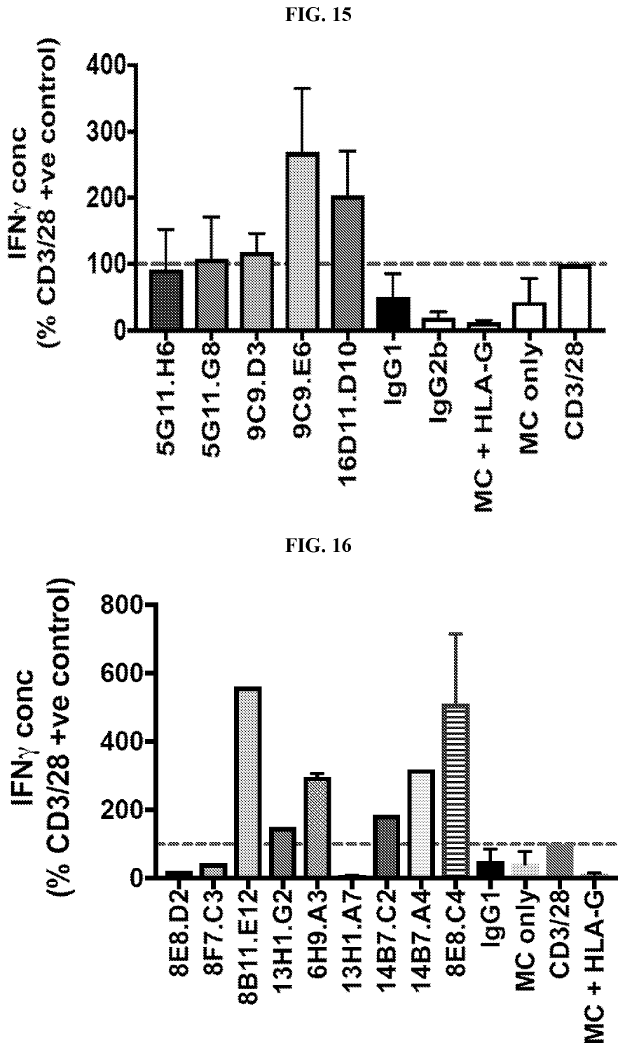

FIG. 15 shows MLR activity of exemplary anti-LILRB antibodies. This set of antibodies was shown to block HLA-G binding in FIG. 6A.

FIG. 16 shows MLR activity of exemplary anti-LILRB antibodies. This set of antibodies was shown to enhance HLA-G binding in FIG. 6A.

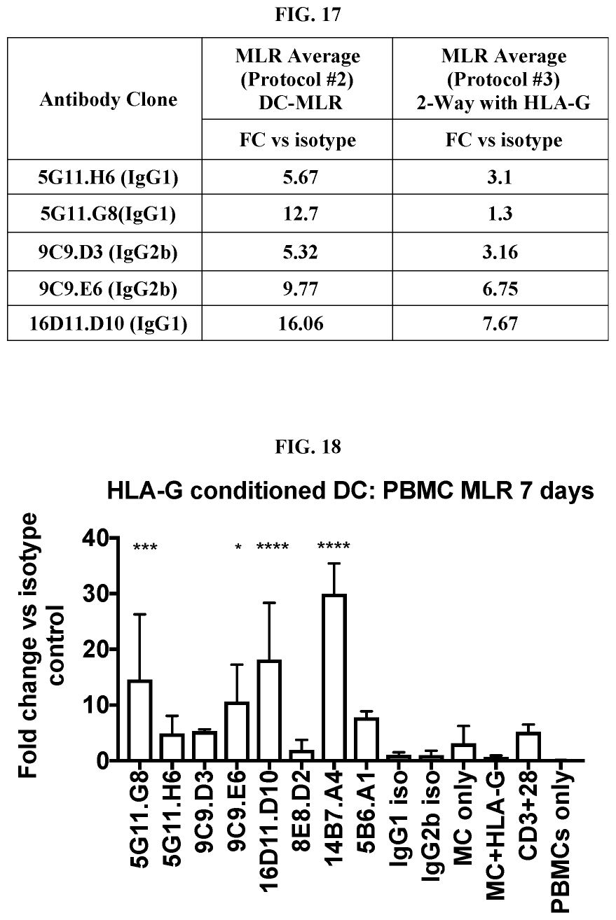

FIG. 17 illustrates the MLR activity of exemplary anti-LILRB antibodies.

FIG. 18 shows the ability of exemplary anti-LILRB antibodies to restore HLA-G induced suppression. IFNγ secretion was measured following 7-day mixed lymphocyte reaction culture of HLA-G conditioned DCs and treated with the test antibodies at 20 μg/mL. MLR was performed at a 1:50 ratio with allogenic responder PBMCs. Data presented show mean+/−standard deviation of two pooled experiments using the same donor: responder pair, with 3-5 replicate measurements per experiment normalized relative to the appropriate isotype control. For conditions with no antibody the average of the isotype controls was taken. Statistical analysis compares HLA-G conditioned DC to each antibody treated condition via 1-way ANOVA with multiple correction performed using Dunnett's method at an FDR cutoff of 5% (**=p<0.01, ****=p<0.0001). The IFNγ production serve as the primary endpoint for Th1 polarization.

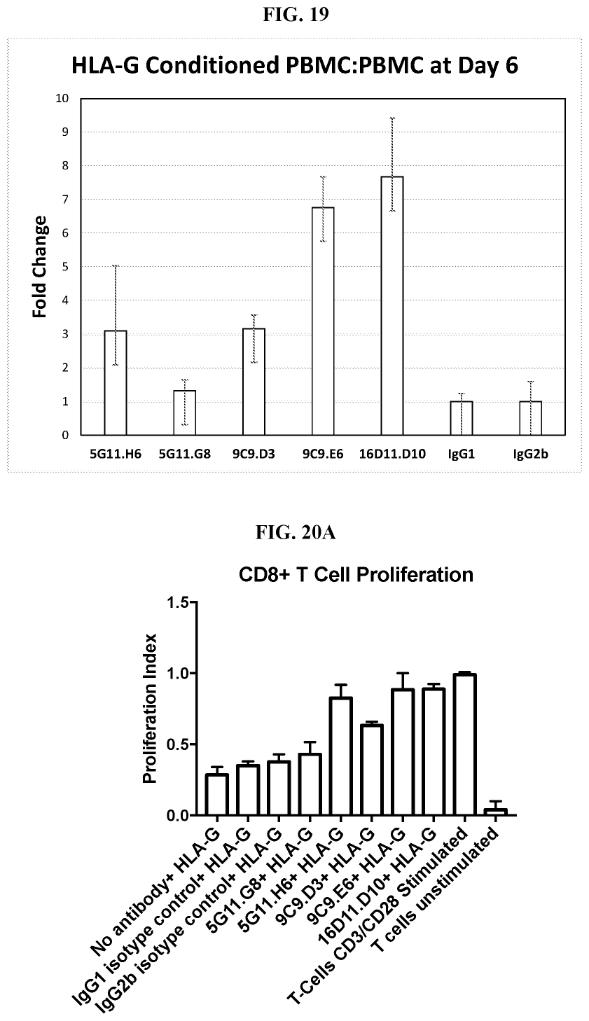

FIG. 19 shows a two-way MLR assay with HLA-G. The two-way MLR was established using PBMC cells from two unrelated donors in the presence of HLA-G and 1 μg/mL of HLA blocking anti-LILRB antibodies or IgG isotype controls was added to the PBMC cells.

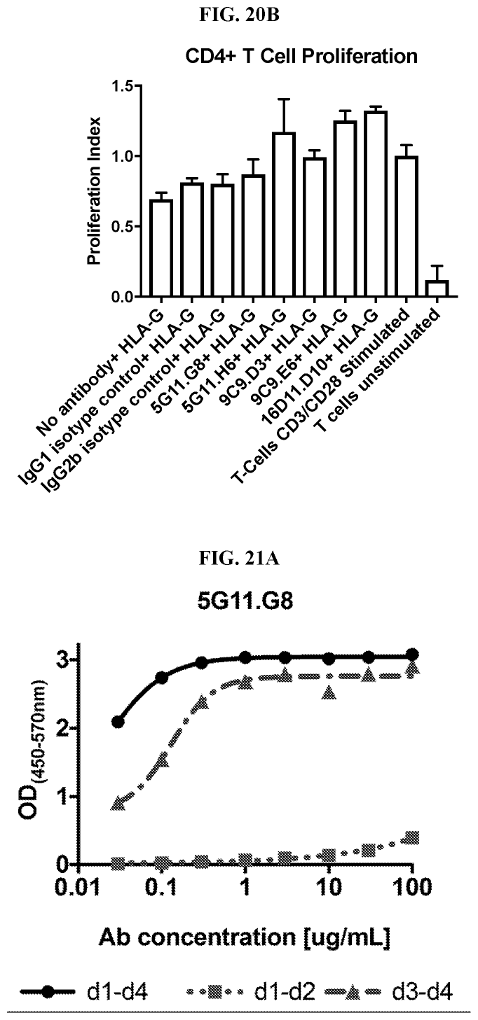

FIG. 20A-FIG. 20B show suppressive function of HLA-G induced CD33⁺CD11b⁺ MDSCs on allogenic T cells in the present of HLA-G blocking antibodies or IgG isotype controls (FIG. 20A: CD8+ T cell; FIG. 20B: CD4+ T cell). T cell proliferation index was determined by normalizing data with average of CD3/CD28 stimulated T cells.

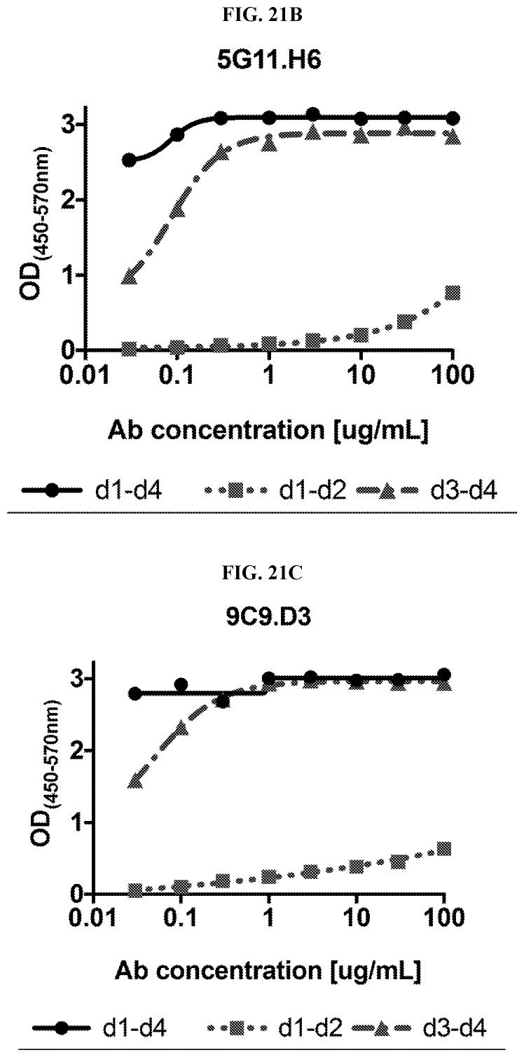

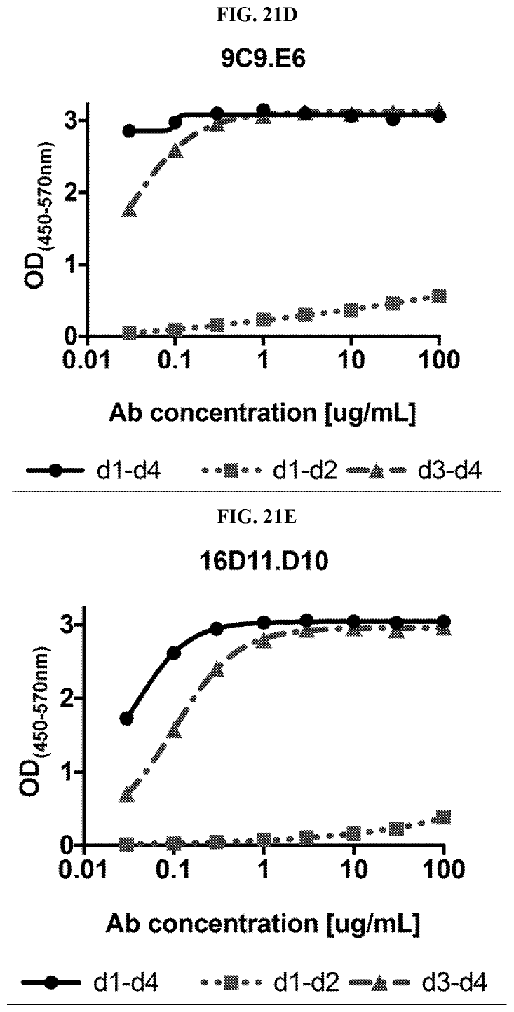

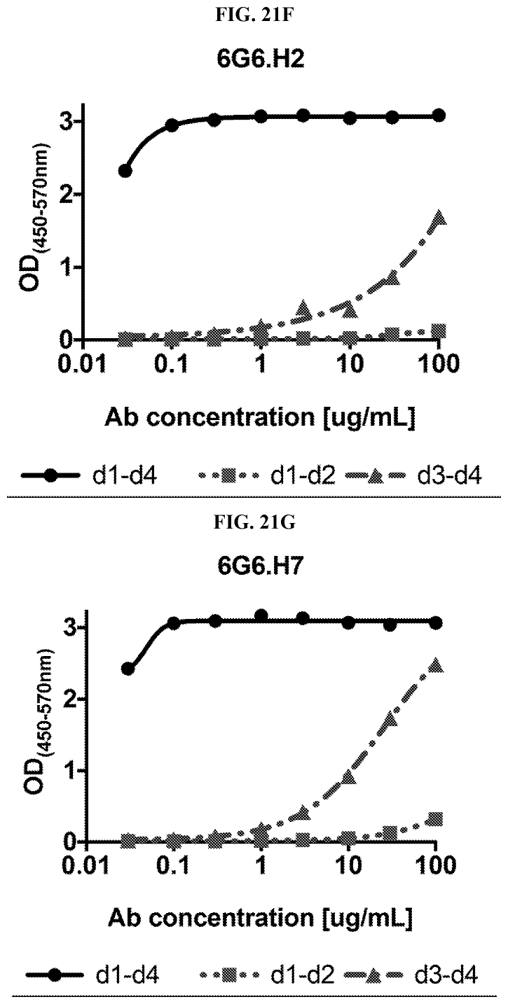

FIG. 21A-FIG. 21G illustrate ELISA binding of exemplary anti-LILRB antibodies to full-length extracellular LILRB2-Fc (d1-d4), LILRB2_d1d2-Fc, or LILRB2_d3d4-Fc proteins. FIG. 21A: antibody 5G11.G8; FIG. 21B: antibody 5G11.H6; FIG. 21C: antibody 9C9.D3; FIG. 21D: antibody 9C9.E6; FIG. 21E: antibody 16D11.D10; FIG. 21F: antibody 6G6.H2; and FIG. 21G: antibody 6G6.H7. These antibodies were shown to block HLA-G binding in FIG. 6A.

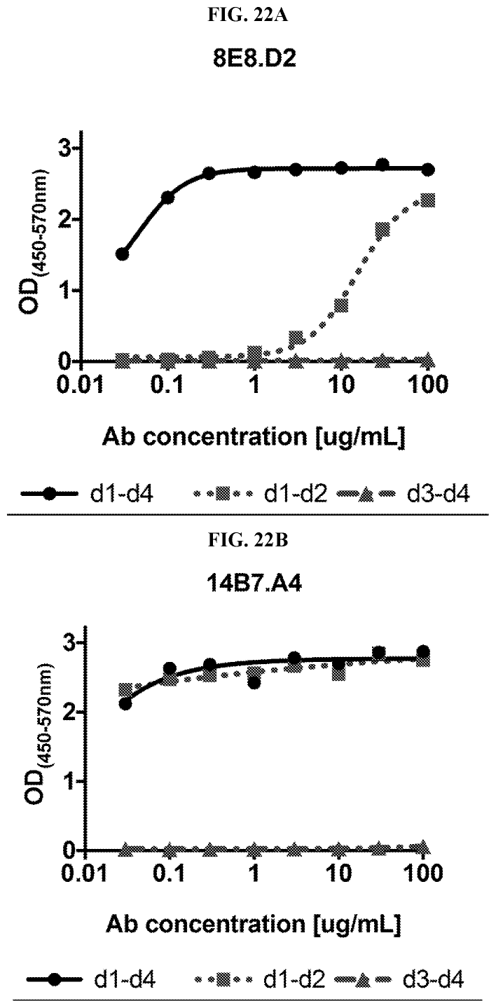

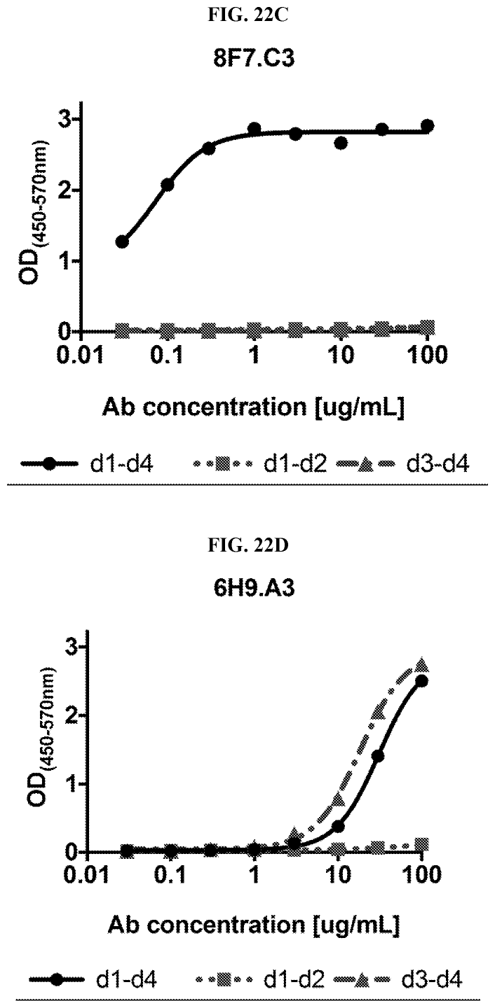

FIG. 22A-FIG. 22D illustrate ELISA binding of exemplary anti-LILRB antibodies to full-length extracellular LILRB2-Fc (d1-d4), LILRB2_d1d2-Fc, or LILRB2_d3d4-Fc proteins. FIG. 22A: antibody 8E8.D2; FIG. 22B: antibody 14B7.A4; FIG. 22C: antibody 8F7.C3; and FIG. 22D: antibody 6H9.A3. These antibodies were shown to enhance HLA-G binding in FIG. 6A.

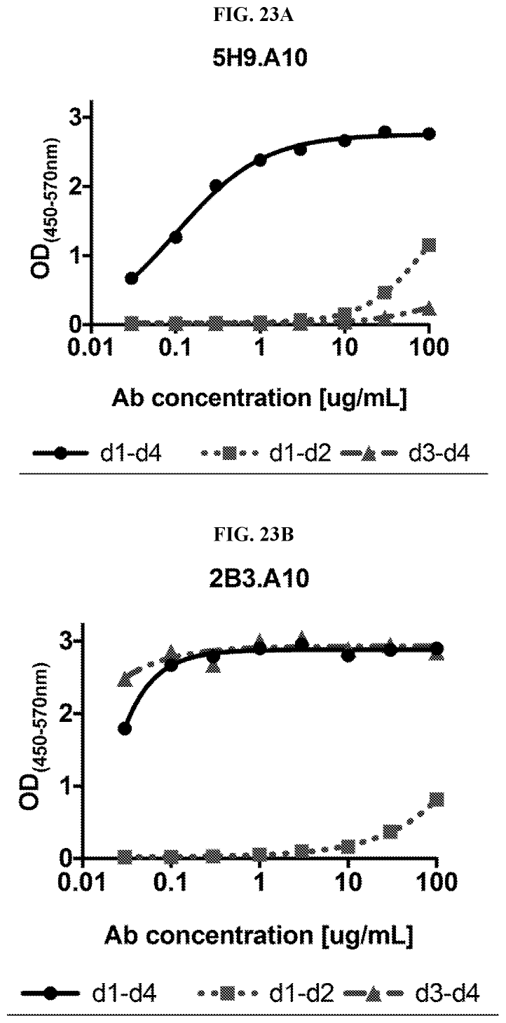

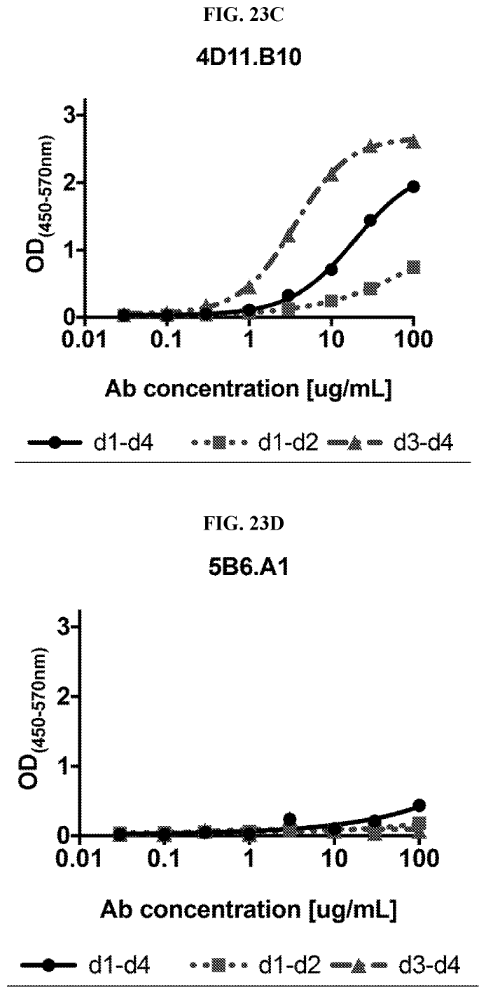

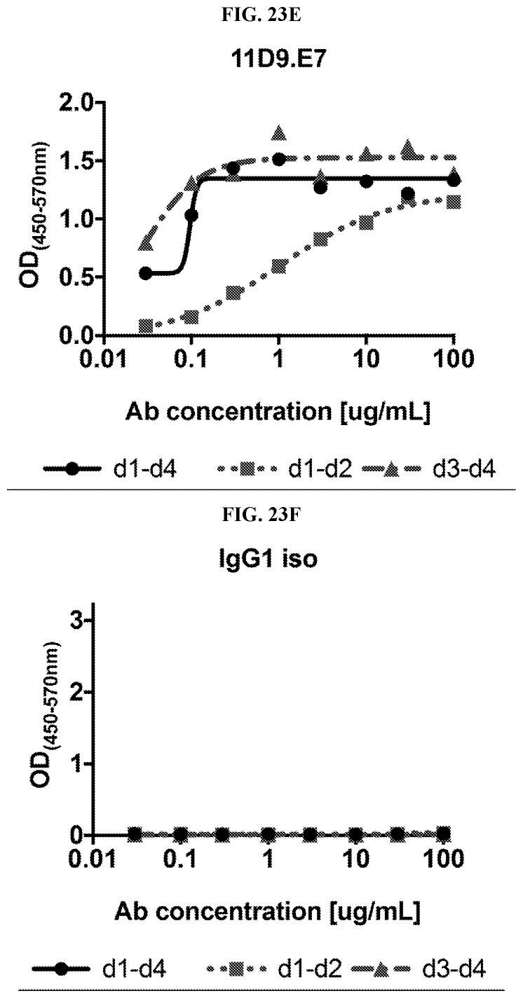

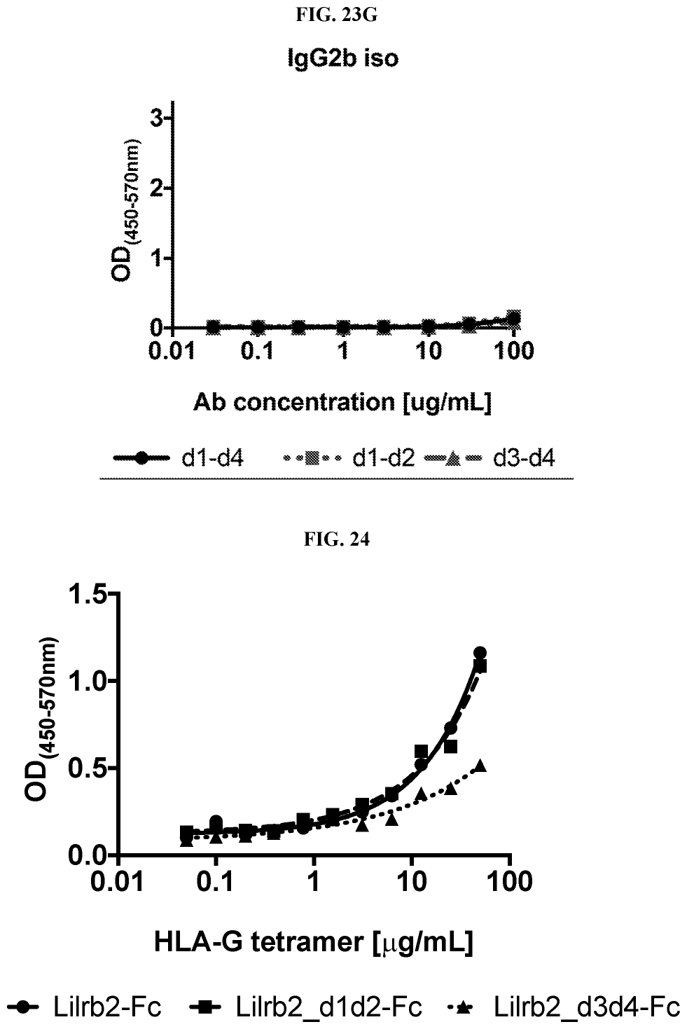

FIG. 23A-FIG. 23G illustrate ELISA binding of exemplary anti-LILRB antibodies to full-length extracellular LILRB2-Fc (d1-d4), LILRB2_d1d2-Fc, or LILRB2_d3d4-Fc proteins. FIG. 23A: antibody 5H9.A10; FIG. 23B: antibody 2B3.A10; FIG. 23C: antibody 4D11.B10; FIG. 23D: antibody 5B6.A1; FIG. 23E: antibody 11D9.E7; FIG. 23F: antibody IgG1; and FIG. 23G: antibody IgG2b. These antibodies were shown to be neutral with respect to HLA-G binding in FIG. 6A.

FIG. 24 shows ELISA binding of HLA-G tetramer to full-length extracellular Lilrb2-Fc, Lilrb2_d1d2-Fc or Lilrb2_d3d4-Fc protein showing that HLA-G tetramer binding to Lilrb2_d1d2-Fc is equivalent to Lilrb2-Fc.

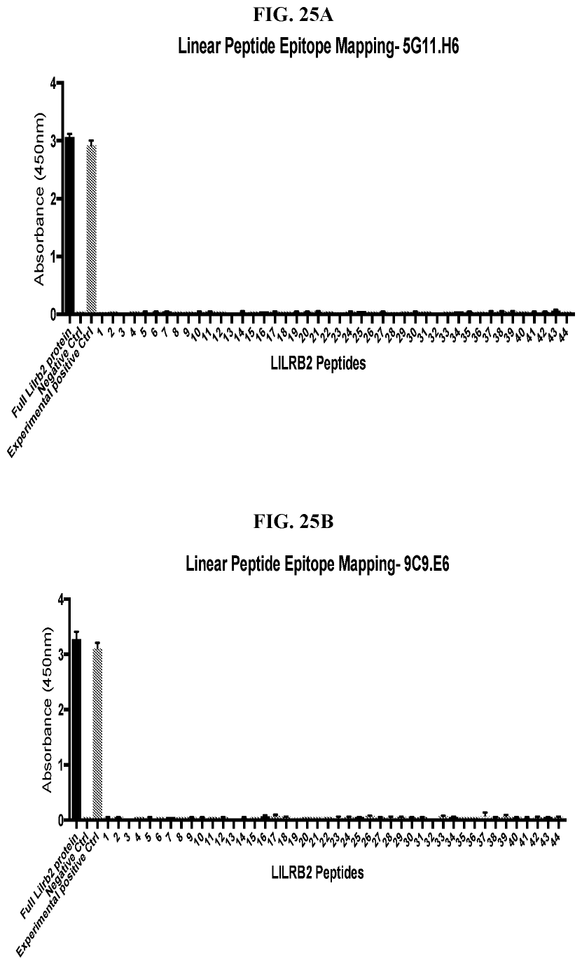

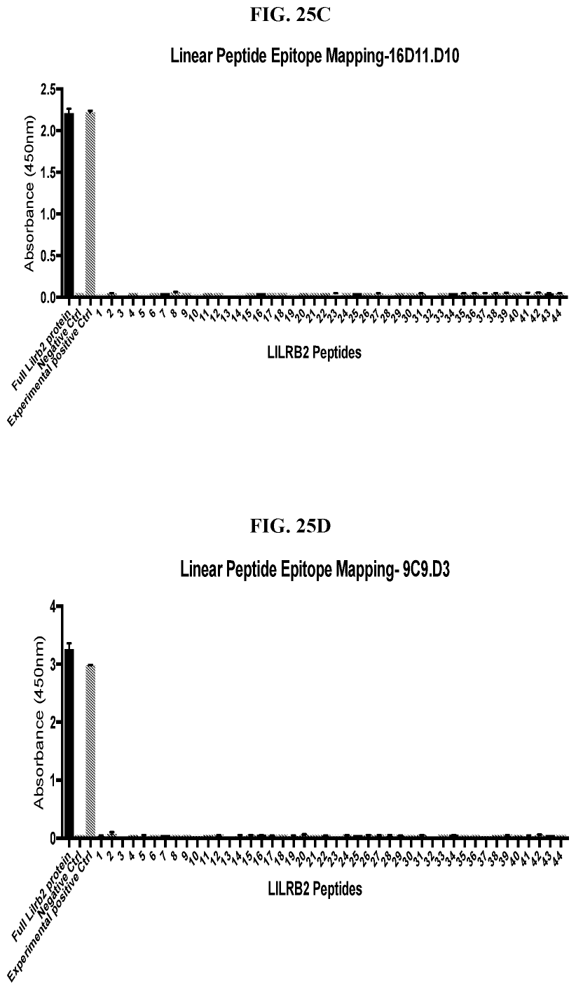

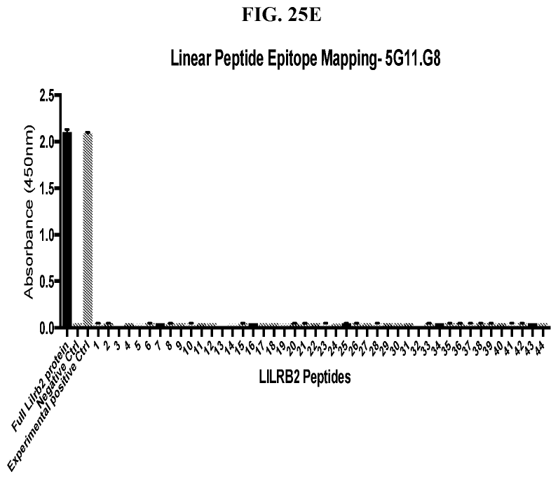

FIG. 25A-FIG. 25E show linear peptide epitope mapping of exemplary anti-LILRB antibodies. The linear peptides cover the full-length of the wild-type LILRB2 protein. As observed from the figures, none of the tested antibodies bound to the linear peptides, indicating that the test antibodies bound to a conformational epitope within D3 and/or D4 of LILRB2 protein. FIG. 25A: antibody 5G11.H6; FIG. 25B: antibody 9C9.E6; FIG. 25C: antibody 16D11.D10; FIG. 25D: antibody 9C9.D3; and FIG. 25E: antibody 5G11.G8.

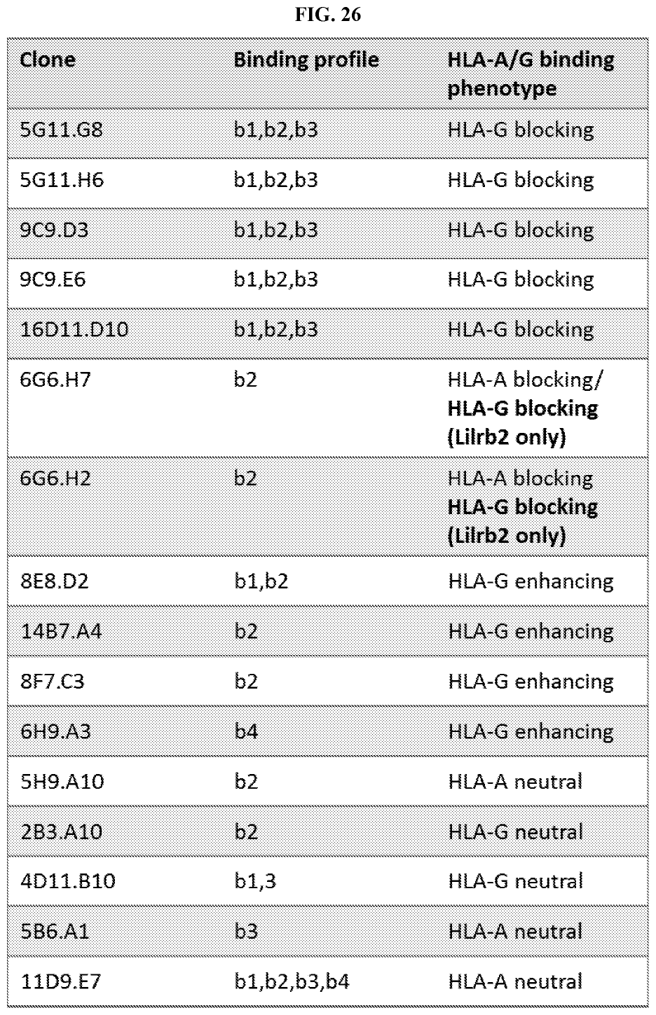

FIG. 26 shows LILRB binding and HLAG and HLA-A binding properties of exemplary anti-LILRB antibodies.

Table 1 illustrates ELISA binding profile of additional exemplary anti-LILRB antibodies to full-length extracellular LILRB1, LILRB2, LILRB3, and LILRB4.

| ANTIBODY | | | | |
|---|---|---|---|---|
| CLONE | LILRB1 | LILRB2 | LILRB3 | LILRB4 |
| 9B11.D3 | *** | *** | ** | nb |
| 9B11.D5 | *** | *** | ** | nb |
| 12F3.A2 | ** | *** | * | * |
| 12F3.G7 | ** | *** | * | * |
| 15D10.C5 | ** | *** | * | * |
| 16D11.A6 | ** | ** | ** | nb |
| 5H5.C8 | *** | *** | * | nb |
| 5H9.A10 | nb | ** | nb | nb |
| 1H10.C4 | nb | nb | ** | nb |
| 1H10.H3 | nb | nb | ** | nb |
| 5B6.B9 | nb | nb | *** | nb |
| 5H9.B6 | nb | nb | *** | nb |
| 5H9.B7 | nb | nb | *** | nb |
| 9B1.B1 | nb | nb | *** | nb |
| 9B1.D3 | nb | nb | *** | nb |
| 16D3.F6 | *** | nb | nb | *** |
| 16D3.G7 | *** | nb | nb | *** |
| 18F9.H2 | nb | nb | nb | *** |
| 18F9.H8 | nb | nb | nb | *** |
| 21G8.A1 | nb | nb | nb | *** |
| 21G8.A3 | nb | nb | nb | *** |
| 22D11.E4 | nb | nb | nb | *** |
| 22D11.F2 | nb | nb | nb | *** |
| 24H8.D2 | nb | nb | nb | *** |
| 24H8.D4 | nb | nb | nb | *** |
| 26E6.E8 | nb | nb | nb | *** |
| 26E6.H6 | nb | nb | nb | *** |
| 28E9.B3 | nb | nb | nb | *** |
| 28E9.H2 | nb | nb | nb | *** |
| 2A4.5F | nb | nb | nb | ** |
| 2A4.A3 | nb | nb | nb | *** |
| 2A4.G11 | nb | nb | nb | ** |
| 32A9.A9 | nb | nb | nb | *** |
| 4F10.C6 | *** | nb | nb | *** |
| 4F10.D5 | *** | nb | nb | *** |
| 11D9.A4 | *** | ** | *** | *** |
| 11D9.E7 | *** | ** | *** | ** |
| 12H7.G5 | nb | nb | nb | ** |
| 12H7.G7 | nb | nb | nb | ** |
| 13B3.D12 | nb | nb | nb | * |
| 13B3.D5 | nb | nb | nb | * |
| 8B11.E11 | *** | ** | ** | ** |

nb (no binding): <0.1
* ≥0.1 to <0.5
** ≥0.5 to <1
*** ≥1

### Example 18

Table 2 illustrates exemplary LILRB sequences disclosed herein.

| | | SEQ ID |
|---|---|---|
| NAME | SEQUENCE | NO: |
| LILRB1_1 | MTPILTVLICLGLSLGPRTHVQAGHLPKPTLWAEPGSVITQGSPVTLR | 1 |
| (isoform 1 | CQGGQETQEYRLYREKKTAPWITRIPQELVKKGQFPIPSITWEHTGR | |
| precursor) | YRCYYGSDTAGRSESSDPLELVVTGAYIKPTLSAQPSPVVNSGGNVT | |
| (NCBI Accession | LQCDSQVAFDGFILCKEGEDEHPQCLNSQPHARGSSRAIFSVGPVSPS | |
| No. NP_006660.4) | RRWWYRCYAYDSNSPYEWSLPSDLLELLVLGVSKKPSLSVQPGPIV | |
| | APEETLTLQCGSDAGYNRFVLYKDGERDFLQLAGAQPQAGLSQANF | |
| | TLGPVSRSYGGQYRCYGAHNLSSEWSAPSDPLDILIAGQFYDRVSLS | |
| | VQPGPTVASGENVTLLCQSQGWMQTFLLTKEGAADDPWRLRSTYQ | |
| | SQKYQAEFPMGPVTSAHAGTYRCYGSQSSKPYLLTHPSDPLELVVSG | |
| | PSGGPSSPTTGPTSTSGPEDQPLTPTGSDPQSGLGRHLGVVIGILVAVI | |
| | LLLLLLLLLFLILRHRRQGKHWTSTQRKADFQHPAGAVGPEPTDRGL | |
| | QWRSSPAADAQEENLYAAVKHTQPEDGVEMDTRSPHDEDPQAVTY | |
| | AEVKHSRPRREMASPPSPLSGEFLDTKDRQAEEDRQMDTEAAASEA | |
| | PQDVTYAQLHSLTLRREATEPPPSQEGPSPAVPSIYATLAIH | |
| LILRB1_2 | MTPILTVLICLGLSLGPRTHVQAGHLPKPTLWAEPGSVITQGSPVTLR | 2 |
| (isoform 2 | CQGGQETQEYRLYREKKTAPWITRIPQELVKKGQFPIPSITWEHTGR | |
| precursor) | YRCYYGSDTAGRSESSDPLELVVTGAYIKPTLSAQPSPVVNSGGNVT | |
| (NCBI Accession | LQCDSQVAFDGFILCKEGEDEHPQCLNSQPHARGSSRAIFSVGPVSPS | |
| No. | RRWWYRCYAYDSNSPYEWSLPSDLLELLVLGVSKKPSLSVQPGPIV | |
| NP_001075106.2) | APEETLTLQCGSDAGYNRFVLYKDGERDFLQLAGAQPQAGLSQANF | |
| | TLGPVSRSYGGQYRCYGAHNLSSEWSAPSDPLDILIAGQFYDRVSLS | |
| | VQPGPTVASGENVTLLCQSQGWMQTFLLTKEGAADDPWRLRSTYQ | |
| | SQKYQAEFPMGPVTSAHAGTYRCYGSQSSKPYLLTHPSDPLELVVSG | |
| | PSGGPSSPTTGPTSTSAGPEDQPLTPTGSDPQSGLGRHLGVVIGILVAV | |
| | ILLLLLLLLLFLILRHRRQGKHWTSTQRKADFQHPAGAVGPEPTDRG | |
| | LQWRSSPAADAQEENLYAAVKHTQPEDGVEMDTRQSPHDEDPQAV | |
| | TYAEVKHSRPRREMASPPSPLSGEFLDTKDRQAEEDRQMDTEAAAS | |
| | EAPQDVTYAQLHSLTLRREATEPPPSQEGPSPAVPSIYATLAIH | |
| LILRB1_3 | MTPILTVLICLGLSLGPRTHVQAGHLPKPTLWAEPGSVITQGSPVTLR | 3 |
| (isoform 3 | CQGGQETQEYRLYREKKTAPWITRIPQELVKKGQFPIPSITWEHTGR | |
| precursor) | YRCYYGSDTAGRSESSDPLELVVTGAYIKPTLSAQPSPVVNSGGNVT | |
| (NCBI Accession | LQCDSQVAFDGFILCKEGEDEHPQCLNSQPHARGSSRAIFSVGPVSPS | |
| No. | RRWWYRCYAYDSNSPYEWSLPSDLLELLVLGVSKKPSLSVQPGPIV | |
| NP_001075107.2) | APEETLTLQCGSDAGYNRFVLYKDGERDFLQLAGAQPQAGLSQANF | |
| | TLGPVSRSYGGQYRCYGAHNLSSEWSAPSDPLDILIAGQFYDRVSLS | |
| | VQPGPTVASGENVTLLCQSQGWMQTFLLTKEGAADDPWRLRSTYQ | |
| | SQKYQAEFPMGPVTSAHAGTYRCYGSQSSKPYLLTHPSDPLELVVSG | |
| | PSGGPSSPTTGPTSTSAGPEDQPLTPTGSDPQSGLGRHLGVVIGILVAV | |
| | ILLLLLLLLLFLILRHRRQGKHWTSTQRKADFQHPAGAVGPEPTDRG | |
| | LQWRSSPAADAQEENLYAAVKHTQPEDGVEMDTRSPHDEDPQAVT | |
| | YAEVKHSRPRREMASPPSPLSGEFLDTKDRQAEEDRQMDTEAAASE | |
| | APQDVTYAQLHSLTLRREATEPPPSQEGPSPAVPSIYATLAIH | |
| LILRB1_4 | MTPILTVLICLGLSLGPRTHVQAGHLPKPTLWAEPGSVITQGSPVTLR | 4 |
| (isoform 4 | CQGGQETQEYRLYREKKTAPWITRIPQELVKKGQFPIPSITWEHTGR | |
| precursor) | YRCYYGSDTAGRSESSDPLELVVTGAYIKPTLSAQPSPVVNSGGNVT | |
| (NCBI Accession | LQCDSQVAFDGFILCKEGEDEHPQCLNSQPHARGSSRAIFSVGPVSPS | |
| No. | RRWWYRCYAYDSNSPYEWSLPSDLLELLVLGVSKKPSLSVQPGPIV | |
| NP_001075108.2 | APEETLTLQCGSDAGYNRFVLYKDGERDFLQLAGAQPQAGLSQANF | |
| | TLGPVSRSYGGQYRCYGAHNLSSEWSAPSDPLDILIAGQFYDRVSLS | |
| | VQPGPTVASGENVTLLCQSQGWMQTFLLTKEGAADDPWRLRSTYQ | |
| | SQKYQAEFPMGPVTSAHAGTYRCYGSQSSKPYLLTHPSDPLELVVSG | |
| | PSGGPSSPTTGPTSTSGPEDQPLTPTGSDPQSGLGRHLGVVIGILVAVI | |
| | LLLLLLLLLFLILRHRRQGKHWTSTQRKADFQHPAGAVGPEPTDRGL | |
| | QWRSSPAADAQEENLYAAVKHTQPEDGVEMDTRQSPHDEDPQAVT | |
| | YAEVKHSRPRREMASPPSPLSGEFLDTKDRQAEEDRQMDTEAAASE | |
| | APQDVTYAQLHSLTLRREATEPPPSQEGPSPAVPSIYATLAIH | |
| LILRB1_5 | MTPILTVLICLGLSLGPRTHVQAGHLPKPTLWAEPGSVITQGSPVTLR | 5 |
| (isoform 5 | CQGGQETQEYRLYREKKTAPWITRIPQELVKKGQFPIPSITWEHTGR | |
| precursor) | YRCYYGSDTAGRSESSDPLELVVTGAYIKPTLSAQPSPVVNSGGNVT | |
| (NCBI Accession | LQCDSQVAFDGFILCKEGEDEHPQCLNSQPHARGSSRAIFSVGPVSPS | |
| No. | RRWWYRCYAYDSNSPYEWSLPSDLLELLVLGVSKKPSLSVQPGPIV | |
| NP_001265327.2) | APEETLTLQCGSDAGYNRFVLYKDGERDFLQLAGAQPQAGLSQANF | |
| | TLGPVSRSYGGQYRCYGAHNLSSEWSAPSDPLDILIAGQFYDRVSLS | |
| | VQPGPTVASGENVTLLCQSQGWMQTFLLTKEGAADDPWRLRSTYQ | |
| | SQKYQAEFPMGPVTSAHAGTYRCYGSQSSKPYLLTHPSDPLELVVSA | |
| | GPEDQPLTPTGSDPQSGLGRHLGVVIGILVAVILLLLLLLLLFLILRHR | |
| | RQGKHWTSTQRKADFQHPAGAVGPEPTDRGLQWRSSPAADAQEEN | |
| | LYAAVKHTQPEDGVEMDTRSPHDEDPQAVTYAEVKHSRPRREMAS | |
| | PPSPLSGEFLDTKDRQAEEDRQMDTEAAASEAPQDVTYAQLHSLTLR | |
| | REATEPPPSQEGPSPAVPSIYATLAIH | |
| LILRB1_6 | MTPILTVLICLGLSLGPRTHVQAGHLPKPTLWAEPGSVITQGSPVTLR | 6 |
| (isoform 6 | CQGGQETQEYRLYREKKTAPWITRIPQELVKKGQFPIPSITWEHTGR | |
| precursor) | YRCYYGSDTAGRSESSDPLELVVTGAYIKPTLSAQPSPVVNSGGNVT | |
| (NCBI Accession | LQCDSQVAFDGFILCKEGEDEHPQCLNSQPHARGSSRAIFSVGPVSPS | |
| No. | RRWWYRCYAYDSNSPYEWSLPSDLLELLVLGVSKKPSLSVQPGPIV | |
| NP_001265328.2) | APEETLTLQCGSDAGYNRFVLYKDGERDFLQLAGAQPQAGLSQANF | |
| | TLGPVSRSYGGQYRCYGAHNLSSEWSAPSDPLDILIAGQFYDRVSLS | |
| | VQPGPTVASGENVTLLCQSQGWMQTFLLTKEGAADDPWRLRSTYQ | |
| | SQKYQAEFPMGPVTSAHAGTYRCYGSQSSKPYLLTHPSDPLELVVSG | |
| | PSGGPSSPTTGPTSTSAGPEDQPLTPTGSDPQSGE | |
| LILRB1_D7 | GGGCACCTCCCCAAGCCCACCCTCTGGGCTGAACCAGGCTCTGTG | 7 |
| | ATCACCCAGGGGAGTCCTGTGACCCTCAGGTGTCAGGGGGGCCA | |
| | GGAGACCCAGGAGTACCGTCTATATAGAGAAAAGAAAACAGCAC | |
| | CCTGGATTACACGGATTCCACAGGAGCTTGTGAAGAAGGGCCAG | |
| | TTCCCCATCCCATCCATCACCTGGGAACACACAGGGCGGTATCGC | |
| | TGTTACTATGGTAGCGACACTGCAGGCCGCTCAGAGAGCAGTGAC | |
| | CCCCTGGAGCTGGTGGTGACAGGAGCCTACATCAAACCCACCCTC | |
| | TCAGCCCAGCCCAGCCCCGTGGTGAACTCAGGAGGGAATGTAAC | |
| | CCTCCAGTGTGACTCACAGGTGGCATTTGATGGCTTCATTCTGTGT | |
| | AAGGAAGGAGAAGATGAACACCCACAATGCCTGAACTCCCAGCC | |
| | CCATGCCCGTGGGTCGTCCCGCGCCATCTTCTCCGTGGGCCCCGT | |
| | GAGCCCGAGTCGCAGGTGGTGGTACAGGTGCTATGCTTATGACTC | |
| | GAACTCTCCCTATGAGTGGTCTCTACCCAGTGATCTCCTGGAGCT | |
| | CCTGGTCCTAGGTGTTTCTAAGAAGCCATCACTCTCAGTGCAGCC | |
| | AGGTCCTATCGTGGCCCCTGAGGAGACCCTGACTCTGCAGTGTGG | |
| | CTCTGATGCTGGCTACAACAGATTTGTTCTGTATAAGGACGGGGA | |
| | ACGTGACTTCCTTCAGCTCGCTGGCGCACAGCCCCAGGCTGGGCT | |
| | CTCCCAGGCCAACTTCACCCTGGGCCCTGTGAGCCGCTCCTACGG | |
| | GGGCCAGTACAGATGCTACGGTGCACACAACCTCTCCTCCGAGTG | |
| | GTCGGCCCCCAGCGACCCCCTGGACATCCTGATCGCAGGACAGTT | |
| | CTATGACAGAGTCTCCCTCTCGGTGCAGCCGGGCCCCACGGTGGC | |
| | CTCAGGAGAGAACGTGACCCTGCTGTGTCAGTCACAGGGATGGA | |
| | TGCAAACTTTCCTTCTGACCAAGGAGGGGGCAGCTGATGACCCAT | |
| | GGCGTCTAAGATCAACGTACCAATCTCAAAAATACCAGGCTGAAT | |
| | TCCCCATGGGTCCTGTGACCTCAGCCCATGCGGGGACCTACAGGT | |
| | GCTACGGCTCACAGAGCTCCAAACCCTACCTGCTGACTCACCCCA | |
| | GTGACCCCCTGGAGCTCGTGGTCTCAGGACCGTCTGGGGGCCCCA | |
| | GCTCCCCGACAACAGGCCCCACCTCCACATCTGCAGGCCCTGAGG | |
| | ACCAGCCCCTCACCCCCACCGGGTCGGACCCCCAGAGTGGTCTGG | |
| | GAAGGCACCTGGGG | |
| LILRB1_P7 | GHLPKPTLWAEPGSVITQGSPVTLRCQGGQETQEYRLYREKKTAPWI | 8 |
| | TRIPQELVKKGQFPIPSITWEHTGRYRCYYGSDTAGRSESSDPLELVV | |
| | TGAYIKPTLSAQPSPVVNSGGNVTLQCDSQVAFDGFILCKEGEDEHP | |
| | QCLNSQPHARGSSRAIFSVGPVSPSRRWWYRCYAYDSNSPYEWSLPS | |
| | DLLELLVLGVSKKPSLSVQPGPIVAPEETLTLQCGSDAGYNRFVLYK | |
| | DGERDFLQLAGAQPQAGLSQANFTLGPVSRSYGGQYRCYGAHNLSS | |
| | EWSAPSDPLDILIAGQFYDRVSLSVQPGPTVASGENVTLLCQSQGWM | |
| | QTFLLTKEGAADDPWRLRSTYQSQKYQAEFPMGPVTSAHAGTYRC | |
| | YGSQSSKPYLLTHPSDPLELVVSGPSGGPSSPTTGPTSTSAGPEDQPLT | |
| | PTGSDPQSGLGRHLG | |
| LILRB2_1 | MTPIVTVLICLGLSLGPRTRVQTGTIPKPTLWAEPDSVITQGSPVTLSC | 9 |
| (isoform 1 | QGSLEAQEYRLYREKKSASWITRIRPELVKNGQFHIPSITWEHTGRYG | |
| precursor) | CQYYSRARWSELSDPLVLVMTGAYPKPTLSAQPSPVVTSGGRVTLQ | |
| (NCBI Accession | CESQVAFGGFILCKEGEDEHPQCLNSQPHARGSSRAIFSVGPVSPNRR | |
| No. NP_005865.3) | WSHRCYGYDLNSPYVWSSPSDLLELLVPGVSKKPSLSVQPGPVMAP | |
| | GESLTLQCVSDVGYDRFVLYKEGERDLRQLPGRQPQAGLSQANFTL | |
| | GPVSRSYGGQYRCYGAHNLSSECSAPSDPLDILITGQIRGTPFISVQPG | |
| | PTVASGENVTLLCQSWRQFHTFLLTKAGAADAPLRLRSIHEYPKYQA | |
| | EFPMSPVTSAHAGTYRCYGSLNSDPYLLSHPSEPLELVVSGPSMGSSP | |
| | PPTGPISTPAGPEDQPLTPTGSDPQSGLGRHLGVVIGILVAVVLLLLLL | |
| | LLLFLILRHRRQGKHWTSTQRKADFQHPAGAVGPEPTDRGLQWRSS | |
| | PAADAQEENLYAAVKDTQPEDGVEMDTRAAASEAPQDVTYAQLHS | |
| | LTLRRKATEPPPSQEREPPAEPSIYATLAIH | |
| LILRB2_2 | MTPIVTVLICLGLSLGPRTRVQTGTIPKPTLWAEPDSVITQGSPVTLSC | 10 |
| (isoform 2 | QGSLEAQEYRLYREKKSASWITRIRPELVKNGQFHIPSITWEHTGRYG | |
| precursor) | CQYYSRARWSELSDPLVLVMTGAYPKPTLSAQPSPVVTSGGRVTLQ | |
| (NCBI Accession | CESQVAFGGFILCKEGEDEHPQCLNSQPHARGSSRAIFSVGPVSPNRR | |
| No. | WSHRCYGYDLNSPYVWSSPSDLLELLVPGVSKKPSLSVQPGPVMAP | |
| NP_001074447.2) | GESLTLQCVSDVGYDRFVLYKEGERDLRQLPGRQPQAGLSQANFTL | |
| | GPVSRSYGGQYRCYGAHNLSSECSAPSDPLDILITGQIRGTPFISVQPG | |
| | PTVASGENVTLLCQSWRQFHTFLLTKAGAADAPLRLRSIHEYPKYQA | |
| | EFPMSPVTSAHAGTYRCYGSLNSDPYLLSHPSEPLELVVSGPSMGSSP | |
| | PPTGPISTPGPEDQPLTPTGSDPQSGLGRHLGVVIGILVAVVLLLLLLL | |
| | LLFLILRHRRQGKHWTSTQRKADFQHPAGAVGPEPTDRGLQWRSSP | |
| | AADAQEENLYAAVKDTQPEDGVEMDTRAAASEAPQDVTYAQLHSL | |
| | TLRRKATEPPPSQEREPPAEPSIYATLAIH | |
| LILRB2_3 | MTGAYPKPTLSAQPSPVVTSGGRVTLQCESQVAFGGFILCKEGEDEH | 11 |
| (isoform 3 | PQCLNSQPHARGSSRAIFSVGPVSPNRRWSHRCYGYDLNSPYVWSSP | |
| precursor) | SDLLELLVPGVSKKPSLSVQPGPVMAPGESLTLQCVSDVGYDRFVLY | |
| (NCBI Accession | KEGERDLRQLPGRQPQAGLSQANFTLGPVSRSYGGQYRCYGAHNLS | |
| No. | SECSAPSDPLDILITGQIRGTPFISVQPGPTVASGENVTLLCQSWRQFH | |
| NP_001265333.2) | TFLLTKAGAADAPLRLRSIHEYPKYQAEFPMSPVTSAHAGTYRCYGS | |
| | LNSDPYLLSHPSEPLELVVSGPSMGSSPPPTGPISTPAGPEDQPLTPTG | |
| | SDPQSGLGRHLGVVIGILVAVVLLLLLLLLLFLILRHRRQGKHWTSTQ | |
| | RKADFQHPAGAVGPEPTDRGLQWRSSPAADAQEENLYAAVKDTQP | |
| | EDGVEMDTRAAASEAPQDVTYAQLHSLTLRRKATEPPPSQEREPPAE | |
| | PSIYATLAIH | |
| LILRB2_4 | MTPIVTVLICLGLSLGPRTRVQTGTIPKPTLWAEPDSVITQGSPVTLSC | 12 |
| (isoform 4 | QGSLEAQEYRLYREKKSASWITRIRPELVKNGQFHIPSITWEHTGRYG | |
| precursor) | CQYYSRARWSELSDPLVLVMTGAYPKPTLSAQPSPVVTSGGRVTLQ | |
| (NCBI Accession | CESQVAFGGFILCKEGEDEHPQCLNSQPHARGSSRAIFSVGPVSPNRR | |
| No. | WSHRCYGYDLNSPYVWSSPSDLLELLVPGVSKKPSLSVQPGPVMAP | |
| NP_001265334.2) | GESLTLQCVSDVGYDRFVLYKEGERDLRQLPGRQPQAGLSQANFTL | |
| | GPVSRSYGGQYRCYGAHNLSSECSAPSDPLDILITGQIRGTPFISVQPG | |
| | PTVASGENVTLLCQSWRQFHTFLLTKAGAADAPLRLRSIHEYPKYQA | |
| | EFPMSPVTSAHAGTYRCYGSLNSDPYLLSHPSEPLELVVSGPSMGSSP | |
| | PPTGPISTPAGPEDQPLTPTGSDPQSGLGRHLGVVIGILVAVVLLLLLL | |
| | LLLFLILRHRRQGKHWTSSPAQLPTPRKKTSMLP | |
| LILRB2_5 | MTPIVTVLICLGLSLGPRTRVQTGTIPKPTLWAEPDSVITQGSPVTLSC | 13 |
| (isoform 5 | QGSLEAQEYRLYREKKSASWITRIRPELVKNGQFHIPSITWEHTGRYG | |
| precursor) | CQYYSRARWSELSDPLVLVMTGAYPKPTLSAQPSPVVTSGGRVTLQ | |
| (NCBI Accession | CESQVAFGGFILCKEGEDEHPQCLNSQPHARGSSRAIFSVGPVSPNRR | |
| No. | WSHRCYGYDLNSPYVWSSPSDLLELLVPGVSKKPSLSVQPGPVMAP | |
| NP_001265335.2) | GESLTLQCVSDVGYDRFVLYKEGERDLRQLPGRQPQAGLSQANFTL | |
| | GPVSRSYGGQYRCYGAHNLSSECSAPSDPLDILITGQIRGTPFISVQPG | |
| | PTVASGENVTLLCQSWRQFHTFLLTKAGAADAPLRLRSIHEYPKYQA | |
| | EFPMSPVTSAHAGTYRCYGSLNSDPYLLSHPSEPLELVVSGPSMGSSP | |
| | PPTGPISTPAGPEDQPLTPTGSDPQSGE | |
| LILRB2_D6 | CAGACAGGGACCATCCCCAAGCCCACCCTGTGGGCTGAGCCAGA | 14 |
| | CTCTGTGATCACCCAGGGGAGTCCCGTCACCCTCAGTTGTCAGGG | |
| | GAGCCTTGAAGCCCAGGAGTACCGTCTATATAGGGAGAAAAAAT | |
| | CAGCATCTTGGATTACACGGATACGACCAGAGCTTGTGAAGAAC | |
| | GGCCAGTTCCACATCCCATCCATCACCTGGGAACACACAGGGCGA | |
| | TATGGCTGTCAGTATTACAGCCGCGCTCGGTGGTCTGAGCTCAGT | |
| | GACCCCCTGGTGCTGGTGATGACAGGAGCCTACCCAAAACCCACC | |
| | CTCTCAGCCCAGCCCAGCCCTGTGGTGACCTCAGGAGGAAGGGTG | |
| | ACCCTCCAGTGTGAGTCACAGGTGGCATTTGGCGGCTTCATTCTG | |
| | TGTAAGGAAGGAGAAGATGAACACCCACAATGCCTGAACTCCCA | |
| | GCCCCATGCCCGTGGGTCGTCCCGCGCCATCTTCTCCGTGGGCCC | |
| | CGTGAGCCCGAATCGCAGGTGGTCGCACAGGTGCTATGGTTATGA | |
| | CTTGAACTCTCCCTATGTGTGGTCTTCACCCAGTGATCTCCTGGAG | |
| | CTCCTGGTCCCAGGTGTTTCTAAGAAGCCATCACTCTCAGTGCAG | |
| | CCGGGTCCTGTCATGGCCCCTGGGGAAAGCCTGACCCTCCAGTGT | |
| | GTCTCTGATGTCGGCTATGACAGATTTGTTCTGTACAAGGAGGGG | |
| | GAACGTGACCTTCGCCAGCTCCCTGGCCGGCAGCCCCAGGCTGGG | |
| | CTCTCCCAGGCCAACTTCACCCTGGGCCCTGTGAGCCGCTCCTAC | |
| | GGGGGCCAGTACAGATGCTACGGTGCACACAACCTCTCCTCTGAG | |
| | TGCTCGGCCCCCAGCGACCCCCTGGACATCCTGATCACAGGACAG | |
| | ATCCGTGGCACACCCTTCATCTCAGTGCAGCCAGGCCCCACAGTG | |
| | GCCTCAGGAGAGAACGTGACCCTGCTGTGTCAGTCATGGCGGCA | |
| | GTTCCACACTTTCCTTCTGACCAAGGCGGGAGCAGCTGATGCCCC | |
| | ACTCCGTCTAAGATCAATACACGAATATCCTAAGTACCAGGCTGA | |
| | ATTCCCCATGAGTCCTGTGACCTCAGCCCACGCGGGGACCTACAG | |
| | GTGCTACGGCTCACTCAACTCCGACCCCTACCTGCTGTCTCACCCC | |
| | AGTGAGCCCCTGGAGCTCGTGGTCTCAGGACCCTCCATGGGTTCC | |
| | AGCCCCCCACCCACCGGTCCCATCTCCACACCTGGCCCTGAGGAC | |
| | CAGCCCCTCACCCCCACTGGGTCGGACCCCCAAAGTGGTCTGGGA | |
| | AGGCACCTGGGGGTTGTG | |
| LILRB2_P6 | QTGTIPKPTLWAEPDSVITQGSPVTLSCQGSLEAQEYRLYREKKSAS | 15 |
| | WITRIRPELVKNGQFHIPSITWEHTGRYGCQYYSRARWSELSDPLVL | |
| | VMTGAYPKPTLSAQPSPVVTSGGRVTLQCESQVAFGGFILCKEGEDE | |
| | HPQCLNSQPHARGSSRAIFSVGPVSPNRRWSHRCYGYDLNSPYVWSS | |
| | PSDLLELLVPGVSKKPSLSVQPGPVMAPGESLTLQCVSDVGYDRFVL | |
| | YKEGERDLRQLPGRQPQAGLSQANFTLGPVSRSYGGQYRCYGAHNL | |
| | SSECSAPSDPLDILITGQIRGTPFISVQPGPTVASGENVTLLCQSWRQF | |
| | HTFLLTKAGAADAPLRLRSIHEYPKYQAEFPMSPVTSAHAGTYRCYG | |
| | SLNSDPYLLSHPSEPLELVVSGPSMGSSPPPTGPISTPGPEDQPLTPTGS | |
| | DPQSGLGRHLGVV | |
| LILRB3_D1 | GGGCCCTTCCCCAAACCCACCCTCTGGGCTGAGCCAGGCTCTGTG | |
| | ATCAGCTGGGGGAGCCCCGTGACCATCTGGTGTCAGGGGAGCCA | |
| | GGAGGCCCAGGAGTACCGACTGCATAAAGAGGGAAGCCCAGAGC | 16 |
| | CCTTGGACAGAAATAACCCACTGGAACCCAAGAACAAGGCCAGA | |
| | TTCTCCATCCCATCCATGACAGAGCACCATGCAGGGAGATACCGC | |
| | TGCCACTATTACAGCTCTGCAGGCTGGTCAGAGCCCAGCGACCCC | |
| | CTGGAGATGGTGATGACAGGAGCCTACAGCAAACCCACCCTCTC | |
| | AGCCCTGCCCAGCCCTGTGGTGGCCTCAGGGGGGAATATGACCCT | |
| | CCGATGTGGCTCACAGAAGGGATATCACCATTTTGTTCTGATGAA | |
| | GGAAGGAGAACACCAGCTCCCCCGGACCCTGGACTCACAGCAGC | |
| | TCCACAGTCGGGGGTTCCAGGCCCTGTTCCCTGTGGGCCCCGTGA | |
| | CCCCCAGCCACAGGTGGAGGTTCACATGCTATTACTATTATACAA | |
| | ACACCCCCTGGGTGTGGTCCCACCCCAGTGACCCCCTGGAGATTC | |
| | TGCCCTCAGGCGTGTCTAGGAAGCCCTCCCTCCTGACCCTGCAGG | |
| | GCCCTGTCCTGGCCCCTGGGCAGAGCCTGACCCTCCAGTGTGGCT | |
| | CTGATGTCGGCTACAACAGATTTGTTCTGTATAAGGAGGGGGAAC | |
| | GTGACTTCCTCCAGCGCCCTGGCCAGCAGCCCCAGGCTGGGCTCT | |
| | CCCAGGCCAACTTCACCCTGGGCCCTGTGAGCCCCTCCAATGGGG | |
| | GCCAGTACAGGTGCTACGGTGCACACAACCTCTCCTCCGAGTGGT | |
| | CGGCCCCCAGCGACCCCCTGAACATCCTGATGGCAGGACAGATCT | |
| | ATGACACCGTCTCCCTGTCAGCACAGCCGGGCCCCACAGTGGCCT | |
| | CAGGAGAGAACGTGACCCTGCTGTGTCAGTCATGGTGGCAGTTTG | |
| | ACACTTTCCTTCTGACCAAAGAAGGGGCAGCCCATCCCCCACTGC | |
| | GTCTGAGATCAATGTACGGAGCTCATAAGTACCAGGCTGAATTCC | |
| | CCATGAGTCCTGTGACCTCAGCCCACGCGGGGACCTACAGGTGCT | |
| | ACGGCTCATACAGCTCCAACCCCCACCTGCTGTCTCACCCCAGTG | |
| | AGCCCCTGGAGCTCGTGGTCTCAGGACACTCTGGAGGCTCCAGCC | |
| | TCCCACCCACAGGGCCGCCCTCCACACCTGGTCTGGGAAGATACC | |
| | TGGAG | |
| LILRB3_P1 | GPFPKPTLWAEPGSVISWGSPVTIWCQGSQEAQEYRLHKEGSPEPLD | 17 |
| | RNNPLEPKNKARFSIPSMTEHHAGRYRCHYYSSAGWSEPSDPLEMV | |
| | MTGAYSKPTLSALPSPVVASGGNMTLRCGSQKGYHHFVLMKEGEH | |
| | QLPRTLDSQQLHSRGFQALFPVGPVTPSHRWRFTCYYYYTNTPWVW | |
| | SHPSDPLEILPSGVSRKPSLLTLQGPVLAPGQSLTLQCGSDVGYNRFV | |
| | LYKEGERDFLQRPGQQPQAGLSQANFTLGPVSPSNGGQYRCYGAHN | |
| | LSSEWSAPSDPLNILMAGQIYDTVSLSAQPGPTVASGENVTLLCQSW | |
| | WQFDTFLLTKEGAAHPPLRLRSMYGAHKYQAEFPMSPVTSAHAGTY | |
| | RCYGSYSSNPHLLSHPSEPLELVVSGHSGGSSLPPTGPPSTPGLGRYLE | |
| LILRB4_D1 | CAGGCAGGGCCCCTCCCCAAACCCACCCTCTGGGCTGAGCCAGGC | 18 |
| | TCTGTGATCAGCTGGGGGAACTCTGTGACCATCTGGTGTCAGGGG | |
| | ACCCTGGAGGCTCGGGAGTACCGTCTGGATAAAGAGGAAAGCCC | |
| | AGCACCCTGGGACAGACAGAACCCACTGGAGCCCAAGAACAAGG | |
| | CCAGATTCTCCATCCCATCCATGACAGAGGACTATGCAGGGAGAT | |
| | ACCGCTGTTACTATCGCAGCCCTGTAGGCTGGTCACAGCCCAGTG | |
| | ACCCCCTGGAGCTGGTGATGACAGGAGCCTACAGTAAACCCACC | |
| | CTTTCAGCCCTGCCGAGTCCTCTTGTGACCTCAGGAAAGAGCGTG | |
| | ACCCTGCTGTGTCAGTCACGGAGCCCAATGGACACTTTTCTTCTG | |
| | ATCAAGGAGCGGGCAGCCCATCCCCTACTGCATCTGAGATCAGA | |
| | GCACGGAGCTCAGCAGCACCAGGCTGAATTCCCCATGAGTCCTGT | |
| | GACCTCAGTGCACGGGGGGACCTACAGGTGCTTCAGCTCACACG | |
| | GCTTCTCCCACTACCTGCTGTCACACCCCAGTGACCCCCTGGAGC | |
| | TCATAGTCTCAGGCTCCTTGGAGGGTCCCAGGCCCTCACCCACAA | |
| | GGTCCGTCTCAACAGCTGCAGGCCCTGAGGACCAGCCCCTCATGC | |
| | CTACAGGGTCAGTCCCCCACAGTGGTCTGAGAAGGCACTGGGAG | |
| LILRB4_P1 | QAGPLPKPTLWAEPGSVISWGNSVTIWCQGTLEAREYRLDKEESPAP | 19 |
| | WDRQNPLEPKNKARFSIPSMTEDYAGRYRCYYRSPVGWSQPSDPLE | |
| | LVMTGAYSKPTLSALPSPLVTSGKSVTLLCQSRSPMDTFLLIKERAAH | |
| | PLLHLRSEHGAQQHQAEFPMSPVTSVHGGTYRCFSSHGFSHYLLSHP | |
| | SDPLELIVSGSLEGPRPSPTRSVSTAAGPEDQPLMPTGSVPHSGLRRH | |
| | WE | |
| LILRB5 | MTLTLSVLICLGLSVGPRTCVQAGTLPKPTLWAEPASVIARGKPVTL | 20 |
| (precursor) | WCQGPLETEEYRLDKEGLPWARKRQNPLEPGAKAKFHIPSTVYDSA | |
| (UniProtKB | GRYRCYYETPAGWSEPSDPLELVATGFYAEPTLLALPSPVVASGGNV | |
| Accession No. | TLQCDTLDGLLTFVLVEEEQKLPRTLYSQKLPKGPSQALFPVGPVTPS | |
| O75023.1) | CRWRFRCYYYYRKNPQVWSNPSDLLEILVPGVSRKPSLLIPQGSVVA | |
| | RGGSLTLQCRSDVGYDIFVLYKEGEHDLVQGSGQQPQAGLSQANFT | |
| | LGPVSRSHGGQYRCYGAHNLSPRWSAPSDPLDILIAGLIPDIPALSVQ | |
| | PGPKVASGENVTLLCQSWHQIDTFFLTKEGAAHPPLCLKSKYQSYRH | |
| | QAEFSMSPVTSAQGGTYRCYSAIRSYPYLLSSPSYPQELVVSGPSGDP | |
| | SLSPTGSTPTPGPEDQPLTPTGLDPQSGLGRHLGVVTGVSVAFVLLLF | |
| | LLLFLLLRHRHQSKHRTSAHFYRPAGAAGPEPKDQGLQKRASPVADI | |
| | QEEILNAAVKDTQPKDGVEMDARAAASEAPQDVTYAQLHSLTLRRE | |
| | ATEPPPSQEREPPAEPSIYAPLAIH | |
| LILRB3_1 | MTPALTALLCLGLSLGPRTRMQAGPFPKPTLWAEPGSVISWGSPVTI | 21 |
| (isoform 1 | WCQGSLEAQEYQLDKEGSPEPWDRNNPLEPKNKARFSIPSMTQHHA | |
| precursor) | GRYRCHYYSSAGWSEPSDPLELVMTGFYNKPTLSALPSPVVASGGN | |
| (NCBI Accession | MTLRCGSQKGYHHFVLMKEGEHQLPRTLDSQQLHSGGFQALFPVGP | |
| No. | VTPSHRWRFTCYYYYTNTPWVWSHPSDPLEILPSGVSRKPSLLTLQG | |
| NP_001074919.2) | PVLAPGQSLTLQCGSDVGYDRFVLYKEGERDFLQRPGQQPQAGLSQ | |
| | ANFTLGPVSRSYGGQYRCYGAHNLSSEWSAPSDPLDILITGQIYDTVS | |
| | LSAQPGPTVASGENMTLLCQSRGYFDTFLLTKEGAAHPPLRLRSMY | |
| | GAHKYQAEFPMSPVTSAHAGTYRCYGSRSSNPHLLSFPSEPLELMVS | |
| | GHSGGSSLPPTGPPSTPGLGRYLEVLIGVSVAFVLLLFLLLFLLLLRQR | |
| | HSKHRTSDQRKTDFQRPAGAAETEPKDRGLLRRSSPAADVQEENLY | |
| | AAVKDTQSEDRVELDSQQSPHDEDPQAVTYAPVKHSSPRREMASPP | |
| | SSLSGEFLDTKDRQVEEDRQMDTEAAASEASQDVTYAQLHSLTLRR | |
| | KATEPPPSQEGEPPAEPSIYATLAIH | |
| LILRB3_2 | MTPALTALLCLGLSLGPRTRMQAGPFPKPTLWAEPGSVISWGSPVTI | 22 |
| (isoform 2 | WCQGSLEAQEYQLDKEGSPEPWDRNNPLEPKNKARFSIPSMTQHHA | |
| precursor) | GRYRCHYYSSAGWSEPSDPLELVMTGFYNKPTLSALPSPVVASGGN | |
| (NCBI Accession. | MTLRCGSQKGYHHFVLMKEGEHQLPRTLDSQQLHSGGFQALFPVGP | |
| No. NP_006855.3) | VTPSHRWRFTCYYYYTNTPWVWSHPSDPLEILPSGVSRKPSLLTLQG | |
| | PVLAPGQSLTLQCGSDVGYDRFVLYKEGERDFLQRPGQQPQAGLSQ | |
| | ANFTLGPVSRSYGGQYRCYGAHNLSSEWSAPSDPLDILITGQIYDTVS | |
| | LSAQPGPTVASGENMTLLCQSRGYFDTFLLTKEGAAHPPLRLRSMY | |
| | GAHKYQAEFPMSPVTSAHAGTYRCYGSRSSNPHLLSFPSEPLELMVS | |
| | GHSGGSSLPPTGPPSTPGLGRYLEVLIGVSVAFVLLLFLLLFLLLLRQR | |
| | HSKHRTSDQRKTDFQRPAGAAETEPKDRGLLRRSSPAADVQEENLY | |
| | AAVKDTQSEDRVELDSQSPHDEDPQAVTYAPVKHSSPRREMASPPSS | |
| | LSGEFLDTKDRQVEEDRQMDTEAAASEASQDVTYAQLHSLTLRRKA | |
| | TEPPPSQEGEPPAEPSIYATLAIH | |
| LILRB3_3 | MTPALTALLCLGLSLGPRTRMQAGPFPKPTLWAEPGSVISWGSPVTI | 23 |
| (isoform 3 | WCQGSLEAQEYQLDKEGSPEPWDRNNPLEPKNKARFSIPSMTQHHA | |
| precursor) | GRYRCHYYSSAGWSEPSDPLELVMTGFYNKPTLSALPSPVVASGGN | |
| (NCBI Accession | MTLRCGSQKGYHHFVLMKEGEHQLPRTLDSQQLHSGGFQALFPVGP | |
| No. | VTPSHRWRFTCYYYYTNTPWVWSHPSDPLEILPSGVSRKPSLLTLQG | |
| NP_001307889.1) | PVLAPGQSLTLQCGSDVGYDRFVLYKEGERDFLQRPGQQPQAGLSQ | |
| | ANFTLGPVSRSYGGQYRCYGAHNLSSEWSAPSDPLDILITGQIYDTVS | |
| | LSAQPGPTVASGENMTLLCQSRGYFDTFLLTKEGAAHPPLRLRSMY | |
| | GAHKYQAEFPMSPVTSAHAGTYRCYGSRSSNPHLLSFPSEPLELMVS | |
| | GHSGGSSLPPTGPPSTPGGPEDQPLNPPGSGPQNGLGRYLEVLIGVSV | |
| | AFVLLLFLLLFLLLLRQRHSKHRTSDQRKTDFQRPAGAAETEPKDRG | |
| | LLRRSSPAADVQEENLYAAVKDTQSEDRVELDSQSPHDEDPQAVTY | |
| | APVKHSSPRREMASPPSSLSGEFLDTKDRQVEEDRQMDTEAAASEAS | |
| | QDVTYAQLHSLTLRRKATEPPPSQEGEPPAEPSIYATLAIH | |
| LILRB4_ 1 | MIPTFTALLCLGLSLGPRTHMQAGPLPKPTLWAEPGSVISWGNSVTI | |
| (isoform 1 | WCQGTLEAREYRLDKEESPAPWDRQNPLEPKNKARFSIPSMTEDYA | 24 |
| precursor) | GRYRCYYRSPVGWSQPSDPLELVMTGAYSKPTLSALPSPLVTSGKSV | |
| (NCBI Accession | TLLCQSRSPMDTFLLIKERAAHPLLHLRSEHGAQQHQAEFPMSPVTS | |
| No. | VHGGTYRCFSSHGFSHYLLSHPSDPLELIVSGSLEGPRPSPTRSVSTAA | |
| NP_001265355.2) | GPEDQPLMPTGSVPHSGLRRHWEVLIGVLVVSILLLSLLLFLLLQHW | |
| | RQGKHRTLAQRQADFQRPPGAAEPEPKDGGLQRRSSPAADVQGENF | |
| | CAAVKNTQPEDGVEMDTRQSPHDEDPQAVTYAKVKHSRPRREMAS | |
| | PPSPLSGEFLDTKDRQAEEDRQMDTEAAASEAPQDVTYARLHSFTLR | |
| | QKATEPPPSQEGASPAEPSVYATLAIH | |
| LILRB4_2 | MIPTFTALLCLGLSLGPRTHMQAGPLPKPTLWAEPGSVISWGNSVTI | |
| (isoform 2 | WCQGTLEAREYRLDKEESPAPWDRQNPLEPKNKARFSIPSMTEDYA | |
| precursor) | GRYRCYYRSPVGWSQPSDPLELVMTGAYSKPTLSALPSPLVTSGKSV | |
| (NCBI Accession | TLLCQSRSPMDTFLLIKERAAHPLLHLRSEHGAQQHQAEFPMSPVTS | |
| No. | VHGGTYRCFSSHGFSHYLLSHPSDPLELIVSGSLEGPRPSPTRSVSTAA | 25 |
| NP_001265356.2) | GPEDQPLMPTGSVPHSGLRRHWEVLIGVLVVSILLLSLLLFLLLQHW | |
| | RQGKHRTLAQRQADFQRPPGAAEPEPKDGGLQRRSSPAADVQGENF | |
| | CAAVKNTQPEDGVEMDTRSPHDEDPQAVTYAKVKHSRPRREMASP | |
| | PSPLSGEFLDTKDRQAEEDRQMDTEAAASEAPQDVTYARLHSFTLR | |
| | QKATEPPPSQEGASPAEPSVYATLAIH | |
| LILRB4_3 | MIPTFTALLCLGLSLGPRTHMQAGPLPKPTLWAEPGSVISWGNSVTI | 26 |
| (isoform 3 | WCQGTLEAREYRLDKEESPAPWDRQNPLEPKNKARFSIPSMTEDYA | |
| precursor) | GRYRCYYRSPVGWSQPSDPLELVMTGAYSKPTLSALPSPLVTSGKSV | |
| (NCBI Accession | TLLCQSRSPMDTFLLIKERAAHPLLHLRSEHGAQQHQAEFPMSPVTS | |
| No. | VHGGTYRCFSSHGFSHYLLSHPSDPLELIVSGSLEGPRPSPTRSVSTAA | |
| NP_001265357.2) | GPEDQPLMPTGSVPHSGLRRHWEVLIGVLVVSILLLSLLLFLLLQHW | |
| | RQGKHRTLAQRQADFQRPPGAAEPEPKDGGLQRRSSPAADVQGENF | |
| | SGAAVKNTQPEDGVEMDTRSPHDEDPQAVTYAKVKHSRPRREMAS | |
| | PPSPLSGEFLDTKDRQAEEDRQMDTEAAASEAPQDVTYARLHSFTLR | |
| | QKATEPPPSQEGASPAEPSVYATLAIH | |
| LILRB4_4 | MEQPHDEKDPASKRPHPVCLFVLPALRTHPSAQLGPLGGDAMIPTFT | |
| (isoform 4 | ALLCLGPLPKPTLWAEPGSVISWGNSVTIWCQGTLEAREYRLDKEES | |
| precursor) | PAPWDRQNPLEPKNKARFSIPSMTEDYAGRYRCYYRSPVGWSQPSD | |
| (NCBI Accession | PLELVMTGAYSKPTLSALPSPLVTSGKSVTLLCQSRSPMDTFLLIKER | |
| No. | AAHPLLHLRSEHGAQQHQAEFPMSPVTSVHGGTYRCFSSHGFSHYL | |
| NP_001265358.2) | LSHPSDPLELIVSGSLEGPRPSPTRSVSTAAGPEDQPLMPTGSVPHSGL | 27 |
| | RRHWEVLIGVLVVSILLLSLLLFLLLQHWRQGKHRTLAQRQADFQRP | |
| | PGAAEPEPKDGGLQRRSSPAADVQGENFSGAAVKNTQPEDGVEMDT | |
| | RQSPHDEDPQAVTYAKVKHSRPRREMASPPSPLSGEFLDTKDRQAEE | |
| | DRQMDTEAAASEAPQDVTYARLHSFTLRQKATEPPPSQEGASPAEPS | |
| | VYATLAIH | |
| LILRB4_5 | MIPTFTALLCLGLSLGPRTHMQAGPLPKPTLWAEPGSVISWGNSVTI | 28 |
| (isoform 5 | WCQGTLEAREYRLDKEESPAPWDRQNPLEPKNKARFSIPSMTEDYA | |
| precursor) | GRYRCYYRSPVGWSQPSDPLELVMTGAYSKPTLSALPSPLVTSGKSV | |
| (NCBI Accession | TLLCQSRSPMDTFLLIKERAAHPLLHLRSEHGAQQHQAEFPMSPVTS | |
| No. | VHGGTYRCFSSHGFSHYLLSHPSDPLELIVSGSLEGPRPSPTRSVSTAA | |
| NP_001265359.2) | GPEDQPLMPTGSVPHSGE | |
| LILRB5_1 | MTLTLSVLICLGLSVGPRTCVQAGTLPKPTLWAEPASVIARGKPVTL | 29 |
| (isoform 1 | WCQGPLETEEYRLDKEGLPWARKRQNPLEPGAKAKFHIPSTVYDSA | |
| precursor) | GRYRCYYETPAGWSEPSDPLELVATGFYAEPTLLALPSPVVASGGNV | |
| (NCBI Accession | TLQCDTLDGLLTFVLVEEEQKLPRTLYSQKLPKGPSQALFPVGPVTPS | |
| No. | CRWRFRCYYYYRKNPQVWSNPSDLLEILVPGVSRKPSLLIPQGSVVA | |
| NP_001074911.1) | RGGSLTLQCRSDVGYDIFVLYKEGEHDLVQGSGQQPQAGLSQANFT | |
| | LGPVSRSHGGQYRCYGAHNLSPRWSAPSDPLDILIAGLIPDIPALSVQ | |
| | PGPKVASGENVTLLCQSWHQIDTFFLTKEGAAHPPLCLKSKYQSYRH | |
| | QAEFSMSPVTSAQGGTYRCYSAIRSYPYLLSSPSYPQELVVSGPSGDP | |
| | SLSPTGSTPTPAGPEDQPLTPTGLDPQSGLGRHLGVVTGVSVAFVLLL | |
| | FLLLFLLLRHRHQSKHRTSAHFYRPAGAAGPEPKDQGLQKRASPVA | |
| | DIQEEILNAAVKDTQPKDGVEMDARAAASEAPQDVTYAQLHSLTLR | |
| | REATEPPPSQEREPPAEPSIYAPLAIH | |
| LILRB5_2 | MTLTLSVLICLGLSVGPRTCVQAGTLPKPTLWAEPASVIARGKPVTL | 30 |
| (isoform 2 | WCQGPLETEEYRLDKEGLPWARKRQNPLEPGAKAKFHIPSTVYDSA | |
| precursor) | GRYRCYYETPAGWSEPSDPLELVATGFYAEPTLLALPSPVVASGGNV | |
| (NCBI Accession | TLQCDTLDGLLTFVLVEEEQKLPRTLYSQKLPKGPSQALFPVGPVTPS | |
| No. NP_006831.1) | CRWRFRCYYYYRKNPQVWSNPSDLLEILVPGVSRKPSLLIPQGSVVA | |
| | RGGSLTLQCRSDVGYDIFVLYKEGEHDLVQGSGQQPQAGLSQANFT | |
| | LGPVSRSHGGQYRCYGAHNLSPRWSAPSDPLDILIAGLIPDIPALSVQ | |
| | PGPKVASGENVTLLCQSWHQIDTFFLTKEGAAHPPLCLKSKYQSYRH | |
| | QAEFSMSPVTSAQGGTYRCYSAIRSYPYLLSSPSYPQELVVSGPSGDP | |
| | SLSPTGSTPTPGPEDQPLTPTGLDPQSGLGRHLGVVTGVSVAFVLLLF | |
| | LLLFLLLRHRHQSKHRTSAHFYRPAGAAGPEPKDQGLQKRASPVADI | |
| | QEEILNAAVKDTQPKDGVEMDARAAASEAPQDVTYAQLHSLTLRRE | |
| | ATEPPPSQEREPPAEPSIYAPLAIH | |
| LILRB5_3 | MTLTLSVLICLGLSVGPRTCVQAGTLPKPTLWAEPASVIARGKPVTL | 31 |
| (isoform 3 | WCQGPLETEEYRLDKEGLPWARKRQNPLEPGAKAKFHIPSTVYDSA | |
| precursor) | GRYRCYYETPAGWSEPSDPLELVATGVSRKPSLLIPQGSVVARGGSL | |
| (NCBI Accession | TLQCRSDVGYDIFVLYKEGEHDLVQGSGQQPQAGLSQANFTLGPVS | |
| No. | RSHGGQYRCYGAHNLSPRWSAPSDPLDILIAGLIPDIPALSVQPGPKV | |
| NP_001074912.1) | ASGENVTLLCQSWHQIDTFFLTKEGAAHPPLCLKSKYQSYRHQAEFS | |
| | MSPVTSAQGGTYRCYSAIRSYPYLLSSPSYPQELVVSGPSGDPSLSPT | |
| | GSTPTPAGPEDQPLTPTGLDPQSGLGRHLGVVTGVSVAFVLLLFLLLF | |
| | LLLRHRHQSKHRTSAHFYRPAGAAGPEPKDQGLQKRASPVADIQEEI | |
| | LNAAVKDTQPKDGVEMDARAAASEAPQDVTYAQLHSLTLRREATE | |
| | PPPSQEREPPAEPSIYAPLAIH | |
| LILRB5_4 | MTLTLSVLICLGLSVGPRTCVQAGTLPKPTLWAEPASVIARGKPVTL | 32 |
| (isoform 4 | WCQGPLETEEYRLDKEGLPWARKRQNPLEPGAKAKFHIPSTVYDSA | |
| precursor) | GRYRCYYETPAGWSEPSDPLELVATALPSPVVASGGNVTLQCDTLD | |
| (NCBI Accession | GLLTFVLVEEEQKLPRTLYSQKLPKGPSQALFPVGPVTPSCRWRFRC | |
| No. | YYYYRKNPQVWSNPSDLLEILVPGVSRKPSLLIPQGSVVARGGSLTL | |
| NP_001291386.1) | QCRSDVGYDIFVLYKEGEHDLVQGSGQQPQAGLSQANFTLGPVSRS | |
| | HGGQYRCYGAHNLSPRWSAPSDPLDILIAGLIPDIPALSVQPGPKVAS | |
| | GENVTLLCQSWHQIDTFFLTKEGAAHPPLCLKSKYQSYRHQAEFSMS | |
| | PVTSAQGGTYRCYSAIRSYPYLLSSPSYPQELVVSGPSGDPSLSPTGST | |
| | PTPAGPEDQPLTPTGLDPQSGLGRHLGVVTGVSVAFVLLLFLLLFLLL | |
| | RHRHQSKHRTSAHFYRPAGAAGPEPKDQGLQKRASPVADIQEEILNA | |
| | AVKDTQPKDGVEMDARQSPHDEDPQAVTYAPVKHSRPRREMASPP | |
| | SPLSGEFLDTKDRQAEEDRQMDTERVLSSPGPQASPTPTTFLPSHSPP | |
| | LQAAASEAPQDVTYAQLHSLTLRREATEPPPSQEREPPAEPSIYAPLAI | |
| | H | |
| Lilrb1-01 | GGGCACCTCCCCAAGCCCACCCTCTGGGCTGAACCAGGCTCTGTG | 33 |
| extracellular | ATCACCCAGGGGAGTCCTGTGACCCTCAGGTGTCAGGGGGGCCA | |
| | GGAGACCCAGGAGTACCGTCTATATAGAGAAAAGAAAACAGCAC | |
| | TCTGGATTACACGTATCCCACAGGAGCTTGTGAAGAAGGGCCAGT | |
| | TCCCCATCCCATCCATCACCTGGGAACACGCAGGGCGGTATCGCT | |
| | GTTACTATGGTAGCGACACTGCAGGCCGCTCAGAGAGCAGTGAC | |
| | CCCCTGGAGCTGGTGGTGACAGGAGCCTACATCAAACCCACCCTC | |
| | TCAGCCCAGCCCAGCCCCGTGGTGAACTCAGGAGGGAATGTAAT | |
| | CCTCCAGTGTGACTCACAGGTGGCATTTGATGGCTTCATTCTGTGT | |
| | AAGGAAGGAGAAGATGAACACCCACAATGCCTGAACTCCCAGCC | |
| | CCATGCCCGTGGGTCGTCCCGCGCCATCTTCTCCGTGGGCCCCGT | |
| | GAGCCCGAGTCGCAGGTGGTGGTACAGGTGCTATGCTTATGACTC | |
| | GAACTCTCCCTATGAGTGGTCTCTACCCAGTGATCTCCTGGAGCT | |
| | CCTGGTCCTAGGTGTTTCTAAGAAGCCATCACTCTCAGTGCAGCC | |
| | AGGTCCTATCGTGGCCCCTGAGGAGACCCTGACTCTGCAGTGTGG | |
| | CTCTGATGCTGGCTACAACAGATTTGTTCTGTATAAGGACGGGGA | |
| | ACGTGACTTCCTTCAGCTCGCTGGCGCACAGCCCCAGGCTGGGCT | |
| | CTCCCAGGCCAACTTCACCCTGGGCCCTGTGAGCCGCTCCTACGG | |
| | GGGCCAGTACAGATGCTACGGTGCACACAACCTCTCCTCCGAGTG | |
| | GTCGGCCCCCAGCGACCCCCTGGACATCCTGATCGCAGGACAGTT | |
| | CTATGACAGAGTCTCCCTCTCGGTGCAGCCGGGCCCCACGGTGGC | |
| | CTCAGGAGAGAACGTGACCCTGCTGTGTCAGTCACAGGGATGGA | |
| | TGCAAACTTTCCTTCTGACCAAGGAGGGGGCAGCTGATGACCCAT | |
| | GGCGTCTAAGATCAACGTACCAATCTCAAAAATACCAGGCTGAAT | |
| | TCCCCATGGGTCCTGTGACCTCAGCCCATGCGGGGACCTACAGGT | |
| | GCTACGGCTCACAGAGCTCCAAACCCTACCTGCTGACTCACCCCA | |
| | GTGACCCCCTGGAGCTCGTGGTCTCAGGACCGTCTGGGGGCCCCA | |
| | GCTCCCCGACAACAGGCCCCACCTCCACATCTGCAGGCCCTGAGG | |
| | ACCAGCCCCTCACCCCCACCGGGTCGGACCCCCAGAGTGGTCTGG | |
| | GAAGGCACCTGGGG | |
| Lilrb1_02 | GGGCACCTCCCCAAGCCCACCCTCTGGGCTGAACCAGGCTCTGTG | 34 |
| (consensus | ATCACCCAGGGGAGTCCTGTGACCCTCAGGTGTCAGGGGGGCCA | |
| sequence) | GGAGACCCAGGAGTACCGTCTATATAGAGAAAAGAAAACAGCAC | |
| | CCTGGATTACACGGATTCCACAGGAGCTTGTGAAGAAGGGCCAG | |
| | TTCCCCATCCCATCCATCACCTGGGAACACACAGGGCGGTATCGC | |
| | TGTTACTATGGTAGCGACACTGCAGGCCGCTCAGAGAGCAGTGAC | |
| | CCCCTGGAGCTGGTGGTGACAGGAGCCTACATCAAACCCACCCTC | |
| | TCAGCCCAGCCCAGCCCCGTGGTGAACTCAGGAGGGAATGTAAC | |
| | CCTCCAGTGTGACTCACAGGTGGCATTTGATGGCTTCATTCTGTGT | |
| | AAGGAAGGAGAAGATGAACACCCACAATGCCTGAACTCCCAGCC | |
| | CCATGCCCGTGGGTCGTCCCGCGCCATCTTCTCCGTGGGCCCCGT | |
| | GAGCCCGAGTCGCAGGTGGTGGTACAGGTGCTATGCTTATGACTC | |
| | GAACTCTCCCTATGAGTGGTCTCTACCCAGTGATCTCCTGGAGCT | |
| | CCTGGTCCTAGGTGTTTCTAAGAAGCCATCACTCTCAGTGCAGCC | |
| | AGGTCCTATCGTGGCCCCTGAGGAGACCCTGACTCTGCAGTGTGG | |
| | CTCTGATGCTGGCTACAACAGATTTGTTCTGTATAAGGACGGGGA | |
| | ACGTGACTTCCTTCAGCTCGCTGGCGCACAGCCCCAGGCTGGGCT | |
| | CTCCCAGGCCAACTTCACCCTGGGCCCTGTGAGCCGCTCCTACGG | |
| | GGGCCAGTACAGATGCTACGGTGCACACAACCTCTCCTCCGAGTG | |
| | GTCGGCCCCCAGCGACCCCCTGGACATCCTGATCGCAGGACAGTT | |
| | CTATGACAGAGTCTCCCTCTCGGTGCAGCCGGGCCCCACGGTGGC | |
| | CTCAGGAGAGAACGTGACCCTGCTGTGTCAGTCACAGGGATGGA | |
| | TGCAAACTTTCCTTCTGACCAAGGAGGGGGCAGCTGATGACCCAT | |
| | GGCGTCTAAGATCAACGTACCAATCTCAAAAATACCAGGCTGAAT | |
| | TCCCCATGGGTCCTGTGACCTCAGCCCATGCGGGGACCTACAGGT | |
| | GCTACGGCTCACAGAGCTCCAAACCCTACCTGCTGACTCACCCCA | |
| | GTGACCCCCTGGAGCTCGTGGTCTCAGGACCGTCTGGGGGCCCCA | |
| | GCTCCCCGACAACAGGCCCCACCTCCACATCTGCAGGCCCTGAGG | |
| | ACCAGCCCCTCACCCCCACCGGGTCGGACCCCCAGAGTGGTCTGG | |
| | GAAGGCACCTGGGG | |
| Lilrb1_03 | GGGCACCTCCCCAAGCCCACCCTCTGGGCTGAACCAGGCTCTGTG | 35 |
| | ATCACCCAGGGGAGTCCTGTGACCCTCAGGTGTCAGGGGGGCCA | |
| | GGAGACCCAGGAGTACCGTCTATATAGAGAAAAGAAAACAGCAC | |
| | CCTGGATTACACGTATCCCACAGGAGCTTGTGAAGAAGGGCCAGT | |
| | TCCCCATCCCATCCATCACCTGGGAACACGCAGGGCGGTATCGCT | |
| | GTTACTATGGTAGCGACACTGCAGGCCGCTCAGAGAGCAGTGAC | |
| | CCCCTGGAGCTGGTGGTGACAGGAGCCTACATCAAACCCACCCTC | |
| | TCAGCCCAGCCCAGCCCCGTGGTGAACTCAGGAGGGAATGTAAC | |
| | CCTCCAGTGTGACTCACAGGTGGCATTTGATGGCTTCATTCTGTGT | |
| | AAGGAAGGAGAAGATGAACACCCACAATGCCTGAACTCCCAGCC | |
| | CCATGCCCGTGGGTCGTCCCGCGCCATCTTCTCCGTGGGCCCCGT | |
| | GAGCCCGAGTCGCAGGTGGTGGTACAGGTGCTATGCTTATGACTC | |
| | GAACTCTCCCTATGAGTGGTCTCTACCCAGTGATCTCCTGGAGCT | |
| | CCTGGTCCTAGGTGTTTCTAAGAAGCCATCACTCTCAGTGCAGCC | |
| | AGGTCCTATCGTGGCCCCTGAGGAGACCCTGACTCTGCAGTGTGG | |
| | CTCTGATGCTGGCTACAACAGATTTGTTCTGTATAAGGACGGGGA | |
| | ACGTGACTTCCTTCAGCTCGCTGGCGCACAGCCCCAGGCTGGGCT | |
| | CTCCCAGGCCAACTTCACCCTGGGCCCTGTGAGCCGCTCCTACGG | |
| | GGGCCAGTACAGATGCTACGGTGCACACAACCTCTCCTCCGAGTG | |
| | GTCGGCCCCCAGCGACCCCCTGGACATCCTGATCGCAGGACAGTT | |
| | CTATGACAGAGTCTCCCTCTCGGTGCAGCCGGGCCCCACGGTGGC | |
| | CTCAGGAGAGAACGTGACCCTGCTGTGTCAGTCACAGGGATGGA | |
| | TGCAAACTTTCCTTCTGACCAAGGAGGGGGCAGCTGATGACCCAT | |
| | GGCGTCTAAGATCAACGTACCAATCTCAAAAATACCAGGCTGAAT | |
| | TCCCCATGGGTCCTGTGACCTCAGCCCATGCGGGGACCTACAGGT | |
| | GCTACGGCTCACAGAGCTCCAAACCCTACCTGCTGACTCACCCCA | |
| | GTGACCCCCTGGAGCTCGTGGTCTCAGGACCGTCTGGGGGCCCCA | |
| | GCTCCCCGACAACAGGCCCCACCTCCACATCTGCAGGCCCTGAGG | |
| | ACCAGCCCCTCACCCCCACCGGGTCGGACCCCCAGAGTGGTCTGG | |
| | GAAGGCACCTGGGG | |
| Lilrb2_01 | CAGACAGGGACCATCCCCAAGCCCACCCTGTGGGCTGAGCCAGA | 36 |
| | CTCTGTGATCACCCAGGGGAGTCCCGTCACCCTCAGTTGTCAGGG | |
| | GAGCCTTGAAGCCCAGGAGTACCGTCTATATAGGGAGAAAAAAT | |
| | CAGCATCTTGGATTACACGGATACGACCAGAGCTTGTGAAGAAC | |
| | GGCCAGTTCCACATCCCATCCATCACCTGGGAACACACAGGGCGA | |
| | TATGGCTGTCAGTATTACAGCCGCGCTCGGTGGTCTGAGCTCAGT | |
| | GACCCCCTGGTGCTGGTGATGACAGGAGCCTACCCAAAACCCACC | |
| | CTCTCAGCCCAGCCCAGCCCTGTGGTGACCTCAGGAGGAAGGGTG | |
| | ACCCTCCAGTGTGAGTCACAGGTGGCATTTGGCGGCTTCATTCTG | |
| | TGTAAGGAAGGAGAAGAAGAACACCCACAATGCCTGAACTCCCA | |
| | GCCCCATGCCCGTGGGTCGTCCCGCGCCATCTTCTCCGTGGGCCC | |
| | CGTGAGCCCGAATCGCAGGTGGTCGCACAGGTGCTATGGTTATGA | |
| | CTTGAACTCTCCCTATGTGTGGTCTTCACCCAGTGATCTCCTGGAG | |
| | CTCCTGGTCCCAGGTGTTTCTAAGAAGCCATCACTCTCAGTGCAG | |
| | CCGGGTCCTGTCGTGGCCCCTGGGGAAAGCCTGACCCTCCAGTGT | |
| | GTCTCTGATGTCGGCTATGACAGATTTGTTCTGTACAAGGAGGGG | |
| | GAACGTGACCTTCGCCAGCTCCCTGGCCGGCAGCCCCAGGCTGGG | |
| | CTCTCCCAGGCCAACTTCACCCTGGGCCCTGTGAGCCGCTCCTAC | |
| | GGGGGCCAGTACAGATGCTACGGTGCATACAACCTCTCCTCTGAG | |
| | TGGTCGGCCCCCAGCGACCCCCTGGACATCCTGATCACAGGACAG | |
| | ATCCATGGCACACCCTTCATCTCAGTGCAGCCAGGCCCCACAGTG | |
| | GCCTCAGGAGAGAACGTGACCCTGCTGTGTCAGTCATGGCGGCA | |
| | GTTCCACACTTTCCTTCTGACCAAGGCGGGAGCAGCTGATGCCCC | |
| | ACTCCGTCTAAGATCAATACACGAATATCCTAAGTACCAGGCTGA | |
| | ATTCCCCATGAGTCCTGTGACCTCAGCCCACGCGGGGACCTACAG | |
| | GTGCTACGGCTCACTCAACTCCGACCCCTACCTGCTGTCTCACCCC | |
| | AGTGAGCCCCTGGAGCTCGTGGTCTCAGGACCCTCCATGGGTTCC | |
| | AGCCCCCCACCCACCGGTCCCATCTCCACACCTGGCCCTGAGGAC | |
| | CAGCCCCTCACCCCCACTGGGTCGGACCCCCAAAGTGGTCTGGGA | |
| | AGGCACCTGGGGGTTGTG | |
| Lilrb2_02 | CAGACAGGGACCATCCCCAAGCCCACCCTGTGGGCTGAGCCAGA | 37 |
| (consensus | CTCTGTGATCACCCAGGGGAGTCCCGTCACCCTCAGTTGTCAGGG | |
| sequence) | GAGCCTTGAAGCCCAGGAGTACCGTCTATATAGGGAGAAAAAAT | |
| | CAGCATCTTGGATTACACGGATACGACCAGAGCTTGTGAAGAAC | |
| | GGCCAGTTCCACATCCCATCCATCACCTGGGAACACACAGGGCGA | |
| | TATGGCTGTCAGTATTACAGCCGCGCTCGGTGGTCTGAGCTCAGT | |
| | GACCCCCTGGTGCTGGTGATGACAGGAGCCTACCCAAAACCCACC | |
| | CTCTCAGCCCAGCCCAGCCCTGTGGTGACCTCAGGAGGAAGGGTG | |
| | ACCCTCCAGTGTGAGTCACAGGTGGCATTTGGCGGCTTCATTCTG | |
| | TGTAAGGAAGGAGAAGATGAACACCCACAATGCCTGAACTCCCA | |
| | GCCCCATGCCCGTGGGTCGTCCCGCGCCATCTTCTCCGTGGGCCC | |
| | CGTGAGCCCGAATCGCAGGTGGTCGCACAGGTGCTATGGTTATGA | |
| | CTTGAACTCTCCCTATGTGTGGTCTTCACCCAGTGATCTCCTGGAG | |
| | CTCCTGGTCCCAGGTGTTTCTAAGAAGCCATCACTCTCAGTGCAG | |
| | CCGGGTCCTGTCATGGCCCCTGGGGAAAGCCTGACCCTCCAGTGT | |
| | GTCTCTGATGTCGGCTATGACAGATTTGTTCTGTACAAGGAGGGG | |
| | GAACGTGACCTTCGCCAGCTCCCTGGCCGGCAGCCCCAGGCTGGG | |
| | CTCTCCCAGGCCAACTTCACCCTGGGCCCTGTGAGCCGCTCCTAC | |
| | GGGGGCCAGTACAGATGCTACGGTGCACACAACCTCTCCTCTGAG | |
| | TGCTCGGCCCCCAGCGACCCCCTGGACATCCTGATCACAGGACAG | |
| | ATCCGTGGCACACCCTTCATCTCAGTGCAGCCAGGCCCCACAGTG | |
| | GCCTCAGGAGAGAACGTGACCCTGCTGTGTCAGTCATGGCGGCA | |
| | GTTCCACACTTTCCTTCTGACCAAGGCGGGAGCAGCTGATGCCCC | |
| | ACTCCGTCTAAGATCAATACACGAATATCCTAAGTACCAGGCTGA | |
| | ATTCCCCATGAGTCCTGTGACCTCAGCCCACGCGGGGACCTACAG | |
| | GTGCTACGGCTCACTCAACTCCGACCCCTACCTGCTGTCTCACCCC | |
| | AGTGAGCCCCTGGAGCTCGTGGTCTCAGGACCCTCCATGGGTTCC | |
| | AGCCCCCCACCCACCGGTCCCATCTCCACACCTGGCCCTGAGGAC | |
| | CAGCCCCTCACCCCCACTGGGTCGGACCCCCAAAGTGGTCTGGGA | |
| | AGGCACCTGGGGGTTGTG | |
| Lilrb2_03 | CAGACAGGGACCATCCCCAAGCCCACCCTGTGGGCTGAGCCAGA | 38 |
| | CTCTGTGATCACCCAGGGGAGTCCCGTCACCCTCAGTTGTCAGGG | |
| | GAGCCTTGAAGCCCAGGAGTACCGTCTATATAGGGAGAAAAAAT | |
| | CAGCATCTTGGATTACACGGATACGACCAGAGCTTGTGAAGAAC | |
| | GGCCAGTTCCACATCCCATCCATCACCTGGGAACACACAGGGCGA | |
| | TATGGCTGTCAGTATTACAGCCGCGCTCGGTGGTCTGAGCTCAGT | |
| | GACCCCCTGGTGCTGGTGATGACAGGAGCCTACCCAAAACCCACC | |
| | CTCTCAGCCCAGCCCAGCCCTGTGGTGACCTCAGGAGGAAGGGTG | |
| | ACCCTCCAGTGTGAGTCACAGGTGGCATTTGGCGGCTTCATTCTG | |
| | TGTAAGGAAGGAGAAGATGAACACCCACAATGCCTGAACTCCCA | |
| | GCCCCATGCCCGTGGGTCGTCCCGCGCCATCTTCTCCGTGGGCCC | |
| | CGTGAGCCCGAATCGCAGGTGGTCGCACAGGTGCTATGGTTATGA | |
| | CTTGAACTCTCCCTATGTGTGGTCTTCACCCAGTGATCTCCTGGAG | |
| | CTCCTGGTCCCAGGTGTTTCTAAGAAGCCATCACTCTCAGTGCAG | |
| | CCGGGTCCTGTCGTGGCCCCTGGGGAAAGCCTGACCCTCCAGTGT | |
| | GTCTCTGATGTCGGCTATGACAGATTTGTTCTGTACAAGGAGGGG | |
| | GAACGTGACCTTCGCCAGCTCCCTGGCCGGCAGCCCCAGGCTGGG | |
| | CTCTCCCAGGCCAACTTCACCCTGGGCCCTGTGAGCCGCTCCTAC | |
| | GGGGGCCAGTACAGATGCTACGGTGCACACAACCTCTCCTCTGAG | |
| | TGCTCGGCCCCCAGCGACCCCCTGGACATCCTGATCACAGGACAG | |
| | ATCCGTGGCACACCCTTCATCTCAGTGCAGCCAGGCCCCACAGTG | |
| | GCCTCAGGAGAGAACGTGACCCTGCTGTGTCAGTCATGGCGGCA | |
| | GTTCCACACTTTCCTTCTGACCAAGGCGGGAGCAGCTGATGCCCC | |
| | ACTCCGTCTAAGATCAATACACGAATATCCTAAGTACCAGGCTGA | |
| | ATTCCCCATGAGTCCTGTGACCTCAGCCCACGCGGGGACCTACAG | |
| | GTGCTACGGCTCACTCAACTCCGACCCCTACCTGCTGTCTCACCCC | |
| | AGTGAGCCCCTGGAGCTCGTGGTCTCAGGACCCTCCATGGGTTCC | |
| | AGCCCCCCACCCACCGGTCCCATCTCCACACCTGGCCCTGAGGAC | |
| | CAGCCCCTCACCCCCACTGGGTCGGACCCCCAAAGTGGTCTGGGA | |
| | AGGCACCTGGGGGTTGTG | |
| Lilrb2_04 | CAGACAGGGACCATCCCCAAGCCCACCCTGTGGGCTGAGCCAGA | 39 |
| | CTCTGTGATCACCCAGGGGAGTCCCGTCACCCTCAGTTGTCAGGG | |
| | GAGCCTTGAAGCCCAGGAGTACCGTCTATATAGGGAGAAAAAAT | |
| | CAGCATCTTGGATTACACGGATACGACCAGAGCTTGTGAAGAAC | |
| | GGCCAGTTCCACATCCCATCCATCACCTGGGAACACACAGGGCGA | |
| | TATGGCTGTCAGTATTACAGCCGCGCTCGGTGGTCTGAGCTCAGT | |
| | GACCCCCTGGTGCTGGTGATGACAGGAGCCTACCCAAAACCCACC | |
| | CTCTCAGCCCAGCCCAGCCCTGTGGTGACCTCAGGAGGAAGGGTG | |
| | ACCCTCCAGTGTGAGTCACAGGTGGCATTTGGCGGCTTCATTCTG | |
| | TGTAAGGAAGGAGAAGAAGAACACCCACAATGCCTGAACTCCCA | |
| | GCCCCATGCCCGTGGGTCGTCCCGCGCCATCTTCTCCGTGGGCCC | |
| | CGTGAGCCCGAATCGCAGGTGGTCGCACAGGTGCTATGGTTATGA | |
| | CTTGAACTCTCCCTATGTGTGGTCTTCACCCAGTGATCTCCTGGAG | |
| | CTCCTGGTCCCAGGTGTTTCTAAGAAGCCATCACTCTCAGTGCAG | |
| | CCGGGTCCTGTCGTGGCCCCTGGGGAAAGCCTGACCCTCCAGTGT | |
| | GTCTCTGATGTCGGCTATGACAGATTTGTTCTGTACAAGGAGGGG | |
| | GAACGTGACCTTCGCCAGCTCCCTGGCCGGCAGCCCCAGGCTGGG | |
| | CTCTCCCAGGCCAACTTCACCCTGGGCCCTGTGAGCCGCTCCTAC | |
| | GGGGGCCAGTACAGATGCTACGGTGCACACAACCTCTCCTCTGAG | |
| | TGCTCGGCCCCCAGCGACCCCCTGGACATCCTGATCACAGGACAG | |
| | ATCCGTGGCACACCCTTCATCTCAGTGCAGCCAGGCCCCACAGTG | |
| | GCCTCAGGAGAGAACGTGACCCTGCTGTGTCAGTCATGGCGGCA | |
| | GTTCCACACTTTCCTTCTGACCAAGGCGGGAGCAGCTGATGCCCC | |
| | ACTCCGTCTAAGATCAATACACGAATATCCTAAGTACCAGGCTGA | |
| | ATTCCCCATGAGTCCTGTGACCTCAGCCCACGCGGGGACCTACAG | |
| | GTGCTACGGCTCACTCAACTCCGACCCCTACCTGCTGTCTCACCCC | |
| | AGTGAGCCCCTGGAGCTCGTGGTCTCAGGACCCTCCATGGGTTCC | |
| | AGCCCCCCACCCACCGGTCCCATCTCCACACCTGGCCCTGAGGAC | |
| | CAGCCCCTCACCCCCACTGGGTCGGACCCCCAAAGTGGTCTGGGA | |
| | AGGCACCTGGGGGTTGTG | |
| Lilrb3_01 | GGGCCCTTCCCCAAACCCACCCTCTGGGCTGAGCCAGGCTCTGTG | 40 |
| (consensus | ATCAGCTGGGGGAGCCCCGTGACCATCTGGTGTCAGGGGAGCCA | |
| sequence) | GGAGGCCCAGGAGTACCGACTGCATAAAGAGGGAAGCCCAGAGC | |
| | CCTTGGACAGAAATAACCCACTGGAACCCAAGAACAAGGCCAGA | |
| | TTCTCCATCCCATCCATGACAGAGCACCATGCAGGGAGATACCGC | |
| | TGCCACTATTACAGCTCTGCAGGCTGGTCAGAGCCCAGCGACCCC | |
| | CTGGAGATGGTGATGACAGGAGCCTACAGCAAACCCACCCTCTC | |
| | AGCCCTGCCCAGCCCTGTGGTGGCCTCAGGGGGGAATATGACCCT | |
| | CCGATGTGGCTCACAGAAGGGATATCACCATTTTGTTCTGATGAA | |
| | GGAAGGAGAACACCAGCTCCCCCGGACCCTGGACTCACAGCAGC | |
| | TCCACAGTCGGGGGTTCCAGGCCCTGTTCCCTGTGGGCCCCGTGA | |
| | CCCCCAGCCACAGGTGGAGGTTCACATGCTATTACTATTATACAA | |
| | ACACCCCCTGGGTGTGGTCCCACCCCAGTGACCCCCTGGAGATTC | |
| | TGCCCTCAGGCGTGTCTAGGAAGCCCTCCCTCCTGACCCTGCAGG | |
| | GCCCTGTCCTGGCCCCTGGGCAGAGCCTGACCCTCCAGTGTGGCT | |
| | CTGATGTCGGCTACAACAGATTTGTTCTGTATAAGGAGGGGGAAC | |
| | GTGACTTCCTCCAGCGCCCTGGCCAGCAGCCCCAGGCTGGGCTCT | |
| | CCCAGGCCAACTTCACCCTGGGCCCTGTGAGCCCCTCCAATGGGG | |
| | GCCAGTACAGGTGCTACGGTGCACACAACCTCTCCTCCGAGTGGT | |
| | CGGCCCCCAGCGACCCCCTGAACATCCTGATGGCAGGACAGATCT | |
| | ATGACACCGTCTCCCTGTCAGCACAGCCGGGCCCCACAGTGGCCT | |
| | CAGGAGAGAACGTGACCCTGCTGTGTCAGTCATGGTGGCAGTTTG | |
| | ACACTTTCCTTCTGACCAAAGAAGGGGCAGCCCATCCCCCACTGC | |
| | GTCTGAGATCAATGTACGGAGCTCATAAGTACCAGGCTGAATTCC | |
| | CCATGAGTCCTGTGACCTCAGCCCACGCGGGGACCTACAGGTGCT | |
| | ACGGCTCATACAGCTCCAACCCCCACCTGCTGTCTCACCCCAGTG | |
| | AGCCCCTGGAGCTCGTGGTCTCAGGACACTCTGGAGGCTCCAGCC | |
| | TCCCACCCACAGGGCCGCCCTCCACACCTGGTCTGGGAAGATACC | |
| | TGGAG | |
| Lilrb3_05 | GGGCCCTTCCCCAAACCCACCCTCTGGGCTGAGCCAGGCTCTGTG | 41 |
| | ATCAGCTGGGGGAGCCCCGTGACCATCTGGTGTCAGGGGAGCCT | |
| | GGAGGCCCAGGAGTACCAACTGGATAAAGAGGGAAGCCCAGAGC | |
| | CCTGGGACAGAAATAACCCACTGGAACCCAAGAACAAGGCCAGA | |
| | TTCTCCATCCCATCCATGACACAGCACCATGCAGGGAGATACCGC | |
| | TGCCACTATTACAGCTCTGCAGGCTGGTCAGAGCCCAGCGACCCC | |
| | CTGGAGCTGGTGATGACAGGATTCTACAACAAACCCACCCTCTCA | |
| | GCCCTGCCCAGCCCTGTGGTGGCCTCAGGGGGGAATATGACCCTC | |
| | CGATGTGGCTCACAGAAGGGATATCACCATTTTGTTCTGATGAAG | |
| | GAAGGAGAACACCAGCTCCCCCGGACCCTGGACTCACAGCAGCT | |
| | CCACAGTGGGGGGTTCCAGGCCCTGTTCCCTGTGGGCCCCGTGAC | |
| | CCCCAGCCACAGGTGGAGGTTCACATGCTATTACTATTATACAAA | |
| | CACCCCCTGGGTGTGGTCCCACCCCAGTGACCCCCTGGAGATTCT | |
| | GCCCTCAGGCGTGTCTAGGAAGCCCTCCCTCCTGACCCTGCAGGG | |
| | CCCTGTCCTGGCCCCTGGGCAGAGCCTGACCCTCCAGTGTGGCTC | |
| | TGATGTCGGCTACGACAGATTTGTTCTGTATAAGGAGGGGGAACG | |
| | TGACTTCCTCCAGCGCCCTGGCCAGCAGCCCCAGGCTGGGCTCTC | |
| | CCAGGCCAACTTCACCCTGGGCCCTGTGAGCCGCTCCTACGGGGG | |
| | CCAGTACAGGTGCTACGGTGCACACAACCTCTCCTCCGAGTGGTC | |
| | GGCCCCCAGTGACCCCCTGGACATCCTGATCACAGGACAGATCTA | |
| | TGACACCGTCTCCCTGTCAGCACAGCCGGGCCCCACAGTGGCCTC | |
| | AGGAGAGAACATGACCCTGCTGTGTCAGTCACGGGGGTATTTTGA | |
| | CACTTTCCTTCTGACCAAAGAAGGGGCAGCCCATCCCCCACTGCG | |
| | TCTGAGATCAATGTACGGAGCTCATAAGTACCAGGCTGAATTCCC | |
| | CATGAGTCCTGTGACCTCAGCCCACGCGGGGACCTACAGGTGCTA | |
| | CGGCTCACGCAGCTCCAACCCCCACCTGCTGTCTTTCCCCAGTGA | |
| | GCCCCTGGAACTCATGGTCTCAGGACACTCTGGAGGCTCCAGCCT | |
| | CCCACCCACAGGGCCGCCCTCCACACCTGGTCTGGGAAGATACCT | |
| | GGAG | |
| LILRA1 | CCCCGGACCCACGTGCAGGCAGGGACCCTCCCCAAGCCCACACTC | 42 |
| | TGGGCTGAGCCAGGCTCTGTGATCACCCAGGGGAGTCCCGTGACC | |
| | CTCTGGTGTCAGGGGATTCTGGAGACCCAGGAGTACCGTCTGTAT | |
| | AGAGAAAAGAAAACAGCACCCTGGATTACACGGATTCCACAGGA | |
| | GATTGTGAAGAAGGGCCAGTTCCCCATCCCATCCATCACCTGGGA | |
| | ACACACAGGGCGGTATCGCTGTTTCTACGGTAGCCACACTGCAGG | |
| | CTGGTCAGAGCCCAGTGACCCCCTGGAGCTGGTGGTGACAGGAG | |
| | CCTACATCAAACCCACCCTCTCAGCTCTACCCAGCCCTGTGGTGA | |
| | CCTCAGGAGGGAACGTGACCCTCCATTGTGTCTCACAGGTGGCAT | |
| | TTGGCAGCTTCATTCTGTGTAAGGAAGGAGAAGATGAACACCCAC | |
| | AATGCCTGAACTCACAGCCCCGTACCCATGGGTGGTCCCGGGCCA | |
| | TCTTCTCTGTGGGCCCCGTGAGCCCGAGTCGCAGGTGGTCGTACA | |
| | GGTGCTATGCTTATGACTCGAACTCTCCCCATGTGTGGTCTCTACC | |
| | CAGTGATCTCCTGGAGCTCCTGGTCCTAGGTGTTTCTAAGAAGCC | |
| | ATCACTCTCAGTGCAGCCAGGTCCTATAGTGGCCCCTGGGGAGAG | |
| | CCTGACCCTCCAGTGTGTTTCTGATGTCAGCTACGACAGATTTGTT | |
| | CTGTATAAGGAGGGAGAACGTGACTTCCTCCAGCTCCCTGGCCCA | |
| | CAGCCCCAGGCTGGGCTCTCCCAGGCCAACTTCACCCTGGGCCCT | |
| | GTGAGCCGCTCCTACGGGGGCCAGTACAGATGCTCCGGTGCATAC | |
| | AACCTCTCCTCCGAGTGGTCGGCCCCCAGCGACCCCCTGGACATC | |
| | CTGATCGCAGGACAGTTCCGTGGCAGACCCTTCATCTCGGTGCAT | |
| | CCGGGCCCCACGGTGGCCTCAGGAGAGAACGTGACCCTGCTGTGT | |
| | CAGTCATGGGGGCCGTTCCACACTTTCCTTCTGACCAAGGCGGGA | |
| | GCAGCTGATGCCCCCCTCCGTCTCAGATCAATACACGAATATCCT | |
| | AAGTACCAGGCTGAATTCCCTATGAGTCCTGTGACCTCAGCCCAC | |
| | TCGGGGACCTACAGGTGCTACGGCTCACTCAGCTCCAACCCCTAC | |
| | CTGCTGTCTCACCCCAGTGACTCCCTGGAGCTCATGGTCTCAGGA | |
| | GCAGCTGAGACCCTCAGCCCACCACAAAACAAGTCCGATTCCAA | |
| | GGCTGGAGCAGCTAACACCCTCAGCCCATCACAAAACAAGACTG | |
| | CCTCACACCCCCAGGATTACACAGTGGAGAAT | |
| LILRA2 | GGGCACCTCCCCAAGCCCACCCTCTGGGCTGAGCCAGGCTCTGTG | 43 |
| | ATCATCCAGGGAAGTCCTGTGACCCTCAGGTGTCAGGGGAGCCTT | |
| | CAGGCTGAGGAGTACCATCTATATAGGGAAAACAAATCAGCATC | |
| | CTGGGTTAGACGGATACAAGAGCCTGGGAAGAATGGCCAGTTCC | |
| | CCATCCCATCCATCACCTGGGAACACGCAGGGCGGTATCACTGTC | |
| | AGTACTACAGCCACAATCACTCATCAGAGTACAGTGACCCCCTGG | |
| | AGCTGGTGGTGACAGGAGCCTACAGCAAACCCACCCTCTCAGCTC | |
| | TGCCCAGCCCTGTGGTGACCTCAGGAGGGAACGTGACCCTCCAGT | |
| | GTGTCTCACAGGTGGCATTTGACGGCTTCATTCTGTGTAAGGAAG | |
| | GAGAAGATGAACACCCACAACGCCTGAACTCCCATTCCCATGCCC | |
| | GTGGGTGGTCCTGGGCCATCTTCTCCGTGGGCCCCGTGAGCCCGA | |
| | GTCGCAGGTGGTCGTACAGGTGCTATGCTTATGACTCGAACTCTC | |
| | CCTATGTGTGGTCTCTACCCAGTGATCTCCTGGAGCTCCTGGTCCC | |
| | AGGTGTTTCTAAGAAGCCATCACTCTCAGTGCAGCCAGGTCCTAT | |
| | GGTGGCCCCTGGGGAGAGCCTGACCCTCCAGTGTGTCTCTGATGT | |
| | CGGCTACGACAGATTTGTTCTGTATAAGGAGGGAGAACGTGACTT | |
| | CCTCCAGCGCCCTGGTTGGCAGCCCCAGGCTGGGCTCTCCCAGGC | |
| | CAACTTCACCCTGGGCCCTGTGAGCCCCTCCCACGGGGGCCAGTA | |
| | CAGATGCTACAGTGCACACAACCTCTCCTCCGAGTGGTCGGCCCC | |
| | CAGTGACCCCCTGGACATCCTGATCACAGGACAGTTCTATGACAG | |
| | ACCCTCTCTCTCGGTGCAGCCGGTCCCCACAGTAGCCCCAGGAAA | |
| | GAACGTGACCCTGCTGTGTCAGTCACGGGGGCAGTTCCACACTTT | |
| | CCTTCTGACCAAGGAGGGGGCAGGCCATCCCCCACTGCATCTGAG | |
| | ATCAGAGCACCAAGCTCAGCAGAACCAGGCTGAATTCCGCATGG | |
| | GTCCTGTGACCTCAGCCCACGTGGGGACCTACAGATGCTACAGCT | |
| | CACTCAGCTCCAACCCCTACCTGCTGTCTCTCCCCAGTGACCCCCT | |
| | GGAGCTCGTGGTCTCAGAAGCAGCTGAGACCCTCAGCCCATCACA | |
| | AAACAAGACAGACTCCACGACTACATCCCTAGGCCAACACCCCC | |
| | AGGATTACACAGTGGAGAAT | |
| LILRA3 | GGGCCCCTCCCCAAGCCCACCCTCTGGGCTGAGCCAGGCTCTGTG | 44 |
| | ATCACCCAAGGGAGTCCTGTGACCCTCAGGTGTCAGGGGAGCCTG | |
| | GAGACGCAGGAGTACCATCTATATAGAGAAAAGAAAACAGCACT | |
| | CTGGATTACACGGATTCCACAGGAGCTTGTGAAGAAGGGCCAGTT | |
| | CCCCATCCTATCCATCACCTGGGAACATGCAGGGCGGTATTGCTG | |
| | TATCTATGGCAGCCACACTGCAGGCCTCTCAGAGAGCAGTGACCC | |
| | CCTGGAGCTGGTGGTGACAGGAGCCTACAGCAAACCCACCCTCTC | |
| | AGCTCTGCCCAGCCCTGTGGTGACCTCAGGAGGGAATGTGACCAT | |
| | CCAGTGTGACTCACAGGTGGCATTTGATGGCTTCATTCTGTGTAA | |
| | GGAAGGAGAAGATGAACACCCACAATGCCTGAACTCCCATTCCC | |
| | ATGCCCGTGGGTCATCCCGGGCCATCTTCTCCGTGGGCCCCGTGA | |
| | GCCCAAGTCGCAGGTGGTCGTACAGGTGCTATGGTTATGACTCGC | |
| | GCGCTCCCTATGTGTGGTCTCTACCCAGTGATCTCCTGGGGCTCCT | |
| | GGTCCCAGGTGTTTCTAAGAAGCCATCACTCTCAGTGCAGCCGGG | |
| | TCCTGTCGTGGCCCCTGGGGAGAAGCTGACCTTCCAGTGTGGCTC | |
| | TGATGCCGGCTACGACAGATTTGTTCTGTACAAGGAGTGGGGACG | |
| | TGACTTCCTCCAGCGCCCTGGCCGGCAGCCCCAGGCTGGGCTCTC | |
| | CCAGGCCAACTTCACCCTGGGCCCTGTGAGCCGCTCCTACGGGGG | |
| | CCAGTACACATGCTCCGGTGCATACAACCTCTCCTCCGAGTGGTC | |
| | GGCCCCCAGCGACCCCCTGGACATCCTGATCACAGGACAGATCCG | |
| | TGCCAGACCCTTCCTCTCCGTGCGGCCGGGCCCCACAGTGGCCTC | |
| | AGGAGAGAACGTGACCCTGCTGTGTCAGTCACAGGGAGGGATGC | |
| | ACACTTTCCTTTTGACCAAGGAGGGGGCAGCTGATTCCCCGCTGC | |
| | GTCTAAAATCAAAGCGCCAATCTCATAAGTACCAGGCTGAATTCC | |
| | CCATGAGTCCTGTGACCTCGGCCCACGCGGGGACCTACAGGTGCT | |
| | ACGGCTCACTCAGCTCCAACCCCTACCTGCTGACTCACCCCAGTG | |
| | ACCCCCTGGAGCTCGTGGTCTCAGGAGCAGCTGAGACCCTCAGCC | |
| | CACCACAAAACAAGTCCGACTCCAAGGCTGGTGAG | |
| LILRA4 | GAAAACCTACTCAAACCCATCCTGTGGGCCGAGCCAGGTCCCGTG | 45 |
| | ATCACCTGGCATAACCCCGTGACCATCTGGTGTCAGGGCACCCTG | |
| | GAGGCCCAGGGGTATCGTCTGGATAAAGAGGGAAACTCAATGTC | |
| | GAGGCACATATTAAAAACACTGGAGTCTGAAAACAAGGTCAAAC | |
| | TCTCCATCCCATCCATGATGTGGGAACATGCAGGGCGATATCACT | |
| | GTTACTATCAGAGCCCTGCAGGCTGGTCAGAGCCCAGCGACCCCC | |
| | TGGAGCTGGTGGTGACAGCCTACAGCAGACCCACCCTGTCCGCAC | |
| | TGCCAAGCCCTGTGGTGACCTCAGGAGTGAACGTGACCCTCCGGT | |
| | GTGCCTCACGGCTGGGACTGGGCAGGTTCACTCTGATTGAGGAAG | |
| | GAGACCACAGGCTCTCCTGGACCCTGAACTCACACCAACACAACC | |
| | ATGGAAAGTTCCAGGCCCTGTTCCCCATGGGCCCCCTGACCTTCA | |
| | GCAACAGGGGTACATTCAGATGCTACGGCTATGAAAACAACACC | |
| | CCATACGTGTGGTCGGAACCCAGTGACCCCCTGCAGCTACTGGTG | |
| | TCAGGCGTGTCTAGGAAGCCCTCCCTCCTGACCCTGCAGGGCCCT | |
| | GTCGTGACCCCCGGAGAGAATCTGACCCTCCAGTGTGGCTCTGAT | |
| | GTCGGCTACATCAGATACACTCTGTACAAGGAGGGGGCCGATGG | |
| | CCTCCCCCAGCGCCCTGGCCGGCAGCCCCAGGCTGGGCTCTCCCA | |
| | GGCCAACTTCACCCTGAGCCCTGTGAGCCGCTCCTACGGGGGCCA | |
| | GTACAGATGCTACGGCGCACACAACGTCTCCTCCGAGTGGTCGGC | |
| | CCCCAGTGACCCCCTGGATATCCTGATCGCAGGACAGATCTCTGA | |
| | CAGACCCTCCCTCTCAGTGCAGCCGGGCCCCACGGTGACCTCAGG | |
| | AGAGAAGGTGACCCTGCTGTGTCAGTCATGGGACCCGATGTTCAC | |
| | TTTCCTTCTGACCAAGGAGGGGGCAGCCCATCCCCCGTTGCGTCT | |
| | GAGATCAATGTACGGAGCTCATAAGTACCAGGCTGAATTCCCCAT | |
| | GAGTCCTGTGACCTCAGCCCACGCGGGGACCTACAGGTGCTACGG | |
| | CTCACGCAGCTCCAACCCCTACCTGCTGTCTCACCCCAGTGAGCC | |
| | CCTGGAGCTCGTGGTCTCAGGAGCAACTGAGACCCTCAATCCAGC | |
| | ACAAAAGAAGTCAGATTCCAAGACTGCCCCACACCTCCAGGATT | |
| | ACACAGTGGAGAAT | |
| LILRA5 | GGGAACCTCTCCAAAGCCACCCTCTGGGCTGAGCCAGGCTCTGTG | 46 |
| | ATCAGCCGGGGGAACTCTGTGACCATCCGGTGTCAGGGGACCCTG | |
| | GAGGCCCAGGAATACCGTCTGGTTAAAGAGGGAAGCCCAGAACC | |
| | CTGGGACACACAGAACCCACTGGAGCCCAAGAACAAGGCCAGAT | |
| | TCTCCATCCCATCCATGACAGAGCACCATGCAGGGAGATACCGCT | |
| | GTTACTACTACAGCCCTGCAGGCTGGTCAGAGCCCAGCGACCCCC | |
| | TGGAGCTGGTGGTGACAGGATTCTACAACAAACCCACCCTCTCAG | |
| | CCCTGCCCAGTCCTGTGGTGACCTCAGGAGAGAACGTGACCCTCC | |
| | AGTGTGGCTCACGGCTGAGATTCGACAGGTTCATTCTGACTGAGG | |
| | AAGGAGACCACAAGCTCTCCTGGACCTTGGACTCACAGCTGACCC | |
| | CCAGTGGGCAGTTCCAGGCCCTGTTCCCTGTGGGCCCTGTGACCC | |
| | CCAGCCACAGGTGGATGCTCAGATGCTATGGCTCTCGCAGGCATA | |
| | TCCTGCAGGTATGGTCAGAACCCAGTGACCTCCTGGAGATTCCGG | |
| | TCTCAGGAGCAGCTGATAACCTCAGTCCGTCACAAAACAAGTCTG | |
| | ACTCTGGGACTGCCTCACACCTTCAGGATTACGCAGTAGAGAATC | |
| | TCATCCGC | |
| LILRB2-d1d2-Fc | GAGCCCAAATCTAGTGACAAAACTCACACATGCCCACCGTGCCCA | 47 |
| | GCACCTGAACTCCTGGGGGGACCGTCAGTCTTCCTCTTCCCCCCA | |
| | AAACCCAAGGACACCCTCATGATCTCCCGGACCCCTGAGGTCACA | |
| | TGCGTGGTGGTGGACGTGAGCCACGAAGACCCTGAGGTCAAGTT | |
| | CAACTGGTACGTGGACGGCGTGGAGGTGCATAATGCCAAGACAA | |
| | AGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAA | |
| | AACCATCTCCAAAGCCAAAGGGCAGCCCCGAGAACCACAGGTGT | |
| | ACACCCTGCCCCCATCCCGGGAGGAGATGACCAAGAACCAGGTC | |
| | AGCCTGACCTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTT | |
| | CTCATGCTCCGTGATGCACGAGGCTCTGCACAACCACTACACGCA | |
| | GAAGAGCCTCTCCCTGTCTCCGGGTGCACGTACGGGCGGCGGCGG | |
| | CAGCGGCGGCGGCGGCAGCCAGACAGGGACCATCCCCAAGCCCA | |
| | CCCTGTGGGCTGAGCCAGACTCTGTGATCACCCAGGGGAGTCCCG | |
| | TCACCCTCAGTTGTCAGGGGAGCCTTGAAGCCCAGGAGTACCGTC | |
| | TATATAGGGAGAAAAAATCAGCATCTTGGATTACACGGATACGA | |
| | CCAGAGCTTGTGAAGAACGGCCAGTTCCACATCCCATCCATCACC | |
| | TGGGAACACACAGGGCGATATGGCTGTCAGTATTACAGCCGCGCT | |
| | CGGTGGTCTGAGCTCAGTGACCCCCTGGTGCTGGTGATGACAGGA | |
| | GCCTACCCAAAACCCACCCTCTCAGCCCAGCCCAGCCCTGTGGTG | |
| | ACCTCAGGAGGAAGGGTGACCCTCCAGTGTGAGTCACAGGTGGC | |
| | ATTTGGCGGCTTCATTCTGTGTAAGGAAGGAGAAGATGAACACCC | |
| | ACAATGCCTGAACTCCCAGCCCCATGCCCGTGGGTCGTCCCGCGC | |
| | CATCTTCTCCGTGGGCCCCGTGAGCCCGAATCGCAGGTGGTCGCA | |
| | CAGGTGCTATGGTTATGACTTGAACTCTCCCTATGTGTGGTCTTCA | |
| | CCCAGTGATCTCCTGGAGCTCCTGGTCCCAGGTGTTTCTAAGAAG | |
| | CCATCACTCTCAGTGTAG | |
| LILRB2-d3d4-Fc | GAGCCCAAATCTAGTGACAAAACTCACACATGCCCACCGTGCCCA | 48 |
| | GCACCTGAACTCCTGGGGGGACCGTCAGTCTTCCTCTTCCCCCCA | |
| | AAACCCAAGGACACCCTCATGATCTCCCGGACCCCTGAGGTCACA | |
| | TGCGTGGTGGTGGACGTGAGCCACGAAGACCCTGAGGTCAAGTT | |
| | CAACTGGTACGTGGACGGCGTGGAGGTGCATAATGCCAAGACAA | |
| | AGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAA | |
| | AACCATCTCCAAAGCCAAAGGGCAGCCCCGAGAACCACAGGTGT | |
| | ACACCCTGCCCCCATCCCGGGAGGAGATGACCAAGAACCAGGTC | |
| | AGCCTGACCTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTT | |
| | CTCATGCTCCGTGATGCACGAGGCTCTGCACAACCACTACACGCA | |
| | GAAGAGCCTCTCCCTGTCTCCGGGTGCACGTACGGGCGGCGGCGG | |
| | CAGCGGCGGCGGCGGCAGCCAGCCGGGTCCTGTCATGGCCCCTG | |
| | GGGAAAGCCTGACCCTCCAGTGTGTCTCTGATGTCGGCTATGACA | |
| | GATTTGTTCTGTACAAGGAGGGGGAACGTGACCTTCGCCAGCTCC | |
| | CTGGCCGGCAGCCCCAGGCTGGGCTCTCCCAGGCCAACTTCACCC | |
| | TGGGCCCTGTGAGCCGCTCCTACGGGGGCCAGTACAGATGCTACG | |
| | GTGCACACAACCTCTCCTCTGAGTGCTCGGCCCCCAGCGACCCCC | |
| | TGGACATCCTGATCACAGGACAGATCCGTGGCACACCCTTCATCT | |
| | CAGTGCAGCCAGGCCCCACAGTGGCCTCAGGAGAGAACGTGACC | |
| | CTGCTGTGTCAGTCATGGCGGCAGTTCCACACTTTCCTTCTGACCA | |
| | AGGCGGGAGCAGCTGATGCCCCACTCCGTCTAAGATCAATACAC | |
| | GAATATCCTAAGTACCAGGCTGAATTCCCCATGAGTCCTGTGACC | |
| | TCAGCCCACGCGGGGACCTACAGGTGCTACGGCTCACTCAACTCC | |
| | GACCCCTACCTGCTGTCTCACCCCAGTGAGCCCCTGGAGCTCGTG | |
| | GTCTCAGGACCCTCCATGGGTTCCAGCCCCCCACCCACCGGTCCC | |
| | ATCTCCACACCTGGCCCTGAGGACCAGCCCCTCACCCCCACTGGG | |
| | TCGGACCCCCAAAGTGGTCTGGGAAGGCACCTGGGGGTTGTGTA | |
| | G | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein maybe employed in practicing the disclosure. Itis intended that the following claims define the scope of the disclosure and that methods and structures within the scope of these claims and their equivalents be covered thereby.

What is claimed is:

1. An anti-LILRB antibody that specifically binds to an epitope on the extracellular domain of LILRB1, an epitope on the extracellular domain of LILRB2, an epitope on the extracellular domain of LILRB3, an epitope on the extracellular domain of LILRB4, or an epitope on the extracellular domain of LILRB5, for the treatment of a proliferative disease, an infectious disease, or a neurological disease or disorder.

2. The anti-LILRB antibody of claim 1, wherein the epitope comprises a peptide sequence within domain D1, D2, D3, or D4, or a combination thereof of a LILRB protein.

3. The anti-LILRB antibody of claim 1, wherein the epitope comprises a peptide sequence within domain D1, D2, D3, or D4, or a combination thereof of LILRB2.

4. The anti-LILRB antibody of claim 3, wherein the epitope comprises a peptide sequence within domain D1 or D2, or a combination thereof of LILRB2, wherein D1 comprises an amino acid region that corresponds to residues 22-110 of SEQ ID NO: 9 and D2 comprises an amino acid region that corresponds to residues 111-229 of SEQ ID NO: 9.

5. The anti-LILRB antibody of claim 3, wherein the epitope comprises a peptide sequence within domain D3 or D4, or a combination thereof of LILRB2, wherein D3 comprises an amino acid region that corresponds to residues 230-318 of SEQ ID NO: 9, and D4 comprises an amino acid region that corresponds to residues 319-419 of SEQ ID NO: 9.

6. The anti-LILRB antibody of claim 5, wherein if the anti-LILRB antibody specifically binds to an epitope within D3 or within D4, or to an epitope within D3 and an epitope within D4, the anti-LILRB antibody further weakly binds to an epitope within D1 or D2.

7. The anti-LILRB antibody of claim 1, wherein the anti-LILRB antibody specifically binds to a conformational epitope.

8. The anti-LILRB antibody of claim 7, wherein the conformational epitope is:
within D1, D2, D3, or D4;
within D1 or D2;
within D2 or D3; or
within D3 or D4.

9. The anti-LILRB antibody of claim 7, wherein the conformational epitope comprises:
at least one peptide sequence from D1 and at least one peptide sequence from D2; or
at least one peptide sequence from D3 and at least one peptide sequence from D4.

10. The anti-LILRB antibody of claim 1, wherein the anti-LILRB antibody is a pan antibody that specifically binds to LILRB1, LILRB2, and LILRB3.

11. The anti-LILRB antibody of claim 10, wherein the pan antibody specifically binds:
to one or more LILRB1 isoforms selected from isoforms 1-6; or
to a LILRB1 encoded by a sequence comprising at least 90%, 95%, 96%, 97%, 98%, 99%, or 100% sequence identity to SEQ ID NOs: 33-35.

12. The anti-LILRB antibody of claim 10, wherein the pan antibody specifically binds:
to one or more LILRB2 isoforms selected from isoforms 1-5; or
to a LILRB2 encoded by a sequence comprising at least 90%, 95%, 96%, 97%, 98%, 99%, or 100% sequence identity to SEQ ID NOs: 36-39.

13. The anti-LILRB antibody of claim 10, wherein the pan antibody specifically binds:
to one or more LILRB3 isoforms selected from isoforms 1-3; or
to a LILRB3 encoded by a sequence comprising at least 90%, 95%, 96%, 97%, 98%, 99%, or 100% sequence identity to SEQ ID NO: 40 or 41.

14. The anti-LILRB antibody of claim 10, wherein the pan antibody further specifically binds to:
LILRB5;
LILRA1, LILRA3, LILRA5, LILRA6, or a combination thereof,
LILRA1, LILRA3, LILRA5, and LILRA6; or
LILRA1, LILRA3, and LILRA6.

15. The anti-LILRB antibody of claim 1, wherein the anti-LILRB antibody is an anti-LILRB2 antibody that specifically binds to LILRB2 and weakly binds to an epitope on the extracellular domain of LILRB1, LILRB3, LILRB4, and LILRB5.

16. The anti-LILRB antibody of claim 15, wherein the anti-LILRB2 antibody weakly binds or does not bind to an LILRA.

17. The anti-LILRB antibody of claim 1, wherein the anti-LILRB antibody is a pan antibody that specifically binds to:
LILRB1, LILRB2, LILRB4, and LILRB5;
LILRB1, LILRB2, LILRB3, and LILRB4;
LILRB1, LILRB2, and LILRB5; or
LILRB1 and LILRB3.

18. The anti-LILRB antibody of claim 1, wherein the anti-LILRB antibody blocks HLA-G binding to a cell expressing a LILRB receptor, blocks HLA-A binding to the cell expressing a LILRB receptor, or a combination thereof.

19. The anti-LILRB antibody of claim 1, wherein the anti-LILRB antibody enhances HLA-G binding to a cell expressing a LILRB receptor.

20. The anti-LILRB antibody of claim 1, wherein the anti-LILRB antibody does not modulate HLA-G binding or HLA-A binding to a cell expressing a LILRB receptor.

21. The anti-LILRB antibody of claim 1, wherein the anti-LILRB antibody comprises a full-length antibody or a binding fragment thereof, optionally comprising a humanized antibody or binding fragment thereof, chimeric antibody or binding fragment thereof, monoclonal antibody or binding fragment thereof, bispecific antibody or binding fragment thereof, monovalent Fab′, divalent Fab2, single-chain variable fragment (scFv), diabody, minibody, nanobody, single-domain antibody (sdAb), or camelid antibody or binding fragment thereof.

22. The anti-LILRB antibody of claim 1, wherein the proliferative disease is cancer.

23. The antibody of claim 22, wherein the cancer is a solid tumor or a hematologic malignancy.

24. The antibody of claim 1, wherein the infectious disease is a viral infection.

25. The antibody of claim 24, wherein the infectious disease is Dengue fever or AIDS.

26. The antibody of claim 1, wherein the infectious disease is caused by a protozoan.

27. The antibody of claim 26, wherein the infectious disease is malaria.

28. The antibody of claim 1, wherein the neurological disease or disorder is a neurodegenerative disease or disorder.

29. The anti-LILRB antibody of claim 28, wherein the neurological disease or disorder is Alzheimer's disease.

30. The anti-LILRB antibody of claim 1, wherein the anti-LILRB antibody inhibits binding of a ligand of LILRB to LILRB by at least 10%, 20%, 30%, 40%, 50%, 60%, 70%, 80%, 90%, 95%, or more.

31. The anti-LILRB antibody of claim 1, wherein the anti-LILRB antibody inhibits binding of a ligand of LILRB to LILRB by about 2-fold, 3-fold, 4-fold, 5-fold, 6-fold, 7-fold, 8-fold, 9-fold, 10-fold, or more.

32. The anti-LILRB antibody of claim 30 or 31, wherein the ligand of LILRB is a natural ligand.

33. The anti-LILRB antibody of claim 32, wherein the natural ligand comprises:
HLA-A, HLA-B, HLA-C, HLA-E, HLA-G, CD1c, CD1d, MAG, ANGPTL1, ANGPTL2, ANGPTL3, ANGPTL4, ANGPTL5, ANGPTL6, ANGPTL7, ANGPTL8, RTN4, or OMgp; or
HLA-A;
oligo Aβ oligomers; or
a pathogen, optionally selected from Dengue, Escherichia coli, or Staphylococcus aureus.

34. The anti-LILRB antibody of claim 1, wherein the anti-LILRB antibody is 5G11.G8, 5G11.H6, 9C9.D3, 9C9.E6, 16D11.D10, 6G6.H7, 6G6.H2, 6H9.A3, 2B3.A10, 4D11.B10, or 11D9.E7.

35. The anti-LILRB antibody of claim 1, wherein the anti-LILRB antibody, when contacted to a plurality of peripheral blood mononuclear cells (PBMCs) comprising T cells, enhances cytotoxic T cell activation relative to a plurality of equivalent PBMCs and equivalent T cells in the absence of the anti-LILRB antibody.

36. The anti-LILRB antibody of claim 1, wherein the anti-LILRB antibody, when contacted to a plurality of peripheral blood mononuclear cells (PBMCs) comprising a macrophage, increases M1 activation of the macrophage relative to a plurality of equivalent PBMCs and an equivalent macrophage in the absence of the anti-LILRB antibody.

37. The anti-LILRB antibody of claim 1, wherein the anti-LILRB antibody, when contacted to a plurality of cells, increases inflammatory cytokine production relative to a plurality of equivalent cells in the absence of the anti-LILRB antibody.

38. The anti-LILRB antibody of claim 37, wherein the inflammatory cytokine comprises TNFα, IFNγ, or a combination thereof.

39. The anti-LILRB antibody of claim 1, wherein the anti-LILRB antibody, when contacted to a plurality of cells comprising PBMCs and tumor cells, decreases tumor cell proliferation relative to a plurality of equivalent cells comprising PBMCs and tumor cells in the absence of the anti-LILRB antibody.

40. The anti-LILRB antibody of claim 1, wherein the anti-LILRB antibody, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the anti-LILRB antibody.

41. The anti-LILRB antibody of claim 1, wherein the anti-LILRB antibody decreases regulatory T cells when administered to a subject in need thereof, relative to a second subject in the absence of the antibody or binding fragment thereof.

42. A pan anti-LILRB antibody that specifically binds to at least one epitope on the extracellular domain of LILRB1, at least one epitope on the extracellular domain of LILRB2, or at least one epitope on the extracellular domain of LILRB3, for the treatment of a proliferative disease, an infectious disease, or a neurological disease or disorder.

43. The pan anti-LILRB antibody of claim 42, wherein the pan anti-LILRB antibody further specifically binds to an epitope on the extracellular domain of LILRB4 or an epitope on the extracellular domain of LILRB5.

44. The pan anti-LILRB antibody of claim 42 or 43, wherein the pan anti-LILRB antibody further specifically binds to:
LILRA1, LILRA3, LILRA5, LILRA6, or a combination thereof,
LILRA1, LILRA3, LILRA5, and LILRA6; or
LILRA1, LILRA3, and LILRA6.

45. The pan anti-LILRB antibody of any one of the claims 42-44, wherein the at least one epitope on the extracellular domain of LILRB2 comprises a peptide sequence within D3, a peptide sequence within D4, or a combination thereof.

46. The pan anti-LILRB antibody of any one of the claims 42-44, wherein the at least one epitope on the extracellular domain of LILRB2 comprises a peptide sequence within D1, a peptide sequence within D2, or a combination thereof.

47. The pan anti-LILRB antibody of any one of the claims 42-45, wherein the at least one epitope on the extracellular domain of LILRB2 comprises a conformational epitope.

48. The pan anti-LILRB antibody of claim 47, wherein the conformational epitope:
is within D3 and comprises at least one peptide sequence;
is within D4 and comprises at least one peptide sequence;
comprises at least one peptide sequence from D1 and at least one peptide sequence from D2; or
comprises at least one peptide sequence from D3 and at least one peptide sequence from D4.

49. The pan anti-LILRB antibody of any one of the claims 42-48, wherein the pan anti-LILRB antibody blocks HLA-G binding to a cell expressing a LILRB receptor.

50. The pan anti-LILRB antibody of any one of the claims 42-49, wherein the pan anti-LILRB antibody comprises a full-length antibody or a binding fragment thereof, optionally comprising a humanized antibody or binding fragment thereof, chimeric antibody or binding fragment thereof, monoclonal antibody or binding fragment thereof, bispecific antibody or binding fragment thereof, monovalent Fab′, divalent Fab2, single-chain variable fragment (scFv), diabody, minibody, nanobody, single-domain antibody (sdAb), or camelid antibody or binding fragment thereof.

51. The pan anti-LILRB antibody of any one of the claims 42-50, wherein the pan anti-LILRB antibody inhibits binding of a ligand of LILRB1 to LILRB1 and/or a ligand of LILRB2 to LILRB2 by at least 10%, 20%, 30%, 40%, 50%, 60%, 70%, 80%, 90%, 95%, or more.

52. The pan anti-LILRB antibody of any one of the claims 42-50, wherein the pan anti-LILRB antibody inhibits binding of a ligand of LILRB1 to LILRB1 and/or a ligand of LILRB2 to LILRB2 by about 2-fold, 3-fold, 4-fold, 5-fold, 6-fold, 7-fold, 8-fold, 9-fold, 10-fold, or more.

53. The pan anti-LILRB antibody of claim 51 or 52, wherein the ligand of LILRB1 and the ligand of LILRB2 are each independently a natural ligand.

54. The pan anti-LILRB antibody of claim 53, wherein the natural ligand comprises:
HLA-A, HLA-B, HLA-C, HLA-E, HLA-G, CD1c, CD1d, MAG, ANGPTL1, ANGPTL2, ANGPTL3, ANGPTL4, ANGPTL5, ANGPTL6, ANGPTL7, ANGPTL8, RTN4, or OMgp;
HLA-A;
oligo Aβ oligomers; or
a pathogen, optionally selected from Dengue, Escherichia coli, or Staphylococcus aureus.

55. The pan anti-LILRB antibody of any one of the claims 42-54, wherein the pan anti-LILRB antibody is 5G11.G8, 5G11.H6, 9C9.D3, 9C9.E6, 16D11.D10, or 11D9.E7.

56. The pan anti-LILRB antibody of any one of the claims 42-55, wherein the pan anti-LILRB antibody, when contacted to a plurality of peripheral blood mononuclear cells (PBMCs) comprising a macrophage, increases M1 activation of the macrophage relative to a plurality of equivalent PBMCs and an equivalent macrophage in the absence of the pan anti-LILRB antibody.

57. The pan anti-LILRB antibody of any one of the claims 42-56, wherein the pan anti-LILRB antibody, when contacted to a plurality of cells, increases inflammatory cytokine production relative to a plurality of equivalent cells in the absence of the pan anti-LILRB antibody.

58. The pan anti-LILRB antibody of claim 57, wherein the inflammatory cytokine comprises TNFα, IFNγ, or a combination thereof.

59. The pan anti-LILRB antibody of any one of the claims 42-58, wherein the pan anti-LILRB antibody, when contacted to a plurality of cells comprising PBMCs and tumor cells, decreases tumor cell proliferation relative to a plurality of equivalent cells comprising PBMCs and tumor cells in the absence of the pan anti-LILRB antibody.

60. The pan anti-LILRB antibody of any one of the claims 42-59, wherein the pan anti-LILRB antibody, when contacted to a plurality of cells comprising myeloid-derived suppressor cells (MDSCs) and T cells, decreases MDSC suppression of cytotoxic T cell proliferation relative to a plurality of equivalent cells comprising MDSCs and T cells in the absence of the pan anti-LILRB antibody.

61. A pharmaceutical composition, comprising:
an anti-LILRB antibody of claims 1-41 or a pan anti-LILRB antibody of claims 42-60; and
a pharmaceutically acceptable excipient.

62. The pharmaceutical composition of claim 61, wherein the pharmaceutical composition is formulated for systemic administration.

63. The pharmaceutical composition of claim 61 or 62, wherein the pharmaceutical composition is formulated for parenteral administration.

64. A method of modulating a macrophage to undergo M1 activation, comprising:
a) contacting a plurality of antigen presenting cells (APCs) comprising a macrophage with an anti-LILRB antibody of claims 1-41 or a pan anti-LILRB antibody of claims 42-60;
b) binding the antibody or binding fragment thereof or the pan antibody or binding fragment thereof to one or more LILRB receptors expressed on at least one APC within the plurality of APCs, thereby inducing the APC to produce a plurality of TNFα and interferons; and
c) contacting the plurality of TNFα and interferons with the plurality of APCs comprising the macrophage to induce M1 activation of the macrophage.

65. The method of claim 64, wherein the interferon is IFNγ or IFNβ.

66. The method of claim 64, wherein the anti-LILRB antibody or the pan anti-LILRB antibody decreases M2 activation of the macrophage.

67. The method of claim 64, wherein the anti-LILRB antibody or the pan anti-LILRB antibody decreases formation of a tumor associate macrophage.

68. The method of claim 64, wherein the APCs further comprise dendritic cells, B cells, or a combination thereof.

69. A method of inducing phagocytosis of a target cell, comprising:
a) incubating a plurality of antigen presenting cells (APCs) comprising a macrophage with an anti-LILRB antibody of claims 1-41 or a pan anti-LILRB antibody of claims 42-60, thereby inducing the macrophage to undergo M1 polarization; and
b) contacting the M1 macrophage to a target cell for a time sufficient to induce phagocytosis of the target cell.

70. The method of claim 69, wherein the APCs further comprise dendritic cells, B cells, or a combination thereof.

71. The method of claim 69, wherein the target cell is a cancer cell.

72. The method of claim 69, wherein the target cell is a cell infected by a pathogen.

73. A method of activating a cytotoxic T cell, comprising
a) incubating a plurality of peripheral blood mononuclear cells (PBMCs) comprising naive T cells with an anti-LILRB antibody of claims 1-41 or a pan anti-LILRB antibody of claims 42-60, thereby stimulating the secretion of a plurality of inflammatory cytokines; and
b) contacting the plurality of inflammatory cytokines with the naïve T cells to activate a cytotoxic T cell.

74. The method of claim 73, wherein the plurality of inflammatory cytokines comprises TNFα, IFNγ, or IFNβ.

75. The method of claim 73, wherein the naïve T cells comprise naïve CD8⁺ T cells.

76. The method of claim 73, wherein the PBMCs comprise antigen presenting cells (APCs), NK cells, and/or CD4 T cells.

77. The method of claim 76, wherein the CD4 T cells comprise activated CD4⁺ helper T cells.

78. The method of claim 76, wherein the APCs comprise B cells and/or dendritic cells.

79. A kit comprising an anti-LILRB antibody of claims 1-41, a pan anti-LILRB antibody of claims 42-60, or a pharmaceutical composition of claim 61-63.
